(19) United States
(12) Patent Application Publication
(10) Pub. No.: US 2023/0026611 A1
(43) Pub. Date: Jan. 26, 2023

(54) ARYL HETEROCYCLIC COMPOUNDS AS Kv1.3 POTASSIUM SHAKER CHANNEL BLOCKERS

(71) Applicant: D. E. Shaw Research, LLC, New York, NY (US)

(72) Inventors: Fabrizio GIORDANETTO, New York, NY (US); Morten Østergaard JENSEN, Copenhagen (DK); Vishwanath JOGINI, Hyderabad (IN); Roger John SNOW, Danbury, CT (US)

(21) Appl. No.: 17/766,832

(22) PCT Filed: Oct. 6, 2020
(86) PCT No.: PCT/US2020/054347

§ 371 Date: Apr. 6, 2022

Related U.S. Application Data
(60) Provisional application No. 62/911,670, filed on Oct. 7, 2019.

(51) Int. Cl. C07D 211/34; C07D 211/22; C07D 295/096; C07D 211/56; C07D 205/04; C07D 243/08; C07D 211/52; C07D 451/02; C07D 207/08; C07D 223/04; C07D 211/28; C07D 211/26; C07D 211/32; C07D 241/04; C07D 211/60; C07D 211/70; C07D 211/58; C07D 211/42; C07D 401/04; C07D 401/06; C07D 471/10; C07D 401/12; C07D 413/04; C07D 405/12; C07D 403/12

(52) U.S. Cl. CPC C07D 211/34; C07D 211/22; C07D 295/096; C07D 211/56; C07D 205/04; C07D 243/08; C07D 211/52; C07D 451/02; C07D 207/08; C07D 223/04; C07D 211/28; C07D 211/26; C07D 211/32; C07D 241/04; C07D 211/60; C07D 211/70; C07D 211/58; C07D 211/42; C07D 401/04; C07D 401/06; C07D 471/10; C07D 401/12; C07D 413/04; C07D 405/12; C07D 403/12

(57) ABSTRACT

A compound of Formula (I), or a pharmaceutically-acceptable salt thereof, is described, wherein the substituents are as defined herein. Pharmaceutical compositions comprising the same and method of using the same are also described.

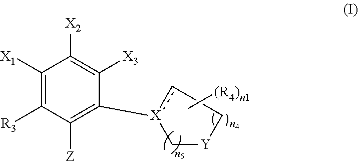

This application claims the benefit and priority of U.S. Provisional Application No. 62/911,670, filed Oct. 7, 2019, the entire contents of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues, and regulate a large number of physiological processes such as neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (interferon-gamma, interleukin 2), and cell proliferation.

Autoimmune disease is a family of disorders resulting from tissue damage caused by attack from the body's own immune system. Such diseases may affect a single organ, as in multiple sclerosis and type I diabetes mellitus, or may involve multiple organs, as in the case of rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of effector memory T cells, known to be involved in the etiology of autoimmune diseases. These inhibitors are thought to be able to ameliorate autoimmune diseases symptoms without compromising the protective immune response. Effector memory T cells (“TEMs”) express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple sclerosis (“MS”) is caused by autoimmune damage to the central nervous system (“CNS”). Symptoms include muscle weakness and paralysis, which severely affect quality of life for patients. MS progresses rapidly and unpredictably and eventually leads to death. The Kv1.3 channel is also highly expressed in auto-reactive TEMs from MS patients (Wulff H., et al., 2003, J. Clin. Invest., 1703-1713; Rus H., et al., 2005, PNAS, 11094-11099). Animal models of MS have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type 2 diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration, is involved in the pathology of chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (“IBDs”) such as ulcerative colitis (“UC”) and Crohn's disease. UC is a chronic IBD characterized by excessive T cell infiltration and cytokine production. UC can impair quality of life and can lead to life-threatening complications. High levels of Kv1.3 channels in CD4 and CD8 positive T cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC. Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in MS brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, Kv1.3 channel may be a therapeutic target for Alzheimer's disease.

Kv1.3 channel blockers could be also useful for ameliorating pathology in cardiovascular disorders such as ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (“shk”) with an unnatural amino acid (shk-186) is the most advanced peptide toxin. Shk has demonstrated efficacy in preclinical models and is currently in a phase I clinical trial for treatment of psoriasis. Shk can suppress proliferation of TEMs, resulting in improved condition in animal models of multiple sclerosis. Unfortunately, Shk also binds to the closely-related Kvi channel subtype found in CNS and heart. There is a need for Kv1.3 channel-selective inhibitors to avoid potential cardio- and neuro-toxicity. Additionally, small peptides like shk-186 are rapidly cleared from the body after administration, resulting in short circulating half-lives and frequent administration events. Thus, there is a need for the development of long-acting, selective Kv1.3 channel inhibitors for the treatment of chronic inflammatory diseases.

Thus, there remains a need for development of novel Kv1.3 channel blockers as pharmaceutical agents.

### SUMMARY OF THE INVENTION

In one aspect, compounds useful as potassium channel blockers having a structure of Formula I

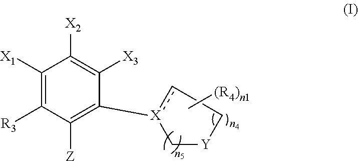

are described, where the various substituents are defined herein. The compounds of Formula I described herein can block Kv1.3 potassium (K⁺) channels and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a CNS disorder, an inflammatory disorder, a gastroenterological disorder, a metabolic disorder, a cardiovascular disorder, a kidney disease, or a combination thereof.

In one aspect, a compound of Formula I or a pharmaceutically-acceptable salt thereof is described,

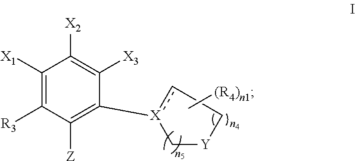

where

[symbol] refers to a single or double bond;

X is C, N, or CR₄ where valence permits;

Y is C(R₄)₂, NR₅, or O; where at least one of X and Y is N optionally substituted by R₅ where valence permits; where Y and either of its adjacent ring atoms are not linked together to form a fused ring system;

Z is ORₐ;

X₁ is H, halogen, CN, alkyl, cycloalkyl, halogenated cycloalkyl, or halogenated alkyl;

X₂ is H, halogen, CN, alkyl, cycloalkyl, halogenated cycloalkyl, or halogenated alkyl;

X₃ is H, halogen, CN, alkyl, cycloalkyl, halogenated cycloalkyl, or halogenated alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₃ is independently H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, optionally substituted saturated heterocycle, optionally substituted aryl, optionally substituted heteroaryl, CN, CF₃, ORₐ, (CR₆R₇)ₙ₃ORₐ, oxo, (C═O)R_{b}, (C═O)OR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, or (C═O)NRₐ(CR₆R₇)ₙ₃OR_{b}, (CR₆R₇)ₙ₃NRₓR_{b}, or (CR₆R₇)ₙ₃(C═O)NRₓR_{b}; wherein Rₓ is Rₐ, (C═O)Rₐ, (C═O)NRₐR_{b}, or SO₂Rₐ;
or two R₄ groups taken together with the carbon atom(s) that they are connected to form a 3-7 membered optionally substituted carbocycle or heterocycle;
each occurrence of R₅ is independently H, alkyl, cycloalkyl, optionally substituted saturated heterocycle, optionally substituted aryl, optionally substituted heteroaryl, Rₐ, NRₐR_{b}, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ; each occurrence of R₆ and R₇ are independently H, alkyl, cycloalkyl, optionally substituted aryl, or optionally substituted heteroaryl;
each occurrence of Rₐ and R_{b} are independently H, alkyl, alkenyl, cycloalkyl, optionally substituted saturated heterocycle comprising 1-3 heteroatoms each selected from the group consisting of N, O, and S, optionally substituted aryl, or optionally substituted heteroaryl; or alternatively Rₐ and R_{b} together with the nitrogen atom that they are connected to form an optionally substituted heterocycle including the nitrogen atom and 0-3 additional heteroatoms each selected from the group consisting of N, O, and S;
the alkyl, cycloalkyl, heterocycle, aryl, and heteroaryl in X₁, X₂, X₃, R₃, R₄, R₅, R₆, R₇, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits;
each occurrence of R₈ is independently H, alkyl, or optionally substituted heterocycle; or alternatively the two R₈ groups together with the nitrogen atom that they are connected to form an optionally substituted heterocycle including the nitrogen atom and 0-3 additional heteroatoms each selected from the group consisting of N, O, and S;
each occurrence of n₁ is independently an integer from 0-3 where valence permits; each occurrence of n₃ is independently an integer from 0-3; and each occurrence of n₄ and n₅ is independently 0, 1 or 2.

In any one of the embodiments described herein, [symbol] is a single bond.

In any one of the embodiments described herein, [symbol] is a double bond.

In any one of the embodiments described herein, the structural moiety

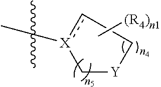

has the structure of

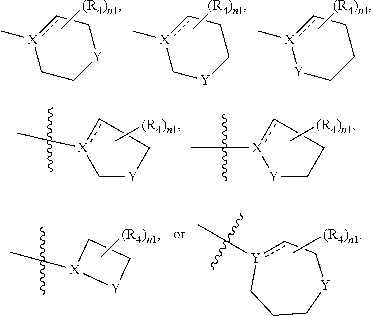

In any one of the embodiments described herein, X is N and Y is C(R₄)₂.

In any one of the embodiments described herein, X is CR₄ and Y is NR₅.

In any one of the embodiments described herein, X is CR₄ and Y is O.

In any one of the embodiments described herein, X is N and Y is NR₅.

In any one of the embodiments described herein, the structural moiety

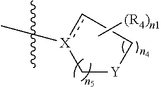

has the structure of

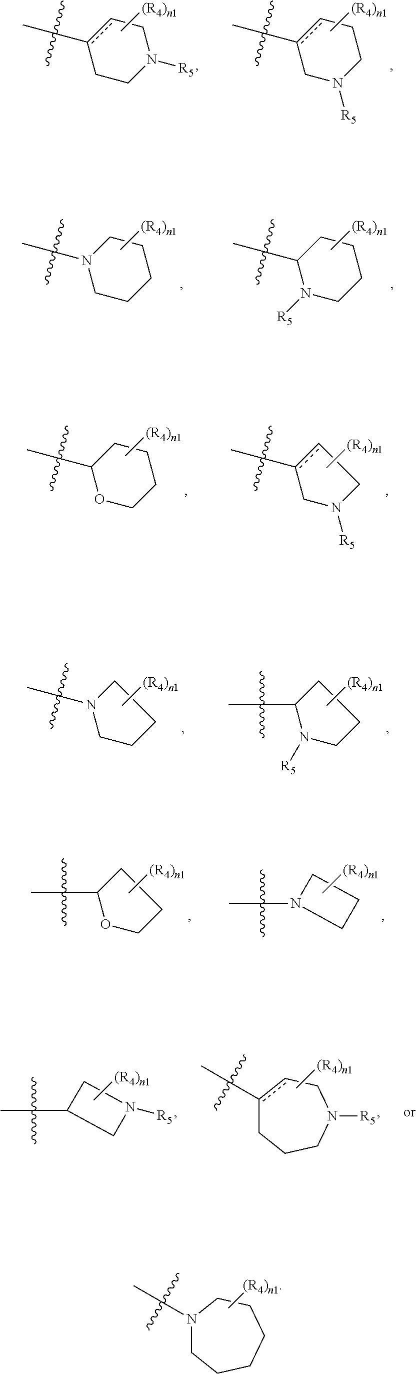

In any one of the embodiments described herein, the structural moiety

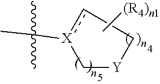

has the structure of

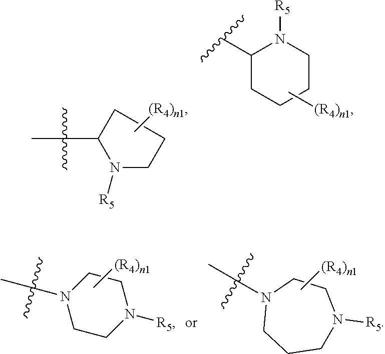

In any one of the embodiments described herein, n₁ is 0 and R₅ is H or alkyl.

In any one of the embodiments described herein, n₁ is 1 and R₅ is H or alkyl.

In any one of the embodiments described herein, R₅ is H.

In any one of the embodiments described herein, at least one occurrence of R₄ is H, CN, alkyl, cycloalkyl, aryl, heteroaryl, CF₃, or ORₐ.

In any one of the embodiments described herein, at least one occurrence of R₄ is (CR₆R₇)ₙ₃ORₐ, oxo, (C═O)R_{b}, (C═O)OR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, or a N-containing heterocycle.

In any one of the embodiments described herein, one or more occurrences of R₄ are H or alkyl.

In any one of the embodiments described herein, one or more occurrences of R₄ are (CR₆R₇)ₙ₃ORₐ or (CR₆R₇)ₙ₃NRₐR_{b}.

In any one of the embodiments described herein, one or more occurrences of R₄ are ORₐ, NRₐR_{b}, —CH₂ORₐ, —CH₂NRₐR_{b}, —CH₂CH₂ORₐ, or —CH₂CH₂NRₐR_{b}.

In any one of the embodiments described herein, at least one occurrence of R₄ is (CR₆R₇)ₙ₃(C═O)NRₐR_{b} or (C═O)NRₐ(CR₆R₇)ₙ₃OR_{b}.

In any one of the embodiments described herein, at least one or more occurrences of R₄ is (C═O)NRₐR_{b} or —CH₂(C═O)NRₐR_{b}.

In any one of the embodiments described herein, R₄ is H, Me, Et, Pr, Bu, or a saturated heterocycle or heteroaryl selected from the group consisting of

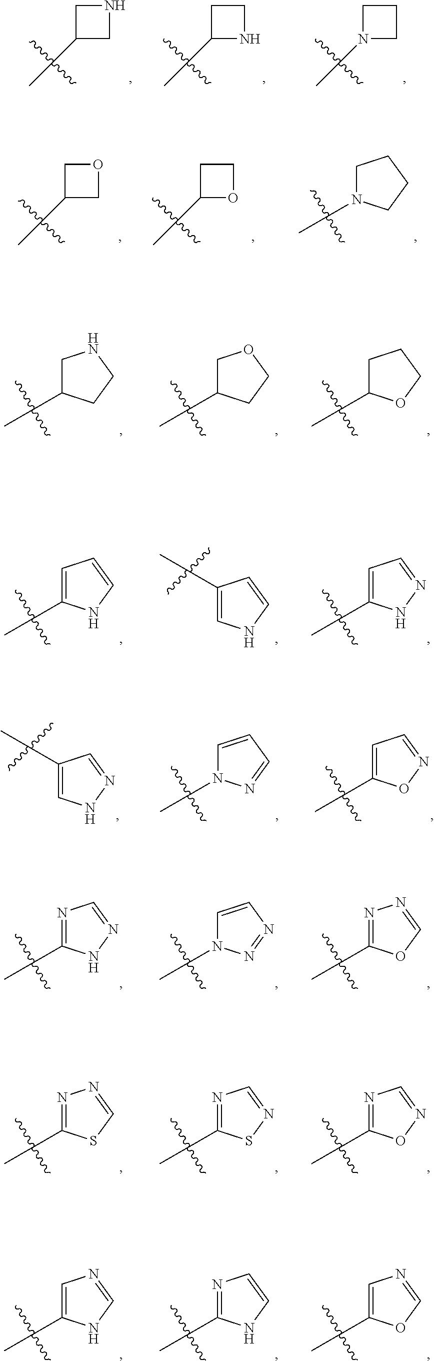

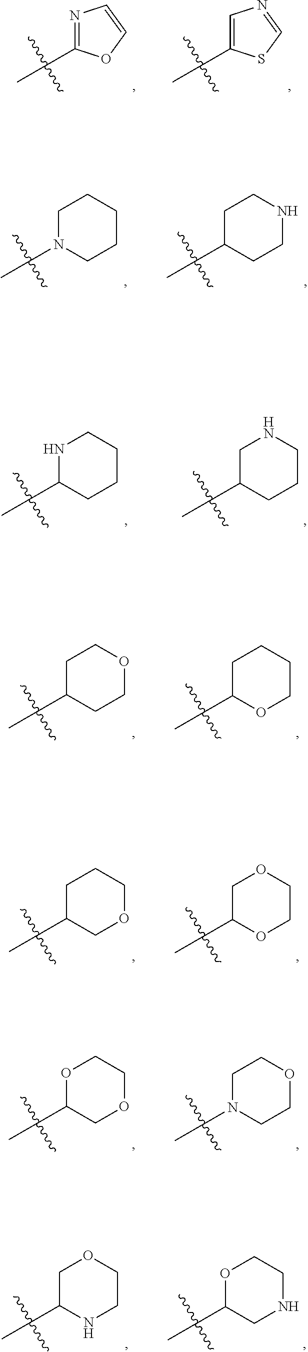

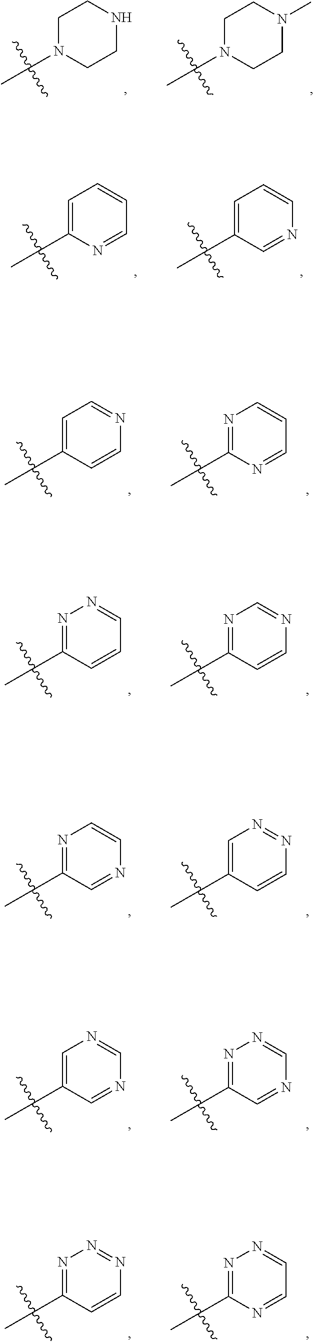

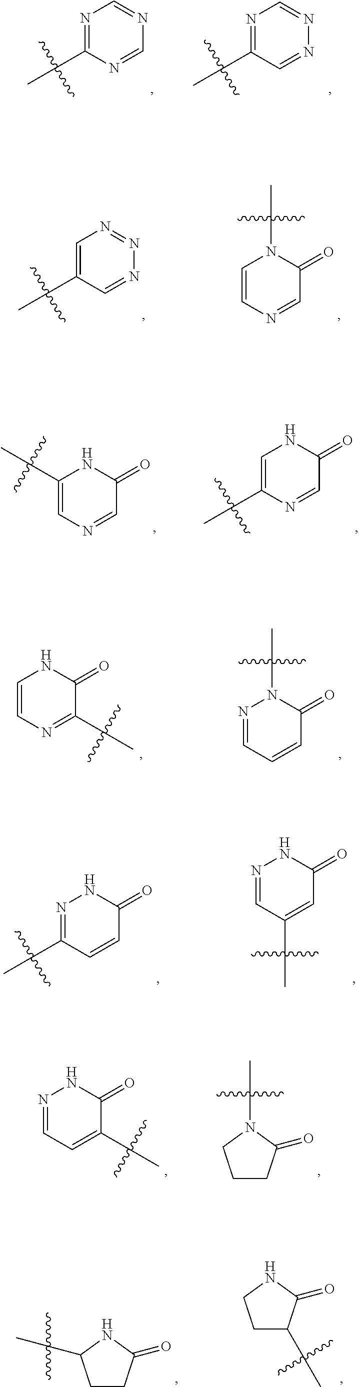

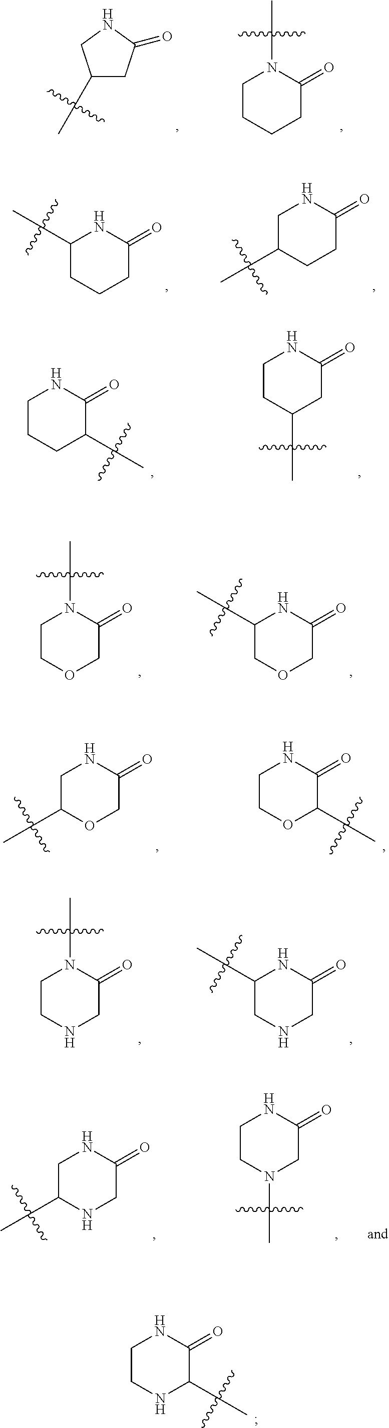

wherein the saturated heterocycle or heteroaryl is optionally substituted by cyano, cycloalkyl, fluorinated alkyl, fluorinated cycloalkyl, halogen, OH, NH₂, oxo, or (C═O)C₁₋₄alkyl where valence permits.

In any one of the embodiments described herein, R₄ is

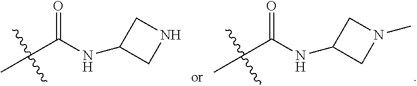

In any one of the embodiments described herein, R₄ is

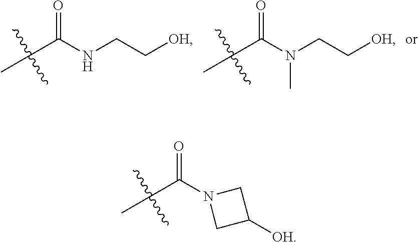

In any one of the embodiments described herein, each occurrence of R₆ and R₇ are independently H or alkyl.

In any one of the embodiments described herein, at least one occurrence of R₅ is H, alkyl, cycloalkyl, aryl, heteroaryl, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ.

In any one of the embodiments described herein, at least one occurrence of R₅ is H, alkyl, or cycloalkyl.

In any one of the embodiments described herein, at least one occurrence of R₅ is (C═O)Rₐ, (C═O)-alkyl-ORₐ, (C═O)-alkyl-NRₐR_{b}, (C═O)NRₐR_{b}, or SO₂Rₐ.

In any one of the embodiments described herein, at least one occurrence of R₅ is (C═O)NRₐR_{b}, (C═O)CH₂NRₐR_{b}, or (C═O)CH₂CH₂NRₐR_{b}.

In any one of the embodiments described herein, the compound has a structure of Formula Ia:

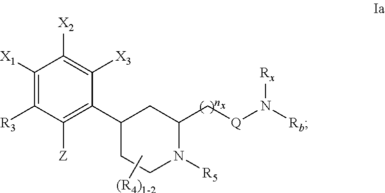

where
nₓ is 0, 1, or 2;
Q is CR₆R₇ or C═O; and
Rₓ is Rₐ, (C═O)Rₐ, (C═O)NRₐR_{b}, or SO₂Rₐ.

In any one of the embodiments described herein, nₓ is 0 or 1.

In any one of the embodiments described herein, R₅ is H or Me.

In any one of the embodiments described herein, Q is C═O and NRₓR_{b} is NH₂, NHMe, NMe₂, NH(C═O)NH₂, NMe(C═O)NH₂, NH(C═O)NHMe, NMe(C═O)NMe, NH(C═O)NMe₂, NMe(C═O)NMe₂, or SO₂Me.

In any one of the embodiments described herein,

[symbol] refers to a single bond;

X is CR₄;

Y is O or NR₅;

R₃ is H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ;

R₄ is H, alkyl, or (C═O)NRₐR_{b};

R₅ is H or alkyl;

n₁ is 1, 2, or 3;

n₄ is 0, 1 or 2; and

n₅ is 0 or 1.

In any one of the embodiments described herein, R₄ is (C═O)NRₐR_{b}.

In any one of the embodiments described herein, the compound has the structure of Formula 1b:

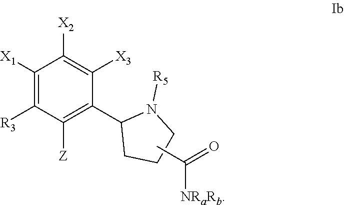

In any one of the embodiments described herein, the compound has the structure of

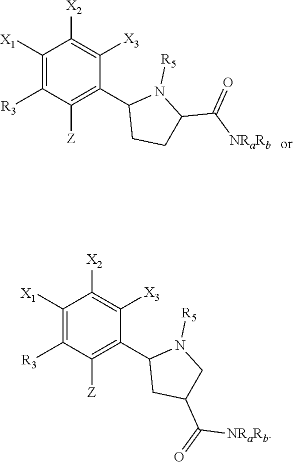

In any one of the embodiments described herein, the compound has the structure of Formula 1c:

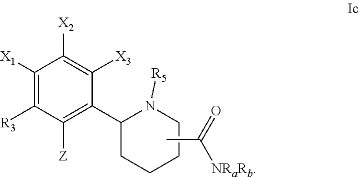

In any one of the embodiments described herein, the compound has the structure of

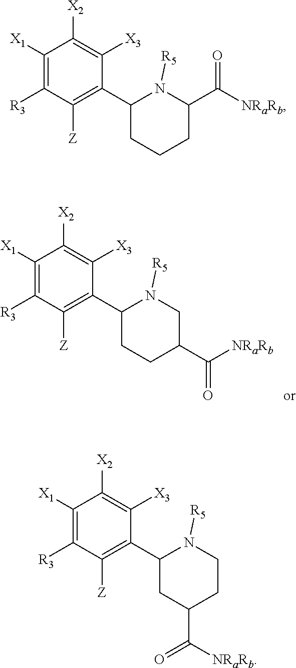

In any one of the embodiments described herein, Z is OH or O(C₁-C₄ alkyl).

In any one of the embodiments described herein, Z is OH.

In any one of the embodiments described herein, X₁ is H, halogen, fluorinated alkyl, or alkyl.

In any one of the embodiments described herein, X₁ is H, F, Cl, Br, Me, CF₂H, CF₂Cl, or CF₃.

In any one of the embodiments described herein, X₁ is H or Cl.

In any one of the embodiments described herein, X₂ is H, halogen, fluorinated alkyl, or alkyl.

In any one of the embodiments described herein, X₂ is H, F, Cl, Br, Me, CF₂H, CF₂Cl, or CF₃.

In any one of the embodiments described herein, X₂ is H or Cl.

In any one of the embodiments described herein, X₃ is H, F, Cl, Br, Me, CF₂H, CF₂Cl, or CF₃.

In any one of the embodiments described herein, X₃ is H or Cl.

In any one of the embodiments described herein, the structural moiety

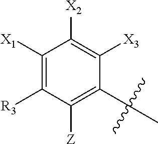

has the structure of

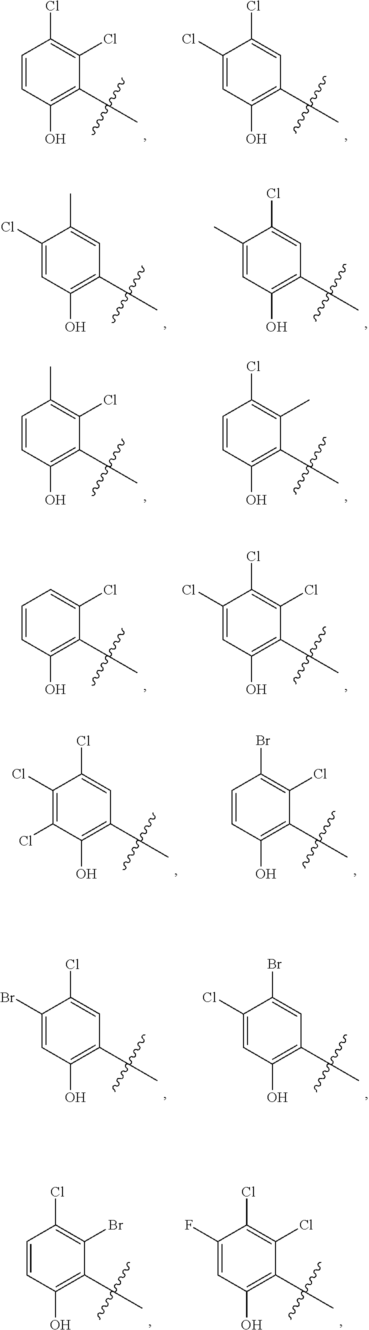

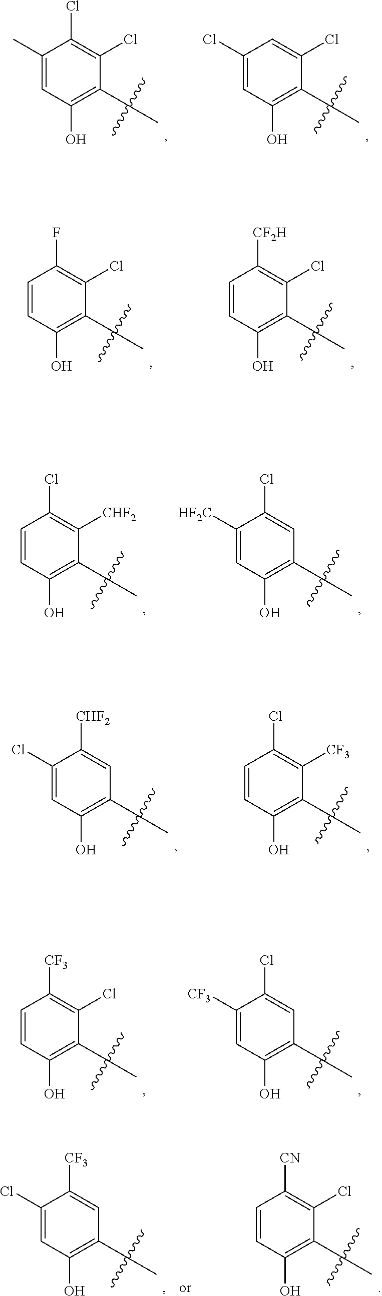

In any one of the embodiments described herein, the compound has a structure of Formula II′ or II:

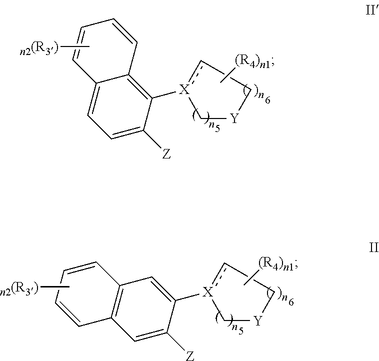

where R_{3′} is independently H, halogen, or alkyl; and

n₂ is an integer from 0-3.

In any one of the embodiments described herein, n₂ is 0, 1, 2, or 3.

In any one of the embodiments described herein, R_{3′} is H or alkyl.

In any one of the embodiments described herein, R_{3′} is halogen.

In any one of the embodiments described herein, Z is ORₐ.

In any one of the embodiments described herein, Z is OH, OMe, or OEt.

In any one of the embodiments described herein, Z is OH.

In any one of the embodiments described herein, R₃ is H, alkyl, cycloalkyl, aryl, heteroaryl, CN, CF₃, ORₐ, SRₐ, halogen, NRₐR_{b}, or NR_{b}(C═O)Rₐ.

In any one of the embodiments described herein, R₃ is H, alkyl, CF₃, ORₐ, SRₐ, halogen, NRₐR_{b}, or NR_{b}(C═O)Rₐ.

In any one of the embodiments described herein, R₃ is H, halogen, fluorinated alkyl, or alkyl.

In any one of the embodiments described herein, n₁ is 0, 1, or 2.

In any one of the embodiments described herein, each occurrence of n₃ is independently 0, 1, or 2.

In any one of the embodiments described herein, each occurrence of n₄ and n₅ are independently 0 or 1.

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b}

is independently H, Me, Et, Pr, or a heterocycle selected from the group consisting of

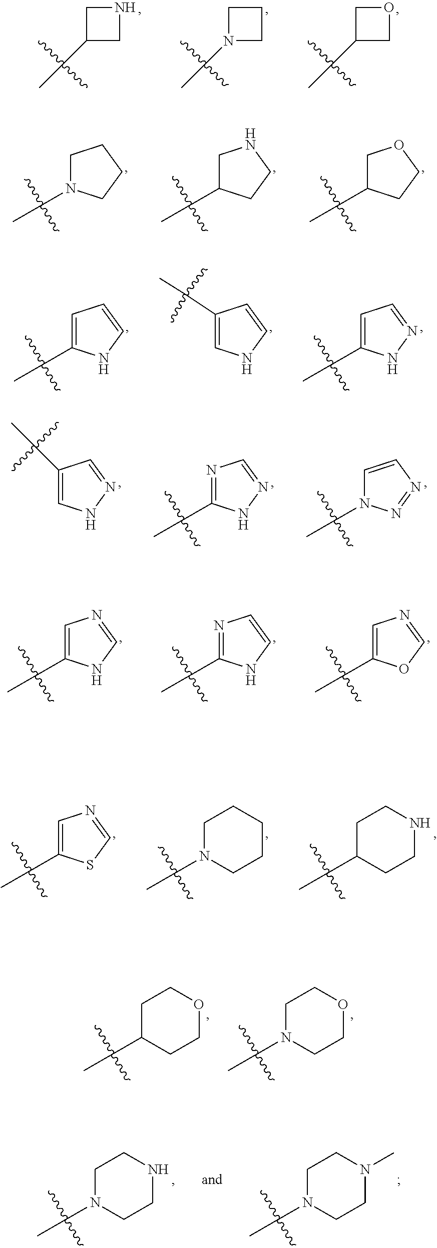

wherein the heterocycle is optionally substituted by alkyl, OH, oxo, or (C═O)C₁₋₄alkyl where valence permits.

In any one of the embodiments described herein, at least one occurrence of Rₐ or R_{b} is H or

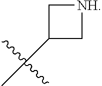 [SMILES: *C1CNC1]

In any one of the embodiments described herein, Rₐ and R_{b} together with the nitrogen atom that they are connected to form an optionally substituted heterocycle including the nitrogen atom and 0-3 additional heteroatoms each selected from the group consisting of N, O, and S.

In any one of the embodiments described herein, the compound is selected from the group consisting of compounds 1-127 as shown in Table 1.

In another aspect, a pharmaceutical composition is described, including at least one compound according to any one of the embodiments described herein or a pharmaceutically-acceptable salt thereof and a pharmaceutically-acceptable carrier or diluent.

In yet another aspect, a method of treating a condition in a mammalian species in need thereof is described, including administering to the mammalian species a therapeutically effective amount of at least one compound according to any one of the embodiments described herein, or a pharmaceutically-acceptable salt thereof, or a pharmaceutical composition thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the Central Nerve System (CNS) disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein, or a pharmaceutically-acceptable salt thereof, or a pharmaceutical composition thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRaC(═O)NR_{b}R_{c}, NR_{d}S(═O)₂NR_{b}R_{c}, NRaP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ, and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}Rₑ, OC(═O)Rₐ, OC(═O)NR_{b}Rₑ, NR_{b}C(═O)ORₑ, NRaC(═O)NR_{b}Rₑ, NR_{d}S(═O)₂NR_{b}Rₑ, NRₐP(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

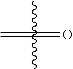 [SMILES: *=O]

The term “oxo” refers to substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure

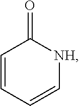

of which also includes its tautomeric form of

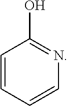

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and thus have desirable cardiovascular safety profiles.

In one aspect, a compound of Formula I or a pharmaceutically-acceptable salt thereof is described,

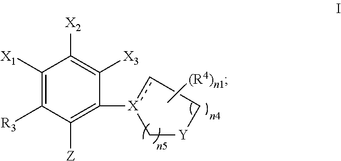

where

[symbol] refers to a single or double bond;

X is C, N, or CR₄ where valence permits;

Y is C(R₄)₂, NR₅, or O; where at least one of X and Y is N optionally substituted by R₅ where valence permits; where Y and either of its adjacent ring atoms are not linked together to form a fused ring system;

Z is ORₐ;

X₁ is H, halogen, CN, alkyl, cycloalkyl, halogenated cycloalkyl, or halogenated alkyl;

X₂ is H, halogen, CN, alkyl, cycloalkyl, halogenated cycloalkyl, or halogenated alkyl;

X₃ is H, halogen, CN, alkyl, cycloalkyl, halogenated cycloalkyl, or halogenated alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₃ is independently H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ;

each occurrence of R₄ is independently H, alkyl, cycloalkyl, optionally substituted saturated heterocycle, optionally substituted aryl, optionally substituted heteroaryl, CN, CF₃, ORₐ, (CR₆R₇)ₙ₃ORₐ, oxo, (C═O)R_{b}, (C═O)OR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, or (C═O)NRₐ(CR₆R₇)ₙ₃OR_{b}, (CR₆R₇)ₙ₃NRₓR_{b}, or (CR₆R₇)ₙ₃(C═O)NRₓR_{b}; wherein Rₓ is Rₐ, (C═O)Rₐ, (C═O)NRₐR_{b}, or SO₂Rₐ;

or two R₄ groups taken together with the carbon atom(s) that they are connected to form a 3-7 membered optionally substituted carbocycle or heterocycle;

each occurrence of R₅ is independently H, alkyl, cycloalkyl, optionally substituted saturated heterocycle, optionally substituted aryl, optionally substituted heteroaryl, Rₐ, NRₐR_{b}, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ;

each occurrence of R₆ and R₇ are independently H, alkyl, cycloalkyl, optionally substituted aryl, or optionally substituted heteroaryl;

each occurrence of Rₐ and R_{b} are independently H, alkyl, alkenyl, cycloalkyl, optionally substituted saturated heterocycle comprising 1-3 heteroatoms each selected from the group consisting of N, O, and S, optionally substituted aryl, or optionally substituted heteroaryl; or alternatively Rₐ and R_{b} together with the nitrogen atom that they are connected to form an optionally substituted heterocycle including the nitrogen atom and 0-3 additional heteroatoms each selected from the group consisting of N, O, and S;

the alkyl, cycloalkyl, heterocycle, aryl, and heteroaryl in X₁, X₂, X₃, R₃, R₄, R₅, R₆, R₇, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits;

each occurrence of R₈ is independently H, alkyl, or optionally substituted heterocycle; or alternatively the two R₈ groups together with the nitrogen atom that they are connected to form an optionally substituted heterocycle including the nitrogen atom and 0-3 additional heteroatoms each selected from the group consisting of N, O, and S;

each occurrence of n₁ is independently an integer from 0-3 where valence permits;

each occurrence of n₃ is independently an integer from 0-3; and

each occurrence of n₄ and n₅ is independently 0, 1 or 2.

In some embodiments, n₁ is an integer from 0-3. In some embodiments, n₁ is an integer from 0-2. In some embodiments, n₁ is an integer from 1-3. In some embodiments, n₁ is an integer from 2-3. In some embodiments, n₁ is 1 or 2. In some embodiments, n₁ is 1. In some embodiments, n₁ is 0.

In some embodiments, n₃ is an integer from 0-3. In some embodiments, n₃ is an integer from 0-2. In some embodiments, n₃ is an integer from 1-3. In some embodiments, n₃ is an integer from 2-3. In some embodiments, n₃ is 0. In some embodiments, n₃ is 1 or 2. In some embodiments, n₃ is 1.

In some embodiments, n₄ is an integer from 0-2. In some embodiments, n₄ is an integer from 0-1. In some embodiments, n₄ is 0. In some embodiments, n₄ is 2. In some embodiments, n₄ is 1.

In some embodiments, n₅ is an integer from 0-2. In some embodiments, n₅ is an integer from 0-1. In some embodiments, n₅ is 0. In some embodiments, n₅ is 2. In some embodiments, n₅ is 1.

In some embodiments, n₄ and n₅ are 0 and 0, respectively. In some embodiments, n₄ and n₅ are 0 and 1, respectively. In some embodiments, n₄ and n₅ are 1 and 0, respectively. In some embodiments, n₄ and n₅ are 1 and 1, respectively. In some embodiments, n₄ and n₅ are 0 and 2, respectively. In some embodiments, n₄ and n₅ are 2 and 0, respectively. In some embodiments, n₄ and n₅ are 2 and 2, respectively. In some embodiments, n₄ and n₅ are 1 and 2, respectively. In some embodiments, n₄ and n₅ are 2 and 1, respectively.

In some embodiments, [symbol] is a single bond. In some embodiments, [symbol] is a double bond.

In some embodiments, X is N and Y is C(R₄)₂. In some embodiments, X is CR₄ and Y is NR₅. In some embodiments, X is CR₄ and Y is O. In some embodiments, X is N and Y is NR₅.

In some embodiments, the structural moiety

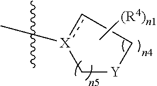

has the structure of

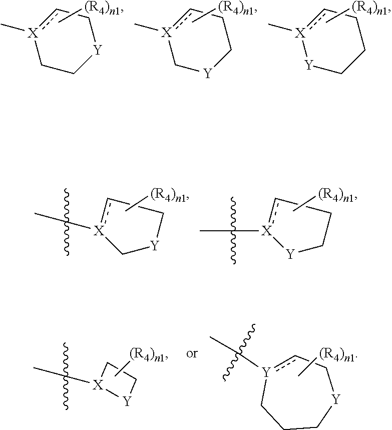

In some embodiments, the structural moiety

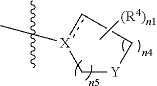

has the structure of

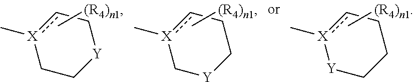

In some embodiments, the structural moiety

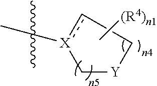

has the structure of

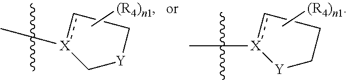

In some embodiments, the structural moiety

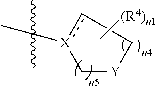

has the structure of

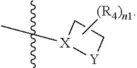

In some embodiments, the structural moiety

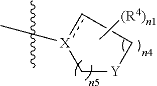

has the structure of

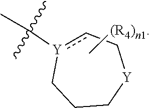

In some embodiments, the structural moiety

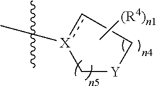

has the structure of

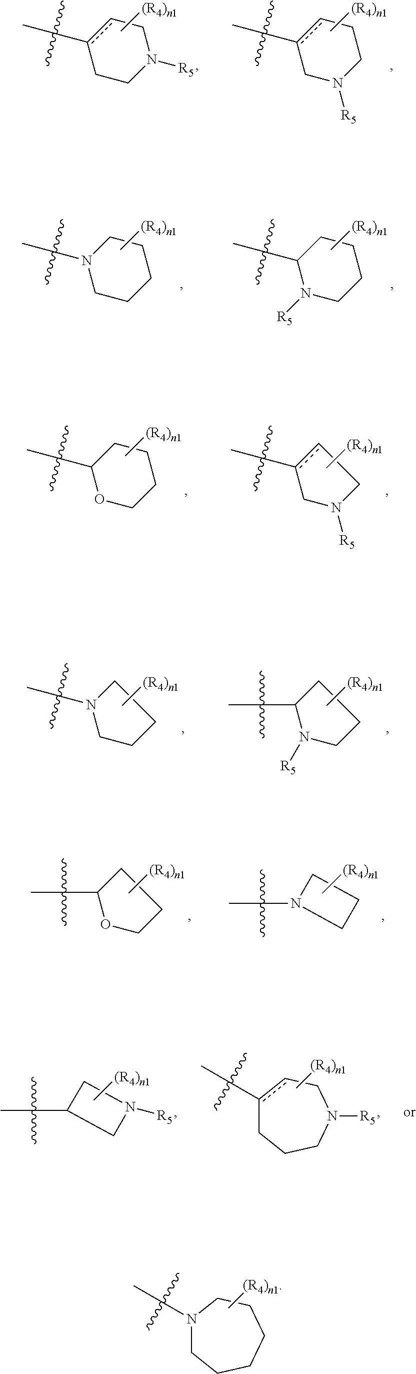

In some embodiments, the structural moiety

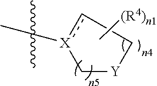

has the structure of

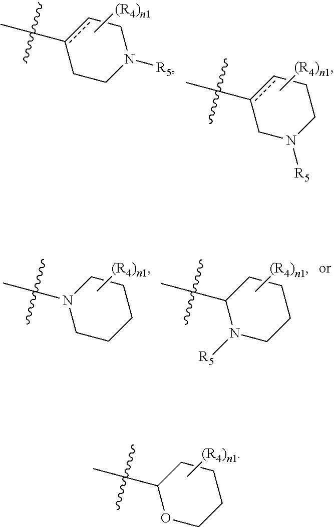

In some embodiments, the structural moiety

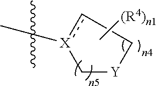

has the structure of

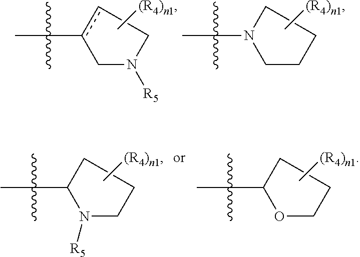

In some embodiments, the structural moiety

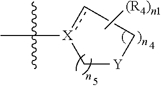

has the structure of

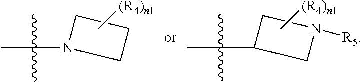

In some embodiments, the structural moiety

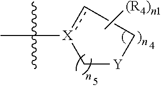

has the structure of

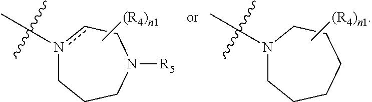

In some embodiments, the structural moiety

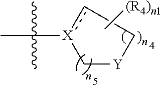

has the structure of

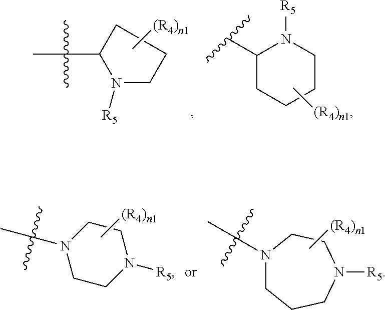

In some embodiments, the structural moiety

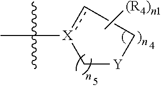

has the structure of

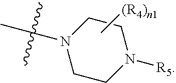

In some embodiments, the structural moiety

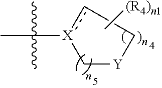

has the structure of

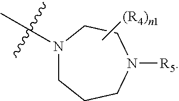

In some embodiments, the structural moiety

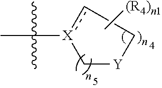

has the structure of

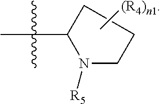

In some embodiments, the structural moiety

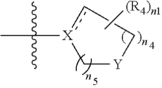

has the structure of

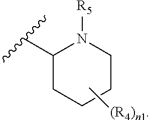

In some specific embodiments, n₁ is 0 and R₅ is H or alkyl. In some specific embodiments, n₁ is 1 and R₅ is H or alkyl.

In some specific embodiments, R₅ is H.

In some embodiments, at least one occurrence of R₄ is H, CN, alkyl, cycloalkyl, aryl, heteroaryl, CF₃, or ORₐ. In some embodiments, at least one occurrence of R₄ is (CR₆R₇)ₙ₃ORₐ, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, or a N-containing heterocycle. In some embodiments, at least one occurrence of R₄ is oxo, (C═O)R_{b} or (C═O)OR_{b}. In some embodiments, at least one occurrence of R₄ is (CR₆R₇)ₙ₃NRₐSO₂R_{b}. In some embodiments, at least one occurrence of R₄ is (CR₆R₇)ₙ₃NRₐ(C═O)R_{b}, (CR₆R₇)ₙ₃NRₐ(C═O)NRₐR_{b}, or (CR₆R₇)ₙ₃(C═O)NRₐR_{b}. In some embodiments, at least one occurrence of R₄ is a N-containing heterocycle. In some embodiments, at least one occurrence of R₄ is H or alkyl. Non-limiting examples of alkyl include methyl, ethyl, propyl, isopropyl, n-butyl, iso-butyl, or sec-butyl, pentyl, hexyl, heptyl, or octyl.

In some embodiments, one or more occurrences of R₄ are (CR₆R₇)ₙ₃ORₐ or (CR₆R₇)ₙ₃NRₐR_{b}. In some embodiments, one or more occurrences of R₄ are ORₐ, NRₐR_{b}, —CH₂ORₐ, —CH₂NRₐR_{b}, —CH₂CH₂ORₐ, or —CH₂CH₂NRₐR_{b}. In some embodiments, at least one occurrence of R₄ is (CR₆R₇)ₙ₃(C═O)NRₐR_{b}. In some embodiments, at least one occurrence of R₄ is (C═O)NRₐ(CR₆R₇)ₙ₃OR_{b}. In some embodiments, at least one or more occurrences of R₄ is (C═O)NRₐR_{b} or —CH₂(C═O)NRₐR_{b}. In some embodiments, at least one or more occurrences of R₄ is (C═O)NRₐR_{b}. In some embodiments, at least one or more occurrences of R₄ is —CH₂(C═O)NRₐR_{b}.

In some embodiments, one or more occurrences of R₄ are (CR₆R₇)ₙ₃NRₓR_{b} or (CR₆R₇)ₙ₃(C═O)NRₓR_{b}; wherein Rₓ is Rₐ, (C═O)Rₐ, (C═O)NRₐR_{b}, or SO₂Rₐ.

In some specific embodiments, at least one occurrence of R₄ is NH₂, CH₂NH₂, CH₂CH₂NH₂, CONH₂, CONHMe₂, CONMe₂, NH(CO)Me, NMe(CO)Me, CH₂CONH₂, CH₂CONHMe₂, CH₂CONMe₂, CH₂NH(CO)Me, or CH₂NMe(CO)Me. In other specific embodiments, at least one occurrence of R₄ is CH₂NH₂,

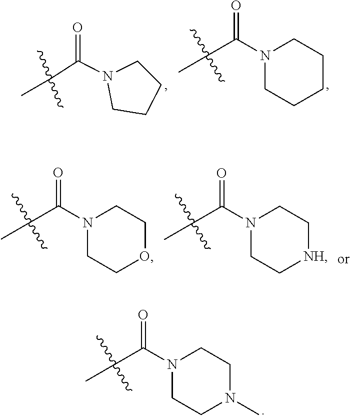

In other specific embodiments, at least one occurrence of R₄ is CH₂OH, CH₂NH₂NH₂,

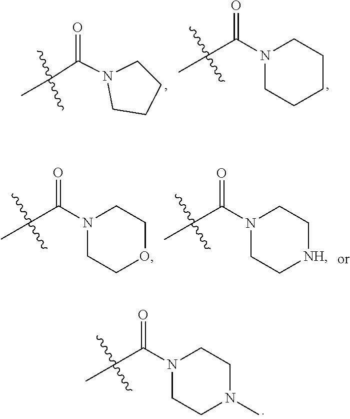

In other specific embodiments, at least one occurrence of R₄ is

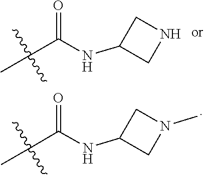

In other specific embodiments, at least one occurrence of R₄ is

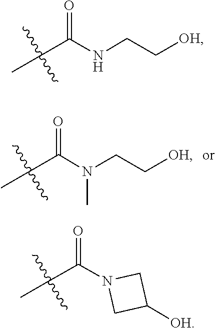

In still other embodiments, at least one occurrence of R₄ is an optionally substituted 4-, 5- or 6-membered heterocycle containing 1-3 heteroatoms each selected from the group consisting of N, O, and S. In further embodiments, at least one occurrence of R₄ is a heterocycle selected from the group consisting of

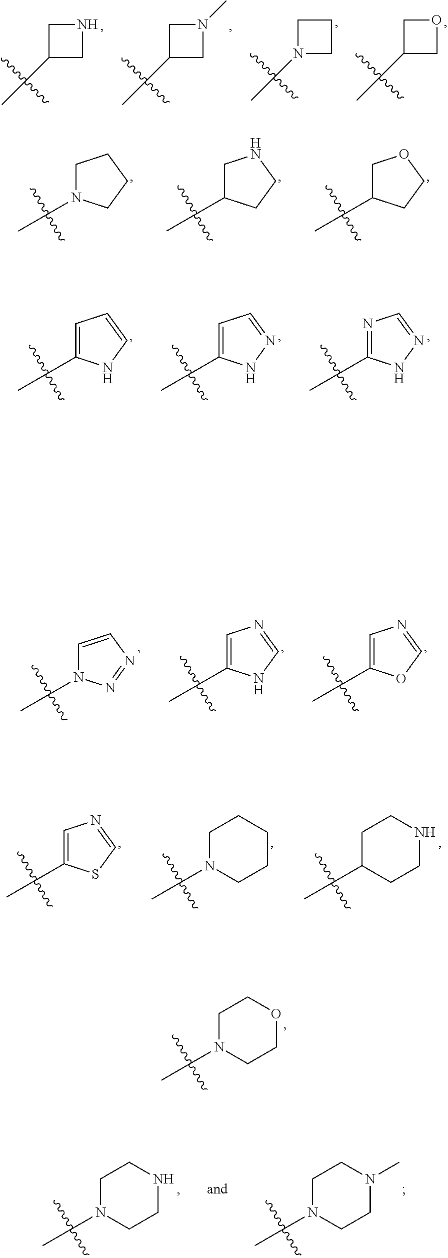

wherein the heterocycle is optionally substituted by alkyl, OH, oxo, or (C═O)C₁₋₄alkyl where valence permits.

In some embodiments, R₄ is H, Me, Et, Pr, Bu, or a saturated heterocycle or heteroaryl selected from the group consisting of

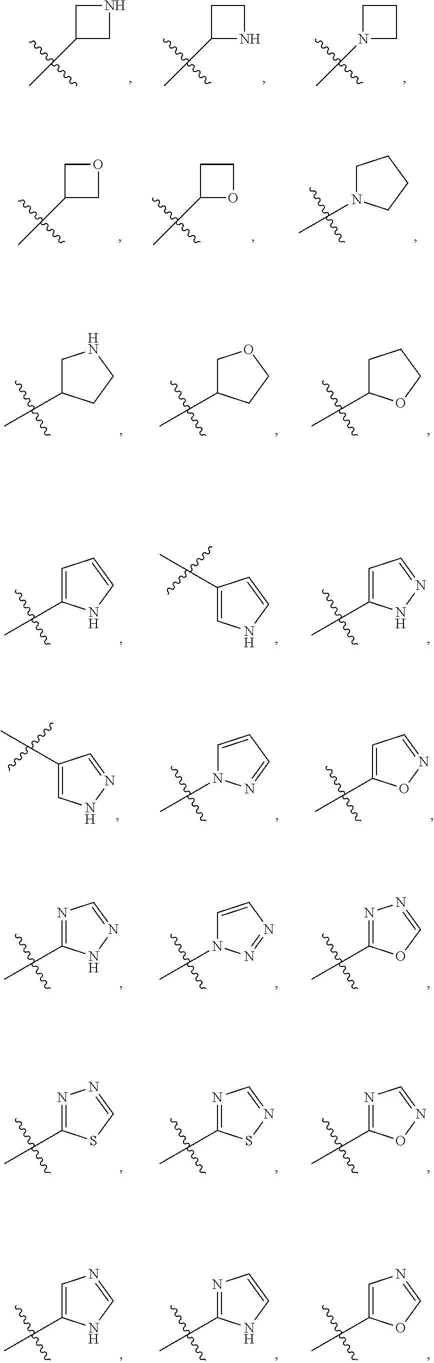

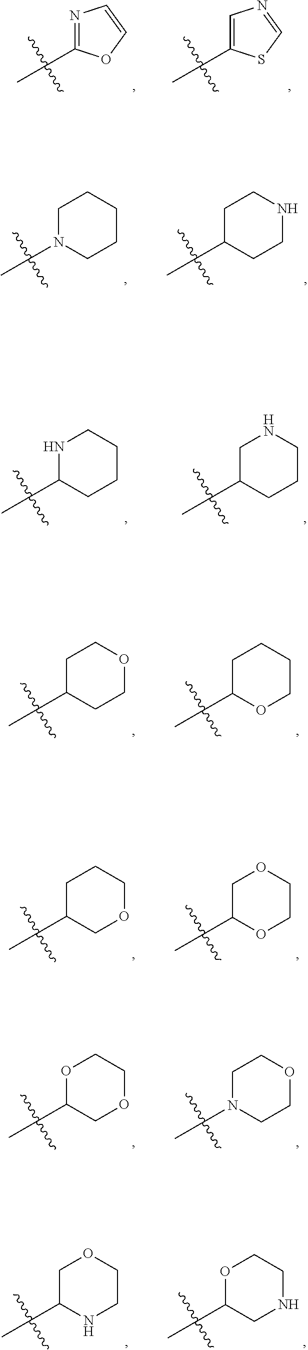

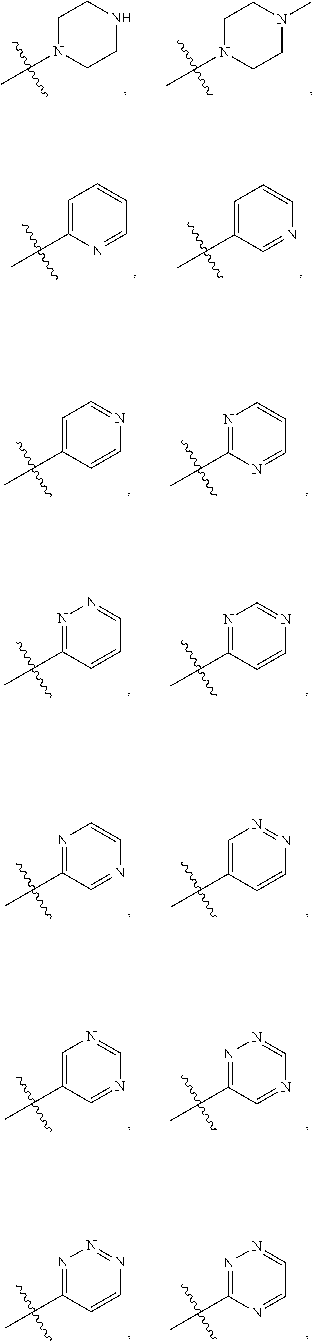

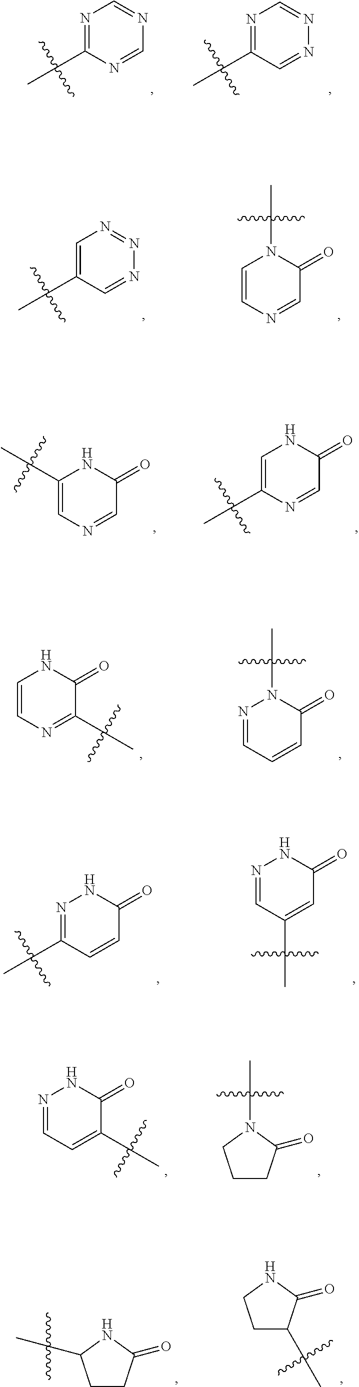

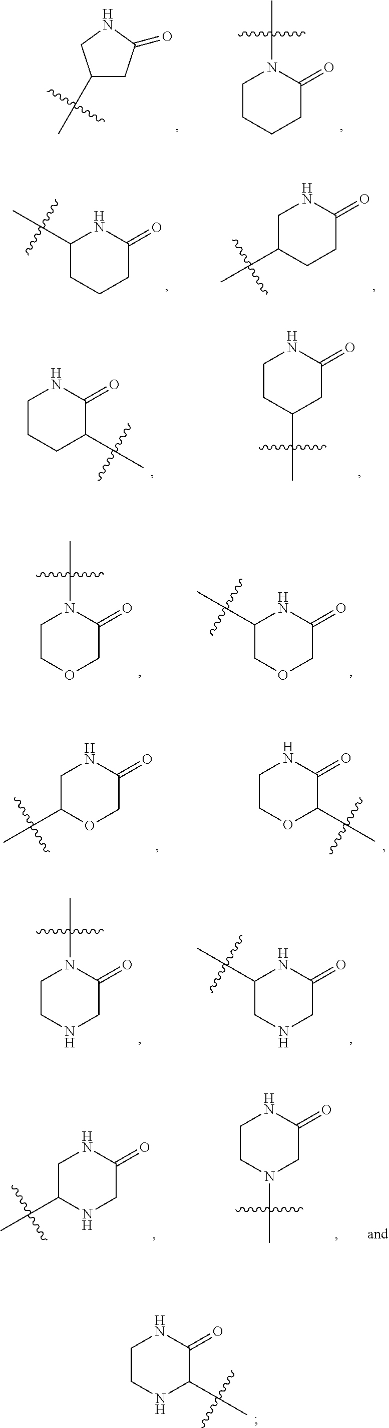

wherein the saturated heterocycle or heteroaryl is optionally substituted by cyano, cycloalkyl, fluorinated alkyl, fluorinated cycloalkyl, halogen, OH, NH₂, oxo, or (C═O)C₁₋₄alkyl where valence permits.

In some specific embodiments, R₄ is H, halogen, alkyl, ORₐ, NRₐR_{b}, or oxo. In other specific embodiments, R₄ is H, F, Cl, Br, Me, Et, Pr, iso-Pr, Bu, iso-Bu, sec-Bu, or tert-Bu. In other specific embodiments, R₄ is OH, NH₂, NHMe, NMe₂, NHEt, NMeEt, NEt₂, or oxo. In still other specific embodiments, at least one occurrence of R₄ is H, halogen, alkyl, OH, NH₂, CN, CF₃, OCF₃, CONH₂, CONHMe₂, or CONMe₂.

In further embodiments, two R₄ groups taken together with the carbon atom(s) that they are connected to form a 3-7 membered optionally substituted carbocycle or heterocycle.

In some embodiments, at least one occurrence of R₅ is H, alkyl, cycloalkyl, aryl, heteroaryl, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ. In some embodiments, at least one occurrence of R₅ is H, alkyl, or cycloalkyl. In some embodiments, at least one occurrence of R₅ is aryl or heteroaryl.

In some specific embodiments, at least one occurrence of R₅ is (C═O)Rₐ, (C═O)-alkyl-ORₐ, (C═O)-alkyl-NRₐR_{b}, (C═O)NRₐR_{b}, or SO₂Rₐ. In some specific embodiments, at least one occurrence of R₅ is (C═O)Rₐ or (C═O)-alkyl-ORₐ. In some specific embodiments, at least one occurrence of R₅ is (C═O)-alkyl-NRₐR_{b} or (C═O)NRₐR_{b}. In some specific embodiments, at least one occurrence of R₅ is (C═O)NRₐR_{b}, (C═O)CH₂NRₐR_{b}, or (C═O)CH₂CH₂NRₐR_{b}.

In some embodiments, each occurrence of R₆ and R₇ are independently H or alkyl. In some specific embodiments, CR₆R₇ is CH₂, CHMe, CMe₂, CHEt, or CEt₂. In some specific embodiments, CR₆R₇ is CH₂.

In some embodiments, the compound has a structure of Formula Ia:

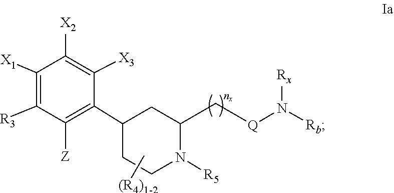

wherein
nₓ is 0, 1, or 2;
Q is CR₆R₇ or C═O; and
Rₓ is Rₐ, (C═O)Rₐ, (C═O)NRₐR_{b}, or SO₂Rₐ.

In some embodiments, nₓ is 0 or 1. In some embodiments, R₅ is H or Me. In some embodiments, Q is C═O. In some embodiments, NRₓR_{b} is NH₂, NHMe, NMe₂, NH(C═O)NH₂, NMe(C═O)NH₂, NH(C═O)NHMe, NMe(C═O)NMe, NH(C═O)NMe₂, NMe(C═O)NMe₂, or SO₂Me. In some embodiments, NRₓR_{b} is NH₂, NHMe, or NMe₂. In some embodiments, NRₓR_{b} is NH(C═O)NH₂, NMe(C═O)NH₂, NH(C═O)NHMe, NMe(C═O)NMe, NH(C═O)NMe₂, or NMe(C═O)NMe₂.

In some embodiments, [symbol] refers to a single bond; X is CR₄; Y is O or NR₅; R₃ is H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ; R₄ is H, alkyl, or (C═O)NRₐR_{b}; R₅ is H or alkyl; n₁ is 1, 2, or 3; n₄ is 0, 1 or 2; and n₅ is 0 or 1. In some embodiments, R₄ is (C═O)NRₐR_{b}.

In some embodiments, the compound has the structure of Formula 1b:

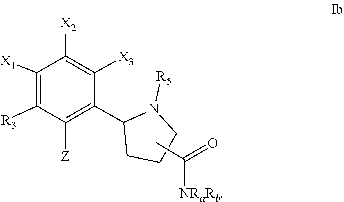

In some embodiments, the compound has the structure of

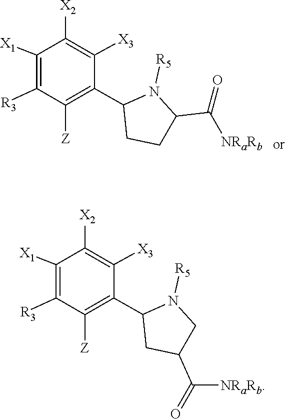

In some embodiments, the compound has the structure of Formula 1c:

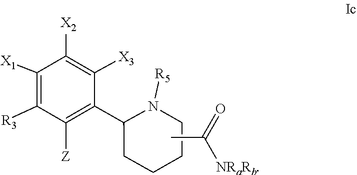

In some embodiments, the compound has the structure of

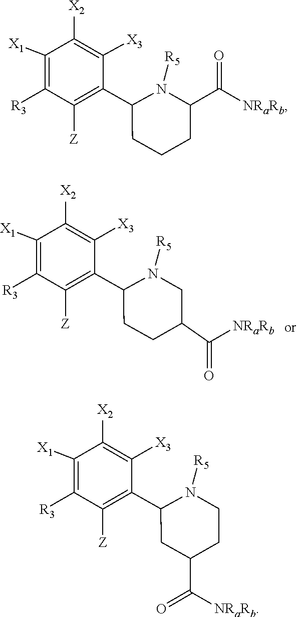

In some embodiments, Z is ORₐ. In some embodiments, Z is OH or (C₁-C₄ alkyl). In some embodiments, Z is OH, OMe, OEt, OPr, Oi-Pr, OBu, Oi-Bu, Osec-Bu, Ot-Bu. In some embodiments, Z is OH.

In some embodiments, X₁ is H, halogen, CN, alkyl, halogenated alkyl, cycloalkyl, or halogenated cycloalkyl. In some embodiments, X₁ is H, halogen, fluorinated alkyl, or alkyl. In some embodiments, X₁ is H or halogen. In other embodiments, X₁ is fluorinated alkyl or alkyl. In other embodiments, X₁ is cycloalkyl. In some embodiments, X₁ is H, F, Cl, Br, Me, CF₂H, CF₂Cl, or CF₃. In some embodiments, X₁ is H, F, or C₁. In some embodiments, X₁ is F or C₁. In some embodiments, X₁ is H or C₁. In some embodiments, X₁ is F. In some embodiments, X₁ is Cl. In some embodiments, X₁ is CF₃ or CF₂H. In some embodiments, X₁ is CF₂Cl. In some embodiments, X₁ is H.

In some embodiments, X₂ is H, halogen, CN, alkyl, halogenated alkyl, cycloalkyl, or halogenated cycloalkyl. In some embodiments, X₂ is H, halogen, fluorinated alkyl, or alkyl. In some embodiments, X₂ is H or halogen. In other embodiments, X₂ is fluorinated alkyl or alkyl. In other embodiments, X₂ is cycloalkyl. In some embodiments, X₂ is H, F, Cl, Br, Me, CF₂H, CF₂Cl, or CF₃. In some embodiments, X₂ is H, F, or C₁. In some embodiments, X₂ is F or C₁. In some embodiments, X₂ is H or C₁. In some embodiments, X₂ is F. In some embodiments, X₂ is C₁. In some embodiments, X₂ is CF₃ or CF₂H. In some embodiments, X₂ is CF₂Cl. In some embodiments, X₂ is H.

In some embodiments, X₃ is H, halogen, CN, alkyl, halogenated alkyl, cycloalkyl, or halogenated cycloalkyl. In some embodiments, X₃ is H, halogen, alkyl, or halogenated alkyl. In some embodiments, X₃ is H, halogen, fluorinated alkyl, or alkyl. In some embodiments, X₃ is H or halogen. In other embodiments, X₃ is fluorinated alkyl or alkyl. In some embodiments, X₃ is H, F, Cl, Br, Me, CF₂H, CF₂Cl, or CF₃. In some embodiments, X₃ is H, F, or C₁. In some embodiments, X₃ is F or C₁. In some embodiments, X₃ is H or C₁. In some embodiments, X₃ is F. In some embodiments, X₃ is C₁. In some embodiments, X₃ is CF₃ or CF₂H. In some embodiments, X₃ is CF₂Cl. In some embodiments, X₃ is H.

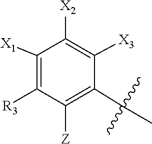

In some embodiments, the structural moiety z has the structure of

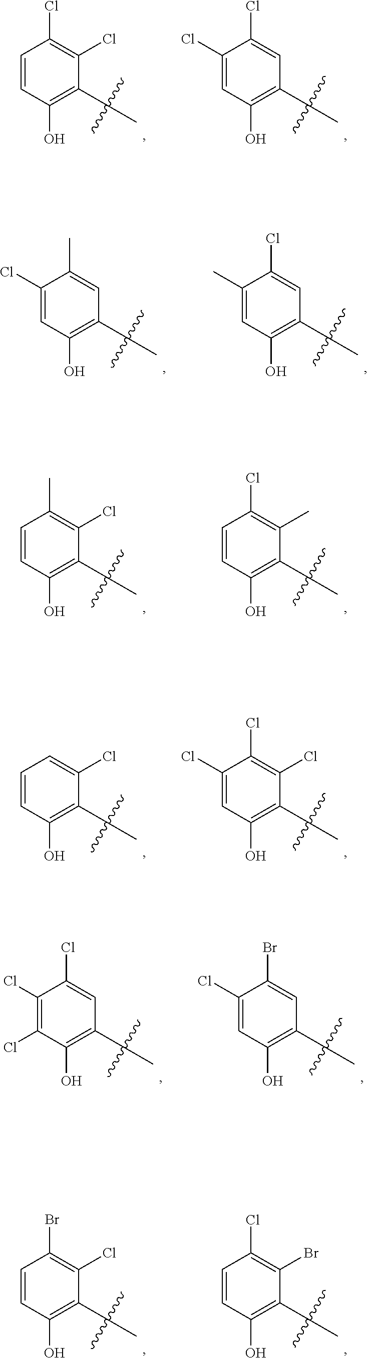

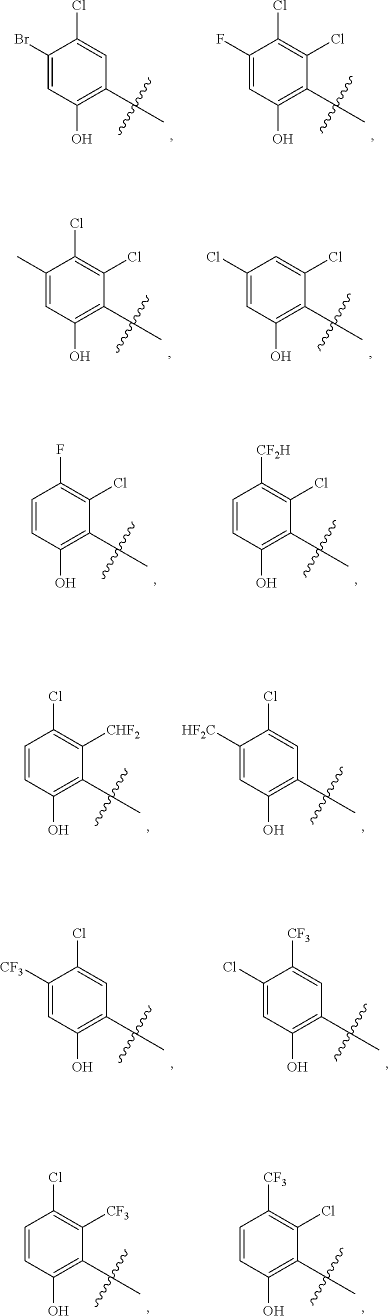

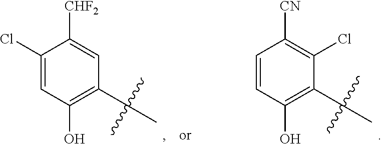 [SMILES: *c1cc(C(F)F)c(Cl)cc1O ; *c1c(O)ccc(C#N)c1Cl]

In some embodiments, the compound of Formula I has a structure of Formula II′,

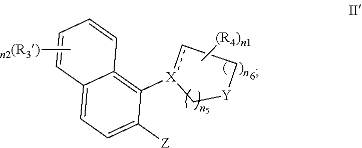

wherein each occurrence of R_{3′} is independently H, halogen, or alkyl; and n₂ is an integer from 0-3 and other substituents are as defined herein. In some embodiments, R_{3′} is H or alkyl. In some embodiments, R_{3′} is halogen.

In some embodiments, the compound of Formula I has a structure of Formula II,

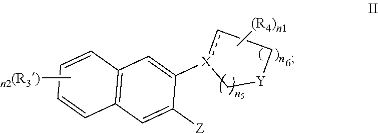

wherein each occurrence of R_{3′} is independently H, halogen, or alkyl; and n₂ is an integer from 0-3 and other substituents are as defined herein. In some embodiments, R_{3′} is H or alkyl. In some embodiments, R_{3′} is halogen.

In some embodiments, n₂ is an integer from 0-3. In some embodiments, n₂ is an integer from 1-3. In some embodiments, n₂ is 0. In some embodiments, n₂ is 1 or 2. In some embodiments, n₂ is 1.

In some embodiments, R₃ is H, alkyl, cycloalkyl, aryl, heteroaryl, CN, CF₃, ORₐ, SRₐ, halogen, NRₐR_{b}, or NR_{b}(C═O)Rₐ. In some embodiments, R₃ is H, alkyl, CF₃, OCF₃, ORₐ, SRₐ, halogen, NRₐR_{b}, or NR_{b}(C═O)Rₐ. In some embodiments, R₃ is H, halogen, fluorinated alkyl, or alkyl. In some embodiments, R₃ is H or halogen. In some embodiments, R₃ is alkyl or fluorinated alkyl. In some embodiments, R₃ is H, Cl, Br, CF₃, CHF₂, or Me. In some embodiments, R₃ is H.

In some embodiments, at least one occurrence of Rₐ or R_{b} is independently H, alkyl, cycloalkyl, saturated heterocycle, aryl, or heteroaryl. In some embodiments, at least one occurrence of Rₐ or R_{b} is independently H or alkyl. In some embodiments, at least one occurrence of Rₐ or R_{b} is independently H, Me, Et, Pr, or Bu. In some embodiments, at least one occurrence of Rₐ or R_{b} is independently a heterocycle selected from the group consisting of

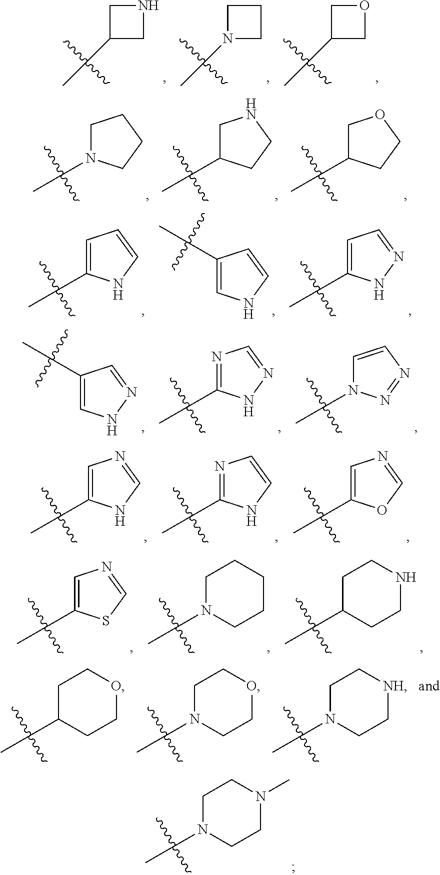

where the heterocycle is optionally substituted by alkyl, OH, oxo, or (C═O)C₁₋₄alkyl where valence permits. In some embodiments, at least one occurrence of Rₐ or R_{b} is independently H or

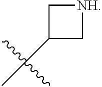 [SMILES: *C1CNC1]

In some embodiments, Rₐ and R_{b} together with the nitrogen atom that they are connected to form an optionally substituted heterocycle including the nitrogen atom and 0-3 additional heteroatoms each selected from the group consisting of N, O, and S.

In some embodiments, the alkyl, cycloalkyl, heterocycle, aryl, and heteroaryl in X₁, X₂, and X₃ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits. In some embodiments, the alkyl, cycloalkyl, heterocycle, aryl, and heteroaryl in R₃ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits. In some embodiments, the alkyl, cycloalkyl, heterocycle, aryl, and heteroaryl in R₄ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits. In some embodiments, the alkyl, cycloalkyl, heterocycle, aryl, and heteroaryl in R₅ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits. In some embodiments, the alkyl, cycloalkyl, heterocycle, aryl, and heteroaryl in R₆ and R₇ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits. In some embodiments, the alkyl, cycloalkyl, heterocycle, aryl, and heteroaryl in Rₐ and R_{b} are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits.

In some embodiments, each occurrence of R₈ is independently H, alkyl, or optionally substituted heterocycle. In some embodiments, each occurrence of R₈ is independently H or alkyl. In some embodiments, each occurrence of R₈ is substituted heterocycle. In some embodiments, the two R₈ groups together with the nitrogen atom that they are connected to form an optionally substituted heterocycle including the nitrogen atom and 0-3 additional heteroatoms each selected from the group consisting of N, O, and S.

In some embodiments, the compound of Formula I is selected from the group consisting of compounds 1-127 as shown in Table 1 below.

### ABBREVIATIONS

ACN Acetonitrile
Boc Tert-butyloxycarbonyl
CDI Carbonyldiimidazole
DAST Diethylaminosulfur trifluoride
DCE Dichloroethane
DCM Dichloromethane
DIBAL or Diisobutylaluminium hydride
DIBAL-H
DIPA Diisopropylamine
DMAP 4-Dimethylaminopyridine
DME Dimethoxyethane
DMF Dimethyl formamide
EA Ethyl acetate
EDCI or EDC 1-Ethyl-3-(3-dimethylaminopropyl)carbodiimide
FA Formic acid
HATU N-[(dimethylamino)(3H-1,2,3-triazolo(4,4-b)pyridin-3-yloxy)methylene]-N-methylmethaneaminium hexafluorophosphate
HOBT Hydroxybenzotriazole
IPA Isopropyl alcohol
LDA Lithium diisopropylamide
PE Petroleum ether
PMB 4-Methoxybenzyl
SEM Trimethylsilylethoxymethyl
TBAF Tetra-n-butylammonium fluoride
TEA Triethylamine
TFA Trifluoroacetic acid
THF Tetrahydrofuran

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-6 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-6 and examples described in the Example section below illustrate methods used for the preparation of the compounds described herein.

Compounds I-1a, I-2, and I-5 as shown in Scheme 1 can be prepared by any method known in the art and/or are commercially available. As shown in Scheme 1, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH or an amine group. Other substituents are defined herein. As shown in Scheme 1, compounds disclosed herein where X is C or CR₄ can be made by reacting a bromobenzene with a boronic acid or a bromoheterocycle. Bromobenzene I-1a undergoes a Suzuki reaction with a vinylboronic acid heterocycle I-2 in the presence of a base such as sodium carbonate and a suitable catalyst such as Pd(PPh₃)₄ to give adduct I-3. The double bond in I-3 is then reduced by hydrogenation in the presence of PtO₂ and HCl in a solvent such as methanol to give intermediate I-4a. Alternatively, I-1a can be reacted with a saturated bromoheterocycle I-5 in a photoredox reaction using tris trimethylsilyl silane, a combination of iridium and nickel catalyst (e.g., Ir[dF(CF₃)ppy]₂(dtbbpy)PF₆ and NiCl₂, respectively) under irradiation with blue LED light to give I-4 directly. After removal of the N-protecting group on the amine of compound I-4a, the amine of I-4a can be modified by acylation, alkylation or reductive amination by methods known in the art. When R₄ is a functional group such as ester or nitrile, it can be converted to other substituents by methods known in the art. Additionally, the double bond in I-3 can be functionalized, e.g., by hydroboration. The OH-protecting group in compound I-4a can be selectively removed.

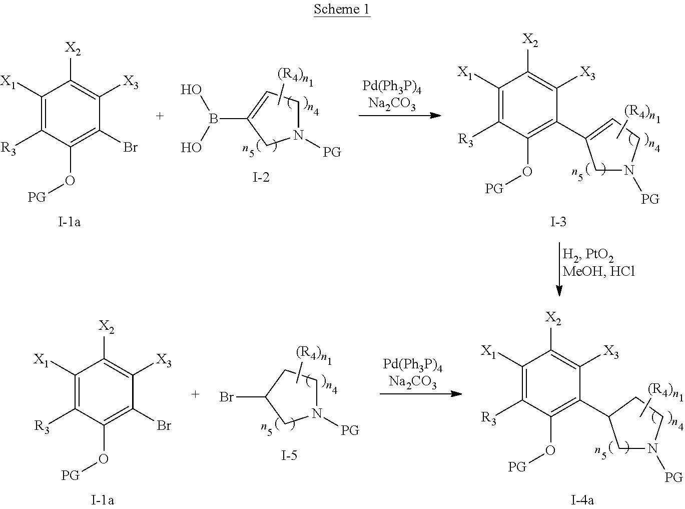

Compounds I-1a and I-6 as shown in Scheme 2 can be prepared by any method known in the art and/or are commercially available. As shown in Scheme 2, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents shown in Scheme 2 are defined herein. For compounds disclosed herein where n₄ is 1 and n₅ is 2, the 6-member ring can be obtained by the synthesis described in Scheme 2. As shown in Scheme 2, a Suzuki reaction between I-1a and a pyridine boronic acid I-6 in the presence of a base such as sodium carbonate and a suitable catalyst such as Pd(PPh₃)₄ gives adduct I-7, which can then be reduced by hydrogenation in the presence of PtO₂ and HCl in a solvent such as methanol to give I-4b. The protecting group in compound I-4b can then be selectively removed to afford a compound of Formula I or a precursor thereof.

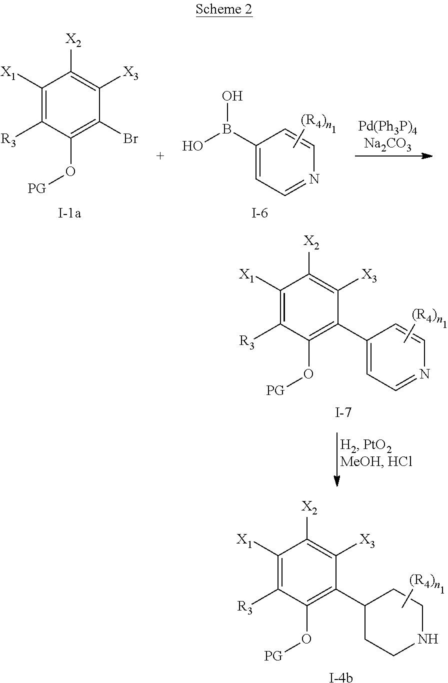

Compounds I-1b and I-8 as shown in Scheme 3 can be prepared by any method known in the art and/or are commercially available. As shown in Scheme 3, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH or an amine group. Other substituents shown in Scheme 3 are defined herein. For compounds disclosed herein where X is CR₄ and R₄ is alkyl, reaction of benzene I-1b with a tertiary alcohol I-8 in the presence of triflic acid yields I-4c (Scheme 3). The protecting groups in compound I-4c can then be optionally removed to afford a compound of Formula I or a precursor thereof.

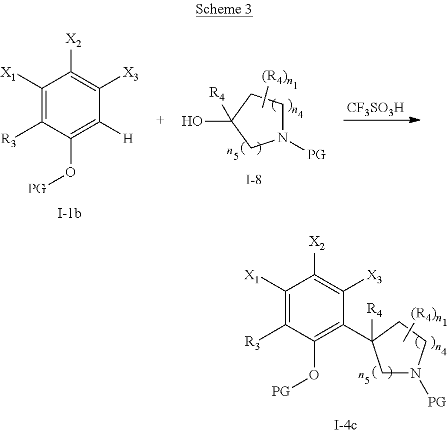

Compounds I-9 and I-10, as shown in Scheme 4, can be prepared by any method known in the art and/or are commercially available. As shown in Scheme 4, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents shown in Scheme 4 are defined herein. For compounds disclosed herein where X is CR₄ and R₄ is a functional group, compounds disclosed herein wherein n₄ is 1 and n₅ is 2 can be obtained by alkylation of phenylacetonitrile I-9 with N-boc-bis-chloroethylamine I-10 in the presence of a base such as NaH in a solvent such as THE to form piperidine nitrile I-4d as shown in Scheme 4. The nitrile can then be converted to other groups such as ester, aminomethyl, hydroymethyl or amine by methods known in the art. The protecting groups in compound I-4c (PG, boc) can then be selectively removed to afford a compound of Formula I or a precursor thereof.

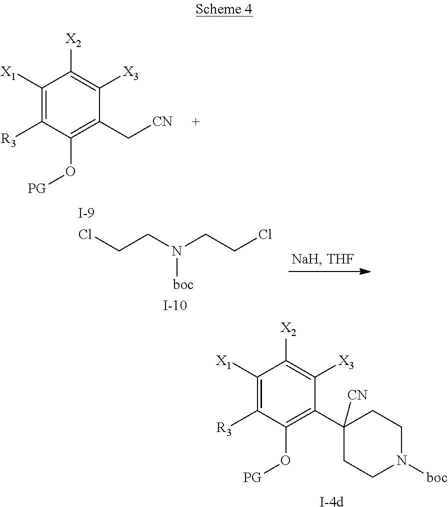

Compounds I-11 and I-12, as shown in Scheme 5, can be prepared by any method known in the art and/or are commercially available. As shown in Scheme 5, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents shown in Scheme 5 are defined herein. For compounds disclosed herein having 5-membered rings (e.g., n₄=n₅₌₁) where X is CH and R₄ is a functional group, the compounds can be formed by a dipolar cycloaddition of a methyl cinnamate I-11 with N-methoxymethyl-N-trimethylsilylmethylbenzyamine I-12 in the presence of an acid such as TFA. The product I-4e thus formed can be debenzylated (e.g., using 1-chloroethyl chloroformate) and the resulting amine can be further derivatized by methods known in the art. The protecting group in compound I-4e can be selectively removed to afford a compound of Formula I or a precursor thereof.

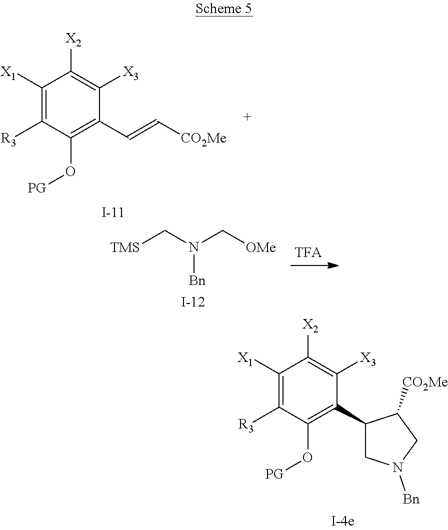

Compounds I-1a and I-13 as shown in Scheme 6 can be prepared by any method known in the art and/or are commercially available. As shown in Scheme 6, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents shown in Scheme 6 are defined herein. For compounds disclosed herein where X is N and Y is CR₄ or suitably substituted or protected N, the compounds can be synthesized from bromobenzene I-1a by a Buchwald-Hartwig reaction with cyclic amine I-13 in the presence of palladium agent (e.g., Pd₂(dba)₃) and a suitable ligand such as Xantphos, X-phos or Ruphos in the presense of a base (e.g., NaOt-Bu) to form I-4f as shown in Scheme 6. The protecting group in compound I-4f can be optionally removed to afford a compound of Formula I or a precursor thereof.

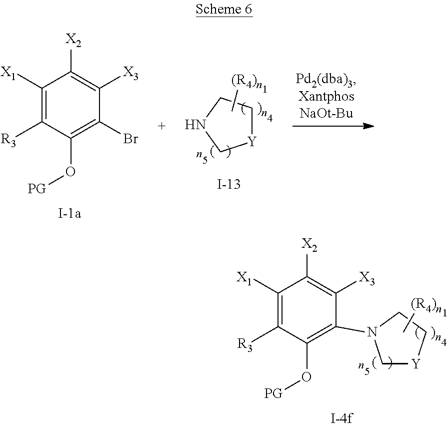

The reactions described in Schemes 1-6 can be carried out in a suitable solvent. Suitable solvents include, but are not limited to, acetonitrile, methanol, ethanol, dichloromethane, DMF, THF, MTBE, or toluene. The reactions described in Schemes 1-6 may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C. or higher or the refluxing/boiling temperature of the solvent used. The reaction mixture may alternatively be cooled in a cold bath at a temperature lower than room temperature, e.g., 0, −10, −20, −30, −40, −50, −78, or −90° C. The reaction may be worked up by removing the solvent or partitioning of the organic solvent phase with one or more aqueous phases, each optionally containing NaCl, NaHCO₃, or NH₄Cl. The solvent in the organic phase can be removed by reduced vacuum evaporation and the resulting residue may be purified using a silica gel column or HPLC.

### Pharmaceutical Compositions

This invention also provides a pharmaceutical composition comprising at least one of the compounds as described herein or a pharmaceutically-acceptable salt or solvate thereof, and a pharmaceutically-acceptable carrier or diluent.

In yet another aspect, the present invention provides a pharmaceutical composition comprising at least one compound selected from the group consisting of compounds of Formula I as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, e.g., Berge et al., (1977) “Pharmaceutical Salts”, J. Pharm. Sci. 66:1-19 (incorporated herein by reference in its entirety).

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method comprising administering to the mammalian species a therapeutically effective amount of at least one compound selected from the group consisting of compounds of Formula I, or a pharmaceutically-acceptable salt thereof or a pharmaceutical composition thereof, wherein the condition is selected from the group consisting of cancer, an immunological disorder, a CNS disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, MS, systemic lupus erythematosus, or type I diabetes mellitus). In some embodiments, the CNS disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically-acceptable salt or pharmaceutical composition thereof.

In some embodiments, the compounds described herein is selective in blocking the Kv 1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference in its entirety.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.; incorporated herein by reference in its entirety.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

Assays for Effectiveness of Kv1.3 potassium channel blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-9 describe various intermediates used in the syntheses of representative compounds of Formula I disclosed herein.

### Example 1. Intermediate 1 (2-bromo-3,4-dichloro-1-methoxybenzene) and Intermediate 2 (1-bromo-4,5-dichloro-2-methoxybenzene)

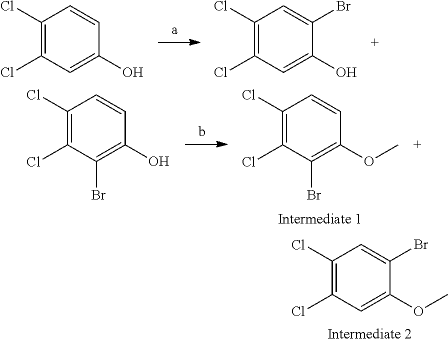

### Step a

To a stirred solution of 3,4-dichlorophenol (100.00 g, 613.49 mmol) in DCM (1000 mL) was added Br₂ (98.04 g, 613.49 mmol) dropwise at 0° C. under nitrogen atmosphere. The reaction solution was stirred for 16 h at room temperature under nitrogen atmosphere. The reaction was quenched with saturated aq. Na₂S₂O₃ (500 mL) at 0° C. The resulting mixture was extracted with EA (6×400 mL). The combined organic layers were washed with brine (2×400 mL) and dried over anhydrous Na₂SO₄. After filtration, the filtrate was concentrated under reduced pressure to afford a mixture of 2-bromo-4,5-dichlorophenol and 2-bromo-3,4-dichlorophenol (100 g, crude) as a yellow oil. The crude product was used in the next step directly without further purification.

### Step b

To a crude mixture of 2-bromo-4,5-dichlorophenol and 2-bromo-3,4-dichlorophenol (32 g, 125.04 mmol, 1 equiv.) and K₂CO₃ (54.9 g, 396.87 mmol, 3 equiv.) in MeCN (210 mL) was added MeI (16.5 mL, 116.05 mmol, 2 equiv.) dropwise at 0° C. The reaction mixture was stirred at 50° C. for 4 h. The reaction mixture was filtered and concentrated. The residue was purified by silica gel column chromatography, eluted with PE to afford Intermediate 1 (2-bromo-3,4-dichloro-1-methoxybenzene) (8.7 g, 25.7%) as a white solid: ¹H NMR (300 MHz, CDCl₃) δ 7.40 (dd, J=9.0, 1.1 Hz, 1H), 6.79 (d, J=8.9 Hz, 1H), 3.92 (s, 3H); and Intermediate 2 (1-bromo-4,5-dichloro-2-methoxybenzene) (24.3 g, 71.77%) as a white solid: ¹H NMR (300 MHz, CDCl₃) δ 7.64 (s, 1H), 6.99 (s, 1H), 3.91 (s, 3H).

### Example 2. Intermediate 3 ([2-(2-bromo-4,5-dichlorophenoxymethoxy)ethyl]trimethylsilane)

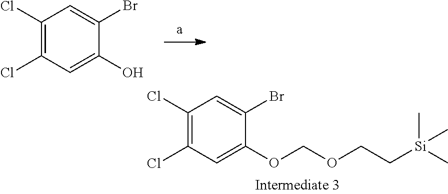

### Step a

To a stirred solution of 2-bromo-4,5-dichlorophenol (31.00 g, 128.15 mmol) and [2-(chloromethoxy)ethyl]trimethylsilane (32.00 g, 192.23 mmol) in DCM (100 mL) was added DIEA (49.70 g, 384.46 mmol) at room temperature. The resulting mixture was stirred at room temperature for 5 h. The reaction was quenched with water (200 mL). The resulting mixture was extracted with DCM (3×400 mL). The combined organic layers were washed with brine (3×200 mL) and dried over Na₂SO₄. After filtration, the filtrate was concentrated under reduced pressure. The residue was purified by silica gel column chromatography, eluted with PE/EA (50/1) to afford Intermediate 3 ([2-(2-bromo-4,5-dichlorophenoxymethoxy)ethyl]trimethylsilane) (44.00 g, 83%) as a light yellow oil: ¹H NMR (300 MHz, DMSO-d₆) δ 7.86 (s, 1H), 7.46 (s, 1H), 5.39 (s, 2H), 3.74 (t, J=6.0 Hz, 2H), 0.79 (t, J=6.0 Hz, 2H), −0.05 (s, 9H).

### Example 3. Intermediate 4 (1-tert-butyl 2-methyl 4-bromopiperidine-1,2-dicarboxylate)

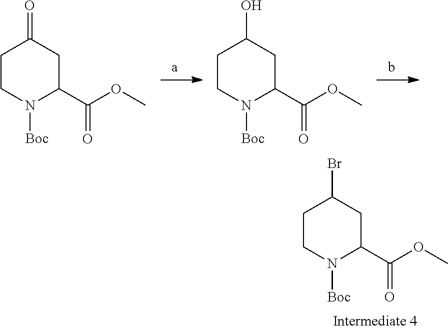

### Step a

To a stirred solution of 1-tert-butyl 2-methyl 4-oxopiperidine-1,2-dicarboxylate (1.00 g, 3.89 mmol) in THE (8 mL) was added NaBH₄ (0.29 g, 7.77 mmol) at 0° C. under nitrogen atmosphere. The resulting mixture was stirred at room temperature for 2 h under nitrogen atmosphere. The reaction was quenched with water (50 mL). The resulting mixture was extracted with EA (3×50 mL). The combined organic layers were washed with brine (2×30 mL), dried over anhydrous Na₂SO₄. After filtration, the filtrate was concentrated under reduced pressure to afford 1-tert-butyl 2-methyl 4-hydroxypiperidine-1,2-dicarboxylate as a light yellow oil (0.90 g, 89%): LCMS (ESI) calc'd for C₁₂H₂₁NO₅ [M+H]⁺: 260 found 260; ¹H NMR (300 MHz, CDCl₃) δ 5.17-4.66 (m, 1H), 4.22-3.83 (m, 1H), 3.76 (s, 3H), 3.71-3.63 (m, 1H), 3.47-2.84 (m, 1H), 2.56-2.36 (m, 1H), 1.99-1.87 (m, 1H), 1.81-1.58 (m, 1H), 1.58-1.40 (m, 10H).

### Step b

To a stirred mixture of 1-tert-butyl 2-methyl 4-hydroxypiperidine-1,2-dicarboxylate (0.90 g, 3.47 mmol) in DCM (8 mL) were added PPh₃ (1.37 g, 5.21 mmol) and CBr₄ (1.73 g, 5.21 mmol) at room temperature. The resulting mixture was stirred for 3 h at room temperature. The reaction was quenched with water (30 mL) at room temperature. The resulting mixture was extracted with EA (3×50 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (1/1) to afford Intermediate 4 (1-tert-butyl 2-methyl 4-bromopiperidine-1,2-dicarboxylate) as a light yellow oil (0.50 g, 40%): LCMS (ESI) calc'd for C₁₂H₂₀BrNO₄ [M+H]⁺: 322, 324 (1:1), found 322, 324 (1:1); ¹H NMR (300 MHz, CD₃OD) δ 4.77-4.67 (m, 1H), 4.67-4.60 (m, 1H), 3.97-3.86 (m, 1H), 3.74 (s, 3H), 3.56-3.34 (m, 1H), 2.75-2.62 (m, 1H), 2.45-2.31 (m, 1H), 2.05-1.94 (m, 2H), 1.46 (s, 9H).

### Example 4. Intermediate 5 (3,4-dichloro-2-iodophenol)

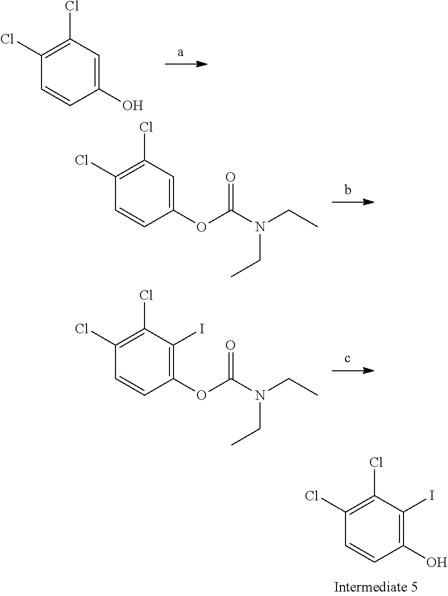

### Step a

To a stirred solution of 3,4-dichlorophenol (50.00 g, 306.75 mmol), DMAP (74.95 g, 613.50 mmol) and Et₃N (62.08 g, 613.50 mmol) in DCM (500 mL) was added diethylcarbamoyl chloride (62.39 g, 460.12 mmol) dropwise at room temperature under nitrogen atmosphere. The reaction mixture was stirred for 2 h at room temperature under nitrogen atmosphere. The resulting mixture was diluted with water (300 mL) at room temperature and extracted with EA (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (40/1) to afford 3,4-dichlorophenyl N,N-diethylcarbamate as a yellow oil (72.00 g, 80%): LCMS (ESI) calc'd for C₁₁H₁₃Cl₂NO₂ [M+H]⁺: 262, 264 (3:2), found 262, 264 (3:2); ¹H NMR (400 MHz, CDCl₃) δ 7.42 (d, J=8.8 Hz, 1H), 7.30 (d, J=2.7 Hz, 1H), 7.03 (dd, J=8.8, 2.7 Hz, 1H), 3.42 (dq, J=14.2, 7.2 Hz, 4H), 1.24 (dt, J=14.8, 7.2 Hz, 6H).

### Step b

To a solution of DIPA (42.46 g, 419.64 mmol) in THE (400 mL) was added n-BuLi (29.32 g, 457.79 mmol, 2.5 M in hexane) dropwise in 0.5 h at −78° C. under nitrogen atmosphere. After stirring for 20 min at −78° C., to resulting solution was added a solution of 3,4-dichlorophenyl N,N-diethylcarbamate (100.00 g, 381.49 mmol) in THE (100 mL) dropwise over 20 min at −78° C. After addition, the resulting mixture was stirred for additional 0.5 h at −78° C. under nitrogen atmosphere. To the above mixture was added a solution of 12 (101.67 g, 400.56 mmol) in THF (50 mL) dropwise over 0.5 h at −78° C. The resulting mixture was stirred for additional 2 h at −78° C. The resulting mixture was quenched with saturated aq. Na₂SO₃ (300 mL) at −78° C. and extracted with EA (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (40/1) to afford 3,4-dichloro-2-iodophenyl NN-diethylcarbamate as an off-white solid (117.00 g, 79%): LCMS (ESI) calc'd for C₁₁H₁₂Cl₂INO₂ [M+H]⁺: 388, 390 (3:2), found 388, 390 (3:2); ¹H NMR (400 MHz, CDCl₃) δ 7.48 (d, J=8.8 Hz, 1H), 7.08 (d, J=8.7 Hz, 1H), 3.55 (q, J=7.1 Hz, 2H), 3.42 (q, J=7.1 Hz, 2H), 1.35 (t, J=7.1 Hz, 3H), 1.25 (t, J=7.1 Hz, 3H).

### Step c

To a stirred solution of 3,4-dichloro-2-iodophenyl N,N-diethylcarbamate (65.80 g, 169.58 mmol) in MeOH (100 mL) was added a solution of NaOH (67.82 g, 1695.75 mmol) in H₂O (200 mL) at 0° C. The resulting mixture was allowed to warm to 50° C. and stirred for 10 h. The pH value of the solution was adjusted to 6-7 with aq. HCl (1 N). The reaction was diluted with water (400 mL) at room temperature and extracted with EA (3×400 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (40/1) to afford Intermediate 5 (3,4-dichloro-2-iodophenol) as a yellow oil (47.00 g, 96%): ¹H NMR (400 MHz, CDCl₃) δ 7.36 (d, J=8.8 Hz, 1H), 6.90 (d, J=8.8 Hz, 1H), 6.09 (s, 1H).

### Example 5. Intermediate 6 ((2-(3,4-dichloro-2-iodophenoxymethoxy)ethyl)trimethylsilane)

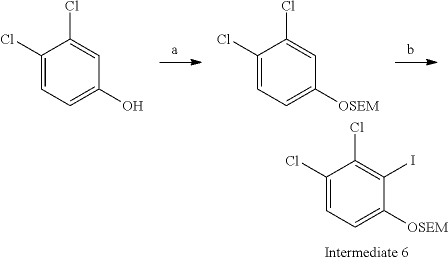

### Step a

To a stirred mixture of 3,4-dichlorophenol (200 g, 1.23 mol) and K₂CO₃ (339 g, 2.45 mol) in DMF (1 L) was added SEMCl (245 g, 1.47 mol) dropwise at 0° C. The reaction mixture was stirred at room temperature for 16 h, diluted with water (1 L) and extracted with EA (3×1 L). The combined organic layers were washed with brine (3×1 L) and dried over anhydrous Na₂SO₄. After filtration, the filtrate was concentrated under reduced pressure. The residue was purified by silica gel column chromatography, eluting with PE/EA (100/1) to afford (2-(3,4-dichlorophenoxymethoxy)ethyl)trimethylsilane) (250 g, 69%): ¹H NMR (400 MHz, CDCl₃) δ 7.35 (d, J=8.8 Hz, 1H), 7.19 (d, J=2.8 Hz, 1H), 6.92 (dd, J=8.9, 2.8 Hz, 1H), 5.21 (s, 2H), 3.80-3.72 (m, 2H), 0.94-0.83 (m, 2H), 0.03 (s, 9H).

### Step b

To a stirred solution of (2-(3,4-dichlorophenoxymethoxy)ethyl)trimethylsilane (22.0 g, 75.0 mmol) in THE (250 mL) was added n-BuLi (60 mL, 0.15 mol, 2.5 M in hexane) dropwise over 30 min at −78° C. under nitrogen atmosphere. After stirring for 1 h, I₂ (19.0 g, 75.0 mmol) was added over 20 minutes. The resulting solution was stirred for 1 h, quenched with saturated aq. NH₄Cl (200 mL) at 0° C., and extracted with EA (3×200 mL). The combined organic layers were washed with brine (3×200 mL) and dried over anhydrous Na₂SO₄. After filtration, the filtrate was concentrated under reduced pressure. The residue was purified by silica gel column chromatography, eluting with PE/EA (12/1) to afford Intermediate 6 ((2-(3,4-dichloro-2-iodophenoxymethoxy)ethyl)trimethylsilane) as a yellow solid (20.0 g, 63%): ¹H NMR (400 MHz, CDCl₃) δ 7.42 (d, J=8.9 Hz, 1H), 6.98 (d, J=8.9 Hz, 1H), 5.31 (s, 2H), 3.84-3.78 (m, 2H), 1.00-0.94 (m, 2H), 0.03 (s, 9H).

### Example 6. Intermediate 7 (ethyl (2R)-2-[(tert-butoxycarbonyl)amino]-5-(2,3-dichloro-6-[[2-(trimethylsilyl)ethoxy]methoxy]phenyl)-5-oxopentanoate) and Intermediate 8 (ethyl (2S)-2-[(tert-butoxycarbonyl)amino]-5-(2,3-dichloro-6-[[2-(trimethylsilyl)ethoxy]methoxy]phenyl)-5-oxopentanoate)

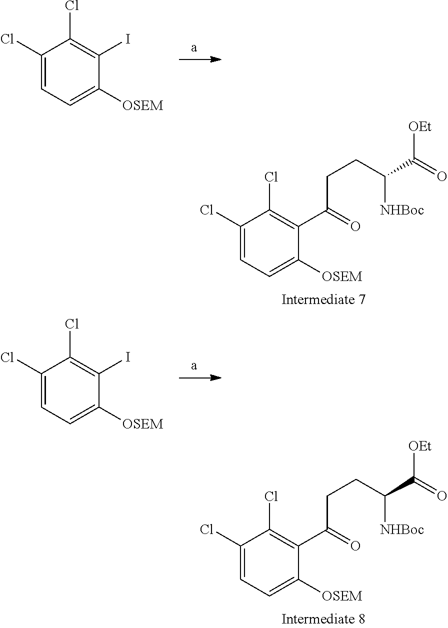

### Step a

To a stirred solution of [2-(3,4-dichloro-2-iodophenoxymethoxy)ethyl]trimethylsilane (Intermediate 6, Example 5) (2.10 g, 5.01 mmol) in THE (15 mL) was added n-BuLi (1.90 mL, 4.75 mmol, 2.5 M in hexane) dropwise at −78° C. under nitrogen atmosphere. The reaction mixture was stirred for 30 minutes and 1-tert-butyl 2-ethyl (2R)-5-oxopyrrolidine-1,2-dicarboxylate (1.00 g, 3.89 mmol) was added. The resulting solution was stirred for 1 h, quenched with saturated aq. NH₄Cl (30 mL), and extracted with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluting with PE/EA (3/1) to afford Intermediate 7 (ethyl (2R)-2-[(tert-butoxycarbonyl)amino]-5-(2,3-dichloro-6-[[2-(trimethylsilyl)ethoxy]methoxy]phenyl)-5-oxopentanoate) as a light-yellow oil (0.350 g, 16%): LCMS (ESI) calc'd for C₂₄H₃₇Cl₂NO₇Si [M+Na]⁺: 572, 574 (3:2) found 572, 574 (3:2); ¹H NMR (300 MHz, CDCl₃) δ 7.40 (d, J=9.0 Hz, 1H), 7.10 (d, J=9.0 Hz, 1H), 5.35-5.32 (m, 1H), 5.21 (s, 2H), 5.17-5.07 (m, 1H), 4.39-4.29 (m, 1H), 4.28-4.18 (m, 2H), 3.78-3.68 (m, 2H), 2.96-2.81 (m, 1H), 2.45-2.25 (m, 1H), 2.18-2.02 (m, 1H), 1.46 (s, 9H), 1.31 (t, J=7.3, 1.6 Hz, 3H), 1.00-0.89 (m, 2H), 0.03 (s, 9H). Intermediate 8 ((ethyl (2S)-2-[(tert-butoxycarbonyl)amino]-5-(2,3-dichloro-6-[[2-(trimethylsilyl)ethoxy]methoxy]phenyl)-5-oxopentanoate) was prepared in the same way as Intermediate 7: LCMS (ESI) calc'd for C₂₄H₃₇Cl₂NO₇Si [M+Na]⁺: 572, 574 (3:2) found 572, 574 (3:2); ¹H NMR (300 MHz, CDCl₃) δ 7.41 (d, J=9.0 Hz, 1H), 7.11 (d, J=9.0 Hz, 1H), 5.21 (s, 2H), 4.40-4.30 (m, 1H), 4.24 (q, J=7.1 Hz, 2H), 3.80-3.68 (m, 2H), 2.95-2.82 (m, 2H), 2.42-2.23 (m, 1H), 2.18-2.09 (m, 1H), 1.46 (s, 9H), 1.36-1.22 (m, 3H), 0.95 (t, J=8.4 Hz, 2H), 0.03 (s, 9H).

### Example 7. Intermediate 9 ((S)—N-[[2,3-dichloro-6-(methoxymethoxy)phenyl]methylidene]-2-methylpropane-2-sulfinamide

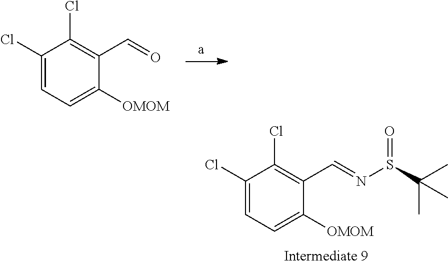

### Step a

To a stirred solution of 2,3-dichloro-6-(methoxymethoxy)benzaldehyde (2.00 g, 8.51 mmol) and (S)-2-methylpropane-2-sulfinamide (1.55 g, 12.8 mmol) in THE (20 mL) was added Ti(OEt)₄ (5.82 g, 25.52 mmol) at room temperature under nitrogen atmosphere. The resulting solution was stirred for 16 h, quenched with saturated aq. NaHCO₃(50 mL), and extracted with EA (3×50 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (3/1) to afford Intermediate 9 ((S)—N-[[2,3-dichloro-6-(methoxymethoxy)phenyl]methylidene]-2-methylpropane-2-sulfinamide) as a light-yellow oil (2.60 g, 81%): LCMS (ESI) calc'd for C₁₃H₁₇Cl₂NO₃S [M+H]⁺: 338, 340 (3:2) found 338, 340 (3:2); ¹H NMR (300 MHz, CDCl₃) δ 8.91 (s, 1H), 7.49 (d, J=9.0 Hz, 1H), 7.13 (d, J=9.0 Hz, 1H), 5.23 (s, 2H), 3.48 (s, 3H), 1.31 (s, 9H).

### Example 8. Intermediate 10 (ethyl (5R)-5-[2,3-dichloro-6-(methoxymethoxy)phenyl]-1-(4-methylbenzenesulfonyl)pyrrolidine-3-carboxylate isomer 1) and Intermediate 11 (ethyl (5R)-5-[2,3-dichloro-6-(methoxymethoxy)phenyl]-1-(4-methylbenzenesulfonyl)pyrrolidine-3-carboxylate isomer 2)

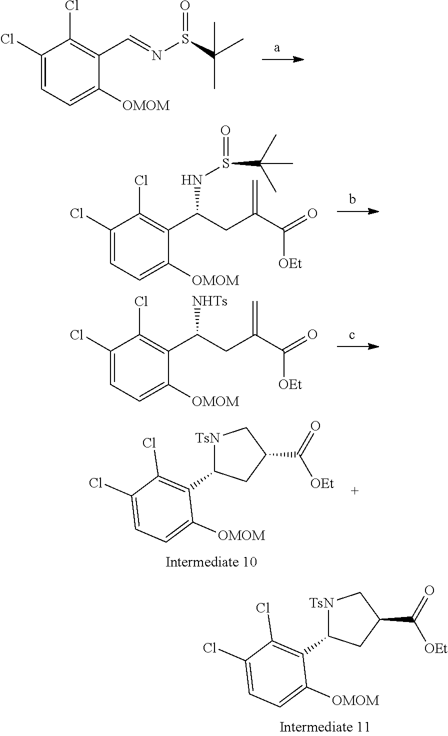

### Step a

To a stirred mixture of (S)—N-[[2,3-dichloro-6-(methoxymethoxy)phenyl]methylidene]-2-methylpropane-2-sulfinamide (Intermediate 9, Example 7) (1.00 g, 2.96 mmol) and ethyl 2-(bromomethyl)prop-2-enoate (1.71 g, 8.87 mmol) in NH₄Cl (8 mL) and THE (2 mL) was added Zn (0.580 g, 8.87 mmol) in portions at room temperature. The reaction mixture was stirred for 5 minutes, diluted with water (20 mL), and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 45% ACN in water (plus 10 mM NH₄HCO₃), to afford ethyl (4R)-4-[2,3-dichloro-6-(methoxymethoxy)phenyl]-2-methylidene-4-[[(S)-2-methylpropane-2-sulfinyl]amino]butanoate as a light-yellow oil (1.40 g, 94%): LCMS (ESI) calc'd for C₁₉H₂₇Cl₂NO₅S [M+H]⁺: 452, 454 (3:2) found 452, 454 (3:2); ¹H NMR (300 MHz, CD₃OD) δ 7.41-7.36 (m, 1H), 7.19-7.13 (m, 1H), 6.08 (d, J=1.4 Hz, 1H), 5.47 (d, J=9.9 Hz, 1H), 5.38-5.31 (m, 2H), 5.29-5.11 (m, 1H), 4.22-4.09 (m, 2H), 3.56 (s, 3H), 3.20-3.01 (m, 2H), 1.29 (q, J=6.8 Hz, 3H), 1.12 (s, 9H).

### Step b

To a stirred solution of ethyl (4R)-4-[2,3-dichloro-6-(methoxymethoxy)phenyl]-2-methylidene-4-[[(S)-2-methylpropane-2-sulfinyl]amino]butanoate (1.56 g, 3.45 mmol) in MeOH (10.50 mL) was added aq. HCl (2 M, 3.50 mL) at room temperature. The reaction mixture was stirred for 1 h, basified with saturated aq. NaHCO₃ to pH 8, and extracted with EA (3×20 mL). The combined organic layers were washed with brine (2×20 mL) and dried over anhydrous Na₂SO₄. After filtration, the filtrate was concentrated under reduced pressure. To a solution of the residue in DCM (10 mL) were added TsCl (0.660 g, 3.45 mmol), DMAP (0.110 g, 0.86 mmol), and TEA (1.00 mL, 7.18 mmol) at room temperature. The resulting solution was stirred for 2 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by silica gel column chromatography, eluting with PE/EA (4/1) to afford ethyl (4R)-4-[2,3-dichloro-6-(methoxymethoxy)phenyl]-4-(4-methylbenzenesulfonamido)-2-methylidenebutanoate as a light-yellow solid (1.10 g, 76%): LCMS (ESI) calc'd for C₂₂H₂₅Cl₂NO₆S [M+Na]⁺: 524, 526 (3:2) found 524, 526 (3:2); ¹H NMR (400 MHz, CDCl₃) δ 7.57-7.51 (m, 2H), 7.14 (d, J=9.0 Hz, 1H), 7.07-7.02 (m, 2H), 6.82 (d, J=9.1 Hz, 1H), 6.22 (d, J=1.2 Hz, 1H), 5.94 (d, J=10.9 Hz, 1H), 5.60 (q, J=1.1 Hz, 1H), 5.30-5.25 (m, 1H), 5.25-5.18 (m, 2H), 4.18 (q, J=7.1 Hz, 2H), 3.57 (s, 3H), 3.00-2.90 (m, 1H), 2.73-2.64 (m, 1H), 2.31 (s, 3H), 1.30 (t, J=7.1 Hz, 3H).

### Step c

To a stirred solution of ethyl (4R)-4-[2,3-dichloro-6-(methoxymethoxy)phenyl]-4-(4-methylbenzenesulfonamido)-2-methylidenebutanoate (0.600 g, 1.19 mmol) in DMF (6 mL) was added NaH (53.0 mg, 0.12 mmol, 60% in oil) at room temperature. The reaction mixture was stirred at 110° C. for 16 h. The resulting mixture was quenched with water (20 mL) at room temperature and extracted with EA (3×20 mL). The combined organic layers were washed with brine (2×20 mL) and dried over anhydrous Na₂SO₄. After filtration, the filtrate was concentrated under reduced pressure. The residue was purified by Prep-TLC (PE/EA 3/1) to afford Intermediate 10 (ethyl (5R)-5-[2,3-dichloro-6-(methoxymethoxy)phenyl]-1-(4-methylbenzenesulfonyl)pyrrolidine-3-carboxylate isomer 1) as a light-yellow solid (0.150 g, 24%): LCMS (ESI) calc'd for C₂₂H₂₅Cl₂NO₆S [M+H]⁺: 502, 504 (3:2) found 502, 504 (3:2); H NMR (300 MHz, CDCl₃) δ 7.73-7.61 (m, 2H), 7.37-7.28 (m, 3H), 7.04-6.91 (m, 1H), 5.52-5.38 (m, 1H), 5.22-5.02 (m, 2H), 4.16 (q, J=7.1 Hz, 2H), 4.14-4.01 (m, 1H), 3.78 (t, J=11.2 Hz, 1H), 3.59-3.46 (m, 4H), 2.79-2.60 (m, 1H), 2.50-2.38 (m, 4H), 1.26 (t, J=7.1 Hz, 3H) and Intermediate 11 (ethyl (5R)-5-[2,3-dichloro-6-(methoxymethoxy)phenyl]-1-(4-methylbenzenesulfonyl)pyrrolidine-3-carboxylate isomer 2) as a light-yellow solid (0.29 g, 46%): LCMS (ESI) calc'd for C₂₂H₂₅Cl₂NO₆S [M+H]⁺: 502, 504 (3:2) found 502, 504 (3:2); H NMR (300 MHz, CDCl₃) δ 7.65 (d, J=7.8 Hz, 2H), 7.32 (d, J=8.9 Hz, 1H), 7.25 (d, J=7.8H, 2H), 7.02 (d, J=9.0 Hz, 1H), 5.60-5.47 (m, 1H), 5.27-5.05 (m, 2H), 4.00-3.85 (m, 4H), 3.55 (s, 3H), 3.26-3.15 (m, 1H), 2.63-2.47 (m, 1H), 2.43 (s, 3H), 2.39-2.24 (m, 1H), 1.22 (t, J=7.1 Hz, 3H).

### Example 9. Intermediate 12 (1-tert-butyl 3-ethyl 6-(2,3-dichloro-6-methoxyphenyl)piperidine-1,3-dicarboxylate)

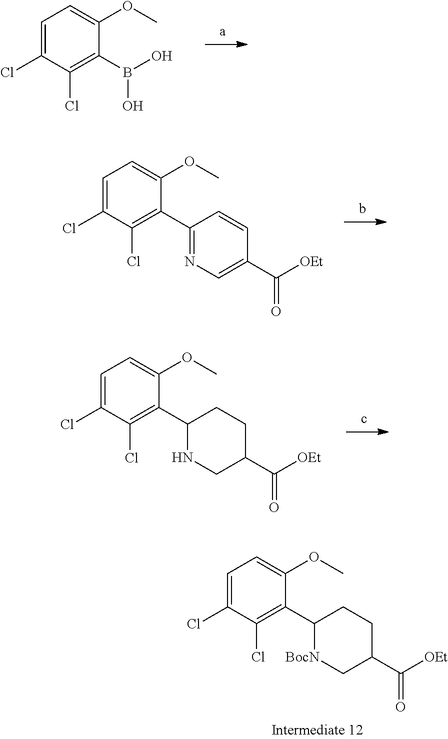 [SMILES: COc1ccc(Cl)c(Cl)c1B(O)O ; CCOC(=O)c1ccc(-c2c(OC)ccc(Cl)c2Cl)nc1 ; CCOC(=O)C1CCC(c2c(OC)ccc(Cl)c2Cl)NC1 ; CCOC(=O)C1CCC(c2c(OC)ccc(Cl)c2Cl)N(C(=O)OC(C)(C)C)C1]

### Step a

To a stirred solution of 2,3-dichloro-6-methoxyphenylboronic acid (1.00 g, 4.53 mmol) and methyl 6-bromopyridine-3-carboxylate (0.980 g, 4.54 mmol) in toluene (32 mL) and EtOH (8 mL) were added K₂CO₃ (1.88 g, 13.6 mmol) and Pd(PPh₃)₄ (0.520 g, 0.45 mmol) at room temperature under nitrogen atmosphere. The reaction mixture was stirred at 90° C. for 2 h, diluted with water (50 mL), and extracted with EA (3×30 mL). The combined organic layers were washed with brine (3×5 mL) and dried over anhydrous Na₂SO₄. After filtration, the filtrate was concentrated under reduced pressure. The residue was purified by reverse phase chromatography, eluting with 50% ACN in water (plus 0.05% TFA) to afford ethyl 6-(2,3-dichloro-6-methoxyphenyl)pyridine-3-carboxylate as a yellow oil (0.600 g, 41%): LCMS (ESI) calc'd for C₁₅H₁₃Cl₂NO₃ [M+H]⁺: 326, 328 (3:2) found 326, 328 (3:2); ¹H NMR (300 MHz, CDCl₃) δ 9.51-9.43 (m, 1H), 8.66 (dd, J=8.2, 2.1 Hz, 1H), 7.64-7.59 (m, 1H), 7.35 (d, J=7.5 Hz, 1H), 6.94 (d, J=9.0 Hz, 1H), 4.05 (s, 2H), 3.76 (s, 3H), 1.32-1.24 (m, 3H).

### Step b

To a stirred solution of ethyl 6-(2,3-dichloro-6-methoxyphenyl)pyridine-3-carboxylate (0.600 g, 1.84 mmol) in AcOH (10 mL) was added PtO₂ (42.0 mg, 0.18 mmol) at room temperature. The reaction mixture was stirred for 16 h under hydrogen atmosphere (1.5 atm). The resulting mixture was filtered and the filter cake washed with MeOH (3×5 mL). The filtrate was concentrated under reduced pressure. The residue was purified by reverse phase chromatography, eluting with 25% ACN in water (plus 0.05% TFA) to afford ethyl 6-(2,3-dichloro-6-methoxyphenyl)piperidine-3-carboxylate as a yellow oil (0.200 g, 33%): LCMS (ESI) calc'd for C₁₅H₁₉Cl₂NO₃ [M+H]⁺: 332, 334 (3:2) found 332, 334 (3:2); ¹H NMR (400 MHz, CDCl₃) δ 7.47 (d, J=9.1 Hz, 1H), 6.85 (d, J=9.1 Hz, 1H), 5.01 (t, J=10.6 Hz, 1H), 4.40-4.20 (m, 2H), 3.98 (s, 3H), 3.80-3.70 (m, 1H), 3.53-3.47 (m, 1H), 3.07-2.99 (m, 1H), 2.44-2.24 (m, 1H), 2.24-2.04 (m, 2H), 1.97-1.83 (m, 1H), 1.36 (t, J=7.1 Hz, 3H).

### Step c

To a stirred solution of ethyl 6-(2,3-dichloro-6-methoxyphenyl)piperidine-3-carboxylate (0.250 g, 0.75 mmol) and TEA (0.150 g, 1.51 mmol) in DCM (2 mL) was added Boc₂O (0.160 g, 0.75 mmol) at room temperature. The reaction mixture was stirred for 1 h, diluted with water (20 mL), and extracted with EA (3×30 mL). The combined organic layers were washed with brine (3×5 mL) and dried over anhydrous Na₂SO₄. After filtration, the filtrate was concentrated under reduced pressure. The residue was purified by reverse phase chromatography, eluting with 60% ACN in water (plus 0.05% TFA) to afford Intermediate 12 (1-tert-butyl 3-ethyl 6-(2,3-dichloro-6-methoxyphenyl)piperidine-1,3-dicarboxylate) as a yellow oil (0.270 g, 59%): LCMS (ESI) calc'd for C₂₀H₂₇Cl₂NO₅ [M+H]⁺: 432, 434 (3:2) found 432, 434 (3:2): ¹H NMR (300 MHz, CDCl₃) δ 7.33 (d, J=8.9 Hz, 1H), 6.79 (d, J=8.9 Hz, 1H), 5.26 (dd, J=11.6, 5.0 Hz, 1H), 4.38-4.26 (m, 1H), 4.20 (q, J=7.0 Hz, 2H), 3.83 (d, J=1.4 Hz, 3H), 3.58-3.44 (m, 1H), 3.03-2.87 (m, 1H), 2.21-1.92 (m, 2H), 1.91-1.69 (m, 2H), 1.30 (t, J=7.1 Hz, 3H), 1.20 (d, J=2.9 Hz, 9H).

Examples 10-81 describe the syntheses of representative compounds of Formula I disclosed herein.

### Example 10. Compound 1 (4,5-dichloro-2-(4-methylpiperidin-4-yl)phenol)

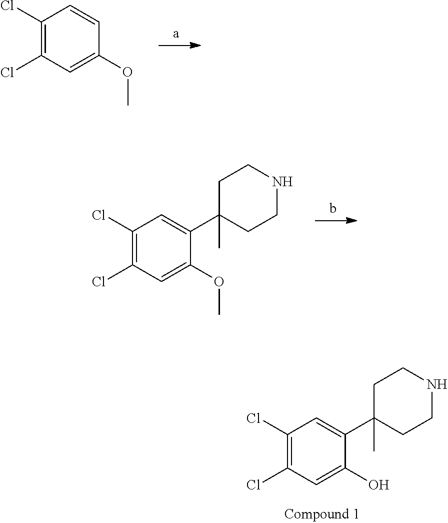

### Step a

To a stirred solution of 1,2-dichloro-4-methoxybenzene (0.30 g, 1.70 mmol) and tert-butyl 4-hydroxy-4-methylpiperidine-1-carboxylate (1.82 g, 8.47 mmol) in DCE (5 mL) was added TfOH (6.36 g, 42.37 mmol) dropwise at room temperature under nitrogen atmosphere. The reaction solution was stirred at room temperature for 24 h. The reaction was quenched with water (50 mL) at room temperature. The reaction mixture was extracted with EA (5×30 mL). The combined organic layers were washed with brine (2×30 mL) and dried over anhydrous Na₂SO₄. After filtration, the filtrate was concentrated under reduced pressure to afford 4-(4,5-dichloro-2-methoxyphenyl)-4-methylpiperidine as a brown solid (0.24 g, crude), which was used in the next step directly without further purification: LCMS (ESI) calc'd for C₁₃H₁₇Cl₂NO [M+H]⁺: 274, 276 (3:2), found 274, 276 (3:2).

### Step b

A solution of 4-(4,5-dichloro-2-methoxyphenyl)-4-methylpiperidine (0.24 g, 0.88 mmol) and BBr₃ (1.76 g, 7.01 mmol) in DCM (0.5 mL) was stirred at room temperature for 2 h. The reaction was quenched with water (1 mL) at 0° C. The resulting mixture was concentrated under reduced pressure. The residue was purified by Prep-HPLC with the following conditions: Column: XBridge C₁₈ OBD Prep Column, 100 Å, 5 μm, 19 mm×250 mm; Mobile Phase A: Water with 10 mmol/L NH₄HCO₃ and 0.1% NH₃.H₂O, Mobile Phase B: ACN; Flow rate: 20 mL/min; Gradient: 30% B to 70% B in 9 min; Detector: UV 254/220 nm; Retention time: 7.54 min. The fractions containing the desired product were collected and concentrated under reduced pressure to afford Compound 1 (4,5-dichloro-2-(4-methylpiperidin-4-yl)phenol) as an off-white solid (100 mg, 50%): LCMS (ESI) calc'd for C₁₂H₁₅Cl₂NO [M+H]⁺: 260, 262 (3:2), found 260,262 (3:2); ¹H NMR (300 MHz, DMSO-d₆) δ 7.19 (s, 1H), 6.96 (s, 1H), 2.82-2.55 (m, 4H), 2.09-1.89 (m, 2H), 1.79-1.57 (m, 2H), 1.26 (s, 3H).

### Example 11. Compound 2 ((2R)-1-[4-(2,3-dichloro-6-hydroxyphenyl)piperidin-1-yl]-2,3-dihydroxypropan-1-one)

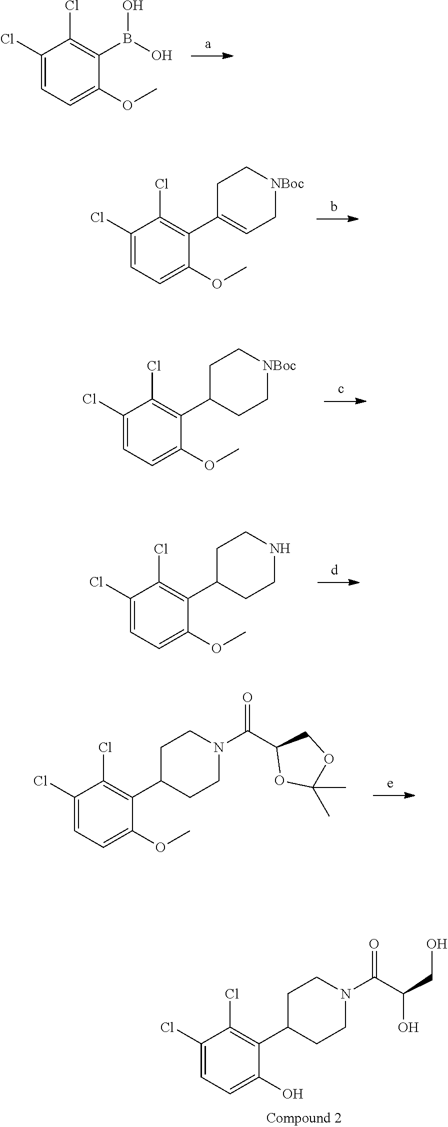 [SMILES: COc1ccc(Cl)c(Cl)c1B(O)O ; COc1ccc(Cl)c(Cl)c1C1=CCN(C(=O)OC(C)(C)C)CC1 ; COc1ccc(Cl)c(Cl)c1C1CCN(C(=O)OC(C)(C)C)CC1 ; COc1ccc(Cl)c(Cl)c1C1CCNCC1 ; COc1ccc(Cl)c(Cl)c1C1CCN(C(=O)[C@H]2COC(C)(C)O2)CC1 ; O=C([C@H](O)CO)N1CCC(c2c(O)ccc(Cl)c2Cl)CC1]

### Step a

To a stirred mixture of 2,3-dichloro-6-methoxyphenylboronic acid (Example 78, step a) (0.50 g, 2.26 mmol) and tert-butyl 4-(trifluoromethanesulfonyloxy)-3,6-dihydro-2H-pyridine-1-carboxylate (0.75 g, 2.26 mmol) in dioxane (8 mL) and H₂O (2 mL) were added Na₂CO₃ (0.72 g, 6.79 mmol) and Pd(dppf)Cl₂ CH₂Cl₂ (0.18 g, 0.27 mmol) at room temperature. The resulting mixture was stirred for 2 h at 80° C. under nitrogen atmosphere. After cooling to room temperature, the resulting mixture was diluted with water (30 mL). The resulting mixture was extracted with EA (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with PE/EA (5/1) to afford tert-butyl 4-(2,3-dichloro-6-methoxyphenyl)-3,6-dihydro-2H-pyridine-1-carboxylate as an off-white solid (0.54 g, 63%): LCMS (ESI) calc'd for C₁₇H₂₁Cl₂NO₃ [M+H−56]⁺ 302, 304 (3:2), found 302, 304 (3:2); ¹H NMR (400 MHz, CD₃OD) δ 7.41 (d, J=8.9 Hz, 1H), 6.96 (d, J=8.9 Hz, 1H), 5.58-5.52 (m, 1H), 4.09-3.99 (m, 2H), 3.81 (s, 3H), 3.71-3.60 (m, 2H), 2.37-2.17 (m, 2H), 1.52 (s, 9H).

### Step b

To a stirred solution of tert-butyl 4-(2,3-dichloro-6-methoxyphenyl)-3,6-dihydro-2H-pyridine-1-carboxylate (0.50 g, 1.40 mmol) and PtO₂ (0.10 g, 0.45 mmol) in MeOH (10 mL) was added HCl (6 N, 1 mL) at room temperature. The resulting mixture was degassed with hydrogen three times and stirred for 2 h at room temperature under hydrogen atmosphere (1.5 atm). The reaction mixture was filtered and the filtrate was concentrated under reduced pressure to afford tert-butyl 4-(2,3-dichloro-6-methoxyphenyl)piperidine-1-carboxylate as a yellow oil (0.50 g, crude), which was used in the next step directly without further purification: LCMS (ESI) calc'd for C₁₇H₂₃Cl₂NO₃ [M+H−56]⁺: 304, 306 (3:2), found 304, 306 (3:2); ¹H NMR (400 MHz, CD₃OD) δ 7.37 (d, J=9.0 Hz, 1H), 6.95 (d, J=9.0 Hz, 1H), 4.24-4.15 (m, 2H), 3.84 (s, 3H), 3.68-3.56 (m, 1H), 3.56-3.45 (m, 1H), 3.20-3.05 (m, 1H), 2.95-2.76 (m, 2H), 2.45-2.24 (m, 2H), 1.51 (s, 9H).

### Step c

To a stirred solution of tert-butyl 4-(2,3-dichloro-6-methoxyphenyl)piperidine-1-carboxylate (0.50 g, 1.39 mmol) in DCM (4 mL) was added TFA (1 mL) at room temperature. The resulting solution was stirred for 1 h at room temperature. The mixture was basified to pH 8 with saturated aq. NaHCO₃. The resulting mixture was extracted with EA (3×20 mL). The combined organic layers were washed with brine (2×20 mL) and dried over anhydrous Na₂SO₄. After filtration, the filtrate was concentrated under reduced pressure to afford 4-(2,3-dichloro-6-methoxyphenyl)piperidine as a yellow oil (0.40 g, crude), which was used in the next step directly without further purification: LCMS (ESI) calc'd for C₁₂H₁₅Cl₂NO [M+H]⁺ 260, 262 (3:2), found 260, 262 (3:2).

### Step d

To a stirred mixture of (4R)-2,2-dimethyl-1,3-dioxolane-4-carboxylic acid (0.34 g, 2.31 mmol) and HATU (0.88 g, 2.31 mmol) in DMF (7 mL) were added 4-(2,3-dichloro-6-methoxyphenyl)piperidine (0.40 g, 1.54 mmol) and Et₃N (0.47 g, 4.61 mmol) at room temperature. The resulting mixture was stirred for 1 h at room temperature. The resulting mixture was diluted with water (300 mL). The resulting mixture was extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluted with 50% ACN in water (plus 0.05% TFA) to afford 4-(2,3-dichloro-6-methoxyphenyl)-1-[(4R)-2,2-dimethyl-1,3-dioxolane-4-carbonyl]piperidine as a light yellow oil (0.43 g, 79% overall three steps): LCMS (ESI) calc'd for C₁₈H₂₃Cl₂NO₄ [M+1]⁺: 388, 390 (3:2), found 388, 390 (3:2); ¹H NMR (400 MHz, CD₃OD) δ 7.38 (d, J=9.0 Hz, 1H), 6.96 (d, J=9.0 Hz, 1H), 5.00-4.88 (m, 1H), 4.68-4.59 (m, 1H), 4.40-4.34 (m, 1H), 4.30-4.17 (m, 2H), 3.83 (d, J=5.6 Hz, 3H), 3.80-3.70 (m, 1H), 3.26-3.09 (m, 1H), 2.81-2.68 (m, 1H), 2.53-2.26 (m, 2H), 1.69-1.54 (m, 2H), 1.43 (d, J=6.4 Hz, 6H).

### Step e

To a stirred mixture of 4-(2,3-dichloro-6-methoxyphenyl)-1-[(4R)-2,2-dimethyl-1,3-dioxolane-4-carbonyl]piperidine (0.43 g, 1.11 mmol) in DCM (3 mL) was added BBr₃ (1.66 g, 6.63 mmol) dropwise at 0° C. The resulting solution was stirred for 30 min at 0° C. The reaction was quenched with saturated aq. NH₄Cl at 0° C. The resulting mixture was concentrated under reduced pressure. The residue was purified by Prep-HPLC with the following conditions: Column: Xselect CSH OBD Column 30×150 mm 5 m; Mobile Phase A: Water (plus 0.05% TFA), Mobile Phase B: ACN; Flow rate: 60 mL/min; Gradient: 22% B to 38% B in 7 min; Detector: UV 254/220 nm; Retention time: 8.25 min. The fractions containing the desired product were collected and concentrated under reduced pressure Compound 2 ((2R)-1-[4-(2,3-dichloro-6-hydroxyphenyl)piperidin-1-yl]-2,3-dihydroxypropan-1-one) as an off-white solid (185.9 mg, 50%). LCMS (ESI) calc'd for C₁₄H₁₇Cl₂NO₄ [M+1]⁺: 334, 346 (3:2), found 334, 346 (3:2); ¹H NMR (400 MHz, CD₃OD) δ 7.19 (d, J=8.8 Hz, 1H), 6.71 (d, J=8.8 Hz, 1H), 4.67 (d, J=13.1 Hz, 1H), 4.62-4.54 (m, 1H), 4.24-4.13 (m, 1H), 3.81-3.60 (m, 3H), 3.20 (t, J=13.0 Hz, 1H), 2.82-2.70 (m, 1H), 2.63-2.40 (m, 2H), 1.70-1.55 (m, 2H).

### Example 12. Compound 3 (4,5-dichloro-2-(piperidin-4-yl)phenol)

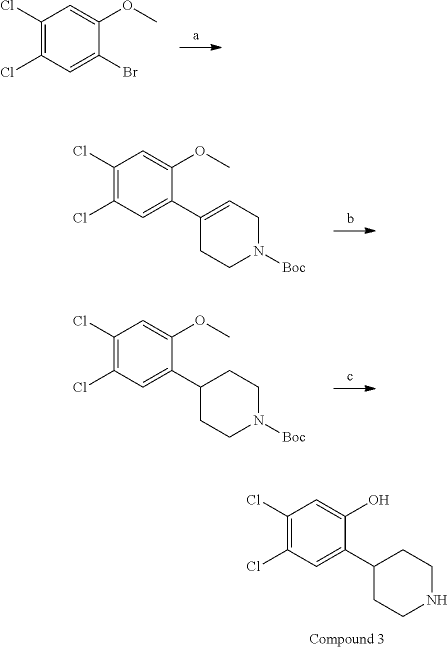 [SMILES: COc1cc(Cl)c(Cl)cc1Br ; COc1cc(Cl)c(Cl)cc1C1=CCN(C(=O)OC(C)(C)C)CC1 ; COc1cc(Cl)c(Cl)cc1C1CCN(C(=O)OC(C)(C)C)CC1 ; Oc1cc(Cl)c(Cl)cc1C1CCNCC1]

### Step a

To a mixture of tert-butyl 4-(tetramethyl-1,3,2-dioxaborolan-2-yl)-1,2,3,6-tetrahydropyridine-1-carboxylate (1.30 g, 4.21 mmol), Intermediate 2 (1.00 g, 3.91 mmol) and K₂CO₃ (1.70 g, 12.30 mmol) in water (5 mL) and 1,4-dioxane (20 mL) was added Pd(dppf)Cl₂ CH₂Cl₂ (54 mg, 0.07 mmol) at room temperature under nitrogen atmosphere. The mixture was warmed to 80° C. and stirred for 2 h under nitrogen atmosphere. After cooling to room temperature, the reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (10/1) to afford to afford tert-butyl 4-(4,5-dichloro-2-methoxyphenyl)-1,2,3,6-tetrahydropyridine-1-carboxylate as a light yellow semi-solid (0.23 g, 80%): LCMS (ESI) calc'd for C₁₇H₂₁Cl₂NO₃ [M+H−15]⁺: 343, 345 (3:2), found: 343, 345 (3:2); ¹H NMR (400 MHz, CDCl₃) δ 7.23 (s, 1H), 6.95 (s, 1H), 5.80 (s, 1H), 4.06 (m, 2H), 3.82 (s, 3H), 3.60 (m, 2H), 2.46 (d, J=4.1 Hz, 2H), 1.52 (s, 9H).

### Step b

To a stirred solution of tert-butyl 4-(4,5-dichloro-2-methoxyphenyl)-1,2,3,6-tetrahydropyridine-1-carboxylate (0.20 g, 0.56 mmol) in MeOH (4 mL) was added PtO₂ (50 mg, 0.22 mmol) at room temperature. The reaction mixture was degassed with hydrogen and stirred at room temperature under hydrogen atmosphere (1.5 atm) for 2 h. The mixture was filtered and the filtrate was concentrated under reduced pressure to afford tert-butyl 4-(4,5-dichloro-2-methoxyphenyl)piperidine-1-carboxylate as a colorless oil (0.16 g, 57%): LCMS (ESI) calc'd for C₁₇H₂₃Cl₂NO₃ [M+H]⁺: 345, 347 (3:2), found 345, 347 (3:2); ¹H NMR (400 MHz, CDCl₃) δ 7.19 (s, 1H), 6.93 (s, 1H), 4.26 (d, J=12.8 Hz, 2H), 3.84 (s, 3H), 3.03 (t, J=12.1, 3.3 Hz, 1H), 2.88-2.76 (m, 2H), 1.82-1.75 (m, 2H), 1.62-1.52 (m, 2H), 1.51 (s, 9H).

### Step c

To a stirred solution of tert-butyl 4-(4,5-dichloro-2-methoxyphenyl)piperidine-1-carboxylate (0.16 g, 0.44 mmol) in DCM (4 mL) was added BBr₃ (0.88 g, 3.53 mmol) at room temperature. The reaction was stirred at room temperature for 10 h. The reaction was quenched with water (1 mL) at room temperature and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 10 mmol/L NH₄HCO₃, Mobile Phase B: ACN; Flow rate: 20 mL/min; Gradient: 20% B to 40% B in 9 min; Detector: UV 254/220 nm; Retention time: 7.58 min. The fractions containing the desired product were collected and concentrated under reduced pressure to afford Compound 3 (4,5-dichloro-2-(piperidin-4-yl)phenol) as a light yellow solid (31.6 mg, 28%): LCMS (ESI) calc'd for C₁₁H₁₃Cl₂NO [M+H]⁺: 246, 248 (3:2), found 246, 248 (3:2); ¹H NMR (300 MHz, DMSO-d₆) δ 7.18 (s, 1H), 6.94 (s, 1H), 3.02-2.96 (m, 2H), 2.92-2.77 (m, 1H), 2.62-2.48 (m, 2H), 1.65-1.57 (m, 2H), 1.43 (m, 2H).

### Example 13. Compound 4 (4,5-dichloro-2-(1-methylpiperidin-4-yl)phenol)

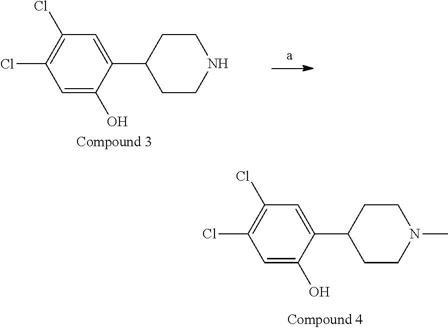 [SMILES: Oc1cc(Cl)c(Cl)cc1C1CCNCC1 ; CN1CCC(c2cc(Cl)c(Cl)cc2O)CC1]

### Step a

To a stirred solution of Compound 3 (Example 12) (4,5-dichloro-2-(piperidin-4-yl)phenol) (0.10 g, 0.41 mmol) and paraformaldehyde (18 mg, 0.60 mmol) in MeOH (2 mL) were added AcOH (24 mg, 0.40 mmol) and NaBH(OAc)₃ (0.26 g, 1.23 mmol) at room temperature. The reaction was stirred for 2 h at room temperature. The reaction was quenched with saturated aq. NH₄Cl (1 mL) and concentrated under reduced pressure. The residue was purified by Prep-HPLC with following conditions: Condition as Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 20 mL/min; Gradient: 30% B to 70% B in 9 min; Detector: UV 254/220 nm; Retention time: 8.11 min. The fractions containing the desired product were collected and concentrated under reduced pressure to afford Compound 4 (4,5-dichloro-2-(1-methylpiperidin-4-yl)phenol) as an off-white solid (50 mg, 47%): LCMS (ESI) calc'd for C₁₂H₁₅Cl₂NO [M+H]⁺: 260, 262 (3:2), found: 260, 262 (3:2); ¹H NMR (400 MHz, DMSO-d₆) δ 10.09 (br, 1H), 7.25 (s, 1H), 6.98 (s, 1H), 2.85 (d, J=11.3 Hz, 2H), 2.75-2.64 (m, 1H), 2.18 (s, 3H), 1.97-1.88 (m, 2H), 1.71-1.53 (m, 4H).

### Example 14. Compound 5 (4,5-dichloro-2-(piperazin-1-yl)phenol)

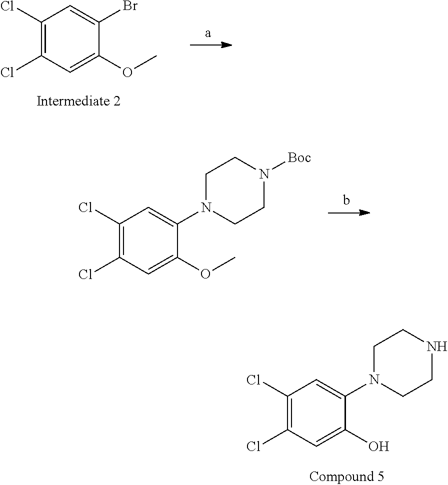 [SMILES: COc1cc(Cl)c(Cl)cc1Br ; COc1cc(Cl)c(Cl)cc1N1CCN(C(=O)OC(C)(C)C)CC1 ; Oc1cc(Cl)c(Cl)cc1N1CCNCC1]

### Step a

To a stirred solution of Intermediate 2 (0.20 g, 0.78 mmol) and tert-butyl piperazine-1-carboxylate (0.22 g, 1.17 mmol) in 1,4-dioxane (8 mL) was added Pd₂(dba)₃ CHCl₃ (81 mg, 0.08 mmol), XantPhos (45 mg, 0.08 mmol), t-BuONa (0.19 g, 2.34 mmol) at room temperature under nitrogen atmosphere. The resulting mixture was stirred at 90° C. for 2 h under nitrogen atmosphere. After cooling to room temperature, the resulting mixture was diluted with co-solvent of EA (30 mL) and water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (4/1) to afford tert-butyl 4-(4,5-dichloro-2-hydroxyphenyl)piperazine-1-carboxylate as a yellow oil (0.17 g, 62%): LCMS (ESI) calc'd for C₁₆H₂₂Cl₂N₂O₃ [M+H]⁺: 361, 363 (3:2), found 361, 363 (3:2); H NMR (300 MHz, CD₃OD) δ 7.03 (s, 1H), 6.96 (s, 1H), 3.82 (s, 3H), 3.55-3.46 (m, 4H), 2.95-2.87 (m, 4H), 1.44 (s, 9H).

### Step b

To a stirred solution of tert-butyl 4-(4,5-dichloro-2-methoxyphenyl)piperazine-1-carboxylate (0.17 g, 0.48 mmol) in DCM (4 mL) was added BBr₃ (0.60 g, 2.41 mmol) at room temperature. The resulting mixture was stirred for 1 h at room temperature. The reaction was quenched with water (2 mL) at room temperature. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 25 mL/min; Gradient: 35% B to 75% B in 6.5 min; Detector: UV 254/220 nm; Retention time: 6.41 min. The fractions containing the desired product were collected and concentrated under reduced pressure to afford Compound 5 (4,5-dichloro-2-(piperazin-1-yl)phenol) as a yellow solid (54 mg, 45%): LCMS (ESI) calc'd for C₁₀H₁₂Cl₂N₂O [M+H]⁺: 247, 249 (3:2), found 247, 249 (3:2); ¹H NMR (300 MHz, CD₃OD) δ 7.04 (s, 1H), 6.92 (s, 1H), 3.37-3.30 (m, 4H), 3.23-3.18 (m, 4H).

The compounds in Table 1A below were prepared in an analogous fashion to that described for Compound 5, starting from Intermediate 2 and the corresponding amines, which were available from commercial sources.

TABLE 1A

| Compound | | | |
|---|---|---|---|
| Number | Structure | Chemical Name | MS: (M + H)⁺ & ¹H MNR |
| 8 | 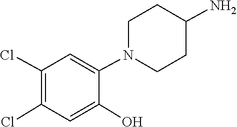 [SMILES: NC1CCN(c2cc(Cl)c(Cl)cc2O)CC1] | 2-(4- aminopiperidin- 1-yl)-4,5- dichlorophenol | [M + H]⁺ 261, 263 (3:2); ¹H NMR (400 MHz, CD₃OD) δ 7.07 (s, 1H), 6.94 (s, 1H), 3.47 (d, J = 12.2 Hz, 2H), 3.29 − 3.18 (m, 1H), 2.72 (t, J = 11.7 Hz, 2H), 2.15 − 2.05 (m, 2H), 1.92 − 1.79 (m, 2H). |
| 23 | 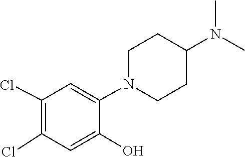 [SMILES: CN(C)C1CCN(c2cc(Cl)c(Cl)cc2O)CC1] | 4,5-dichloro-2-[4- (dimethylamino) piperidin-1- yl]phenol | [M + H]⁺: 289, 291 (3:2); ¹H NMR (300 MHz, CD₃OD) δ 7.01 (s, 1H), 6.87 (s, 1H), 3.47 − 3.30 (m, 2H), 2.63 − 2.47 (m, 2H), 2.39 − 2.24 (m, 7H), 1.96 − 1.86 (m, 2H), 1.76 − 1.60 (m, 2H). |
| 28 | 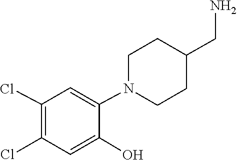 [SMILES: NCC1CCN(c2cc(Cl)c(Cl)cc2O)CC1] | 2-[4- (aminomethyl)pip- eridin-1-yl]-4,5- dichlorophenol | [M + H]⁺: 275, 277 (3:2); ¹H NMR (400 MHz, CD₃OD) δ 7.06 (s, 1H), 6.91 (s, 1H), 3.39 − 3.34 (m, 2H), 2.65 (d, J = 6.3 Hz, 2H), 2.58 (t, J = 11.5 Hz, 2H), 1.85 (dd, J = 12.3, 3.3 Hz, 2H), 1.56 − 1.37 (m, 3H). |
| 25 | 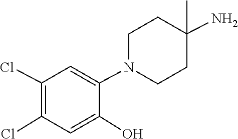 [SMILES: CC1(N)CCN(c2cc(Cl)c(Cl)cc2O)CC1] | 2-(4-amino-4- methylpiperidin- l-yl)-4,5- dichlorophenol | [M + H]⁺: 275, 277 (3:2); ¹H NMR (300 MHz, CD₃OD) δ 7.06 (s, 1H), 6.86 (s, 1H), 3.05 − 2.94 (m, 2H), 2.94 − 2.82 (m, 2H), 1.78 − 1.61 (m, 4H), 1.19 (s, 3H). |
| 24 | 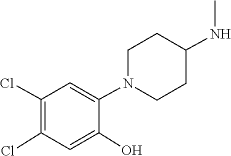 [SMILES: CNC1CCN(c2cc(Cl)c(Cl)cc2O)CC1] | 4,5-dichloro-2-[4- (methylamino)pip- eridin-1- yl]phenol | [M + H]⁺: 275, 277 (3:2); ¹H NMR (300 MHz, CD₃OD) δ 7.01 (s, 1H), 6.86 (s, 1H), 3.35 − 3.30 (m, 2H), 2.64 − 2.45 (m, 3H), 2.40 (s, 3H), 2.01 − 1.92 (m, 2H), 1.63 − 1.46 (m, 2H) |
| 31 | 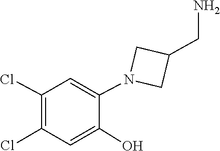 [SMILES: NCC1CN(c2cc(Cl)c(Cl)cc2O)C1] | 2-(3- (aminomethyl)aze- tidin-1-yl)-4,5- dichlorophenol | [M + H]⁺: 247, 249 (3:2); ¹H NMR (300 MHz, CD₃OD) δ 6.76 (s, 1H), 6.48 (s, 1H), 4.06 (t, J = 7.9 Hz, 2H), 3.70 (dd, J = 8.1, 5.1 Hz, 2H), 3.26 (d, J = 7.4 Hz, 2H), 2.92 − 2.81 (m, 1H) |
| 33 | 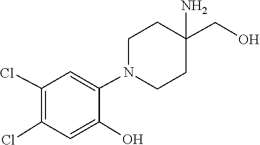 [SMILES: NC1(CO)CCN(c2cc(Cl)c(Cl)cc2O)CC1] | 2-[4-amino-4- (hydroxymethyl) piperidin-1-yl]- 4,5- dichlorophenol | [M + H]⁺: 291, 293 (3:2); ¹H NMR (400 MHz, CD₃OD) δ 7.12 (s, 1H), 6.92 (s, 1H), 3.58 − 3.46 (m, 2H), 3.07 − 2.93 (m, 4H), 1.95 − 1.77 (m, 2H), 1.74 − 1.57 (m, 2H) |
| 37 | 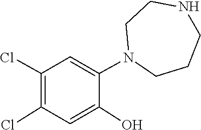 [SMILES: Oc1cc(Cl)c(Cl)cc1N1CCCNCC1] | 4,5-dichloro-2- (1,4-diazepan-1- yl)phenol | [M + H] 261, 263 (3:2); ¹H NMR (300 MHz, CD₃OD) δ 7.05 (s, 1H), 6.92 (s, 1H), 3.51 − 3.46 (m, 2H), 3.46 − 3.39 (m, 4H), 3.31 − 3.25 (m, 2H), 2.25 − 2.15 (m, 2H) |

### Example 15. Compound 7 ((3R,4R)-rel-4-(4,5-dichloro-2-hydroxyphenyl)piperidin-3-ol) and Compound 59 (4-(4,5-dichloro-2-hydroxyphenyl)piperidin-4-ol)

### Step a

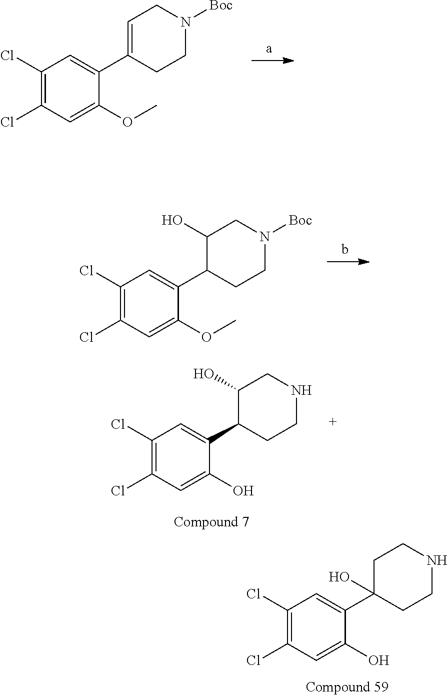

To a stirred solution of tert-butyl 4-(4,5-dichloro-2-methoxyphenyl)-5,6-dihydropyridine-1(2H)-carboxylate (0.50 g, 1.40 mmol) in THE (5 mL) was added BH₃.THF (2.1 mL, 21.94 mmol, 1 M in THF) dropwise at 0° C. under nitrogen atmosphere. The solution was stirred at room temperature for 3 h. After cooling to 0° C., H₂O₂ (3 mL) and aq. NaOH (1 M, 8 mL) were added dropwise. The resulting mixture was allowed to warm to room temperature and stirred for 3 h. After that, the reaction was concentrated under reduced pressure. The residue was purified by silica gel column chromatography, eluted with PE/EA (1/1) to afford tert-butyl 4-(4,5-dichloro-2-methoxyphenyl)-3-hydroxypiperidine-1-carboxylate as a pale yellow solid (0.42 g, 79%): LCMS (ESI) calc'd for C₁₇H₂₃Cl₂NO₄ [M+H−15]⁺: 361, 363 (3:2), found 361, 363 (3:2); ¹H NMR (300 MHz, CDCl₃) δ 7.51 (s, 1H), 6.94 (s, 1H), 4.44-4.29 (m, 1H), 4.23-4.06 (m, 1H), 3.81 (s, 3H), 3.77-3.63 (m, 1H), 3.05-2.92 (m, 1H), 2.79-2.53 (m, 2H), 1.77-1.63 (m, 2H), 1.46 (s, 9H).

### Step b

To a stirred solution of tert-butyl 4-(4,5-dichloro-2-methoxyphenyl)-3-hydroxypiperidine-1-carboxylate (0.40 g, 1.06 mmol) in DCM (3 mL) was added BBr₃ (0.83 g, 6.36 mmol) dropwise at 0° C. The resulting mixture was stirred at room temperature for 1.5 h. The reaction was quenched with water (1 mL) at room temperature and concentrated under reduced pressure. The residue was purified by Prep-HPLC with following conditions: Column: XBridge C₁₈ OBD Prep 100 Å, 5 μm, 19 mm×250 mm; Mobile Phase A: Water with 20 mmol/L NH₄HCO₃; Mobile Phase B: ACN; Flow rate: 20 mL/min; Gradient: 24% B to 25% B in 9 min; Detector: UV 254/220 nm; Retention time: RT₁: 4.90 min; RT₂: 7.54 min.

The faster-eluting isomer Compound 59 (4-(4,5-dichloro-2-hydroxyphenyl)piperidin-4-ol) at 4.90 min was obtained as an off-white solid: LCMS (ESI) calc'd for C₁₁H₁₃Cl₂NO₂ [M+H]⁺: 262, 264 (3:2), found 262, 264 (3:2); ¹H NMR (300 MHz, CD₃OD) δ 7.25 (d, J=1.8 Hz, 1H), 6.78 (d, J=1.4 Hz, 1H), 3.39-3.26 (m, 2H), 3.21-3.08 (m, 2H), 2.34-2.19 (m, 2H), 1.93 (d, J=14.0 Hz, 2H).

The slower-eluting isomer Compound 7 (3R,4R)-rel-4-(4,5-dichloro-2-hydroxyphenyl)piperidin-3-ol at 7.54 min was obtained as an off-white solid: LCMS (ESI) calc'd for C₁₁H₁₃Cl₂NO₂ [M+H]⁺: 262, 264 (3:2), found 262, 264 (3:2); ¹H NMR (300 MHz, CD₃OD) δ 7.24 (s, 1H), 6.86 (s, 1H), 3.79 (td, J=10.3, 4.6 Hz, 1H), 3.25-3.13 (m, 1H), 3.06-2.84 (m, 2H), 2.60 (td, J=12.5, 2.9 Hz, 1H), 2.46 (dd, J=12.0, 10.3 Hz, 1H), 1.88-1.70 (m, 1H), 1.70-1.54 (m, 1H).

### Example 16. Compound 9 (4,5-dichloro-2-(1,2,3,6-tetrahydropyridin-4-yl)phenol)

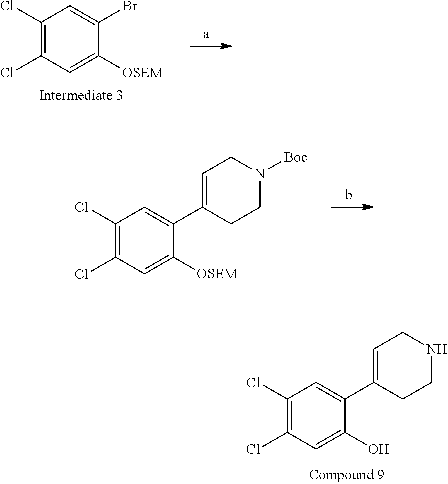

### Step a

To a mixture of Intermediate 3 (0.20 g, 0.54 mmol), Na₂CO₃ (0.17 g, 1.61 mmol) and tert-butyl 4-(tetramethyl-1,3,2-dioxaborolan-2-yl)-1,2,3,6-tetrahydropyridine-1-carboxylate (0.25 g, 0.81 mmol) in 1,4-dioxane (4 mL) and H₂O (1 mL) was added Pd(dppf)Cl₂.CH₂Cl₂ (39 mg, 0.05 mmol) at room temperature under argon atmosphere. The resulting mixture was degassed with argon three times and stirred at 85° C. for 16 h. After cooling to room temperature, the reaction was diluted with water (20 mL) and extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (10/1) afford tert-butyl 4-(4,5-dichloro-2-((2-(trimethylsilyl)ethoxy)methoxy)phenyl)-5,6-dihydropyridine-1(2H)-carboxylate as a light orange oil (0.20 g, 78%): LCMS (ESI) calc'd for C₂₂H₃₃Cl₂NO₄Si [M+H]⁺: 474, 476 (3:2), found 474, 476 (3:2); ¹H NMR (300 MHz, CDCl₃) δ 7.24 (s, 1H), 7.22 (s, 1H), 5.77 (s, 1H), 5.19 (s, 2H), 4.04 (d, J=2.9 Hz, 2H), 3.79-3.68 (m, 2H), 3.58 (t, J=5.6 Hz, 2H), 2.49-2.41 (m, 2H), 1.50 (s, 9H), 1.03-0.89 (m, 2H), 0.01 (s, 9H).

### Step b

To a stirred solution of tert-butyl 4-(4,5-dichloro-2-[[2-(trimethylsilyl)ethoxy]methoxy]phenyl)-1,2,3,6-tetrahydropyridine-1-carboxylate (0.20 g, 0.42 mmol) in DCM (2 mL) was added TFA (2 mL) at 0° C. The resulting solution was stirred for 1 h at room temperature. The reaction was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 20 mL/min; Gradient: 20% B to 55% B in 9 min; Detector: UV 254/220 nm; Retention time: 7.74 min. The fractions containing the desired product were collected and concentrated under reduced pressure to afford Compound 9 (4,5-dichloro-2-(1,2,3,6-tetrahydropyridin-4-yl)phenol) as an off-white solid (15 mg, 14%): LCMS (ESI) calc'd for C₁₁H₁₁Cl₂NO [M+H]⁺: 244, 246 (3:2), found 244, 246 (3:2); ¹H NMR (400 MHz, DMSO-d₆) δ 7.22 (s, 1H), 6.99 (s, 1H), 5.99-5.92 (m, 1H), 3.39-3.29 (m, 2H), 2.87 (t, J=5.6 Hz, 2H), 2.32-2.26 (m, 2H).

The compounds in Table 1B below were prepared in an analogous fashion to that described for Compound 9, starting from Intermediate 3 and the corresponding boronic acids or esters, which were available from commercial sources.

TABLE 1B

| Compound | | | |
|---|---|---|---|
| Number | Structure | Chemical Name | MS: (M + H)⁺ & ¹H MNR |
| 19 | 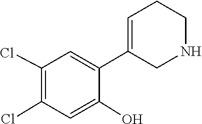 [SMILES: Oc1cc(Cl)c(Cl)cc1C1=CCCNC1] | 4,5-dichloro-2- (1,2,3,6- tetrahydropyridin- 5-yl)phenol | [M + H]⁺: 244, 246 (3:2); ¹H NMR (300 MHz, CD₃OD) δ 7.13 (s, 1H), 6.83 (s, 1H), 5.94 − 5.84 (m, 1H), 3.69-3.61 (m, 2H), 3.03 (t, J = 6.0 Hz, 2H), 2.35 − 2.24 (m, 2H)/ |
| 20 | 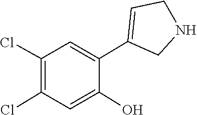 [SMILES: Oc1cc(Cl)c(Cl)cc1C1=CCNC1] | 4,5-dichloro-2- (2,5-dihydro-1H- pyrrol-3-yl)phenol | [M + H] 230, 232 (3:2); ¹H NMR (300 MHz, CD₃OD) δ 7.22 (s, 1H), 6.93 (s, 1H), 6.63 − 6.51 (m, 1H), 4.18 − 4.09 (m, 2H), 3.98 − 3.89 (m, 2H). |
| 17 | 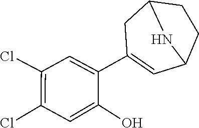 [SMILES: Oc1cc(Cl)c(Cl)cc1C1=CC2CCC(C1)N2] | 2-[8- azabicyclo[3.2.1] oct-2-en-3-yl]-4,5- dichlorophenol | [M + H]⁺: 270, 272 (3:2); ¹H NMR (300 MHz, CD₃OD) δ 7.06 (s, 1H), 6.79 (s, 1H), 6.11 (dt, J = 5.8, 1.7 Hz, 1H), 3.90 − 3.82 (m, 2H), 2.99 − 2.87 (m, 1H), 2.34 − 2.22 (m, 1H), 2.19 − 2.01 (m, 2H), 2.01 − 1.76 (m, 2H). |

### Example 17. Compound 15 (2-[8-azabicyclo[3.2.1]octan-3-yl]-4,5-dichlorophenol isomer 1) and Compound 11 (2-[8-azabicyclo[3.2.1]octan-3-yl]-4,5-dichlorophenol isomer 2)

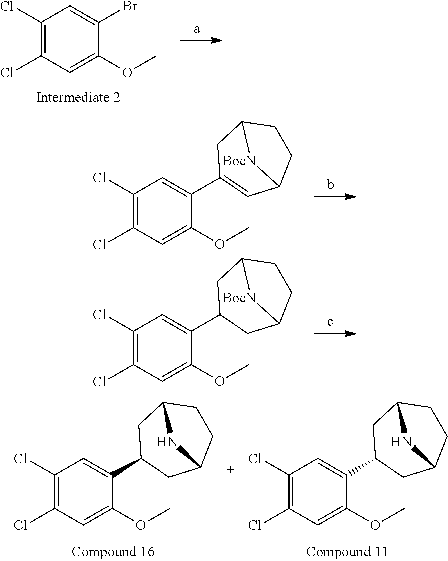

The absolute configurations for Compounds 11 and 15 were arbitrarily assigned.

### Step a

To a stirred solution of Intermediate 2 (0.53 g, 2.07 mmol) and tert-butyl 3-(tetramethyl-1,3,2-dioxaborolan-2-yl)-8-azabicyclo[3.2.1]oct-2-ene-8-carboxylate (0.83 g, 2.48 mmol,) in 1,4-dioxane (5 mL) and water (1 mL) were added Pd(PPh₃)₄ (48 mg, 0.04 mmol) and Na₂CO₃ (0.66 g, 6.23 mmol) at room temperature under nitrogen atmosphere. The resulting mixture was stirred for 2.5 h at 80° C. under nitrogen atmosphere. After cooling to room temperature, the reaction was diluted with EA (50 mL) and water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford tert-butyl 3-(4,5-dichloro-2-methoxyphenyl)-8-azabicyclo[3.2.1]oct-2-ene-8-carboxylate as a yellow oil (0.52 g, 66%): LCMS (ESI) calc'd for C₁₉H₂₃Cl₂NO₃ [M+H]⁺: 384, 386 (3:2), found: 384, 386 (3:2); ¹H NMR (300 MHz, CD₃OD) δ 7.13 (s, 1H), 7.06 (s, 1H), 6.07-6.01 (m, 1H), 4.37 (t, J=5.1 Hz, 1H), 4.34-4.25 (m, 1H), 3.76 (s, 3H), 3.08-2.95 (m, 1H), 2.26-2.05 (m, 2H), 2.04-1.92 (m, 2H), 1.86-1.75 (m, 1H), 1.45 (s, 9H).

### Step b

A degassed mixture of tert-butyl 3-(4,5-dichloro-2-methoxyphenyl)-8-azabicyclo[3.2.1]oct-2-ene-8-carboxylate (0.20 g, 0.52 mmol) and PtO₂ (18 mg, 0.08 mmol) in MeOH (2 mL) was stirred for 20 h at room temperature under hydrogen atmosphere (1.5 atm). The resulting mixture was filtered and the filtrate was concentrated under reduced pressure to afford tert-butyl 3-(4,5-dichloro-2-methoxyphenyl)-8-azabicyclo[3.2.1]octane-8-carboxylate as an off-white solid (0.17 g, 85%): LCMS (ESI) calc'd for C₁₉H₂₅Cl₂NO₃ [M+H]⁺: 386, 388 (3:2), found: 386, 388 (3:2).

### Step c

To a stirred solution of tert-butyl 3-(4,5-dichloro-2-methoxyphenyl)-8-azabicyclo[3.2.1]octane-8-carboxylate (0.17 g, 0.440 mmol) in DCM (2 mL) was added BBr₃ (1.10 g, 4.39 mmol) at room temperature. The resulting mixture was stirred for 1 h at room temperature. The reaction was quenched with saturated aq. Na₂CO₃ (10 mL) at room temperature. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 20 mL/min; Gradient: 20% B to 80% B in 9 min; Detector: UV 254/220 nm; Retention time: RT₁: 8.41 min, RT₂: 8.55 min. The fractions containing the desired product at 8.41 min were collected and concentrated under reduced pressure to afford Compound 15 (2-[8-azabicyclo[3.2.1]octan-3-yl]-4,5-dichlorophenol) isomer 1 as a light yellow solid (18.1 mg, 15%): LCMS (ESI) calc'd for C₁₃H₁₅Cl₂NO [M+H]⁺: 272, 274 (3:2), found: 272, 274 (3:2); ¹H NMR (400 MHz, CD₃OD) δ 7.21 (s, 1H), 6.85 (s, 1H), 3.78-3.71 (m, 2H), 3.55-3.41 (m, 1H), 2.01-1.96 (m, 4H), 1.87-1.70 (m, 4H). Fractions containing the desired product at 8.55 min were collected and concentrated under reduced pressure to afford Compound 11 (2-[8-azabicyclo[3.2.1]octan-3-yl]-4,5-dichlorophenol) isomer 2 as a light yellow solid (20.2 mg, 17%): LCMS (ESI) calc'd for C₁₃H₁₅Cl₂NO [M+H]⁺: 272, 274 (3:2), found: 272, 274 (3:2); ¹H NMR (400 MHz, CD₃OD) δ 7.22 (s, 1H), 6.85 (s, 1H), 3.68-3.63 (m, 2H), 3.28-3.17 (m, 1H), 2.36-2.26 (m, 2H), 1.99-1.90 (m, 2H), 1.80-1.71 (m, 2H), 1.61-1.52 (m, 2H).

### Example 18. Compound 12 (4-chloro-5-methyl-2-(piperidin-4-yl)phenol)

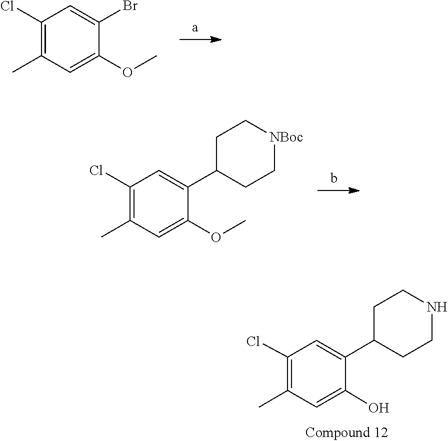

### Step a

To a stirred solution of 1-bromo-5-chloro-2-methoxy-4-methylbenzene (0.20 g, 0.85 mmol), tert-butyl 4-bromopiperidine-1-carboxylate (0.25 g, 0.93 mmol), Ir[DF(CF₃)PPY]₂(DTBPY)PF₆ (10 mg, 0.01 mmol), and 1,1,1,3,3,3-hexamethyl-2-(trimethylsilyl)trisilane (0.21 g, 0.85 mmol) in DME (1 mL) was added Na₂CO₃ (0.18 g, 1.70 mmol) at room temperature under argon atmosphere to afford mixture A. 1,2-dimethoxyethane dichloronickel (0.9 mg, 0.004 mmol) and dtbbpy (1 mg, 0.004 mmol) were dissolved in DME (1 mL) under argon atmosphere to afford mixture B. Then the mixture B was added into the mixture A under argon atmosphere. After that, the resulted mixture was stirred and irradiated with 34W blue LEDs for 2.5 h. The reaction solution was diluted with water (20 mL) and the resulting solution was extracted with EA (3×30 mL). The combined organic layers were washed with brine (3×30 mL) and dried over anhydrous Na₂SO₄. After filtration, the filtrate was concentered under reduced pressured. The residue was purified by the Prep-TLC (PE/EA 8/1) to afford tert-butyl 4-(5-chloro-2-methoxy-4-methylphenyl)piperidine-1-carboxylate as a light yellow oil (63 mg, 22%); LCMS (ESI) calc'd for C₁₈H₂₆ClNO₃ [M+H−56]⁺: 284, 286 (3:1), found 284, 286 (3:1); ¹H NMR (300 MHz, CD₃OD) δ 7.06 (s, 1H), 6.85 (s, 1H), 4.19 (d, J=13.2 Hz, 2H), 3.81 (s, 3H), 3.11-2.97 (m, 1H), 2.92-2.77 (m, 2H), 2.32 (s, 3H), 1.76 (d, J=12.9 Hz, 2H), 1.62-1.52 (m, 1H), 1.49-1.43 (m, 10H).

### Step b

A solution of tert-butyl 4-(5-chloro-2-methoxy-4-methylphenyl)piperidine-1-carboxylate (60 mg, 0.18 mmol) and BBr₃ (0.22 g, 0.88 mmol) in DCM (1 mL) was stirred at room temperature for 1 h. The reaction was quenched with water (1 mL) at room temperature and concentrated under reduced pressure. The residue purified was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 20 mL/min; Gradient: 30% B to 80% B in 9 min; Detector: UV 254/220 nm; Retention time: 7.77 min. The fractions containing the desired product were collected and concentrated under reduced pressure to afford Compound 12 (4-chloro-5-methyl-2-(piperidin-4-yl)phenol) as an off-white solid (21 mg, 53%): LCMS (ESI) calc'd for C₁₂H₁₆ClNO [M+H]⁺: 226, 228 (3:1), found 226, 228 (3:1); ¹H NMR (400 MHz, CD₃OD) δ 7.06 (s, 1H), 6.68 (s, 1H), 3.19 (d, J=12.6 Hz, 2H), 3.03 (t, J=12.2 Hz, 1H), 2.79 (t, J=12.4 Hz, 2H), 2.25 (s, 3H), 1.84 (d, J=13.2 Hz, 2H), 1.71-1.57 (m, 2H).

### Example 19. Compound 13 (4-(4,5-dichloro-2-hydroxyphenyl)piperidine-4-carbonitrile)

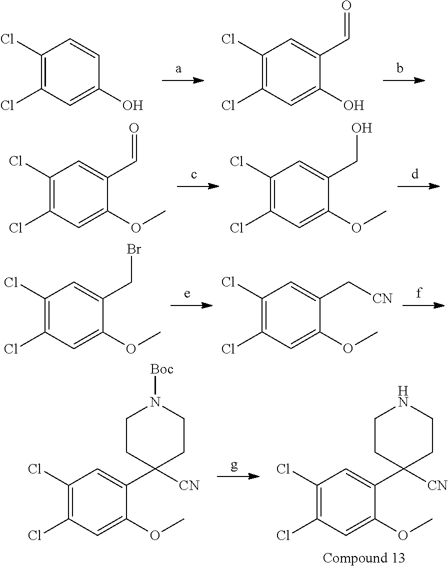

### Step a

To a solution of 3,4-dichlorophenol (10 g, 61.35 mmol) in methylsulfonic acid (70 mL) was added hexamethylenetetramine (9.46 g, 67.48 mmol) in small portions. The mixture was then heated to 110° C. and stirred for 30 min. After cooling to room temperature, the reaction was poured into iced-water (500 mL). The mixture was extracted with DCM (3×100 mL) and the combined organic layer was washed with brine (2×80 mL) and dried over anhydrous Na₂SO₄ and filtered. The filtrate was concentrated under reduced pressure. The residue was purified by silica gel column chromatography, eluted with PE/DCM (10/1) to afford 4,5-dichloro-2-hydroxybenzaldehyde as a light yellow solid (1.8 g, 15%): ¹H NMR (300 MHz, CDCl₃) δ 10.96 (s, 1H), 9.83 (s, 1H), 7.64 (s, 1H), 7.15 (s, 1H).

### Step b

To a stirred mixture of 4,5-dichloro-2-hydroxybenzaldehyde (2.00 g, 10.47 mmol) and K₂CO₃ (2.90 g, 20.94 mmol) in DMF (10 mL) was added MeI (2.20 g, 15.71 mmol) dropwise at 0° C. The reaction mixture was allowed to warm to room temperature and stirred for 2 h. The resulting mixture was diluted with water (30 mL) and extracted with EA (3×50 mL). The combined organic layer was dried over anhydrous Na₂SO₄. After filtration, the filtrate was concentrated under reduced pressure. The residue was purified by silica gel column chromatography, eluted with PE/EA (5/1) to afford 4,5-dichloro-2-methoxybenzaldehyde as a light yellow solid (2.00 g, 76%): ¹H NMR (300 MHz, CDCl₃) δ 10.32 (s, 1H), 7.86 (s, 1H), 7.09 (s, 1H), 3.92 (s, 3H).

### Step c

To a stirred solution of 4,5-dichloro-2-methoxybenzaldehyde (0.50 g, 2.44 mmol) in EtOH (40 mL) and THE (5 mL) was added NaBH₄ (0.20 g, 5.43 mmol) at room temperature under nitrogen atmosphere. The reaction solution was stirred at room temperature for 1 h. The resulting solution was quenched with water (50 mL) and extracted with EA (3×80 mL). The combined organic layers were washed with brine (3×80 mL) and dried over anhydrous Na₂SO₄. After filtration, the filtrate was concentrated under reduced pressure to afford (4,5-dichloro-2-methoxyphenyl)methanol as a light-yellow solid (0.50 g, crude), which was used in next step directly without further purification.

### Step d

To a stirred solution of (4,5-dichloro-2-methoxyphenyl)methanol (0.50 g, 2.41 mmol) in CH₂Cl₂ (5 mL) was added PBr₃ (1.30 g, 4.83 mmol) at room temperature. The reaction solution was stirred for 1 h at room temperature. The resulting solution was quenched with water (50 mL) and extracted with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (4/1) to afford 1-(bromomethyl)-4,5-dichloro-2-methoxybenzene as a colorless oil (0.35 g, 48%): ¹H NMR (400 MHz, CDCl₃) δ 7.37 (s, 1H), 6.93 (s, 1H), 4.42 (s, 2H), 3.86 (s, 3H).

### Step e

To a stirred solution of 1-(bromomethyl)-4,5-dichloro-2-methoxybenzene (2.50 g, 9.26 mmol) in EtOH (30 mL) was added KCN (1.20 g, 18.43 mmol) at room temperature. The resulting mixture was stirred for 5 h at 90° C. The reaction mixture was quenched with saturate aq. FeSO₄ (100 mL) at room temperature. The resulting mixture was extracted with EA (3×80 mL). The combined organic layers were washed with saturate aq. NaHCO₃(3×50 mL) and dried over anhydrous Na₂SO₄. After filtration, the filtrate was concentrated under reduced pressure. The residue was purified by silica gel column chromatography, eluted with PE/EA (9/1) to afford 2-(4,5-dichloro-2-methoxyphenyl)acetonitrile as an off-white solid (1.60 g, 60%): ¹H NMR (300 MHz, CD₃OD) δ 7.45 (s, 1H), 7.18 (s, 1H), 3.87 (s, 3H), 3.73 (s, 2H).

### Step f

To a mixture of NaH (0.28 g, 11.67 mmol, 60% in mineral oil) in DMF (6 mL) was added 2-(4,5-dichloro-2-methoxyphenyl)acetonitrile (0.80 g, 3.70 mmol) at room temperature under nitrogen atmosphere. The reaction was stirred for 30 min at room temperature under nitrogen atmosphere. Then a solution of tert-butyl N,N-bis(2-chloroethyl)carbamate (0.87 g, 3.60 mmol) in THE (2 mL) was added dropwise at room temperature under nitrogen atmosphere. The reaction was stirred for 5 h at 80° C. under nitrogen atmosphere. After cooling to room temperature, the reaction was quenched with water (50 mL) at room temperature. The resulting mixture was extracted with EA (3×50 mL). The combined organic layers were washed with brine (3×50 mL) and dried over anhydrous Na₂SO₄. After filtration, the filtrate was concentrated under reduced pressure. The residue was purified by silica gel column chromatography, eluted with PE/EA (10/1) to afford tert-butyl 4-cyano-4-(4,5-dichloro-2-methoxyphenyl)piperidine-1-carboxylate as a light yellow solid (0.70 g, 49%): LCMS (ESI) calc'd for C₁₈H₂₂Cl₂N₂O₃ [M+H−15]⁺: 370, 372 (3:2), found 370, 372 (3:2); ¹H NMR (300 MHz, CD₃OD) δ 7.44 (s, 1H), 7.27 (s, 1H), 4.21 (d, J=14.2 Hz, 2H), 3.91 (s, 3H), 3.20-3.12 (m, 2H), 2.28 (d, J=12.8 Hz, 2H), 1.97-1.89 (m, 2H), 1.45 (s, 9H).

### Step g

To a stirred solution of tert-butyl 4-cyano-4-(4,5-dichloro-2-methoxyphenyl)piperidine-1-carboxylate (0.10 g, 0.26 mmol) in DCM (4 mL) was added BBr₃ (0.65 g, 2.60 mmol) at room temperature. The reaction mixture was stirred for 48 h at room temperature. The reaction was quenched with water (3 mL) at room temperature. The result solution was concentrated under the reduced pressure. The residue was purified by Prep-HPLC with the following conditions: Column: XBridge C₁₈ OBD Prep Column 100 Å, 10 m, 19 mm×250 mm; Mobile Phase A: Water with 20 mmol/L NH₄HCO₃, Mobile Phase B: ACN; Flow rate: 20 mL/min; Gradient: 30% B to 70% B in 9 min; Detector: UV 254/220 nm; Retention time: 8.11 min. The fractions containing the desired product were collected and concentrated under reduced pressure to afford Compound 13 (4-(4,5-dichloro-2-hydroxyphenyl)piperidine-4-carbonitrile) as an off-white solid (2.7 mg, 4%). LCMS (ESI) calc'd for C₁₂H₁₂Cl₂N₂O [M+H]⁺: 271, 273 (3:2), found 271, 273 (3:2); ¹H NMR (400 MHz, CD₃OD) δ 7.41 (s, 1H), 7.02 (s, 1H), 3.23-3.13 (m, 2H), 3.14-3.02 (m, 2H), 2.36 (dd, J=13.5, 2.4 Hz, 2H), 2.14-2.02 (m, 2H).

### Example 20. Compound 14 (2-[(2R,4S)-rel-4-(2,3-dichloro-6-hydroxyphenyl)piperidin-2-yl]-N-methylacetamide isomer 1) and Compound 30 (2-[(2R,4S)-rel-4-(2,3-dichloro-6-hydroxyphenyl)piperidin-2-yl]-N-methylacetamide isomer 2)

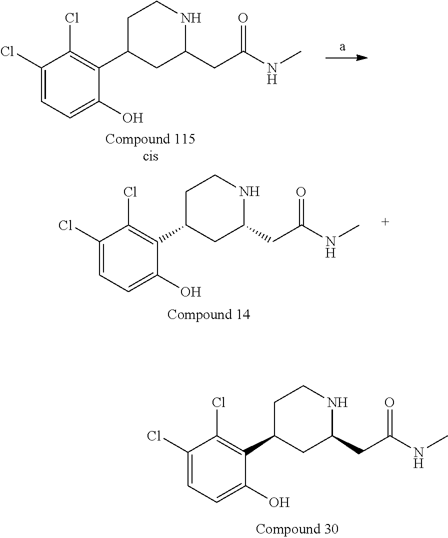 [SMILES: CNC(=O)CC1CC(c2c(O)ccc(Cl)c2Cl)CCN1 ; CNC(=O)C[C@@H]1C[C@H](c2c(O)ccc(Cl)c2Cl)CCN1 ; CNC(=O)C[C@H]1C[C@@H](c2c(O)ccc(Cl)c2Cl)CCN1]

### Step a

2-[4-(2,3-dichloro-6-hydroxyphenyl)piperidin-2-yl]-N-methylacetamide (Compound 115, Example 76 below) (81 mg, 0.19 mmol) was separated by Prep Chiral-HPLC with the following conditions: Column: Chiralpak ID-2, 2×25 cm, 5 μm; Mobile Phase A: Hex (plus 0.1% TFA), Mobile Phase B: EtOH; Flow rate: 20 mL/min; Gradient: 10% B to 10% B in 25 min; Detector: UV: 220/254 nm; Retention time; RT₁: 9.09 min; RT₂: 17.95 min; Injection Volume: 0.9 mL; Number Of Runs: 5.

The faster-eluting enantiomer Compound 14 (2-[(2R,4S)-rel-4-(2,3-dichloro-6-hydroxyphenyl)piperidin-2-yl]-N-methylacetamide isomer 1) at 9.09 min was obtained as an off-white solid (28.9 mg, 36%): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.25 (d, J=8.8 Hz, 1H), 6.75 (d, J=8.8 Hz, 1H), 4.10-3.71 (m, 1H), 3.67-3.57 (m, 1H), 3.57-3.48 (m, 1H), 3.39-3.34 (m, 1H), 3.25-3.05 (m, 1H), 3.00-2.72 (m, 4H), 2.72-2.53 (m, 2H), 1.88-1.59 (m, 2H.

The slower-eluting enantiomer Compound 30 (2-[(2R,4S)-rel-4-(2,3-dichloro-6-hydroxyphenyl)piperidin-2-yl]-N-methylacetamide isomer 2) at 17.95 min was obtained as an off-white solid (26.8 mg, 33%): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.25 (d, J=8.8 Hz, 1H), 6.76 (d, J=8.8 Hz, 1H), 3.83-3.70 (m, 1H), 3.67-3.57 (m, 1H), 3.57-3.49 (m, 1H), 3.25-3.12 (m, 1H), 2.83-2.66 (m, 4H), 2.66-2.50 (m, 3H), 1.88-1.79 (m, 2H).

### Example 21. Compound 16 (4,5-dichloro-2-(piperidin-3-yl)phenol)

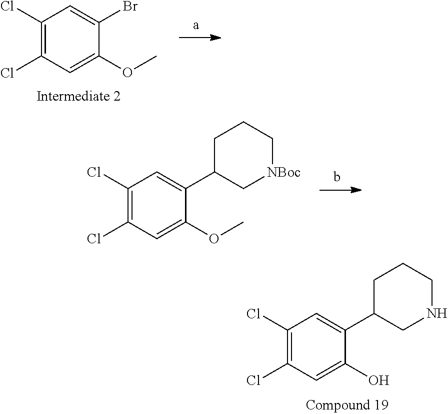 [SMILES: COc1cc(Cl)c(Cl)cc1Br ; COc1cc(Cl)c(Cl)cc1C1CCCN(C(=O)OC(C)(C)C)C1 ; Oc1cc(Cl)c(Cl)cc1C1CCCNC1]

### Step a

To a solution of Intermediate 2 (0.44 g, 1.70 mmol), tert-butyl 3-bromopiperidine-1-carboxylate (0.30 g, 1.14 mmol), Ir[F(CF₃)PPY]₂(DTBPY)PF₆ (13 mg, 0.01 mmol) and tris(trimethylsilyl)silane (0.28 g, 1.14 mmol) was added Na₂CO₃ (0.24 g, 2.27 mmol) at room temperature under argon atmosphere to afford the mixture A. Nickel chloride dimethoxyethane adduct (1 mg, 0.01 mmol) and dtbbpy (1.52 mg, 0.01 mmol) were dissolved in DME (1 mL) under argon atmosphere to afford the mixture B. Then the mixture B was added into mixture A under argon atmosphere. The resulted mixture was stirred and irradiated with 34W blue LEDs for 3 hours. The reaction solution was diluted with water (20 mL). The resulted mixture was extracted with EA (3×30 mL). The combined organic layers were washed with brine (2×30 mL) and dried over anhydrous Na₂SO₄. After filtration, the filtrate was concentrated under reduced pressure. The residue was purified by the Prep-TLC, eluted with PE/EA (2/1) to afford tert-butyl 3-[(4,5-dichloro-2-methoxyphenyl)methyl]-4-methylpiperazine-1-carboxylate as a light yellow oil (0.20 g, 45%); LCMS (ESI) calc'd for C₁₇H₂₃Cl₂NO₃ [M+H−56]⁺: 304, 306 (3:2), found 304, 306 (3:2); ¹H NMR (400 MHz, CD₃OD) δ 7.33 (s, 1H), 7.13 (s, 1H), 4.09 (t, J=13.2 Hz, 2H), 3.88 (s, 3H), 3.71-3.57 (m, 1H), 3.51-3.41 (m, 1H), 3.06-2.97 (m, 1H), 2.91-2.70 (m, 2H), 1.91 (d, J=12.8 Hz, 1H), 1.84-1.67 (m, 1H), 1.49 (s, 9H).

### Step b

To a solution of tert-butyl 3-(4,5-dichloro-2-methoxyphenyl)piperidine-1-carboxylate (0.10 g, 0.28 mmol) in DCM (1 mL) was added BBr₃ (0.83 mL, 0.84 mmol, 1 M in DCM). The mixture was stirred for 3 h at room temperature. The reaction was quenched with MeOH (2 mL) at room temperature. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 20 mmol/L NH₄HCO₃, Mobile Phase B: ACN; Flow rate: 20 mL/min; Gradient: 20% B to 80% B in 9 min; Detector: UV 254/220 nm; Retention time: 8.10 min. The fractions containing the desired product were collected and concentrated under reduced pressure to afford Compound 16 (4,5-dichloro-2-(piperidin-3-yl)phenol) as an off-white solid (23.9 mg, 35%): LCMS (ESI) calc'd for C₁₁H13Cl₂NO [M+H]⁺: 246, 248 (3:2), found 246, 248 (3:2); ¹H NMR (300 MHz, DMSO-d₆) δ 7.19 (s, 1H), 6.83 (s, 1H), 3.00-2.81 (m, 2H), 2.81-2.56 (m, 3H), 1.82-1.65 (m, 1H), 1.65-1.29 (m, 3H).

The compounds in Table 1C below were prepared in an analogous fashion to that described for Compound 16, starting from the corresponding bromides, which were prepared as described herein or were available from commercial sources.

TABLE 1C

| Compound | | | |
|---|---|---|---|
| Number | Structure | Chemical Name | MS: (M + H)⁺ & ¹H MNR |
| 10 | 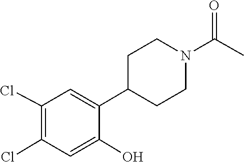 [SMILES: CC(=O)N1CCC(c2cc(Cl)c(Cl)cc2O)CC1] | 1-[4-(4,5- dichloro-2- hydroxyphenyl) piperidin-1- yl]ethan-1-one | [M + H]⁺: 288, 290 (3:2); ¹H NMR (400 MHz, CD₃OD) δ 7.21 (s, 1H), 6.92 (s, 1H), 4.69 (d, J = 13.3 Hz, 1H), 4.05 (d, J = 13.7 Hz, 1H), 3.26 − 3.06 (m, 2H), 2.77 − 2.65 (m, 1H), 2.15 (s, 3H), 1.89 (t, J = 16.2 Hz, 2H), 1.74 − 1.45 (m, 2H). |
| 21 | 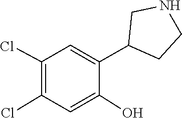 [SMILES: Oc1cc(Cl)c(Cl)cc1C1CCNC1] | 4,5-dichloro-2- (pyrrolidin-3- yl)phenol | [M + H]⁺: 232, 234 (3:2); ¹H NMR (400 MHz, CD₃OD) δ 7.13 (s, 1H), 6.81 (s, 1H), 3.51 − 3.41 (m, 1H), 3.38 − 3.35 (m, 1H), 3.25 (dd, J = 10.8, 7.8 Hz, 1H), 3.08 − 2.96 (m, 2H), 2.37 − 2.26 (m, 1H), 1.98 − 1.86 (m, 1H). |
| 22 | 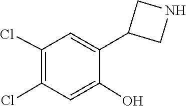 [SMILES: Oc1cc(Cl)c(Cl)cc1C1CNC1] | 2-(azetidin-3-yl)- 4,5- dichlorophenol | [M + H]⁺: 218, 220 (3:2); ¹H NMR (300 MHz, DMSO-d₆) δ 7.48 (s, 1H), 7.02 (s, 1H), 4.60 (t, J = 9.0 Hz, 1H), 4.43 (dd, J = 9.0, 5.9 Hz, 1H), 3.63 − 3.40 (m, 1H), 2.84 − 2.53 (m, 2H). |
| 39 | 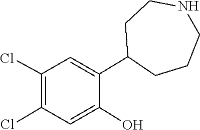 [SMILES: Oc1cc(Cl)c(Cl)cc1C1CCCNCC1] | 2-(azepan-4-yl)- 4,5- dichlorophenol | [M + H]⁺: 260, 262 (3:2); ¹H NMR (400 MHz, CD₃OD) δ 7.18 (s, 1H), 6.85 (s, 1H), 3.21 − 3.12 (m, 2H), 3.12 − 2.88 (m, 3H), 2.04 − 1.72 (m, 6H). |
| 40 | 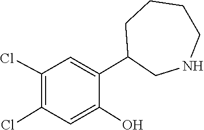 [SMILES: Oc1cc(Cl)c(Cl)cc1C1CCCCNC1] | 2-(azepan-3-yl)- 4,5- dichlorophenol | [M + H]⁺: 260, 262 (3:2); ¹H NMR (300 MHz, DMSO-d₆) δ 10.53 (brs, 1H), 8.77 (d, J = 89.5 Hz, 2H), 7.36 (s, 1H), 7.03 (s, 1H), 3.33 − 3.22 (m, 2H), 3.22 − 3.12 (m, 3H), 1.95 − 1.72 (m, 5H), 1.66 − 1.49 (m, 1H). |

### Example 22. Compound 18 (1-(piperidin-4-yl)naphthalen-2-ol)

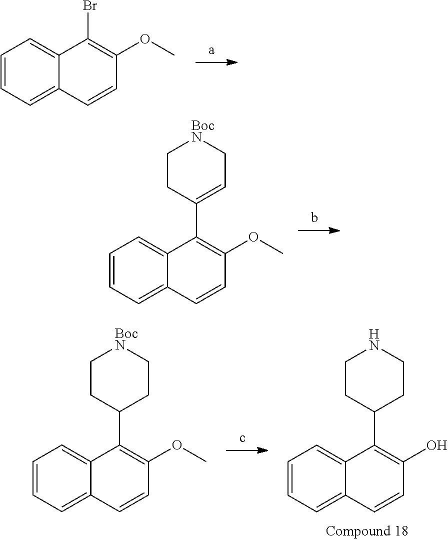

### Step a

To a stirred solution of 1-bromo-2-methoxynaphthalene (1.00 g, 4.22 mmol) and tert-butyl 4-(tetramethyl-1,3,2-dioxaborolan-2-yl)-1,2,3,6-tetrahydropyridine-1-carboxylate (1.57 g, 5.06 mmol) and Na₂CO₃ (1.34 g, 12.65 mmol) in 1,4-dioxane (8 mL) and H₂O (2 mL) was added Pd(dppf)Cl₂.CH₂Cl₂ (0.15 g, 0.21 mmol) under nitrogen atmosphere. The resulting mixture was stirred under nitrogen at 80° C. for 2 h under nitrogen atmosphere. The mixture was allowed to cool down to room temperature. The reaction was diluted with water (50 mL). The resulting mixture was extracted with EA (3×80 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (4/1) to afford tert-butyl 4-(2-methoxynaphthalen-1-yl)-1,2,3,6-tetrahydropyridine-1-carboxylate as a yellow oil (1.20 g, 84%). LCMS (ESI) calc'd for C₂₁H₂₅NO₃ [M+Na]⁺: 362, found 362; ¹H NMR (300 MHz, CD₃OD) δ 7.82-7.69 (m, 3H), 7.41-7.24 (m, 3H), 5.59-5.50 (m, 1H), 4.17-4.00 (m, 2H), 3.88 (s, 3H), 3.73-3.65 (m, 2H), 2.58-2.43 (m, 1H), 2.25-2.09 (m, 1H), 1.50 (s, 9H).

### Step b

To a stirred solution of tert-butyl 4-(2-methoxynaphthalen-1-yl)-1,2,3,6-tetrahydropyridine-1-carboxylate (1.00 g, 2.95 mmol) in MeOH (50 mL) was added Pt/C (0.57 g, 10%) in a pressure tank. The mixture was hydrogenated at room temperature under hydrogen pressure of 20 atm. for 24 h. The reaction was solution was filtered through celite and the filtrate was concentrated under reduced pressure. The residue was purified by Prep-TLC, eluted with PE/EA (4/1) to afford tert-butyl 4-(2-methoxynaphthalen-1-yl)piperidine-1-carboxylate as a light-yellow oil (0.20 g, 20%). LCMS (ESI) calc'd for C₂₁H₂₇NO₃ [M+H]⁺: 342, found 342.

### Step c

To a stirred solution of tert-butyl 4-(2-methoxynaphthalen-1-yl)piperidine-1-carboxylate (0.20 g, 0.59 mmol) in DCM (2 mL) was added BBr₃ (0.74 g, 2.93 mmol) at 0° C. under nitrogen atmosphere. The resulting mixture was stirred under nitrogen at room temperature for 2 h. The reaction was quenched with water (5 mL) at 0° C. The mixture was basified to pH=7 with saturated aq. NaHCO₃. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃), Mobile Phase B: ACN; Flow rate: 25 mL/min; Gradient: 20% B to 60% B in 6.5 min; Detector: UV 254/220 nm; Retention time: 5.35 min. The fractions containing the desired product were collected and concentrated under reduced pressure to afford Compound 18 (1-(piperidin-4-yl)naphthalen-2-ol) as an off-white solid (30 mg, 23%): LCMS (ESI) calc'd for C₁₅H₁₇NO [M+H]⁺: 228, found 228; ¹H NMR (300 MHz, CD₃OD) δ 8.15 (d, J=8.7 Hz, 1H), 7.73 (d, J=8.1 Hz, 1H), 7.60 (d, J=8.8 Hz, 1H), 7.43 (t, J=7.7 Hz, 1H), 7.25 (t, J=7.4 Hz, 1H), 7.07 (d, J=8.9 Hz, 1H), 3.80-3.64 (m, 1H), 3.38-3.33 (m, 1H), 3.31-3.26 (m, 1H), 3.10-2.88 (m, 2H), 2.88-2.67 (m, 2H), 1.71 (d, J=13.5 Hz, 2H).

### Example 23. Compound 26 (4,5-dichloro-2-[4-(hydroxymethyl)piperidin-4-yl]phenol)

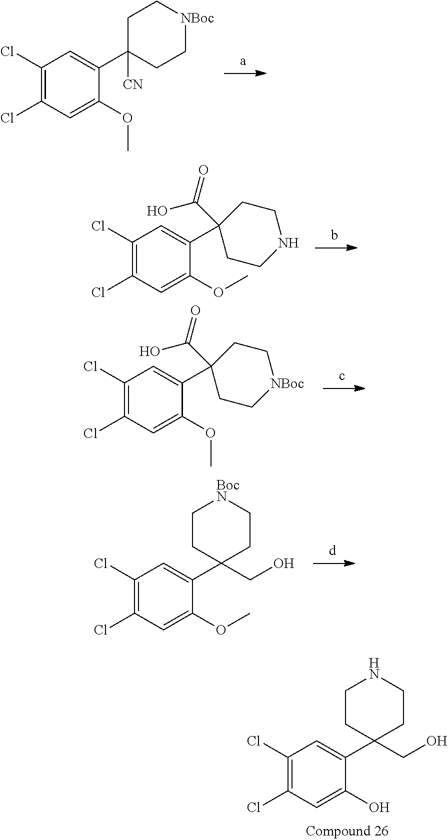

### Step a

A mixture of tert-butyl 4-cyano-4-(4,5-dichloro-2-methoxyphenyl)piperidine-1-carboxylate (from Example 19 step f) (0.10 g, 0.26 mmol) in conc. HCl (5 mL) was stirred for 48 h at 80° C. The reaction solution was concentrated under reduced pressure to afford 4-(4,5-dichloro-2-methoxyphenyl)piperidine-4-carboxylic acid as a light yellow solid (0.17 g, crude), which was used to next step directly without further purification: LCMS (ESI) calc'd for C₁₃H₁₅Cl₂NO₃ [M+H]⁺: 304, 306 (3:2), found 304, 306 (3:2); ¹H NMR (400 MHz, CD₃OD) δ 7.47 (s, 1H), 7.22 (s, 1H), 3.84 (s, 3H), 3.48-3.37 (m, 4H), 2.59 (d, J=14.6 Hz, 2H), 2.17 (m, 2H).

### Step b

To a stirred solution of 4-(4,5-dichloro-2-methoxyphenyl)piperidine-4-carboxylic acid (0.10 g, 0.33 mmol) and NaOH (20 mg, 0.50 mmol) in MeOH (3 mL) was added Boc₂O (0.22 g, 1.00 mmol) at room temperature. The solution was stirred at room temperature for 2 h. The solution was acidified to pH 4 with saturated aq. citric acid (20 mL). The resulting mixture was extracted with DCM (3×30 mL). The combined organic layers were washed with brine (3×30 mL), dried over anhydrous Na₂SO₄. After filtration, the filtrate was concentrated under reduced pressure to afford 1-[(tert-butoxy)carbonyl]-4-(4,5-dichloro-2-methoxyphenyl)piperidine-4-carboxylic acid as a yellow oil (0.13 g, crude), which was used to next step directly without further purification: LCMS (ESI) calc'd for C₁₈H₂₃Cl₂NO₅ [M+H−56]⁺: 348, 350 (3:2), found 348, 350 (3:2); ¹H NMR (400 MHz, CDCl₃) δ 7.37 (s, 1H), 6.98 (s, 1H), 3.94-3.76 (m, 5H), 3.45-3.29 (m, 2H), 2.42-2.28 (m, 2H), 1.96-1.82 (m, 2H), 1.51 (s, 9H).

### Step c

To a stirred solution of 1-[(tert-butoxy)carbonyl]-4-(4,5-dichloro-2-methoxyphenyl)piperidine-4-carboxylic acid (0.13 g, 0.32 mmol) in THE (1 mL) was added BH3-THF (1.29 mL, 1.29 mmol, 1 M in THF) at 0° C. under nitrogen atmosphere. The solution was stirred at room temperature under nitrogen atmosphere for 6 h. The reaction was quenched with saturated aq. NH₄Cl (10 mL) at room temperature. The resulting mixture was diluted with water (30 mL). The resulting mixture was extracted with EA (3×30 mL). The combined organic layers were washed with brine (3×30 mL), dried over anhydrous Na₂SO₄. After filtration, the filtrate was concentrated under reduced pressure. The residue was purified by Prep-TLC, eluted with PE/EA (5/1) to afford tert-butyl 4-(4,5-dichloro-2-methoxyphenyl)-4-(hydroxymethyl)piperidine-1-carboxylate as an off-white solid (40 mg, 40% overall three steps): LCMS (ESI) calc'd for C₁₈H₂₅Cl₂NO₄ [M+H−56]⁺: 334, 336 (3:2), found 334, 336 (3:2); ¹H NMR (400 MHz, CD₃OD) δ 7.35 (s, 1H), 7.17 (s, 1H), 3.86 (s, 3H), 3.79 (s, 2H), 3.72-3.64 (m, 2H), 3.22-3.09 (m, 2H), 2.36-2.28 (m, 2H), 1.88-1.79 (m, 2H), 1.47 (s, 9H).

### Step d

To a stirred mixture of tert-butyl 4-(4,5-dichloro-2-methoxyphenyl)-4-(hydroxymethyl)piperidine-1-carboxylate (40 mg, 0.10 mmol) in DCM (1 mL) was added BBr₃ (0.19 g, 0.74 mmol) dropwise at room temperature. The resulting mixture was stirred for 4 h at room temperature. The reaction was quenched with water (5 mL) at room temperature. The mixture was basified to pH 7 with saturated aq. NaHCO₃. The resulting mixture was concentrated under reduced pressure. The residue was purified by Prep-HPLC with the following conditions: Column: SunFire C₁₈ OBD Prep Column, 100 Å, 5 μm, 19 mm×250 mm; Mobile Phase A: Water (plus 0.05% TFA), Mobile Phase B: ACN; Flow rate: 20 mL/min; Gradient: 15% B to 40% B in 8 min; Detector: UV 254/210 nm; Retention time: 7.5 min. The fractions containing the desired product were collected and concentrated under reduced pressure to afford Compound 26 (4,5-dichloro-2-[4-(hydroxymethyl)piperidin-4-yl]phenol) as an off white solid (7 mg, 18%): LCMS (ESI) calc'd for C₁₂H₁₅Cl₂NO₂ [M+H]⁺: 276, 278 (3:2), found 276, 278 (3:2); ¹H NMR (400 MHz, DMSO-d₆) δ 10.47 (brs, 1H), 8.36 (brs, 2H), 7.28 (s, 1H), 7.04 (s, 1H), 4.81 (brs, 1H), 3.69-3.48 (m, 2H), 3.24-3.11 (m, 2H), 2.89-2.69 (m, 2H), 2.49-2.40 (m, 2H), 1.97 (t, J=12.6 Hz, 2H).

### Example 24. Compound 27 (4,5-dichloro-2-[2-(hydroxymethyl)piperidin-4-yl]phenol)

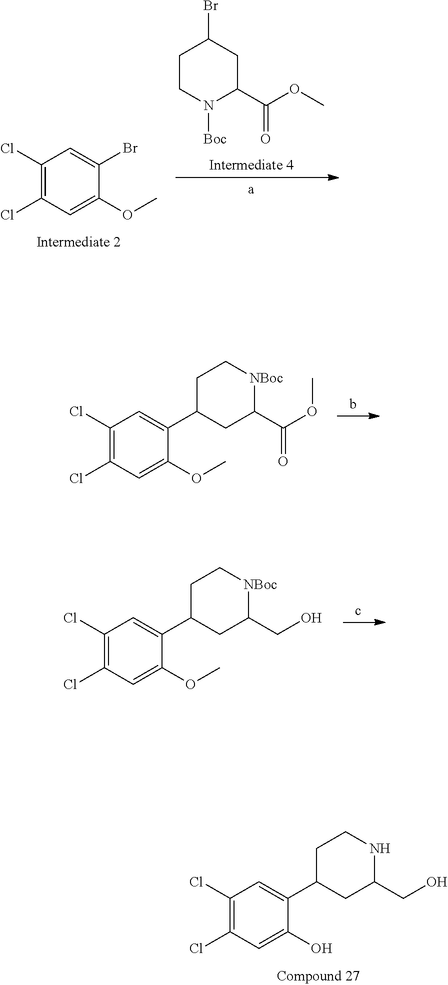 [SMILES: COC(=O)C1CC(Br)CCN1C(=O)OC(C)(C)C ; COc1cc(Cl)c(Cl)cc1Br ; COC(=O)C1CC(c2cc(Cl)c(Cl)cc2OC)CCN1C(=O)OC(C)(C)C ; COc1cc(Cl)c(Cl)cc1C1CCN(C(=O)OC(C)(C)C)C(CO)C1 ; OCC1CC(c2cc(Cl)c(Cl)cc2O)CCN1]

### Step a

To a stirred solution of Intermediate 2 (0.30 g, 1.17 mmol) and 1-tert-butyl 2-methyl 4-bromopiperidine-1,2-dicarboxylate (Intermediate 4) (0.45 g, 1.41 mmol) Ir[F(CF₃)PPY]₂(DTBPY)PF₆ (13 mg, 0.01 mmol), 1,1,1,3,3,3-hexamethyl-2-(trimethylsilyl)trisilane (0.29 g, 1.17 mmol) in DME (3 mL) was added Na₂CO₃ (0.25 g, 2.34 mmol) at room temperature under argon atmosphere to afford the mixture A. Dtbbpy (1.5 mg, 0.01 mmol) and 1,2-dimethoxyethane dihydrochloride nickel (1.3 mg, 0.01 mmol) were dissolved in DME (2 mL) under argon atmosphere to afford the mixture B. Then the mixture B was added into mixture A under argon atmosphere, the resulted mixture was stirred and irradiated with 34W blue LEDs for 2 h. The reaction mixture was poured into water (50 mL) and extracted with EA (3×50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (5/1) to afford 1-tert-butyl 2-methyl 4-(4,5-dichloro-2-methoxyphenyl)piperidine-1,2-dicarboxylate as light yellow oil (0.20 g, 41%): LCMS (ESI) calc'd for C₁₉H₂₅Cl₂NO₅ [M+H−15]⁺: 403, 405 (3:2), found 403, 405 (3:2); ¹H NMR (300 MHz, CD₃OD) δ 7.26 (s, 1H), 7.10 (s, 1H), 4.98-4.91 (m, 1H), 4.75-4.53 (m, 1H), 3.84 (s, 3H), 3.79 (d, J=2.8 Hz, 3H), 3.75-3.70 (m, 1H), 2.94-2.79 (m, 1H), 2.42-2.27 (m, 1H), 2.05-1.96 (m, 1H), 1.90-1.71 (m, 2H), 1.45 (s, 9H).

### Step b

To a stirred solution of 1-tert-butyl 2-methyl 4-(4,5-dichloro-2-methoxyphenyl)piperidine-1,2-dicarboxylate (0.20 g, 0.48 mmol) in THE (5 mL) was added DIBAl-H (1.42 mL, 1.43 mmol, 1 M in toluene) dropwise at 0° C. under nitrogen atmosphere. The resulting solution was stirred at room temperature for 5 h under nitrogen atmosphere. The reaction was quenched with water (20 mL) at room temperature. The resulting mixture was extracted with EA (4×30 mL). The combined organic layers were washed with brine (2×30 mL) and dried over anhydrous Na₂SO₄. After filtration, the filtrate was concentrated under reduced pressure. The residue was purified by Prep-TLC, eluted with PE/EA (1/2) to afford tert-butyl4-(4,5-dichloro-2-methoxyphenyl)-2-(hydroxymethyl)piperidine-1-carboxylate as yellow oil (80 mg, 41%): LCMS (ESI) calc'd for C₁₈H₂₅Cl₂NO₄ [M+H]⁺: 390, 392 (3:2), found 390, 392 (3:2).

### Step c

To a stirred mixture of tert-butyl 4-(4,5-dichloro-2-methoxyphenyl)-2-(hydroxymethyl)piperidine-1-carboxylate (80 mg, 0.20 mmol) in DCM (1 mL) was added BBr₃ (0.41 g, 1.64 mmol) at 0° C. under nitrogen atmosphere. The resulting mixture was stirred for 2 h at room temperature. The reaction was quenched with water (2 mL) at room temperature. The mixture was neutralized to pH 7 with saturated aq. NaHCO₃. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 20 mL/min; Gradient: 20% B to 65% B in 6.5 min; Detector: UV 254/210 nm; Retention time: 5.48 min to afford Compound 27 (4,5-dichloro-2-[2-(hydroxymethyl)piperidin-4-yl]phenol) as an off-white solid (15 mg, 27%): LCMS (ESI) calc'd for C₁₂H¹⁵Cl₂NO₂ [M+H]⁺: 276, 278 (3:2), found 276, 278 (3:2); ¹H NMR (300 MHz, CD₃OD) δ 7.25 (s, 1H), 6.93 (s, 1H), 4.04 (dd, J=11.8, 10.2 Hz, 1H), 3.77 (dd, J=11.8, 4.8 Hz, 1H), 3.68-3.52 (m, 1H), 3.39-3.33 (m, 1H), 3.30-3.24 (m, 2H), 2.16-1.93 (m, 4H).

### Example 25. Compound 29 (4-chloro-2-(piperidin-4-yl)-5-(trifluoromethyl)phenol)

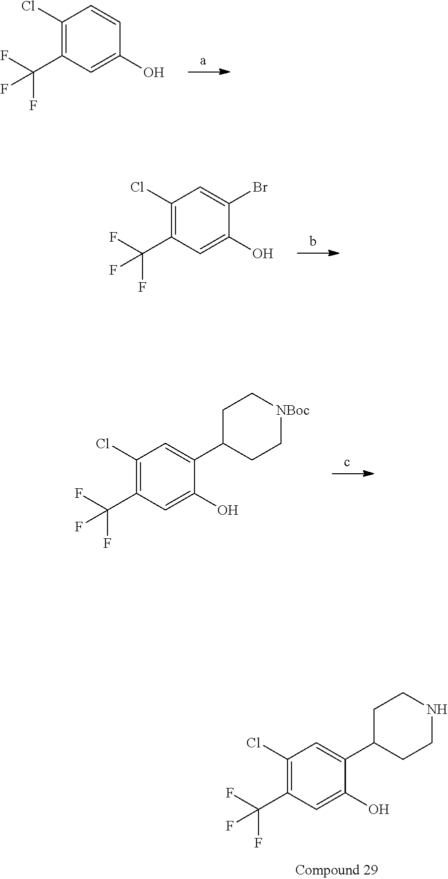 [SMILES: Oc1ccc(Cl)c(C(F)(F)F)c1 ; Oc1cc(C(F)(F)F)c(Cl)cc1Br ; CC(C)(C)OC(=O)N1CCC(c2cc(Cl)c(C(F)(F)F)cc2O)CC1 ; Oc1cc(C(F)(F)F)c(Cl)cc1C1CCNCC1]

### Step a

To a stirred solution of 4-chloro-3-(trifluoromethyl)phenol (4.00 g, 20.35 mmol) in HOAc (40 mL) was added Br₂ (6.50 g, 40.70 mmol) dropwise at 0° C. The reaction was stirred at room temperature for 2 h. The reaction was diluted with EA (80 mL) and water (80 mL). The aqueous solution was extracted with EA (3×80 mL). The combined organic layers were washed with brine (3×80 mL) and dried over anhydrous Na₂SO₄. After filtration, the filtrate was concentrated under reduced pressure. The residue was purified by silica gel column chromatography, eluted with PE/EA (10/1) to afford 2-bromo-4-chloro-5-(trifluoromethyl)phenol as a light yellow solid (2.40 g, 39%): LCMS (ESI) calc'd for C₇H₃BrClF₃O [M−1]⁺: 273, 275, 277 (2:3:1), found 273, 275, 277 (2:3:1); ¹H NMR (300 MHz, CDCl₃) δ 7.63 (s, 1H), 7.35 (s, 1H), 5.73 (s, 1H).

### Step b

To a stirred mixture of 2-bromo-4-chloro-5-(trifluoromethyl)phenol (0.20 g, 0.73 mmol) and tert-butyl 4-bromopiperidine-1-carboxylate (0.29 g, 1.09 mmol) in DME (1 mL) were added 1,1,1,3,3,3-hexamethyl-2-(trimethylsilyl)trisilane (0.18 g, 0.73 mmol), Na₂CO₃ (230.9 mg, 2.18 mmol) and Ir[df(CF₃)ppy]₂(dtbpy)PF₆ (8 mg, 0.01 mmol) at room temperature under argon atmosphere to afford mixture A. Dtbbpy (1 mg, 0.004 mmol) and 1,2-dimethoxyethane dichloronickel (1 mg, 0.004 mmol) were dissolved in DME (1 mL) under argon atmosphere to afford mixture B. Then the mixture B was added into mixture A under argon atmosphere. The resulted mixture was stirred and irradiated with 34W blue LEDs for 3 h. The reaction solution was diluted with water (20 mL). The resulted mixture was extracted with EA (3×30 mL). The combined organic layers were washed with brine (2×30 mL) and dried over anhydrous Na₂SO₄. After filtration, the filtrate was concentrated under reduced pressure. The residue was purified by the Prep-TLC, eluted with PE/EA (2/1) to afford tert-butyl 4-[5-chloro-2-hydroxy-4-(trifluoromethyl)phenyl]piperidine-1-carboxylate as an off-white solid (35 mg, 7%); LCMS (ESI) calc'd for C₁₇H₂₁ClF₃NO₃ [M+1−15]⁺ 365, 367 (3:1), found 365, 367 (3:1).

### Step c

To a stirred solution of tert-butyl 4-[5-chloro-2-hydroxy-4-(trifluoromethyl)phenyl]piperidine-1-carboxylate (35 mg, 0.09 mmol) in DCM (1 mL) was added TFA (1 mL) at room temperature. The reaction was stirred at room temperature for 1 h. The reaction was concentrated under reduced pressure. The residue was purified by the following conditions: XBridge C₁₈ OBD Prep Column 100 Å, 10 m, 19 mm×250 mm; Mobile Phase A: Water with 20 mmoL/L NH₄HCO₃, Mobile Phase B: ACN; Flow rate: 20 mL/min; Gradient: 25% B to 40% B in 9 min; Detector: UV 254/210 nm; Retention time: 7.67 min. The fractions containing the desired product were collected and concentrated under reduced pressure to afford Compound 29 (4-chloro-2-(piperidin-4-yl)-5-(trifluoromethyl)phenol) as an off-white solid (8.8 mg, 32%): LCMS (ESI) calc'd for C₁₂H₁₃ClF₃NO [M+1]⁺: 280, 282 (3:1), found 280, 282 (3:1). ¹H NMR (400 MHz, CD₃OD) δ 7.28 (s, 1H), 7.11 (s, 1H), 3.32-3.25 (m, 2H), 3.25-3.10 (m, 1H), 2.93-2.88 (m, 2H), 2.00-1.88 (m, 2H), 1.84-1.64 (m, 2H).

### Example 26. Compound 34 (4,5-dichloro-2-((2R,4S)-rel-2-(hydroxymethyl)piperidin-4-yl)phenol isomer 1) and Compound 35 (4,5-dichloro-2-((2R,4S)-rel-2-(hydroxymethyl)piperidin-4-yl)phenol isomer 2)

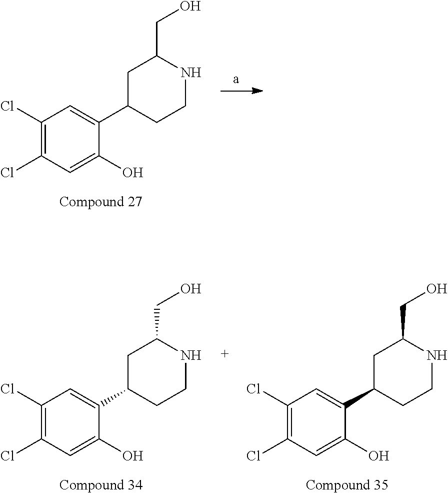 [SMILES: OCC1CC(c2cc(Cl)c(Cl)cc2O)CCN1 ; OC[C@H]1C[C@@H](c2cc(Cl)c(Cl)cc2O)CCN1 ; OC[C@@H]1C[C@H](c2cc(Cl)c(Cl)cc2O)CCN1]

The absolute configurations for Compounds 34 and 35 were arbitrarily assigned.

### Step a

4,5-dichloro-2-(2-(hydroxymethyl)piperidin-4-yl)phenol (Compound 27, Example 24) (20 mg, 0.07 mmol) was separated by Chiral Prep-HPLC with the following conditions: Column: Chiralpak AD-H, 2.0 cm I.D.×25 cm; Mobile Phase A: Hex (plus 0.1% DEA)-HPLC; Mobile Phase B: EtOH-HPLC; Flow rate: 20 mL/min; Gradient: 20% B to 20% B in 17 min; Detector: UV: 220/254 nm; Retention time: RT₁: 8.24 min; RT₂: 13.44 min; Temperature: 25° C.

The faster-eluting enantiomer Compound 34 (4,5-dichloro-2-((2R,4R)-rel-2-(hydroxymethyl)piperidin-4-yl)phenol isomer 1) at 8.24 min was obtained as an off-white solid (5 mg, 25%): LCMS (ESI) calc'd for C₁₂H₁₅Cl₂NO₂ [M+1]⁺: 276, 278 (3:2), found 276, 278 (3:2); ¹H NMR (300 MHz, CD₃OD) δ 7.23 (s, 1H), 6.88 (s, 1H), 3.93 (dd, J=11.2, 9.2 Hz, 1H), 3.63 (dd, J=11.2, 5.4 Hz, 1H), 3.29-3.16 (m, 2H), 3.11-3.00 (m, 1H), 3.00-2.90 (m, 1H), 1.93-1.65 (m, 4H).

The slower-eluting enantiomer Compound 35 (4,5-dichloro-2-((2R,4S)-rel-2-(hydroxymethyl)piperidin-4-yl)phenol isomer 2) at 13.44 min was obtained as an off-white solid (6 mg, 30%): LCMS (ESI) calc'd for C₁₂H₁₅Cl₂NO₂ [M+1]⁺: 276, 278 (3:2), found 276, 278 (3:2); ¹H NMR (300 MHz, CD₃OD) δ 7.23 (s, 1H), 6.88 (s, 1H), 3.93 (dd, J=11.2, 9.2 Hz, 1H), 3.63 (dd, J=11.2, 5.5 Hz, 1H), 3.29-3.15 (m, 2H), 3.11-2.99 (m, 1H), 2.98-2.89 (m, 1H), 1.95-1.65 (m, 4H).

### Example 27. Compound 36 (4,5-dichloro-2-(5-(hydroxymethyl)pyrrolidin-3-yl)phenol)

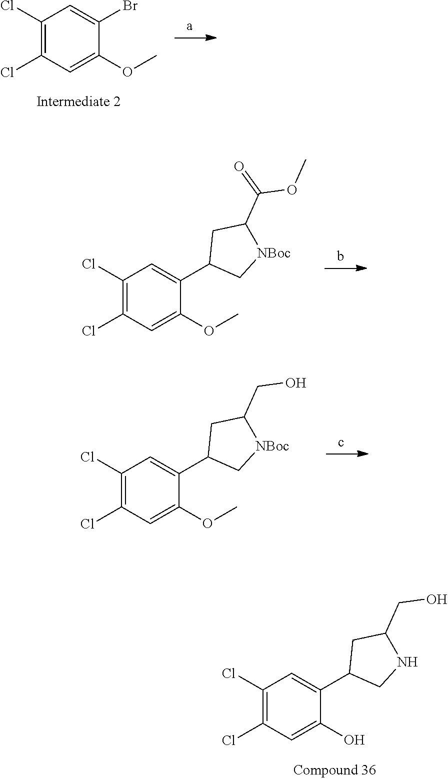 [SMILES: COc1cc(Cl)c(Cl)cc1Br ; COC(=O)C1CC(c2cc(Cl)c(Cl)cc2OC)CN1C(=O)OC(C)(C)C ; COc1cc(Cl)c(Cl)cc1C1CC(CO)N(C(=O)OC(C)(C)C)C1 ; OCC1CC(c2cc(Cl)c(Cl)cc2O)CN1]

### Step a

To a stirred solution of Intermediate 2 (0.50 g, 1.97 mmol) and 1-tert-butyl 2-methyl 4-bromopyrrolidine-1,2-dicarboxylate (0.61 g, 1.97 mmol) in DME (5 mL) were added Ir[F(CF₃)PPY]₂(DTBPY)PF₆ (22 mg, 0.02 mmol), 1,1,1,3,3,3-hexamethyl-2-(trimethylsilyl)trisilane (0.49 g, 1.97 mmol) and Na₂CO₃ (0.42 g, 3.94 mmol) at room temperature under argon atmosphere to afford the mixture A. Dtbbpy (3 mg, 0.01 mmol) and 1,2-dimethoxyethane dihydrochloride nickel (2 mg, 0.01 mmol) were dissolved in DME (2 mL) under argon atmosphere to afford the mixture B. Then the mixture B was added into mixture A under argon atmosphere, the resulted mixture was stirred and irradiated with 34W blue LEDs for 2 h. The reaction mixture was poured into water (50 mL) and extracted with EA (3×50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by Prep-TLC, eluted with PE/EA (5/1) to afford 1-tert-butyl 2-methyl 4-(4,5-dichloro-2-methoxyphenyl)pyrrolidine-1,2-dicarboxylate as a light yellow oil (0.23 g, 29%): LCMS (ESI) calc'd for C₁₈H₂₃Cl₂NO₅ [M+H]⁺: 404, 406 (3:2), found 404, 406 (3:2).

### Step b

To a stirred solution of 1-tert-butyl 2-methyl 4-(4,5-dichloro-2-methoxyphenyl)pyrrolidine-1,2-dicarboxylate (0.15 g, 0.37 mmol) in THE (2 mL) was added DIBAl-H (1.14 mL, 1.13 mmol, 1 M in toluene) dropwise at 0° C. under nitrogen atmosphere. The resulting solution was stirred at room temperature for 5 h under nitrogen atmosphere. The reaction was quenched with water (20 mL) at room temperature. The resulting mixture was extracted with EA (4×30 mL). The combined organic layers were washed with brine (2×30 mL) and dried over anhydrous Na₂SO₄. After filtration, the filtrate was concentrated under reduced pressure. The residue was purified by Prep-TLC, eluted with PE/EA (1/1) to afford tert-butyl 4-(4,5-dichloro-2-methoxyphenyl)-2-(hydroxymethyl)pyrrolidine-1-carboxylate as a yellow oil (50 mg, 36%): LCMS (ESI) calc'd for C₁₇H₂₃Cl₂NO₄ [M+H]⁺: 376, 378 (3:2), found 376, 378 (3:2); ¹H NMR (300 MHz, CD₃OD) δ 7.29 (s, 1H), 7.12 (s, 1H), 4.16-3.80 (m, 4H), 3.82-3.53 (m, 4H), 2.32-2.04 (m, 2H), 2.03-1.73 (m, 1H), 1.47 (s, 9H).

### Step c

To a stirred mixture of tert-butyl 4-(4,5-dichloro-2-methoxyphenyl)-2-(hydroxymethyl)pyrrolidine-1-carboxylate (50 mg, 0.13 mmol) in DCM (1 mL) was added BBr₃ (0.27 g, 1.06 mmol) at 0° C. under nitrogen atmosphere. The resulting mixture was stirred for 2 h at room temperature. The reaction was quenched with water (2 mL) at room temperature. The mixture was neutralized to pH 7 with saturated aq. NaHCO₃. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 20 mL/min; Gradient: 20% B to 70% B in 6.5 min; Detector: UV 254/210 nm; Retention time: 5.03 min. The fractions containing the desired product were collected and concentrated under reduced pressure to afford Compound 36 (4,5-dichloro-2-(5-(hydroxymethyl)pyrrolidin-3-yl)phenol) as a grey solid (13.9 mg, 28%): LCMS (ESI) calc'd for C₁₁H₁₃Cl₂NO₂ [M+H]⁺: 262, 264 (3:2), found 262, 264 (3:2); ¹H NMR (300 MHz, CD₃OD) δ 7.35 (s, 1H), 6.99 (s, 1H), 4.07-3.94 (m, 1H), 3.94-3.79 (m, 1H), 3.79-3.61 (m, 3H), 3.47-3.36 (m, 1H), 2.43-2.13 (m, 2H).

### Example 28. Compound 41 (4-chloro-5-(difluoromethyl)-2-(piperidin-4-yl)phenol)

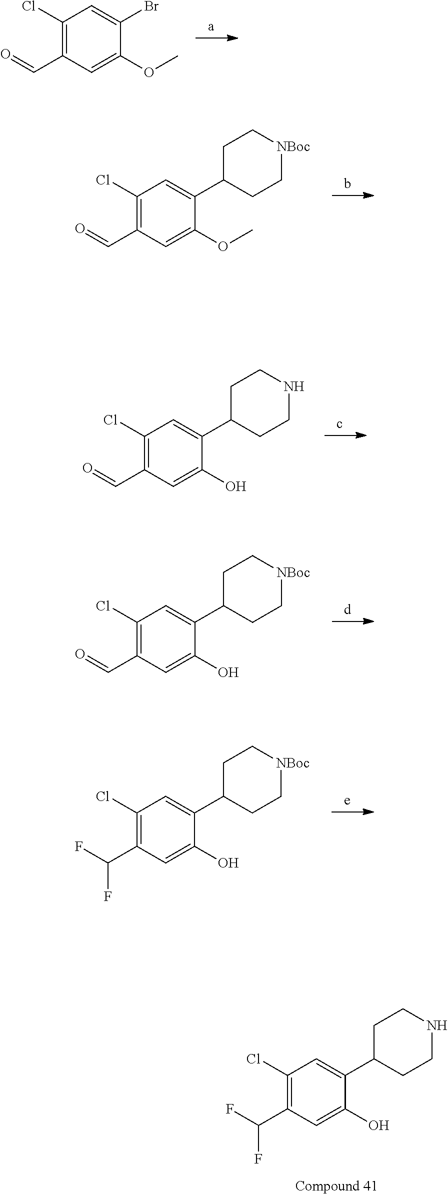 [SMILES: COc1cc(C=O)c(Cl)cc1Br ; COc1cc(C=O)c(Cl)cc1C1CCN(C(=O)OC(C)(C)C)CC1 ; O=Cc1cc(O)c(C2CCNCC2)cc1Cl ; CC(C)(C)OC(=O)N1CCC(c2cc(Cl)c(C=O)cc2O)CC1 ; CC(C)(C)OC(=O)N1CCC(c2cc(Cl)c(C(F)F)cc2O)CC1 ; Oc1cc(C(F)F)c(Cl)cc1C1CCNCC1]

### Step a

To a solution of 4-bromo-2-chloro-5-methoxybenzaldehyde (0.56 g, 2.24 mmol) and tert-butyl 4-bromopiperidine-1-carboxylate (0.71 g, 2.69 mmol) in DME (5 mL) were added 1,1,1,3,3,3-hexamethyl-2-(trimethylsilyl)trisilane (0.56 g, 2.24 mmol), Ir[F(CF₃)PPY]₂(DTBPY)PF₆ (25 mg, 0.02 mmol) and Na₂CO₃ (0.48 g, 4.49 mmol) at room temperature under argon atmosphere to afford the mixture A. Dtbbpy (3 mg, 0.01 mmol) and 1,2-dimethoxyethane dihydrochloride nickel (3 mg, 0.01 mmol) were dissolved in DME (1 mL) under argon atmosphere to afford the mixture B. Then the mixture B was added into mixture A under argon atmosphere, the resulted mixture was stirred and irradiated with 34W blue LEDs for 2 h. The reaction mixture was poured into water (50 mL) and extracted with EA (3×50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by Prep-TLC, eluted with PE/EA (8/1) to afford tert-butyl 4-(5-chloro-4-formyl-2-methoxyphenyl)piperidine-1-carboxylate as a light yellow oil (0.30 g, 34%): LCMS (ESI) calc'd for C₁₈H₂₄ClNO₄ [M+H−15]⁺: 339, 341 (3:1), found 339, 341 (3:1); ¹H NMR (300 MHz, CDCl₃) δ 10.42 (s, 1H), 7.39 (s, 1H), 7.22 (s, 1H), 4.35-4.24 (m, 2H), 3.90 (s, 3H), 3.21-3.06 (m, 1H), 2.92-2.76 (m, 2H), 1.86-1.73 (m, 2H), 1.71-1.57 (m, 2H), 1.51 (s, 9H).

### Step b

To a stirred solution of tert-butyl 4-(5-chloro-4-formyl-2-methoxyphenyl)piperidine-1-carboxylate (0.30 g, 0.85 mmol) in DCM (1 mL) was added BBr₃ (1.27 g, 5.09 mmol) at 0° C. under nitrogen atmosphere. The resulting mixture was stirred for 2 h at room temperature. The reaction was quenched with water (20 mL) at room temperature. The mixture was concentrated under reduced pressure. The residue was purified by reverse phase chromatography, eluted with 35% ACN in water (plus 0.05% TFA) to afford 2-chloro-5-hydroxy-4-(piperidin-4-yl)benzaldehyde as a colorless oil (0.10 g, 34%): LCMS (ESI) calc'd for C₁₂H₁₄ClNO₂ [M+H]⁺: 240, 242 (3:1), found 240, 242 (3:1).

### Step c

To a stirred mixture of 2-chloro-5-hydroxy-4-(piperidin-4-yl)benzaldehyde (0.20 g, 0.83 mmol) and Boc₂O (0.27 g, 1.25 mmol) in DCM (3 mL) was added Et₃N (0.17 g, 1.67 mmol) at room temperature. The resulting mixture was stirred at room temperature for 2 h. The reaction was diluted with water (20 mL). The resulting mixture was extracted with EA (3×50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by Prep-TLC, eluted with PE/EA (5/1) to afford tert-butyl 4-(5-chloro-4-formyl-2-hydroxyphenyl)piperidine-1-carboxylate as a yellow oil (0.12 g, 38%): LCMS (ESI) calc'd for C₁₇H₂₂C₁NO₄ [M+H−15]⁺: 325, 327 (3:1), found 325, 327 (3:1); ¹H NMR (300 MHz, CDCl₃) δ 10.38 (s, 1H), 7.32 (s, 1H), 7.23 (s, 1H), 4.29 (d, J=13.4 Hz, 2H), 3.17-3.03 (m, 1H), 2.94-2.77 (m, 2H), 1.91-1.82 (m, 2H), 1.70-1.62 (m, 2H), 1.51 (s, 9H).

### Step d

To a stirred solution of tert-butyl 4-(5-chloro-4-formyl-2-hydroxyphenyl)piperidine-1-carboxylate (30 mg, 0.09 mmol) in DCM (1 mL) was added DAST (43 mg, 0.26 mmol) at 0° C. The reaction was stirred for 2 h at room temperature. The reaction was quenched with water (20 mL). The resulting mixture was extracted with EA (3×15 mL). The combined organic layers were washed with brine (3×10 mL) and dried over anhydrous Na₂SO₄. After filtration, the filtrate was concentrated under reduced pressure. The residue was purified by Prep-TLC, eluted with PE/EA (5/1) to afford tert-butyl 4-[5-chloro-4-(difluoromethyl)-2-hydroxyphenyl]piperidine-1-carboxylate as a yellow oil (20 mg, 56%): LCMS (ESI) calc'd for C₁₇H₂₂ClF₂NO₃ [M+H−15]⁺: 347, 349 (3:1), found 347, 349 (3:1); ¹H NMR (300 MHz, CDCl₃) δ 7.43 (s, 1H), 7.32 (s, 1H), 6.90 (t, J=54.9 Hz, 1H), 4.38-4.20 (m, 2H), 2.93-2.65 (m, 3H), 1.89-1.62 (m, 4H), 1.50 (s, 9H).

### Step e

A solution of tert-butyl 4-[5-chloro-4-(difluoromethyl)-2-hydroxyphenyl]piperidine-1-carboxylate (20 mg, 0.06 mmol) in TFA (1 mL) and DCM (1 mL) was stirred for 1 h at room temperature. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 20 mmol/L NH₄HCO₃, Mobile Phase B: ACN; Flow rate: 20 mL/min; Gradient: 35% B to 65% B in 6.5 min; Detector: UV: 254/210 nm; Retention time: 5.41 min. The fractions containing the desired product were collected and concentrated under reduced pressure to afford Compound 41 (4-chloro-5-(difluoromethyl)-2-(piperidin-4-yl)phenol) as an off-white solid (10 mg, 65.67%): LCMS (ESI) calc'd for C₁₂H₁₄ClF₂NO [M+H]⁺: 262, 264 (3:1), found 262, 264 (3:1); ¹H NMR (300 MHz, DMSO-d₆) δ 7.19 (s, 1H), 7.09 (s, 1H), 7.07 (t, J=54.7 Hz, 1H), 3.07-2.86 (m, 3H), 2.64-2.54 (m, 2H), 1.72-1.55 (m, 2H), 1.55-1.36 (m, 2H).

### Example 29. Compound 42 ((3R,4R)-rel-4-(4,5-dichloro-2-hydroxyphenyl)piperidine-3-carboxamide) and Compound 50 ((3R,4S)-rel-4-(4,5-dichloro-2-hydroxyphenyl)piperidine-3-carboxamide)

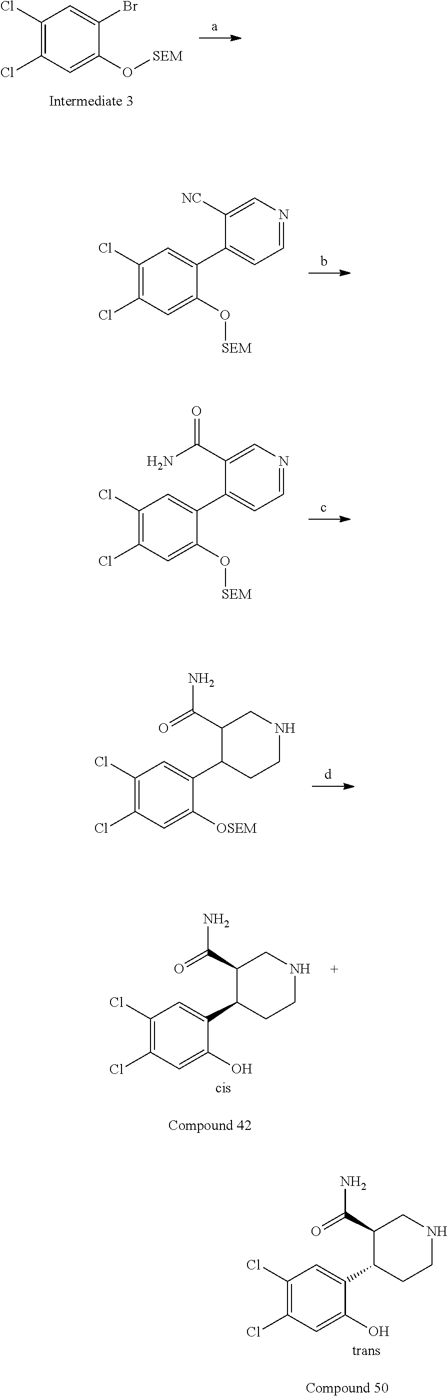

### Step a

A mixture of [2-[(2-bromo-4,5-dichlorophenoxy)methoxy]ethyl]trimethylsilane (Intermediate 3) (2.62 g, 7.04 mmol), 4-(4,4,5,5-tetramethyl-1,3,2-dioxaborolan-2-yl)pyridine-3-carbonitrile (1.90 g, 8.45 mmol), Pd(crotyl)(JohnPhos)Cl (0.35 g, 0.70 mmol) and Na₂CO₃ (2.20 g, 21.12 mmol) in 1,4-dioxane (16 mL) and water (4 mL) was stirred for 3 h at 80° C. under nitrogen atmosphere. The reaction mixture was allowed to cool to room temperature and diluted with water (50 mL) and extracted with EA (3×50 mL). The combined organic layers were washed with brine (2×40 mL) and dried over anhydrous Na₂SO₄. After filtration, the filtrate was concentrated under reduced pressure. The residue was purified by silica gel column chromatography, eluted with PE/EA (2/1) to afford 4-(4,5-dichloro-2-[[2-(trimethylsilyl)ethoxy]methoxy]phenyl)pyridine-3-carbonitrile as a light yellow oil (0.99 g, 32%): LCMS (ESI) calc'd for C₁₈H₂₀Cl₂N₂O₂Si [M+H]⁺: 395, 397 (3:2), found 395, 397 (3:2); ¹H NMR (400 MHz, CD₃OD) δ 8.98 (s, 1H), 8.86 (dd, J=5.2, 1.0 Hz, 1H), 7.61-7.54 (m, 3H), 5.28 (s, 2H), 3.75 (t, J=8.0 Hz, 2H), 0.99-0.86 (m, 2H), 0.03-0.01 (m, 9H).

### Step b

To a mixture of 4-(4,5-dichloro-2-[[2-(trimethylsilyl)ethoxy]methoxy]phenyl)pyridine-3-carbonitrile (0.80 g, 2.02 mmol) and NaOH (0.81 g, 20.23 mmol) in MeOH (10 mL) was added H₂O₂ (0.69 g, 20.23 mmol, 30%) at room temperature. The reaction was stirred for 1 h at room temperature. The reaction mixture was quenched with saturated aq. Na₂S203 (30 mL) and extracted with EA (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with PE/EA (1/3) to afford 4-(4,5-dichloro-2-[[2-(trimethylsilyl)ethoxy]methoxy]phenyl)pyridine-3-carboxamide as a light yellow oil (0.73 g, 70%): LCMS (ESI) calc'd for C₁₈H₂₂Cl₂N₂O₃Si [M+H]⁺: 413, 415 (3:2), found 413, 415 (3:2); ¹H NMR (400 MHz, CD₃OD) δ 8.78 (s, 1H), 8.69 (d, J=5.2 Hz, 1H), 7.50 (s, 1H), 7.47 (s, 1H), 7.43 (d, J=5.1 Hz, 1H), 5.20 (s, 2H), 3.69 (t, J=7.9 Hz, 2H), 0.92 (t, J=8.0 Hz, 2H), 0.00 (s, 9H).

### Step c

To a mixture of 4-(4,5-dichloro-2-[[2-(trimethylsilyl)ethoxy]methoxy]phenyl)pyridine-3-carboxamide (0.73 g, 1.77 mmol) in MeOH (5 mL) was added aq. HCl (6 N, 0.5 mL) at room temperature. The reaction was stirred for 5 h at 30° C. under hydrogen (50 atm.) atmosphere. The reaction mixture was filtered and the filtrate was adjusted pH to 8 with saturated aq. NaHCO₃. The resulting solution was concentrated under reduced pressure to afford 4-(4,5-dichloro-2-[[2-(trimethylsilyl)ethoxy]methoxy]phenyl)piperidine-3-carboxamide (0.93 g, crude), which was directly used in next step without further purification: LCMS (ESI) calc'd for C₁₈H₂₈Cl₂N₂O₃Si [M+H]⁺: 419, 421 (3:2), found 419, 421 (3:2); ¹H NMR (300 MHz, DMSO-d₆) δ 7.35 (s, 1H), 7.21 (s, 1H), 5.41 (d, J=6.8 Hz, 1H), 5.27 (d, J=6.8 Hz, 1H), 3.76 (t, J=8.0 Hz, 2H), 3.07-2.97 (m, 1H), 2.86 (t, J=11.9 Hz, 1H), 2.79-2.64 (m, 2H), 2.48-2.36 (m, 2H), 1.61-1.48 (m, 1H), 1.11-1.01 (m, 1H), 0.97-0.87 (m, 2H), 0.00 (s, 9H).

### Step d

To a stirred solution of 4-(4,5-dichloro-2-[[2-(trimethylsilyl)ethoxy]methoxy]phenyl)piperidine-3-carboxamide (80 mg, 0.19 mmol) in DCM (1 mL) was added TFA (1 mL) at room temperature. The reaction was stirred at room temperature for 1 h. The reaction was concentrated under reduced pressure. The residue was dissolved in THE (1 mL) and NH₃.H₂O (0.5 mL, 30%) was added. The resulting solution was stirred at room temperature for 1 h. The reaction was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 25 mL/min; Gradient: 19% B to 26% B in 8 min; Detector: UV 254/220 nm; Retention time: RT₁: 6.38 min, RT₂: 6.45 min. The fractions containing the desired product at 6.38 min were collected and concentrated under reduced pressure to afford Compound 42 ((3R,4R)-rel-4-(4,5-dichloro-2-hydroxyphenyl)piperidine-3-carboxamide (cis isomer)) as an off-white solid (14.2 mg, 26%): LCMS (ESI) calc'd for C₁₂H₁₄Cl₂N₂O₂ [M+H]⁺: 289, 291 (3:2), found 289, 291 (3:2); ¹H NMR (300 MHz, CD₃OD) δ 7.15 (s, 1H), 6.91 (s, 1H), 3.54-3.36 (m, 3H), 3.05 (dd, J=13.4, 3.7 Hz, 1H), 2.97-2.78 (m, 2H), 2.62-2.39 (m, 1H), 1.70-1.50 (m, 1H). Fractions containing the desired product at 6.45 min were collected and concentrated under reduced pressure to afford Compound 50 ((3R,4S)-rel-4-(4,5-dichloro-2-hydroxyphenyl)piperidine-3-carboxamide (trans isomer)) as an off-white solid (1.5 mg, 3%): LCMS (ESI) calc'd for C₁₂H₁₄Cl₂N₂O₂ [M+H]⁺: 289, 291 (3:2), found 289, 291 (3:2); ¹H NMR (300 MHz, CD₃OD) δ 7.27 (s, 1H), 6.89 (s, 1H), 3.29-3.06 (m, 3H), 2.96-2.66 (m, 3H), 1.87-1.61 (m, 2H).

### Example 30. Compound 43 (4,5-dichloro-2-(2-cyclopropylpiperidin-4-yl)phenol)

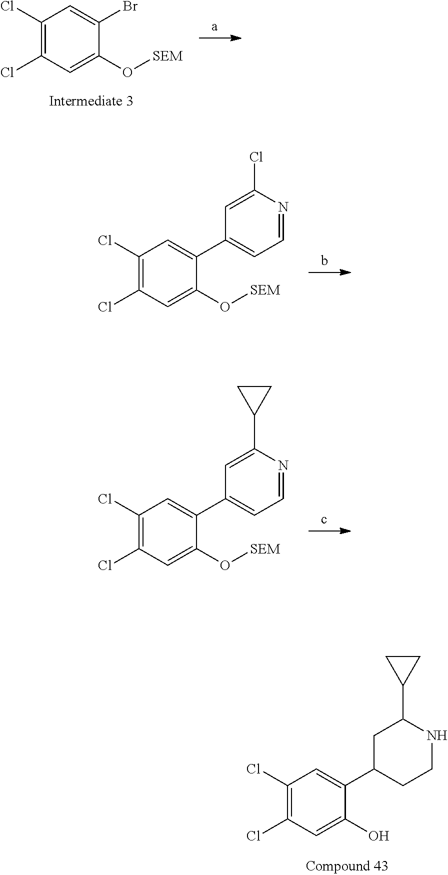 [SMILES: C[Si](C)(C)CCOCOc1cc(Cl)c(Cl)cc1Br ; C[Si](C)(C)CCOCOc1cc(Cl)c(Cl)cc1-c1ccnc(Cl)c1 ; C[Si](C)(C)CCOCOc1cc(Cl)c(Cl)cc1-c1ccnc(C2CC2)c1 ; Oc1cc(Cl)c(Cl)cc1C1CCNC(C2CC2)C1]

### Step a

To a stirred solution of Intermediate 3 (2.00 g, 5.37 mmol) and 2-chloro-4-(tetramethyl-1,3,2-dioxaborolan-2-yl)pyridine (1.54 g, 6.45 mmol) in 1,4-dioxane (25 mL) and water (5 mL) were added Pd(dppf)Cl₂ (0.39 g, 0.54 mmol) and Na₂CO₃ (1.70 g, 16.11 mmol) at room temperature under argon atmosphere. The reaction was stirred at 80° C. for 16 h. The reaction was diluted with EA (50 mL) and water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford 2-chloro-4-(4,5-dichloro-2-[[2-(trimethylsilyl)ethoxy]methoxy]phenyl)pyridine as a light yellow oil (1.70 g, 78%): LCMS (ESI) calc'd for C₁₇H₂₀Cl₃NO₂Si [M+H]⁺: 404, 406 (3:2), found 404, 406 (3:2); ¹H NMR (400 MHz, CDCl₃) δ 8.45 (d, J=5.1 Hz, 1H), 7.48 (d, J=1.5 Hz, 1H), 7.43 (s, 2H), 7.35 (dd, J=5.1, 1.5 Hz, 1H), 5.24 (s, 2H), 3.75-3.65 (m, 2H), 0.99-0.90 (m, 2H), 0.02 (s, 9H).

### Step b

To a stirred solution of 2-chloro-4-(4,5-dichloro-2-[[2-(trimethylsilyl)ethoxy]methoxy]phenyl)pyridine (0.50 g, 1.24 mmol) and cyclopropylboronic acid (0.16 g, 1.85 mmol) in toluene (5 mL) and water (1 mL) were added tricyclohexylphosphane (35 mg, 0.12 mmol), K₃PO₄ (0.52 g, 2.47 mmol) and (acetyloxy)palladio acetate (28 mg, 0.12 mmol) at room temperature under argon atmosphere. The reaction was stirred at 90° C. for 16 h under argon atmosphere. The reaction was diluted with EA (50 mL) and water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (3/1) to afford 4-(4-chloro-5-cyclopropyl-2-[[2-(trimethylsilyl)ethoxy]methoxy]phenyl)-2-cyclopropylpyridine as a light yellow oil (80 mg, 16%): LCMS (ESI) calc'd for C₂₀H₂₅Cl₂NO₂Si [M+H]⁺: 410, 412 (3:2), found 410, 412 (3:2); ¹H NMR (400 MHz, CDCl₃) δ 8.48 (d, J=5.1 Hz, 1H), 7.41 (d, J=5.9 Hz, 2H), 7.22 (s, 1H), 7.16 (dd, J=5.1, 1.6 Hz, 1H), 5.20 (s, 2H), 3.74-3.64 (m, 2H), 2.13-2.03 (m, 1H), 1.12-1.01 (m, 4H), 0.99-0.90 (m, 2H), 0.01 (s, 9H).

### Step c

To a stirred solution of 2-cyclopropyl-4-(4,5-dichloro-2-[[2-(trimethylsilyl)ethoxy]methoxy]phenyl)pyridine (40 mg, 0.10 mmol) in MeOH (3 mL) were added PtO₂ (22 mg, 0.10 mmol) and aq. HCl (6 N, 0.3 mL) at room temperature. The reaction was stirred at 30° C. for 16 h under hydrogen atmosphere (50 atm). The reaction was filtered and the filtrate was concentrated under reduced pressure. The residue was purified by Prep-HPLC with following conditions: Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 25 mL/min; Gradient: 40% B to 80% B in 6.5 min; Detector: UV 254/210 nm; Retention time: 5.25 min. The fractions containing the desired product were collected and concentrated under reduced pressure to afford Compound 43 (4,5-dichloro-2-(2-cyclopropylpiperidin-4-yl)phenol) as an off-white solid (21.3 mg, 76%): LCMS (ESI) calc'd for C₁₄H₁₇Cl₂NO [M+H]⁺: 286, 288 (3:2), found 286,288 (3:2); ¹H NMR (300 MHz, CD₃OD) δ 7.19 (s, 1H), 6.86 (s, 1H), 3.27-3.18 (m, 1H), 3.12-2.94 (m, 1H), 2.86-2.68 (m, 1H), 2.04-1.77 (m, 3H), 1.70-1.39 (m, 2H), 0.90-0.79 (m, 1H), 0.60-0.49 (m, 2H), 0.38-0.18 (m, 2H).

### Example 31. Compound 44 (3,4,5-trichloro-2-(piperidin-4-yl)phenol)

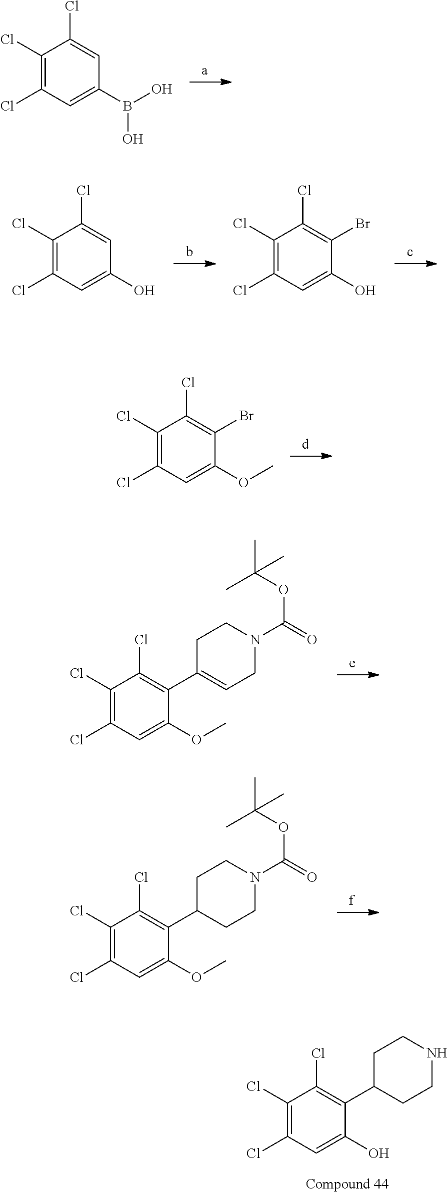

### Step a

To a stirred solution of (3,4,5-trichlorophenyl)boronic acid (5.00 g, 22.20 mol) in THE (15 mL) were added H₂O₂ (1.51 g, 44.39 mmol, 30%) and NaOH (1.78 g, 44.39 mmol) at room temperature. The resulting mixture was stirred for 2 h at room temperature. The reaction was quenched with water (50 mL) at room temperature. The mixture was acidified to pH 3 with aq. HCl (6 N). The resulting mixture was extracted with EA (3×80 mL). The combined organic layers were washed with brine (2×80 mL) and dried over anhydrous Na₂SO₄. After filtration, the filtrate was concentrated under reduced pressure. The residue was purified by silica gel column chromatography, eluted with PE/EA (5/1) to afford 3,4,5-trichlorophenol as a light yellow solid (4.60 g, 100%): LCMS (ESI) calc'd for C₆H₃C₁₃₀ [M−H]⁺: 195, 197, 199 (3:3:1), found 195, 197, 199 (3:3:1); ¹H NMR (400 MHz, CDCl₃) δ 6.92 (s, 2H).

### Step b

To a stirred solution of 3,4,5-trichlorophenol (4.60 g, 23.30 mol) in AcOH (20 mL) was added Br₂ (3.70 g, 23.15 mol) dropwise at room temperature under argon atmosphere. The reaction was quenched with saturate aq. Na₂SO₃ (50 mL). The mixture was extracted with EA (3×80 mL). The combined organic layer was washed with brine (3×80 mL) and dried over anhydrous Na₂SO₄. After filtration, the filtrate was concentrated under reduced pressure. The residue was purified by silica gel column chromatography, eluted with PE/EA (15/1) to afford 2-bromo-3,4,5-trichlorophenol as an off-white solid (2.40 g, 37%): LCMS (ESI) calc'd for C₆H₂BrCl₃O [M−H]⁺: 273, 275, 277 (1:2:1), found 273, 275, 277 (1:2:1); ¹H NMR (400 MHz, DMSO-d₆) δ 11.43 (s, 1H), 7.15 (s, 1H).

### Step c

To a stirred solution of 2-bromo-3,4,5-trichlorophenol (2.40 g, 8.69 mmol) and K₂CO₃ (2.40 g, 17.37 mmol) in DMF (15 mL) was added MeI (3.70 g, 26.07 mmol) at room temperature. The reaction was stirred at 50° C. for 1 h. The reaction was diluted with EA (80 mL) and water (80 mL). The partitioned aqueous solution was extracted with EA (3×80 mL). The combined organic layer was washed with brine (6×50 mL) and dried over anhydrous Na₂SO₄. After filtration, the filtrate was concentrated under reduced pressure. The residue was purified by silica gel column chromatography, eluted with PE/EA (15/1) to afford 2-bromo-3,4,5-trichloro-1-methoxybenzene as an off-white solid (1.80 g, 71%): ¹H NMR (400 MHz, CDCl₃) δ 6.98 (s, 1H), 3.94 (s, 3H).

### Step d

To a mixture of 2-bromo-3,4,5-trichloro-1-methoxybenzene (0.10 g, 0.344 mmol) and tert-butyl 4-(4,4,5,5-tetramethyl-1,3,2-dioxaborolan-2-yl)-1,2,3,6-tetrahydropyridine-1-carboxylate (0.12 g, 0.34 mmol) and Na₂CO₃ (0.11 g, 1.04 mmol) in water (0.5 mL) and 1,4-dioxane (2 mL) was added Pd(dppf)Cl₂.CH₂Cl₂ (28 mg, 0.03 mmol) at room temperature under nitrogen atmosphere. The mixture was stirred 80° C. for 8 h under nitrogen atmosphere. The reaction mixture was poured into water (30 mL) and extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (6/1) to afford to afford tert-butyl 4-(2,3,4-trichloro-6-methoxyphenyl)-1,2,3,6-tetrahydropyridine-1-carboxylate as a light yellow semi-solid (0.23 g, 80%): LCMS (ESI) calc'd for C₁₇H₂₀Cl₃NO₃ [M+H−15]⁺: 377, 379, 381 (3:3:1), found: 377, 379, 381 (3:3:1); ¹H NMR (400 MHz, CDCl₃) δ 6.94 (s, 1H), 5.55 (s, 1H), 4.07 (s, 2H), 3.80 (s, 3H), 3.74-3.54 (m, 2H), 2.37-2.15 (m, 2H), 1.52 (s, 9H).

### Step e

To a solution of tert-butyl 4-(2,3,4-trichloro-6-methoxyphenyl)-1,2,3,6-tetrahydropyridine-1-carboxylate (50 mg, 0.13 mmol) in MeOH (2 mL) was added PtO₂ (15 mg, 0.07 mmol) at room temperature. The reaction mixture was stirred at room temperature under hydrogen atmosphere (1.5 atm.) for 2 h. The mixture was filtered and the filtrate was concentrated under reduced pressure to afford tert-butyl 4-(2,3,4-trichloro-6-methoxyphenyl)piperidine-1-carboxylate as a light yellow solid (48 mg, 96%): LCMS (ESI) calc'd for C₁₇H₂₂C₁₃NO₃ [M+H−15]⁺: 379, 381, 383 (3:3:1), found 379, 381, 383 (3:3:1); ¹H NMR (400 MHz, CDCl₃) δ 6.92 (s, 1H), 4.23 (d, J=13.2 Hz, 2H), 3.82 (s, 3H), 3.58-3.37 (m, 1H), 2.79 (t, J=12.9 Hz, 2H), 2.41-2.18 (m, 2H), 1.65-1.53 (m, 2H), 1.52 (s, 9H).

### Step f

To a stirred solution of tert-butyl 4-(2,3,4-trichloro-6-methoxyphenyl)piperidine-1-carboxylate (48 mg, 0.12 mmol) in DCM (1 mL) was added BBr₃ (0.30 g, 1.19 mmol) at room temperature. The reaction was stirred at room temperature for 1 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 30% B to 40% B in 5.3 min; Detector: UV 254/210 nm; Retention time: 4.65 min. The fractions containing the desired product were collected and concentrated under reduced pressure to afford Compound 44 (3,4,5-trichloro-2-(piperidin-4-yl)phenol) as an off-white solid (11.9 mg, 24% overall two steps): LCMS (ESI) calc'd for C₁₁H₁₂Cl₃NO [M+H]⁺: 280, 282, 284 (3:3:1), found 280, 282, 284 (3:3:1); ¹H NMR (400 MHz, CD₃OD) δ 6.97 (s, 1H), 3.68 (t, J=12.7 Hz, 1H), 3.49 (d, J=12.7 Hz, 2H), 3.19-3.01 (m, 2H), 2.85-2.65 (m, 2H), 1.83 (d, J=14.2 Hz, 2H).

### Example 32. Compound 38 (4,5-dichloro-2-(pyrrolidin-3-yl)phenol, isomer 1) and Compound 45 (4,5-dichloro-2-(pyrrolidin-3-yl)phenol, isomer 2)

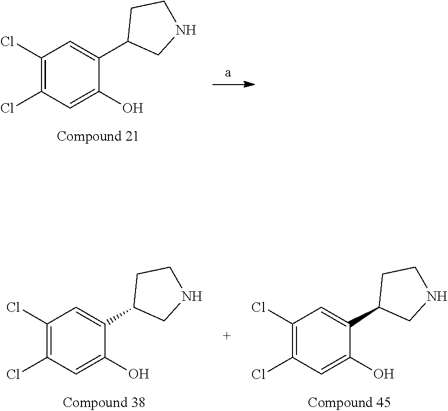 [SMILES: Oc1cc(Cl)c(Cl)cc1C1CCNC1 ; Oc1cc(Cl)c(Cl)cc1[C@@H]1CCNC1 ; Oc1cc(Cl)c(Cl)cc1[C@H]1CCNC1]

The absolute configurations for Compounds 38 and 45 were arbitrarily assigned.

### Step a

4,5-Dichloro-2-(pyrrolidin-3-yl)phenol (40 mg, 0.17 mmol) (Compound 21, Example 21) was separated by Prep SFC with the following conditions: Column: Lux Su Cellulose-4, AXIA Packed, 2.12×25 cm, 5 μm; Mobile Phase A: CO₂, Mobile Phase B: MeOH (plus 0.1% DEA)-HPLC; Flow rate: 45 mL/min; Gradient: 25% B; Detector: UV: 220/254 nm; Retention time: RT₁:6.95 min; RT₂: 7.59 min; Injection Volume: 0.5 mL; Number Of Runs: 12.

The faster-eluting enantiomer Compound 38 (4,5-dichloro-2-(pyrrolidin-3-yl)phenol isomer 1) at 6.95 min was obtained as an off-white solid (9.6 mg, 24%): LCMS (ESI) calc'd for C₁₀H₁₁Cl₂NO [M+H]⁺: 232, 234 (3:2), found 232, 234 (3:2); ¹H NMR (400 MHz, CD₃OD) δ 7.14 (s, 1H), 6.81 (s, 1H), 3.52-3.42 (m, 1H), 3.39-3.35 (m, 1H), 3.27 (dd, J=10.9, 7.8 Hz, 1H), 3.09-2.97 (m, 2H), 2.37-2.28 (m, 1H), 2.01-1.88 (m, 1H).

The slower-eluting enantiomer Compound 45 (4,5-dichloro-2-(pyrrolidin-3-yl)phenol isomer 2) at 7.59 min was obtained as an off-white solid (12.6 mg, 32%): LCMS (ESI) calc'd for C₁₀H₁₁Cl₂NO [M+H]⁺: 232, 234 (3:2), found 232, 234 (3:2); ¹H NMR (400 MHz, CD₃OD) δ 7.14 (s, 1H), 6.81 (s, 1H), 3.52-3.42 (m, 1H), 3.39-3.34 (m, 1H), 3.27 (dd, J=10.9, 7.8 Hz, 1H), 3.09-2.97 (m, 2H), 2.38-2.26 (m, 1H), 2.00-1.87 (m, 1H).

### Example 33. Compound 46 (3,4,5-trichloro-2-(1,2,3,6-tetrahydropyridin-4-yl)phenol)

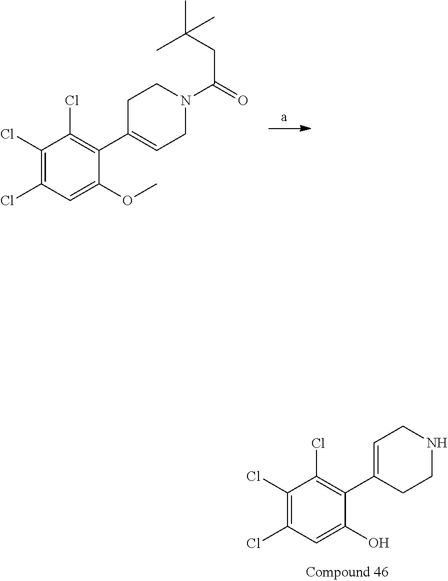 [SMILES: COc1cc(Cl)c(Cl)c(Cl)c1C1=CCN(C(=O)CC(C)(C)C)CC1 ; Oc1cc(Cl)c(Cl)c(Cl)c1C1=CCNCC1]

### Step a

To solution of tert-butyl 4-(2,3,4-trichloro-6-methoxyphenyl)-1,2,3,6-tetrahydropyridine-1-carboxylate (Example 31, step d) (50 mg, 0.13 mmol) in DCM (1 mL) was added BBr₃ (0.30 g, 1.197 mmol) at room temperature. The reaction was stirred at room temperature for 1 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 24% B to 48% B in 6.5 min; Detector: UV 254/210 nm; Retention time: 5.68 min. The fractions containing the desired product were collected and concentrated under reduced pressure to afford Compound 46 (3,4,5-trichloro-2-(1,2,3,6-tetrahydropyridin-4-yl)phenol) as an off-white solid (6.9 mg, 14%): LCMS (ESI) calc'd for C₁₁H₁₀Cl₃NO [M+H]⁺: 278, 280, 282 (3:3:1), found 278, 280, 282 (3:3:1); ¹H NMR (400 MHz, CD₃OD) δ 7.02 (s, 1H), 5.77-5.67 (m, 1H), 3.91-3.83 (m, 2H), 3.47 (t, J=6.1 Hz, 2H), 2.65-2.52 (m, 2H).

### Example 34. Compound 47 ((3R,4S)-rel-2-[3-(aminomethyl)piperidin-4-yl]-4,5-dichlorophenol) and Compound 48 ((3R,4R)-rel-2-[3-(aminomethyl)piperidin-4-yl]-4,5-dichlorophenol)

### Step a

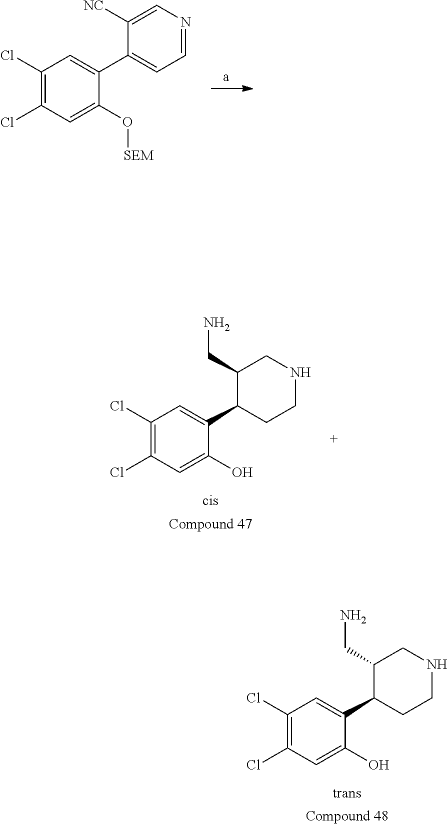 [SMILES: C[Si](C)(C)CCOCOc1cc(Cl)c(Cl)cc1-c1ccncc1C#N ; NC[C@H]1CNCC[C@H]1c1cc(Cl)c(Cl)cc1O ; NC[C@@H]1CNCC[C@H]1c1cc(Cl)c(Cl)cc1O]

A mixture of 4-(4,5-dichloro-2-[[2-(trimethylsilyl)ethoxy]methoxy]phenyl)pyridine-3-carbonitrile (Example 29, step a) (0.10 g, 0.25 mmol) and PtO₂ (12 mg, 0.05 mmol) in MeOH (5 mL) was added aq. HCl (6 N, 0.5 mL) at room temperature. The reaction was stirred for 6.5 h at 30° C. under hydrogen atmosphere (50 atm.). The reaction mixture was filtered and the filtrate was adjusted pH to 8 with saturated aq. NaHCO₃. The resulting solution was concentrated under reduced pressure. The residue was purified by Prep-HPLC with the flowing conditions: Column: XSelect CSH Prep C₁₈ OBD Column, 19×250 mm, 5 m; Mobile Phase A: Water (plus 0.05% TFA), Mobile Phase B: ACN; Flow rate: 25 mL/min; Gradient: 10% B to 25% B in 18 min; Detector: UV 254/220 nm; Retention time: RT₁:14.2 min, RT₂: 15.0 min; The fractions containing the desired product at 14.2 min were collected and concentrated under reduced pressure to afford Compound 47 ((3R,4S)-rel-2-[3-(aminomethyl)piperidin-4-yl]-4,5-dichlorophenol (cis isomer)) as an off-white solid (4.2 mg, 6%): LCMS (ESI) calc'd for C₁₂H₁₆Cl₂N₂O [M+H]⁺: 275, 277 (3:2), found 275, 277 (3:2); ¹H NMR (300 MHz, CD₃OD) δ 7.21 (s, 1H), 6.83 (s, 1H), 3.24-3.09 (m, 2H), 3.09-2.93 (m, 1H), 2.76-2.54 (m, 3H), 2.54-2.38 (m, 1H), 1.91-1.66 (m, 3H). Fractions containing the desired product at 15.0 min were collected and concentrated under reduced pressure to afford Compound 48 ((3R,4R)-rel-2-[3-(aminomethyl)piperidin-4-yl]-4,5-dichlorophenol (trans isomer)) as a brown solid (2.3 mg, 3%): LCMS (ESI) calc'd for C₁₂H₁₆Cl₂N₂O [M+H]⁺: 275, 277 (3:2), found 275, 277 (3:2); ¹H NMR (300 MHz, CD₃OD) δ 7.23 (s, 1H), 7.02 (s, 1H), 3.74-3.55 (m, 3H), 3.50-3.39 (m, 1H), 3.31-3.19 (m, 2H), 2.91-2.79 (m, 1H), 2.65 (dd, J=13.6, 3.2 Hz, 1H), 2.37-2.17 (m, 1H), 1.98-1.85 (m, 1H).

### Example 35. Compound 49 ((3R,4R)-rel-2-(3-aminopiperidin-4-yl)-4,5-dichlorophenol) and Compound 51 ((3R,4S)-rel-2-(3-aminopiperidin-4-yl)-4,5-dichlorophenol)

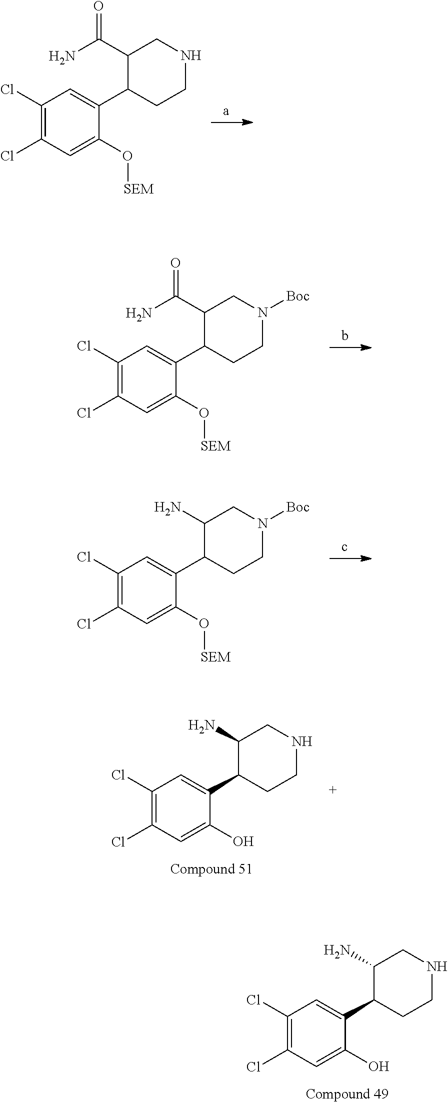 [SMILES: C[Si](C)(C)CCOCOc1cc(Cl)c(Cl)cc1C1CCNCC1C(N)=O ; CC(C)(C)OC(=O)N1CCC(c2cc(Cl)c(Cl)cc2OCOCC[Si](C)(C)C)C(C(N)=O)C1 ; CC(C)(C)OC(=O)N1CCC(c2cc(Cl)c(Cl)cc2OCOCC[Si](C)(C)C)C(N)C1 ; N[C@H]1CNCC[C@H]1c1cc(Cl)c(Cl)cc1O ; N[C@@H]1CNCC[C@H]1c1cc(Cl)c(Cl)cc1O]

### Step a

To a stirred solution of 4-(4,5-dichloro-2-[[2-(trimethylsilyl)ethoxy]methoxy]phenyl)piperidine-3-carboxamide (Example 29, step c) (0.11 g, 0.26 mmol) and Et₃N (53 mg, 0.53 mmol) in DCM (3 mL) was added Boc₂O (84 mg, 0.39 mmol) at room temperature. The reaction was stirred for 1 h at room temperature. The reaction was diluted with water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford tert-butyl 3-carbamoyl-4-(4,5-dichloro-2-[[2-(trimethylsilyl)ethoxy]methoxy]phenyl)piperidine-1-carboxylate as an off-white solid (0.11 g, 81%): LCMS (ESI) calc'd for C₂₃H₃₆Cl₂N₂O₅Si [M+H]⁺: 519, 521 (3:2), found: 519, 521 (3:2).

### Step b

To a stirred mixture of tert-butyl 3-carbamoyl-4-(4,5-dichloro-2-[[2-(trimethylsilyl)ethoxy]methoxy]phenyl)piperidine-1-carboxylate (95 mg, 0.18 mmol) and KOH (46 mg, 0.82 mmol) in ACN (2 mL) and water (0.5 mL) was added 1,3-dibromo-5,5-dimethylimidazolidine-2,4-dione (29 mg, 0.10 mmol) at 0° C. The reaction was stirred at room temperature for 2 h. The resulting solution was concentrated under reduced pressure. The residue was extracted with DCM/MeOH (10/1, 3×30 mL). The combined organic layers were dried over anhydrous Na₂SO₄ and filtered. The filtrate was concentrated under reduced pressure to afford tert-butyl 3-amino-4-(4,5-dichloro-2-[[2-(trimethylsilyl)ethoxy]methoxy]phenyl)piperidine-1-carboxylate as a light yellow solid (0.15 g, crude), which was directly used in next step without further purification: LCMS (ESI) calc'd for C₂₂H₃₆Cl₂N₂O₄Si [M+H]⁺: 491, 493 (3:2), found 491, 493 (3:2).

### Step c

To a stirred mixture of tert-butyl 3-amino-4-(4,5-dichloro-2-[[2-(trimethylsilyl)ethoxy]methoxy]phenyl)piperidine-1-carboxylate (0.15 g, 0.31 mmol) in DCM (1 mL) was added TFA (1 mL) at room temperature. The reaction was stirred at room temperature for 1 h. The reaction was concentrated under reduced pressure. The residue was purified by Prep-HPLC with the following conditions: Column: Sunfire Prep C₁₈ OBD Column, 10 m, 19×250 mm; Mobile Phase A: Water (plus 0.05% TFA), Mobile Phase B: ACN; Flow rate: 20 mL/min; Gradient: 13% B to 20% B in 8 min; Detector: UV 254/220 nm; Retention time: RT₁: 6.37 min; RT₂: 7.05 min. The fractions containing the desired product at 6.37 min were collected and concentrated under reduced pressure to afford Compound 49 ((3R,4R)-rel-2-(3-aminopiperidin-4-yl)-4,5-dichlorophenol (trans isomer)) as a light yellow solid (5.8 mg, 23% overall two steps): LCMS (ESI) calc'd for C₁₁H₁₄Cl₂N₂O [M+H]⁺: 261, 263 (3:2), found: 261, 263 (3:2); ¹H NMR (400 MHz, CD₃OD) δ 7.38 (s, 1H), 7.06 (s, 1H), 4.24-3.95 (m, 1H), 3.86-3.68 (m, 1H), 3.68-3.51 (m, 1H), 3.24-3.04 (m, 2H), 2.47-2.23 (m, 1H), 2.18-1.91 (m, 2H). Fractions containing the desired product at 7.05 min were collected and concentrated under reduced pressure to afford Compound 51 ((3R,4S)-rel-2-(3-aminopiperidin-4-yl)-4,5-dichlorophenol (cis isomer)) as a light yellow solid (15.8 mg, 17% overall two steps): LCMS (ESI) calc'd for C₁₁H₁₄Cl₂N₂O [M+H]⁺: 261, 263 (3:2), found: 261, 263 (3:2); ¹H NMR (400 MHz, CD₃OD) δ 7.32 (s, 1H), 7.06 (s, 1H), 4.27-4.16 (m, 1H), 3.79-3.57 (m, 5H), 2.68-2.46 (m, 1H), 2.13-1.94 (m, 1H).

### Example 36. Compound 52 ((3R,4S)-rel-4-(4,5-dichloro-2-hydroxyphenyl)pyrrolidine-3-carboxamide)

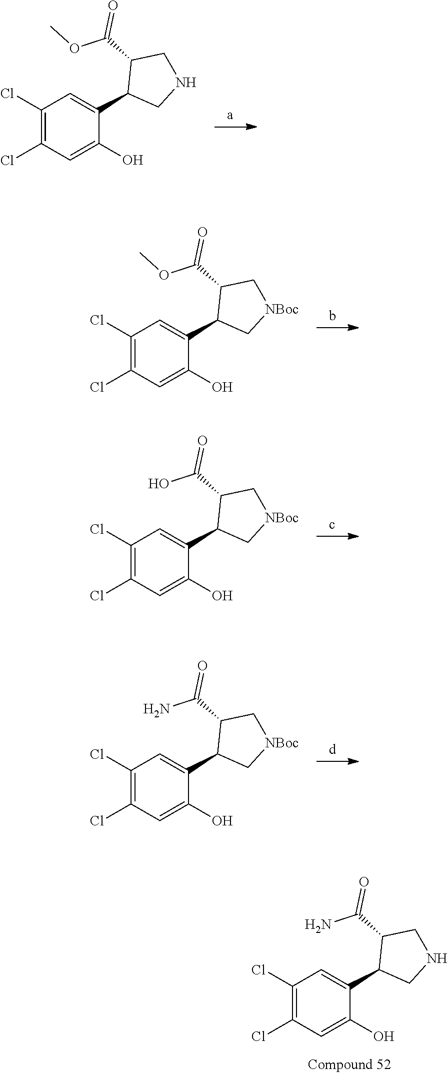 [SMILES: COC(=O)[C@@H]1CNC[C@H]1c1cc(Cl)c(Cl)cc1O ; COC(=O)[C@@H]1CN(C(=O)OC(C)(C)C)C[C@H]1c1cc(Cl)c(Cl)cc1O ; CC(C)(C)OC(=O)N1C[C@@H](C(=O)O)[C@H](c2cc(Cl)c(Cl)cc2O)C1 ; CC(C)(C)OC(=O)N1C[C@@H](C(N)=O)[C@H](c2cc(Cl)c(Cl)cc2O)C1 ; NC(=O)[C@@H]1CNC[C@H]1c1cc(Cl)c(Cl)cc1O]

### Step a

To a stirred mixture of methyl 4-(4,5-dichloro-2-hydroxyphenyl)pyrrolidine-3-carboxylate trans isomer (0.32 g, 1.10 mmol) and Boc₂O (0.14 g, 0.66 mmol) in DCM (5 mL) was added Et₃N (0.35 g, 3.42 mmol) at room temperature. The reaction was diluted with water (30 mL) at room temperature. The resulting mixture was extracted with EA (3×15 mL). The combined organic layers were washed with brine (3×30 mL) and dried over anhydrous Na₂SO₄. After filtration, the filtrate was concentrated under reduced pressure. The residue was purified by Prep-TLC, eluted with PE/EA (4/1) to afford 1-(tert-butyl) 3-methyl 4-(4,5-dichloro-2-hydroxyphenyl)pyrrolidine-1,3-dicarboxylate trans isomer as a light yellow oil (0.18 g, 42%): LCMS (ESI) calc'd for C₁₇H₂₁Cl₂NO₅ [M+H−15]⁺: 375, 375 (3:2), found 375, 375 (3:2).

### Step b

To a stirred solution of 1-(tert-butyl) 3-methyl 4-(4,5-dichloro-2-hydroxyphenyl)pyrrolidine-1,3-dicarboxylate trans isomer (0.10 g, 0.26 mmol) in MeOH (3 mL) and water (0.5 mL) was added NaOH (21 mg, 0.51 mmol) at room temperature. The reaction was stirred at 40° C. for 1 h. The reaction acidified to pH 4 with citric acid. The mixture was diluted with water (30 mL). The aqueous solution was extracted with EA (3×20 mL). The combined organic layers were washed with brine (3×20 mL) and dried over anhydrous Na₂SO₄. After filtration, the filtrate was concentrated under reduced pressure to afford 1-(tert-butoxycarbonyl)-4-(4,5-dichloro-2-hydroxyphenyl)pyrrolidine-3-carboxylic acid trans isomer as an off-white solid (0.10 g, crude), which was used to next step directly without further purification: LCMS (ESI) calc'd for C₁₆H₁₉Cl₂NO₅ [M+H]⁺: 376, 378 (3:2), found 376, 378 (3:2).

### Step c

To a stirred solution of 1-(tert-butoxycarbonyl)-4-(4,5-dichloro-2-hydroxyphenyl)pyrrolidine-3-carboxylic acid (0.10 g, 0.27 mmol) and EDCI (0.10 g, 0.54 mmol) in DMF (2 mL) were added HOBT (73 mg, 0.54 mmol) and NH₄Cl (71 mg, 1.35 mmol) and Et₃N (0.11 g, 1.08 mmol) at room temperature. The reaction was stirred at room temperature for 1 h diluted with water (30 mL) and EA (30 mL). The isolated aqueous solution was extracted with EA (3×20 mL). The combined organic layers were washed with brine (3×20 mL) and dried over anhydrous Na₂SO₄. After filtration, the filtrate was concentrated under reduced pressure. The residue was purified by Prep-TLC, eluted with PE/EA (1/1) to afford tert-butyl 3-carbamoyl-4-(4,5-dichloro-2-hydroxyphenyl)pyrrolidine-1-carboxylate trans isomer (61 mg, 63% overall two steps) as a light yellow oil: LCMS (ESI) calc'd for C₁₆H₂₀Cl₂N₂O₄ [M+H]⁺: 375, 377 (3:2), found 375, 377 (3:2).

### Step d

To a stirred solution of tert-butyl 3-carbamoyl-4-(4,5-dichloro-2-hydroxyphenyl)pyrrolidine-1-carboxylate trans isomer (61 mg, 0.13 mmol) in DCM (2 mL) was added TFA (2 mL) at room temperature. The reaction was concentrated under reduced pressure. The residue was purified by Prep-HPLC with the following conditions: Column: XBridge C₁₈ OBD Prep Column, 100 Å, 10 μm, 19 mm×250 mm; Mobile Phase A: Water with 20 mmol/L NH₄HCO₃, Mobile Phase B: ACN; Flow rate: 25 mL/min; Gradient: 13% B to 26% B in 6.5 min; Detector: UV: 254/210 nm; Retention Time: 5.35 min. The fractions containing the desired product were collected and concentrated under reduced pressure to afford Compound 52 ((3R,4S)-rel-4-(4,5-dichloro-2-hydroxyphenyl)pyrrolidine-3-carboxamide (trans isomer)) as an off-white solid (18.5 mg, 42%): LCMS (ESI) calc'd for C₁₁H₁₂Cl₂N₂O₂ [M+H]⁺: 275, 277 (3:2), found 275, 277 (3:2); ¹H NMR (300 MHz, CD₃OD) δ 7.25 (s, 1H), 6.90 (s, 1H), 3.62 (q, J=7.0 Hz, 1H), 3.52-3.41 (m, 1H), 3.41-3.34 (m, 1H), 3.22-3.07 (m, 3H).

### Example 37. Compound 53 ((3R,4S)-rel-methyl 4-(4,5-dichloro-2-hydroxyphenyl)pyrrolidine-3-carboxylate)

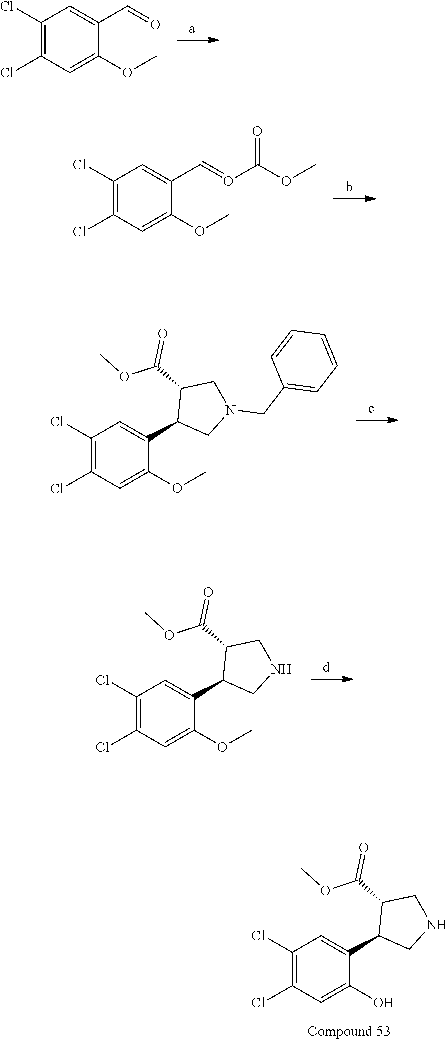

### Step a

To a stirred solution of 4,5-dichloro-2-methoxybenzaldehyde (1.00 g, 4.88 mol) in THE (30 mL) was added methyl (2-triphenylphosphoranylidene)acetate (3.26 g, 9.75 mol) at room temperature under nitrogen atmosphere. The resulting mixture was stirred for 1 h at room temperature under nitrogen atmosphere. The reaction was quenched with water (50 mL) at room temperature. The resulting mixture was extracted with EA (3×80 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (5/1) to afford methyl (2E)-3-(4,5-dichloro-2-methoxyphenyl)prop-2-enoate as an off-white solid (0.88 g, 69%): LCMS (ESI) calc'd for C₁₁H₁₀Cl₂O₃ [M+H]⁺: 261, 263 (3:2), found: 261, 263 (3:2); ¹H NMR (400 MHz, CDCl₃) δ 7.86 (d, J=16.2 Hz, 1H), 7.57 (s, 1H), 7.02 (s, 1H), 6.52 (d, J=16.1 Hz, 1H), 3.91 (s, 3H), 3.83 (s, 3H).

### Step b

To a stirred solution of benzyl(methoxymethyl)[(trimethylsilyl)methyl]amine (0.87 g, 3.68 mmol) in DCM (8 mL) was added methyl (2E)-3-(4,5-dichloro-2-methoxyphenyl)prop-2-enoate (0.80 g, 3.06 mmol) at room temperature under nitrogen atmosphere. The mixture was stirred for additional 16 h at room temperature. The reaction was quenched with water (50 mL) at room temperature. The resulting mixture was extracted with DCM (3×50 mL). The combined organic layers were washed with brine (2×30 mL) and dried over anhydrous Na₂SO₄. After filtration, the filtrate was concentrated under reduced pressure. The residue was purified by reverse phase chromatography, eluted with 65% ACN in water (plus 0.05% TFA) to afford (3R,4S)-rel-methyl 1-benzyl-4-(4,5-dichloro-2-methoxyphenyl)pyrrolidine-3-carboxylate (trans isomer) as a colorless oil (1.08 g, 89%): LCMS (ESI) calc'd for C₂₀H₂₁Cl₂NO₃ [M+H]⁺: 394, 396 (3:2), found 394, 396 (3:2); ¹H NMR (300 MHz, CD₃OD) δ 7.61-7.43 (m, 6H), 7.22 (s, 1H), 4.49 (s, 2H), 3.93-3.78 (m, 4H), 3.78-3.71 (m, 2H), 3.71-59 (m, 4H), 3.58-3.46 (m, 2H).

### Step c

To a stirred solution of 1-benzyl-4-(4,5-dichloro-2-methoxyphenyl)pyrrolidine-3-carboxylate trans isomer (1.00 g, 2.54 mmol) in toluene (10 mL) was added 1-chloroethyl chloroformate (0.73 g, 5.07 mmol) at room temperature under nitrogen atmosphere. The resulting mixture was stirred for 5 h at 100° C. under nitrogen atmosphere. The resulting mixture was quenched with MeOH (3 mL) at room temperature. The resulting mixture was concentrated under reduced pressure. The residue was purified by reverse phase chromatography, eluted with 65% ACN in water (plus 0.05% TFA) to afford methyl 4-(4,5-dichloro-2-methoxyphenyl)pyrrolidine-3-carboxylate trans isomer as a colorless oil (0.70 g, 66%): LCMS (ESI) calc'd for C₁₃H₁₅Cl₂NO₃ [M+H]⁺: 304, 306 (3:2), found 304, 306 (3:2); ¹H NMR (300 MHz, CD₃OD) δ 7.43 (s, 1H), 7.09 (s, 1H), 3.92-3.71 (m, 4H), 3.71-3.57 (m, 4H), 3.18-3.06 (m, 1H), 3.05-2.89 (m, 3H).

### Step d

To a stirred solution of methyl 4-(4,5-dichloro-2-methoxyphenyl)pyrrolidine-3-carboxylate trans isomer (0.80 g, 1.91 mmol) in DCM (5 mL) was added BBr₃ (3.85 g, 15.35 mmol) at room temperature. The resulting mixture was stirred for 2 h at room temperature. The reaction was quenched with water (3 mL) at room temperature. The resulting mixture was concentrated under reduced pressure. The residue was purified by reverse phase chromatography, eluted with 50% ACN in water (plus 0.05% TFA) to afford Compound 53 ((3R,4S)-rel-methyl 4-(4,5-dichloro-2-hydroxyphenyl)pyrrolidine-3-carboxylate (trans isomer)) as an off-white solid (0.50 g, 65%): LCMS (ESI) calc'd for C₁₂H₁₃Cl₂NO₃ [M+H]⁺: 290, 292 (3:2), found 290, 292 (3:2); ¹H NMR (400 MHz, D₂O) δ 7.36 (s, 1H), 7.03 (s, 1H), 3.84-3.72 (m, 2H), 3.72-3.63 (m, 2H), 3.62 (s, 3H), 3.61-3.46 (m, 2H).

### Example 38. Compound 54 (2,3,4-trichloro-6-(piperidin-4-yl)phenol)

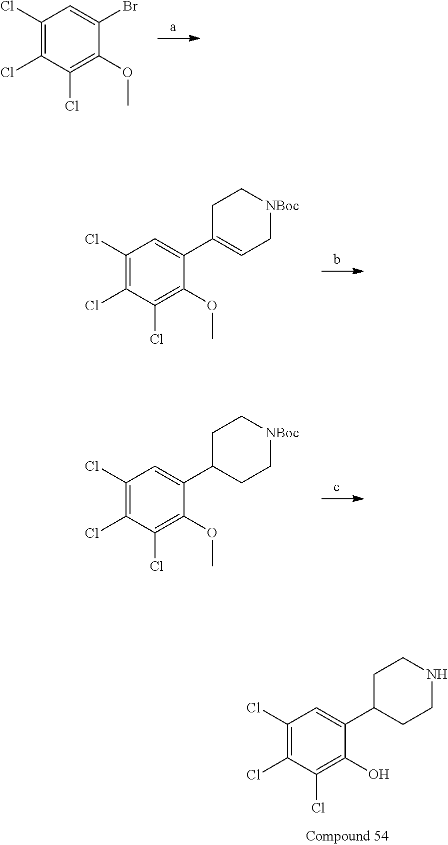 [SMILES: COc1c(Br)cc(Cl)c(Cl)c1Cl ; COc1c(C2=CCN(C(=O)OC(C)(C)C)CC2)cc(Cl)c(Cl)c1Cl ; COc1c(C2CCN(C(=O)OC(C)(C)C)CC2)cc(Cl)c(Cl)c1Cl ; Oc1c(C2CCNCC2)cc(Cl)c(Cl)c1Cl]

### Step a

To a mixture of 1-bromo-3,4,5-trichloro-2-methoxybenzene (0.20 g, 0.69 mmol), tert-butyl 4-(4,4,5,5-tetramethyl-1,3,2-dioxaborolan-2-yl)-1,2,3,6-tetrahydropyridine-1-carboxylate (0.24 g, 0.78 mmol) and Na₂CO₃ (0.22 g, 2.08 mmol) in water (1 mL) and 1,4-dioxane (5 mL) was added Pd(PPh₃)₄ (20 mg, 0.02 mmol) at room temperature under nitrogen atmosphere. The mixture was stirred 80° C. for 3 h under nitrogen atmosphere. The reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford to afford tert-butyl 4-(3,4,5-trichloro-2-methoxyphenyl)-1,2,3,6-tetrahydropyridine-1-carboxylate as a light oil (0.20 g, 73%): LCMS (ESI) calc'd for C₁₇H₂₀Cl₃NO₃ [M+H−15]⁺: 377, 379, 381 (3:3:1), found 377, 379, 381 (3:3:1); ¹H NMR (400 MHz, CDCl₃) δ 7.22 (s, 1H), 5.88 (s, 1H), 4.09-4.04 (m, 2H), 3.75 (s, 3H), 3.60 (t, J=5.6 Hz, 2H), 2.50-2.43 (m, 2H), 1.50 (s, 9H).

### Step b

To a solution of tert-butyl 4-(3,4,5-trichloro-2-methoxyphenyl)-1,2,3,6-tetrahydropyridine-1-carboxylate (0.10 g, 0.25 mmol) in MeOH (4 mL) was added PtO₂ (50 mg, 0.22 mmol) at room temperature. The reaction mixture was stirred at room temperature under hydrogen atmosphere (1.5 atm) for 3 h. The mixture was filtered and the filtrate was concentrated under reduced pressure to afford tert-butyl 4-(3,4,5-trichloro-2-methoxyphenyl)piperidine-1-carboxylate as a colorless oil (87 mg, 78%): LCMS (ESI) calc'd for C₁₇H₂₂Cl₃NO₃ [M+H−15]⁺: 379, 381, 383 (3:3:1), found 379, 381, 383 (3:3:1).

### Step c

To a stirred solution of tert-butyl 4-(3,4,5-trichloro-2-methoxyphenyl)piperidine-1-carboxylate (80 mg, 0.20 mmol) in DCM (0.5 mL) was added BBr₃ (0.40 g, 1.60 mmol) at room temperature. The reaction was stirred at room temperature for 1 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 25 mL/min; Gradient: 16% B to 52% B in 7 min; Detector: UV 254/220 nm; Retention time: 6.58 min. The fractions containing the desired product were collected and concentrated under reduced pressure to afford Compound 54 (2,3,4-trichloro-6-(piperidin-4-yl)phenol) as an off-white solid (25 mg, 42%): LCMS (ESI) calc'd for C₁₁H₁₂Cl₃NO [M+H]⁺: 280, 282, 284 (3:3:1), found 280, 282, 284 (3:3:1); ¹H NMR (300 MHz, CD₃OD) δ 6.98 (s, 1H), 3.43-3.33 (m, 1H), 3.10-2.95 (m, 3H), 2.03-1.92 (m, 3H), 1.80-1.65 (m, 2H).

### Example 39. Compound 55 (3,4-dichloro-2-(piperidin-4-yl)phenol)

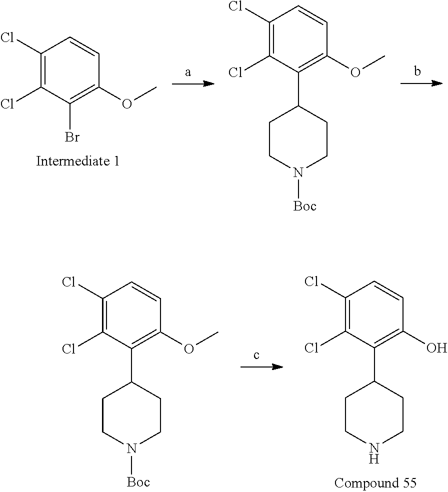 [SMILES: COc1ccc(Cl)c(Cl)c1Br ; COc1ccc(Cl)c(Cl)c1C1CCN(C(=O)OC(C)(C)C)CC1 ; COc1ccc(Cl)c(Cl)c1C1CCN(C(=O)OC(C)(C)C)CC1 ; Oc1ccc(Cl)c(Cl)c1C1CCNCC1]

### Step a

To a mixture of Intermediate 1 (0.30 g, 1.17 mmol) and tert-butyl 4-(4,4,5,5-tetramethyl-1,3,2-dioxaborolan-2-yl)-3,6-dihydropyridine-1(2H)-carboxylate (0.44 g, 1.41 mmol) and Na₂CO₃ (0.38 g, 3.54 mmol) in water (1 mL) and 1,4-dioxane (5 mL) was added Pd(dppf)Cl₂.CH₂Cl₂ (20 mg, 0.02 mmol) at room temperature under nitrogen atmosphere. The mixture was stirred 80° C. for 3 h under nitrogen atmosphere. After cooling to room temperature, the reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford to afford tert-butyl 4-(2,3-dichloro-6-methoxyphenyl)-1,2,3,6-tetrahydropyridine-1-carboxylate as a light solid (0.38 g, 81%): LCMS (ESI) calc'd for C₁₇H₂₁Cl₂NO₃ [M+H−15]⁺: 343, 345 (3:2), found 343, 345 (3:2).

### Step b

To a stirred solution of tert-butyl 4-(2,3-dichloro-6-methoxyphenyl)-1,2,3,6-tetrahydropyridine-1-carboxylate (0.20 g, 0.56 mmol) in MeOH (4 mL) was added PtO₂ (50 mg, 0.22 mmol) at room temperature. The reaction mixture was stirred at room temperature under hydrogen atmosphere (1.5 atm) for 2 h. The mixture was filtered and the filtrate was concentrated under reduced pressure. The residue was purified by Prep-TLC, eluted with DCM/MeOH (40/1) to afford tert-butyl 4-(2,3-dichloro-6-methoxyphenyl)piperidine-1-carboxylate as a yellow solid (95 mg, 38%): LCMS (ESI) calc'd for C₁₇H₂₃Cl₂NO₃ [M+H−15]⁺: 345, 347 (3:2), found 345, 347 (3:2);

### Step c

To a stirred solution of tert-butyl 4-(2,3-dichloro-6-methoxyphenyl)piperidine-1-carboxylate (94 mg, 0.26 mmol) in DCM (1 mL) was added BBr₃ (0.52 g, 2.08 mmol) at room temperature. The reaction was stirred at room temperature for 1 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water (plus 0.05% TFA), Mobile Phase B: ACN; Flow rate: 25 mL/min; Gradient: 5% B to 60% B in 9 min; Detector: UV 254/210 nm; Retention time: 7.83 min. The fractions containing the desired product were collected and concentrated under reduced pressure to afford Compound 55 (3,4-dichloro-2-(piperidin-4-yl)phenol) as an off-white solid (16 mg, 27%): LCMS (ESI) calc'd for C₁₁H₁₃Cl₂NO [M+H]⁺: 246, 248 (3:2), found 246, 248 (3:2); ¹H NMR (300 MHz, CD₃OD) δ 7.24 (d, J=8.8 Hz, 1H), 6.75 (d, J=8.8 Hz, 1H), 3.71 (t, J=12.5 Hz, 1H), 3.49 (d, J=12.5 Hz, 2H), 3.17-3.06 (m, 2H), 2.89-2.73 (m, 2H), 1.82 (d, J=14.2 Hz, 2H).

### Example 40. Compound 57 (3,5-dichloro-2-(piperidin-4-yl)phenol)

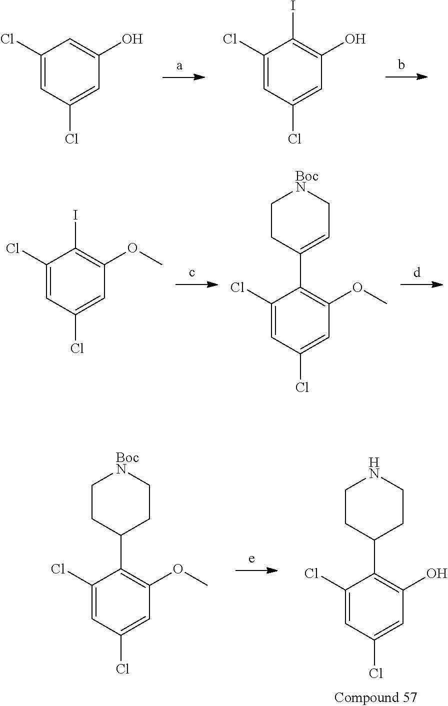 [SMILES: Oc1cc(Cl)cc(Cl)c1 ; Oc1cc(Cl)cc(Cl)c1I ; COc1cc(Cl)cc(Cl)c1I ; COc1cc(Cl)cc(Cl)c1C1=CCN(C(=O)OC(C)(C)C)CC1 ; COc1cc(Cl)cc(Cl)c1C1CCN(C(=O)OC(C)(C)C)CC1 ; Oc1cc(Cl)cc(Cl)c1C1CCNCC1]

### Step a

To a stirred solution of 3,5-dichlorophenol (2.50 g, 15.34 mmol) in THE (20 mL) was slowly added NaH (1.23 g, 30.75 mmol, 60%) at 0° C. under nitrogen atmosphere. The reaction mixture was warmed to room temperature and stirred for 20 min. After cooling to 0° C., 12 (3.89 g, 15.33 mmol) was added, and then the reaction mixture was stirred at room temperature for 3 h. The reaction was quenched with saturated aq. Na₂S₂O₃(20 mL) at 0° C. The mixture was acidified to pH 7 with aq. HCl (5 mL, 2 N). The resulting mixture was extracted with EA (3×50 mL). The combined organic layers were washed with brine (2×50 mL) and dried over anhydrous Na₂SO₄. After filtration, the filtrate was concentrated under reduced pressure. The residue was purified by Prep-HPLC with the following conditions: Column: XBridge C₁₈ OBD Prep Column 100 Å, 10 m, 19 mm×250 mm; Mobile Phase A: water (plus 0.05% TFA), Mobile Phase B: ACN; Flow rate: 25 mL/min; Gradient: 60% B to 90% B in 6.5 min; Detector: UV: 254/210 nm; Retention time: 4.68 min. The fractions containing the desired product were collected and concentrated under reduced pressure to afford 3,5-dichloro-2-iodophenol as a yellow solid (0.50 g, 11%): LCMS (ESI) calc'd for C₆H₃C₁₂IO [M−H]⁺: 287, 289 (3:2), found 287, 289 (3:2); ¹H NMR (300 MHz, CDCl₃) δ 7.10 (d, J=2.3 Hz, 1H), 6.94 (d, J=2.3 Hz, 1H).

### Step b

To a stirred mixture of 3,5-dichloro-2-iodophenol (0.46 g, 1.59 mmol) and K₂CO₃ (0.66 g, 4.78 mmol) in DMF (5 mL) was added MeI (0.45 g, 3.18 mmol) at room temperature. The resulting mixture was stirred for 1 h at room temperature. The reaction was diluted with EA (30 mL) and water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (15/1) to afford 1,5-dichloro-2-iodo-3-methoxybenzene as a yellow solid (0.43 g, 80%): ¹H NMR (300 MHz, CDCl₃) δ 7.14 (d, J=2.1 Hz, 1H), 6.69 (d, J=2.1 Hz, 1H), 3.90 (s, 3H).

### Step c

To a stirred mixture of 1,5-dichloro-2-iodo-3-methoxybenzene (0.43 g, 1.43 mmol), Pd(dppf)Cl₂ (0.10 g, 0.14 mmol) and Na₂CO₃ (0.45 g, 4.28 mmol) in 1,4-dioxane (4 mL) and H₂O (1 mL) was added tert-butyl 4-(4-amino-4,5,5-trimethyl-1,3,2-dioxaborolan-2-yl)-1,2,3,6-tetrahydropyridine-1-carboxylate (0.66 g, 2.14 mmol) at room temperature under argon atmosphere. The resulting mixture was stirred for 2 h at 80° C. under argon atmosphere. The reaction mixture was poured into water (30 mL) and extracted with EA (3×30 mL). The combined organic layers were washed with brine (2×30 mL) and dried over anhydrous Na₂SO₄. After filtration, the filtrate was concentrated under reduced pressure. The residue was purified by Prep-TLC, eluted with PE/EA (5/1) to afford tert-butyl 4-(2,4-dichloro-6-methoxyphenyl)-1,2,3,6-tetrahydropyridine-1-carboxylate as a yellow solid (0.25 g, 44%): LCMS (ESI) calc'd for C₁₇H₂₁Cl₂NO₃ [M+H−15]⁺: 343, 345 (3:2), found: 343, 345 (3:2); ¹H NMR (400 MHz, CDCl₃) δ 7.05 (d, J=1.9 Hz, 1H), 6.79 (d, J=1.9 Hz, 1H), 5.56 (s, 1H), 4.09-4.02 (m, 2H), 4.00-3.94 (m, 1H), 3.67-3.59 (m, 2H), 3.46 (t, J=5.6 Hz, 1H), 2.31-2.21 (m, 3H), 1.52 (s, 9H).

### Step d

To a solution of tert-butyl 4-(2,4-dichloro-6-methoxyphenyl)-1,2,3,6-tetrahydropyridine-1-carboxylate (0.32 g, 0.89 mmol) in MeOH (4 mL) was added PtO₂ (32 mg, 0.14 mmol) at room temperature. The reaction mixture was stirred at room temperature under hydrogen atmosphere (1.5 atm) for 2 h. The mixture was filtered and the filtrate was concentrated under reduced pressure to afford tert-butyl 4-(2,4-dichloro-6-methoxyphenyl)piperidine-1-carboxylate as a colorless oil (0.32 g, crude), which was used to next step directly without further purification: LCMS (ESI) calc'd for C₁₇H₂₃Cl₂NO₃ [M+H]⁺: 345, 347 (3:2), found 345, 347 (3:2);

### Step e

To a stirred solution of tert-butyl 4-(2,4-dichloro-6-methoxyphenyl)piperidine-1-carboxylate (0.32 g, 0.89 mmol) in DCM (4 mL) was added BBr₃ (1.78 g, 7.11 mmol) at room temperature. The reaction was stirred at room temperature for 2 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 25 mL/min; Gradient: 20% B to 70% B in 6.5 min; Detector: UV 254/210 nm; Retention time: 4.63 min. The fractions containing the desired product were collected and concentrated under reduced pressure to afford Compound 57 (3,5-dichloro-2-(piperidin-4-yl)phenol) as an off-white solid. (105.4 mg, 46%): LCMS (ESI) calc'd for C₁₁H₁₃Cl₂NO [M+H]⁺: 246, 248 (3:2), found 246, 248 (3:2); ¹H NMR (300 MHz, CD₃OD) δ 6.76 (d, J=2.1 Hz, 1H), 6.68 (d, J=2.2 Hz, 1H), 3.47 (t, J=11.7 Hz, 1H), 3.26-3.18 (m, 2H), 2.86-2.72 (m, 2H), 2.67-2.50 (m, 2H), 1.57 (d, J=12.8 Hz, 2H).

### Example 41. Compound 56 (3R,4R)-rel-4-(4,5-dichloro-2-hydroxyphenyl)piperidine-3-carboxamide isomer 1) and Compound 58 (3R,4R)-rel-4-(4,5-dichloro-2-hydroxyphenyl)piperidine-3-carboxamide isomer 2)

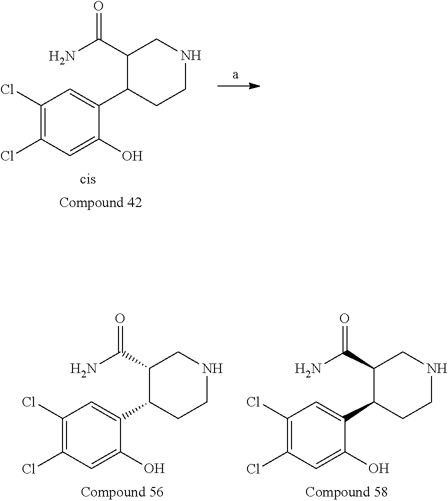 [SMILES: NC(=O)C1CNCCC1c1cc(Cl)c(Cl)cc1O ; NC(=O)[C@@H]1CNCC[C@@H]1c1cc(Cl)c(Cl)cc1O ; NC(=O)[C@H]1CNCC[C@H]1c1cc(Cl)c(Cl)cc1O]

The absolute configurations for Compounds 56 and 58 were arbitrarily assigned.

### Step a

4-(4,5-dichloro-2-hydroxyphenyl)piperidine-3-carboxamide cis isomer (Compound 42, Example 29) (0.10 g, 0.34 mmol) was separated by Prep Chiral HPLC with the following conditions: Column: Chiralpak IG, 20×250 mm, 5 m; Mobile Phase A: Hex (plus 0.2% IPA), Mobile Phase B: EtOH; Flow rate: 20 mL/min; Gradient: 30% B to 30% B in 13 min; Detector: UV 254/220 nm; Retention time: RT₁: 7.19 min; RT₂: 11.84 min; Injection Volume: 1.5 mL; Number Of Runs: 4. The faster-eluting enantiomer Compound 56 ((3R,4R)-rel-4-(4,5-dichloro-2-hydroxyphenyl)piperidine-3-carboxamide isomer 1) at 7.19 min was obtained as an off-white solid (30 mg, 30%): LCMS (ESI) calc'd for C₁₂H₁₄Cl₂N₂O₂ [M+H]⁺: 289, 291 (3:2), found 289, 291 (3:2); ¹H NMR (300 MHz, DMSO-d₆) δ 7.61 (s, 1H), 7.11 (s, 1H), 6.97 (s, 1H), 6.81 (s, 1H), 3.22-3.07 (m, 3H), 2.85-2.70 (m, 1H), 2.68-2.53 (m, 2H), 2.39-2.21 (m, 1H), 1.38 (d, J=12.6 Hz, 1H). The slower-eluting Compound 58 ((3R,4R)-rel-4-(4,5-dichloro-2-hydroxyphenyl)piperidine-3-carboxamide isomer 2) at 11.84 min was obtained as an off-white solid (30 mg, 30%): LCMS (ESI) calc'd for C₁₂H₁₄Cl₂N₂O₂ [M+H]⁺: 289, 291 (3:2), found 289, 291 (3:2); ¹H NMR (300 MHz, DMSO-d₆) δ 7.64 (d, J=3.4 Hz, 1H), 7.11 (s, 1H), 6.95 (s, 1H), 6.77 (s, 1H), 3.21-3.00 (m, 3H), 2.76 (dd, J=12.8, 3.5 Hz, 1H), 2.65-2.53 (m, 2H), 2.37-2.14 (m, 1H), 1.46-1.27 (m, 1H).

### Example 42. Compound 60 (4-(2,3,4-trichloro-6-hydroxyphenyl)piperidine-1-carboxamide)

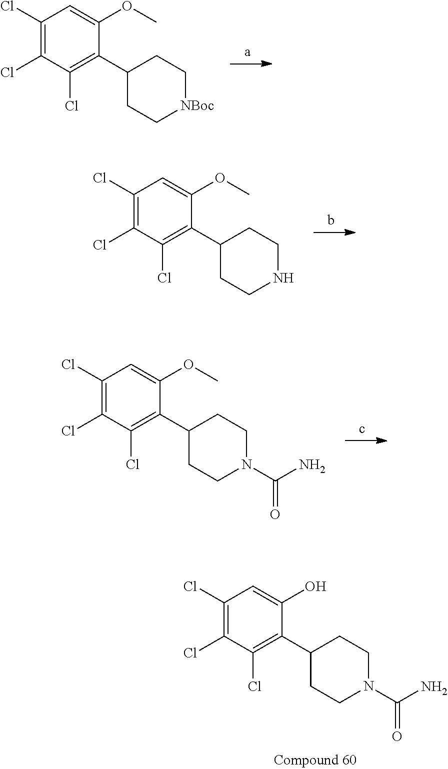 [SMILES: COc1cc(Cl)c(Cl)c(Cl)c1C1CCN(C(=O)OC(C)(C)C)CC1 ; COc1cc(Cl)c(Cl)c(Cl)c1C1CCNCC1 ; COc1cc(Cl)c(Cl)c(Cl)c1C1CCN(C(N)=O)CC1 ; NC(=O)N1CCC(c2c(O)cc(Cl)c(Cl)c2Cl)CC1]

### Step a

To a mixture of tert-butyl 4-(2,3,4-trichloro-6-methoxyphenyl)piperidine-1-carboxylate (Example 31, step e) (0.17 g, 0.43 mmol) in DCM (2 mL) was added TFA (1 mL) at room temperature. The reaction was stirred at room temperature for 1 h. The reaction was concentrated under reduced pressure to afford 4-(2,3,4-trichloro-6-methoxyphenyl)piperidine as a light yellow solid (0.17 g, crude), which was used to next step directly without further purification: LCMS (ESI) calc'd for C₁₂H₁₄Cl₃NO [M+H]⁺: 294, 296, 298 (3:3:1), found 294, 296, 298 (3:3:1); ¹H NMR (300 MHz, CD₃OD) δ 7.18 (s, 1H), 3.87 (s, 3H), 3.29-3.19 (m, 1H), 2.92-2.77 (m, 2H), 2.58-2.38 (m, 2H), 1.68-1.56 (m, 2H), 0.99-0.83 (m, 2H).

### Step b

To a stirred mixture of 4-(2,3,4-trichloro-6-methoxyphenyl)piperidine (0.17 g, 0.42 mmol) and Et₃N (84 mg, 0.83 mmol) in DCM (3 mL) was added TMSNCO (72 mg, 0.62 mmol) at room temperature. The reaction mixture was stirred at room temperature for 2 h. The reaction was diluted with EA (30 mL) and water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (5/1) to afford 4-(2,3,4-trichloro-6-methoxyphenyl)piperidine-1-carboxamide as an off-white solid (80 mg, 57% overall two steps): LCMS (ESI) calc'd for C₁₃H₁₅C₁₃N₂O₂ [M+H]⁺: 337, 339, 341 (3:3:1), found 337, 339, 341 (3:3:1); ¹H NMR (300 MHz, CDCl₃) δ 6.91 (s, 1H), 4.22-3.98 (m, 1H), 3.81 (s, 3H), 3.62-3.43 (m, 2H), 3.04-2.80 (m, 1H), 2.48-2.19 (m, 2H), 1.69-1.50 (m, 2H), 1.37-1.16 (m, 1H).

### Step c

To a stirred solution of 4-(2,3,4-trichloro-6-methoxyphenyl)piperidine-1-carboxamide (80 mg, 0.24 mmol) in DCM (1 mL) was added BBr₃ (0.35 g, 1.43 mmol) at room temperature. The reaction was stirred at room temperature for 1 h. The reaction was quenched with water (1 mL) and the mixture was adjusted pH to 7-8 with saturated aq. NaHCO₃. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 25 mL/min; Gradient: 40% B to 55% B in 7 min; Detector: UV 254/210 nm; Retention time: 6.27 min. The fractions containing the desired product were collected and concentrated under reduced pressure to afford Compound 60 (4-(2,3,4-trichloro-6-hydroxyphenyl)piperidine-1-carboxamide) as an off-white solid (37 mg, 46%): LCMS (ESI) calc'd for C₁₂H₁₃C₁₃N₂O₂ [M+H]⁺: 323, 325, 327 (3:3:1), found 323, 325, 327 (3:3:1); ¹H NMR (300 MHz, CD₃OD) δ 6.91 (s, 1H), 4.14 (d, J=13.4 Hz, 2H), 3.65-3.43 (m, 1H), 3.02-2.74 (m, 2H), 2.58-2.28 (m, 2H), 1.54 (d, J=13.0 Hz, 2H).

### Example 43. Compound 61 ((2R,4S)-rel-4-(2,3,4-trichloro-6-hydroxyphenyl)piperidine-2-carboxamide cis isomer)

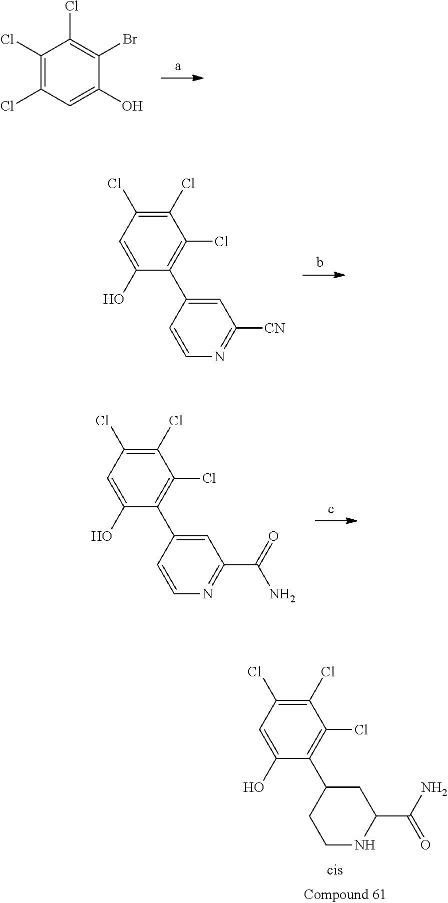 [SMILES: Oc1cc(Cl)c(Cl)c(Cl)c1Br ; N#Cc1cc(-c2c(O)cc(Cl)c(Cl)c2Cl)ccn1 ; NC(=O)c1cc(-c2c(O)cc(Cl)c(Cl)c2Cl)ccn1 ; NC(=O)C1CC(c2c(O)cc(Cl)c(Cl)c2Cl)CCN1]

### Step a

To a solution of 2-bromo-3,4,5-trichlorophenol (Example 31, step b) (1.50 g, 5.43 mmol) and 4-(4,4,5,5-tetramethyl-1,3,2-dioxaborolan-2-yl)pyridine-2-carbonitrile (1.50 g, 6.52 mmol) in 1,4-dioxane (10 mL) and water (2 mL) were added Na₂CO₃ (1.70 g, 16.28 mmol) and Pd(dppf)Cl₂.CH₂Cl₂ (0.45 g, 0.54 mmol) at room temperature under nitrogen atmosphere. The reaction mixture was stirred for 3 h at 80° C. under nitrogen atmosphere. The reaction mixture was allowed to cool to room temperature and diluted with water (50 mL) and extracted with EA (3×50 mL). The combined organic layers were washed with brine (2×40 mL) and dried over anhydrous Na₂SO₄. After filtration, the filtrate was concentrated under reduced pressure. The residue was purified by silica gel column chromatography, eluted with PE/EA (3/1) to afford 4-(2,3,4-trichloro-6-hydroxyphenyl)pyridine-2-carbonitrile as a light yellow solid (1.40 g, 69%): LCMS (ESI) calc'd for C₁₂H₅C₁₃N₂O [M+H]⁺: 299, 301, 303 (3:3:1), found 299, 301, 303 (3:3:1); ¹H NMR (400 MHz, CD₃OD) δ 8.79 (dd, J=5.0, 0.9 Hz, 1H), 7.88 (dd, J=1.6, 0.9 Hz, 1H), 7.65 (dd, J=5.1, 1.6 Hz, 1H), 7.11 (s, 1H).

### Step b

To a stirred solution of 4-(2,3,4-trichloro-6-hydroxyphenyl)pyridine-2-carbonitrile (0.90 g, 3.00 mmol) in MeOH (10 mL) were added a solution of NaOH (1.20 g, 30.05 mmol) in water (3 mL) and H₂O₂ (1.02 g, 30.05 mmol, 30%) dropwise at room temperature. The reaction was stirred for 1 h at room temperature. The reaction mixture was quenched with saturated aq. Na₂S₂O₃ (30 mL) and extracted with EA (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with DCM/MeOH (10/1) to afford 4-(2,3,4-trichloro-6-hydroxyphenyl)pyridine-2-carboxamide as an off-white solid (0.79 g, 66%): LCMS (ESI) calc'd for C₁₂H₇C₁₃N₂O₂ [M+H]⁺: 317, 319, 321 (3:3:1), found 317, 319, 321 (3:3:1); ¹H NMR (400 MHz, CD₃OD) δ 8.72 (dd, J=4.9, 0.9 Hz, 1H), 8.03 (dd, J=1.7, 0.8 Hz, 1H), 7.50 (dd, J=5.0, 1.7 Hz, 1H), 7.12 (s, 1H).

### Step c

To a stirred mixture of 4-(2,3,4-trichloro-6-hydroxyphenyl)pyridine-2-carboxamide (0.30 g, 0.95 mmol) in MeOH (5 mL) was added aq. HCl (6 N, 0.5 mL) at room temperature. The reaction was stirred for 5 h at 30° C. under hydrogen (50 atm) atmosphere. The reaction mixture was filtered and the filtrate was adjusted to pH 8 with saturated aq. NaHCO₃. The resulting solution was concentrated under reduced pressure. The residue was purified by Prep-HPLC with following conditions: Column: XBridge C₁₈ OBD Prep Column 100 Å, 10 μm, 19 mm×250 mm; Mobile Phase A: Water with 20 mmol/L NH₄HCO₃, Mobile Phase B: ACN; Flow rate: 25 mL/min; Gradient: 25% B to 58% B in 6 min; Detector: UV 254/210 nm; Retention time: 5.87 min. The fractions containing the desired product were collected and concentrated under reduced pressure to afford Compound 61 ((2R,4S)-rel-4-(2,3,4-trichloro-6-hydroxyphenyl)piperidine-2-carboxamide (cis isomer)) as an off-white solid (0.15 g, 47%): LCMS (ESI) calc'd for C₁₂H₁₃C₁₃N₂O₂ [M+H]⁺: 323, 325, 327 (3:3:1), found 323, 325, 327 (3:3:1); ¹H NMR (400 MHz, CD₃OD) δ 6.91 (s, 1H), 3.67-3.52 (m, 1H), 3.44-3.35 (m, 1H), 3.29-3.22 (m, 1H), 2.84-2.73 (m, 1H), 2.53-2.38 (m, 2H), 1.90-1.81 (m, 1H), 1.58-1.50 (m, 1H).

### Example 44. Compound 62 (4,5-dichloro-2-[3-(1H-1,2,3-triazol-1-yl)piperidin-4-yl]phenol)

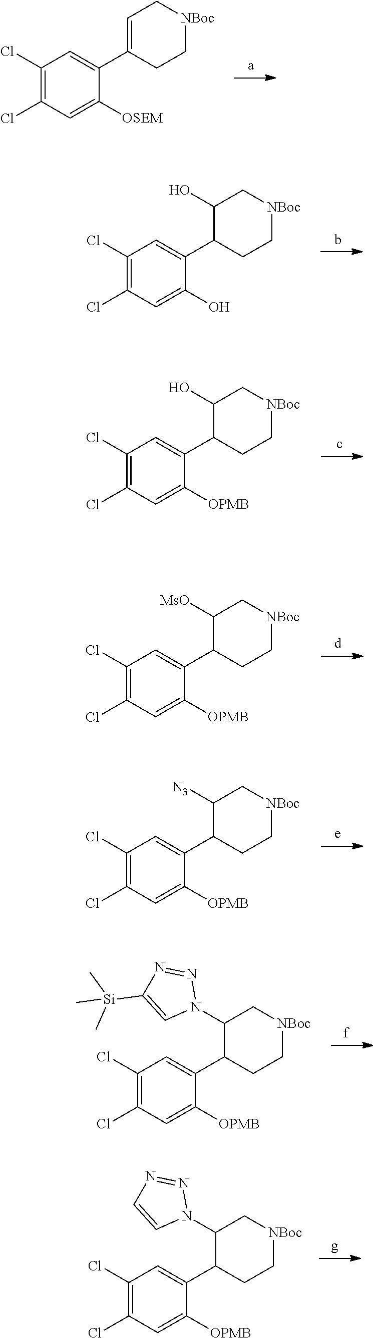

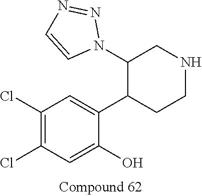 [SMILES: Oc1cc(Cl)c(Cl)cc1C1CCNCC1n1ccnn1]

### Step a

To a stirred solution of tert-butyl 4-(4,5-dichloro-2-[[2-(trimethylsilyl)ethoxy]methoxy]phenyl)-1,2,3,6-tetrahydropyridine-1-carboxylate (Example 16, step a) (6.00 g, 12.65 mmol) in THE (50 mL) was added BH₃.THF (42.9 mL, 42.90 mmol, 1 M in THF) dropwise at 0° C. under nitrogen atmosphere. The solution was stirred at room temperature for 3 h. Then H₂O₂ (3.2 mL, 137.35 mmol, 30%) and NaOH (2.50 g, 62.50 mmol) in H₂O (10 mL) were added dropwise at 0° C. The resulting solution was stirred at room temperature for 16 h. The reaction was quenched with saturated aq. Na₂SO₃ (50 mL) at 0° C. The resulting mixture was extracted with EA (3×100 mL). The combined organic layers were washed with brine (3×50 mL) and dried over anhydrous Na₂SO₄. After filtration, the filtrate was concentrated under reduced pressure to afford tert-butyl 4-(4,5-dichloro-2-hydroxyphenyl)-3-hydroxypiperidine-1-carboxylate as a light yellow solid (5.60 g, crude), which was directly used in next step without further purification: LCMS (ESI) calc'd for C₁₆H₂₁Cl₂NO₄ [M+H−56]⁺: 306, 308 (3:2), found 306, 308 (3:2); ¹H NMR (300 MHz, CD₃OD) δ 7.25 (s, 1H), 6.91 (s, 1H), 4.35-4.26 (m, 1H), 4.16-4.04 (m, 1H), 3.86-3.73 (m, 1H), 3.01-2.88 (m, 1H), 2.89-2.52 (m, 2H), 1.85-1.74 (m, 1H), 1.74-1.56 (m, 1H), 1.50 (s, 9H).

### Step b

To a stirred solution of tert-butyl 4-(4,5-dichloro-2-hydroxyphenyl)-3-hydroxypiperidine-1-carboxylate (5.60 g, 15.46 mmol) in DMF (10 mL) were added K₂CO₃ (4.29 g, 31.04 mmol) and PMBCl (2.71 g, 17.30 mmol) at room temperature. The reaction was stirred at 50° C. for 16 h. After cooling to room temperature, the reaction was diluted with water (50 mL) at room temperature. The resulting mixture was extracted with EA (3×50 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (3/1) to afford tert-butyl 4-[4,5-dichloro-2-[(4-methoxyphenyl)methoxy]phenyl]-3-hydroxy piperidine-1-carboxylate as an off-white solid (4.20 g, 70% overall two steps): LCMS (ESI) calc'd for C₂₄H₂₉Cl₂NO₅ [M+Na]⁺: 504, 506 (3:2), found 504, 506 (3:2); ¹H NMR (300 MHz, CDCl₃) δ 7.37-7.29 (m, 2H), 7.07 (s, 1H), 6.98-6.89 (m, 3H), 4.65 (s, 2H), 4.45-4.34 (m, 1H), 4.26-4.04 (m, 1H), 3.84 (s, 3H), 3.78-3.65 (m, 1H), 3.13-2.96 (m, 1H), 2.78-2.53 (m, 2H), 1.82-1.54 (m, 2H), 1.49 (s, 9H).

### Step c

To a stirred solution of tert-butyl 4-[4,5-dichloro-2-[(4-methoxyphenyl)methoxy]phenyl]-3-hydroxypiperidine-1-carboxylate (0.50 g, 1.04 mmol) in DCM (10 mL) were added TEA (0.21 g, 2.07 mmol), DMAP (13 mg, 0.10 mmol) and MsCl (0.24 g, 2.07 mmol) at room temperature. The resulting mixture was stirred for 2 h at room temperature under nitrogen atmosphere. The reaction was diluted with water (30 mL) at room temperature. The resulting mixture was extracted with EA (3×30 mL). The combined organic layers were washed with brine (2×20 mL) and dried over anhydrous Na₂SO₄. After filtration, the filtrate was concentrated under reduced pressure to afford tert-butyl 4-[4,5-dichloro-2-[(4-methoxyphenyl)methoxy]phenyl]-3-(methanesulfonyloxy)piperidine-1-carboxylate as a yellow oil (0.55 g, crude), which was used in the next step directly without further purification: LCMS (ESI) calc'd for C₂₅H₃₁Cl₂NO₇S [M+Na]⁺: 582, 584 (3:2), found 582, 584 (3:2).

### Step d

To a stirred solution of tert-butyl 4-[4,5-dichloro-2-[(4-methoxyphenyl)methoxy]phenyl]-3-(methanesulfonyloxy)piperidine-1-carboxylate (0.55 g, 0.98 mmol) in DMF (50 mL) was added NaN₃ (0.13 g, 1.96 mmol) at room temperature under nitrogen atmosphere. The resulting mixture was stirred for 16 h at 80° C. under nitrogen atmosphere. The mixture was allowed to cool down to room temperature and quenched with saturated aq. NaHCO₃(50 mL) at room temperature. The resulting mixture was extracted with EA (3×50 mL). The combined organic layers were washed with brine (2×30 mL) and dried over anhydrous Na₂SO₄. After filtration, the filtrate was concentrated under reduced pressure to afford tert-butyl 3-azido-4-[4,5-dichloro-2-[(4-methoxyphenyl)methoxy]phenyl]piperidine-1-carboxylate as a yellow oil (0.45 g, crude), which was used in the next step directly without further purification: LCMS (ESI) calc'd for C₂₄H₂₈Cl₂N₄O₄ [M+Na]⁺: 529, 531 (3:2), found 529, 531 (3:2).

### Step e

A mixture of tert-butyl 3-azido-4-[4,5-dichloro-2-[(4-methoxyphenyl)methoxy]phenyl]piperidine-1-carboxylate (0.45 g, 0.890 mmol) in ethynyltrimethylsilane (3 mL) was irradiated with microwave radiation for 2 h at 120° C. After cooling to room temperature, the reaction was diluted with water (30 mL) at room temperature. The resulting mixture was extracted with EA (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with PE/EA (1/1) to afford tert-butyl 4-[4,5-dichloro-2-[(4-methoxyphenyl)methoxy]phenyl]-3-(1H-1,2,3-triazol-1-yl)piperidine-1-carboxylate as an off-white foam (0.25 g, 40% overall three steps): LCMS (ESI) calc'd for C₂₉H₃₈Cl₂N₄O₄Si [M+H]⁺: 605, 607 (3:2), found 605, 607 (3:2).

### Step f

To a stirred solution of tert-butyl 4-[4,5-dichloro-2-[(4-methoxyphenyl)methoxy]phenyl]-3-[4-(trimethylsilyl)-1H-1,2,3-triazol-1-yl]piperidine-1-carboxylate (0.25 g, 0.41 mmol) in THE (5 mL) was added TBAF (0.54 g, 2.06 mmol) at room temperature. The resulting mixture was stirred for 16 h at room temperature. The reaction was quenched with the addition of water (30 mL) at room temperature. The resulting mixture was extracted with EA (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with PE/EA (5/1) to afford tert-butyl 4-[4,5-dichloro-2-[(4-methoxyphenyl)methoxy]phenyl]-3-(1H-1,2,3-triazol-1-yl)piperidine-1-carboxylate as a light yellow oil (0.15 g, 68%): LCMS (ESI) calc'd for C₂₆H₃₀Cl₂N₄O₄ [M+H]⁺: 533, 535 (3:2), found 533, 535 (3:2).

### Step g

To a stirred solution of tert-butyl 4-[4,5-dichloro-2-[(4-methoxyphenyl)methoxy]phenyl]-3-(1H-1,2,3-triazol-1-yl)piperidine-1-carboxylate (0.15 g, 0.282 mmol) in DCM (3 mL) was added TFA (0.5 mL) at room temperature. The resulting solution was stirred for 1 h at room temperature. The reaction was concentrated under reduced pressure. The residue was purified by Prep-HPLC with the following conditions: Column: X Bridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 20% B to 25% B in 5.3 min; Detector: UV 254/210 nm; Retention time: 4.93 min. The fractions containing the desired product were collected and concentrated under reduced pressure to afford Compound 62 (4,5-dichloro-2-[3-(1H-1,2,3-triazol-1-yl)piperidin-4-yl]phenol) as an off-white solid (48 mg, 55%): LCMS (ESI) calc'd for C₁₃H₁₄Cl₂N₄O [M+H]⁺: 313, 315 (3:2), found 313, 315 (3:2); ¹H NMR (300 MHz, CD₃OD) δ 7.63 (d, J=1.1 Hz, 1H), 7.25 (d, J=1.1 Hz, 1H), 7.03 (s, 1H), 6.43 (s, 1H), 5.50-5.38 (m, 1H), 4.00-3.83 (m, 3H), 3.83-3.73 (m, 1H), 3.50-3.37 (m, 1H), 2.66-2.41 (m, 1H), 2.03-1.83 (m, 1H).

### Example 45. Compound 63 (2R,4S)-rel-4-(2,3,4-trichloro-6-hydroxyphenyl)piperidine-2-carboxamide isomer 2) and Compound 65 ((2R,4S)-rel-4-(2,3,4-trichloro-6-hydroxyphenyl)piperidine-2-carboxamide isomer 1)

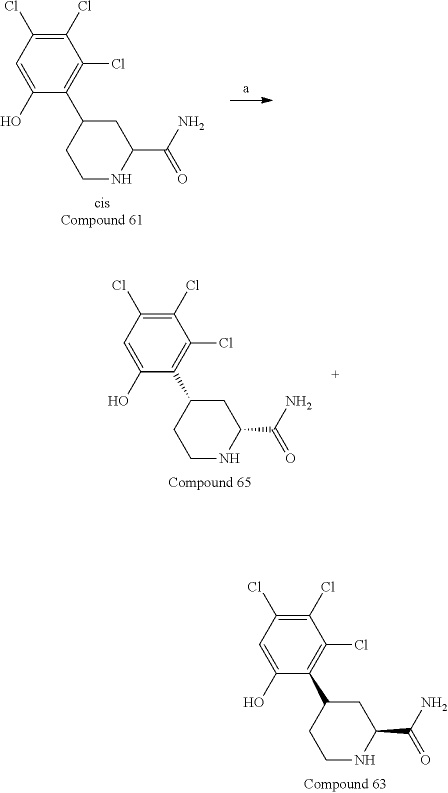

The absolute configurations for Compounds 63 and 65 were arbitrarily assigned.

### Step a

4-(2,3,4-trichloro-6-hydroxyphenyl)piperidine-2-carboxamide cis isomer (Compound 61, Example 43) (0.15 g, 0.46 mmol) was separated by Prep-Chiral HPLC with the following conditions: Column: Chiralpak ID, 2×25 cm, 5 μm; Mobile Phase A: Hex (plus 0.2% IPA), Mobile Phase B: IPA; Flow rate: 20 mL/min; Gradient: 10% B to 10% B in 20 min; Detector: UV 254/220 nm; Retention time: RT₁: 11.3 min; RT₂: 14.9 min; Injection Volume: 0.5 mL; Number Of Runs: 12. The faster-eluting enantiomer Compound 65 ((2R,4S)-rel-4-(2,3,4-trichloro-6-hydroxyphenyl)piperidine-2-carboxamide isomer 1) at 11.3 min was obtained as an off-white solid (15.6 mg, 10%): LCMS (ESI) calc'd for C₁₂H₁₃C₁₃N₂O₂ [M+H]⁺: 323, 325, 327 (3:3:1), found 323, 325, 327 (3:3:1); ¹H NMR (400 MHz, CD₃OD) δ 6.92 (s, 1H), 3.68-3.54 (m, 1H), 3.42 (dd, J=11.7, 3.0 Hz, 1H), 3.30-3.21 (m, 1H), 2.87-2.74 (m, 1H), 2.56-2.40 (m, 2H), 1.87 (d, J=12.9 Hz, 1H), 1.56 (d, J=13.2 Hz, 1H). The slower-eluting enantiomer Compound 63 ((2R,4S)-rel-4-(2,3,4-trichloro-6-hydroxyphenyl)piperidine-2-carboxamide (isomer 2)) at 11.84 min was obtained as an off-white solid (16.3 mg, 11%): LCMS (ESI) calc'd for C₁₂H₁₃C₁₃N₂O₂ [M+H]⁺: 323, 325, 327 (3:3:1), found 323, 325, 327 (3:3:1); ¹H NMR (400 MHz, CD₃OD) δ 6.92 (s, 1H), 3.70-3.52 (m, 1H), 3.42 (dd, J=11.8, 3.0 Hz, 1H), 3.30-3.21 (m, 1H), 2.86-2.73 (m, 1H), 2.57-2.38 (m, 2H), 1.87 (d, J=13.0 Hz, 1H), 1.56 (d, J=13.3 Hz, 1H).

### Example 46. Compound 64 ((3R,4R)-rel-4-(4,5-dichloro-2-hydroxyphenyl)pyrrolidine-3-carboxamide)

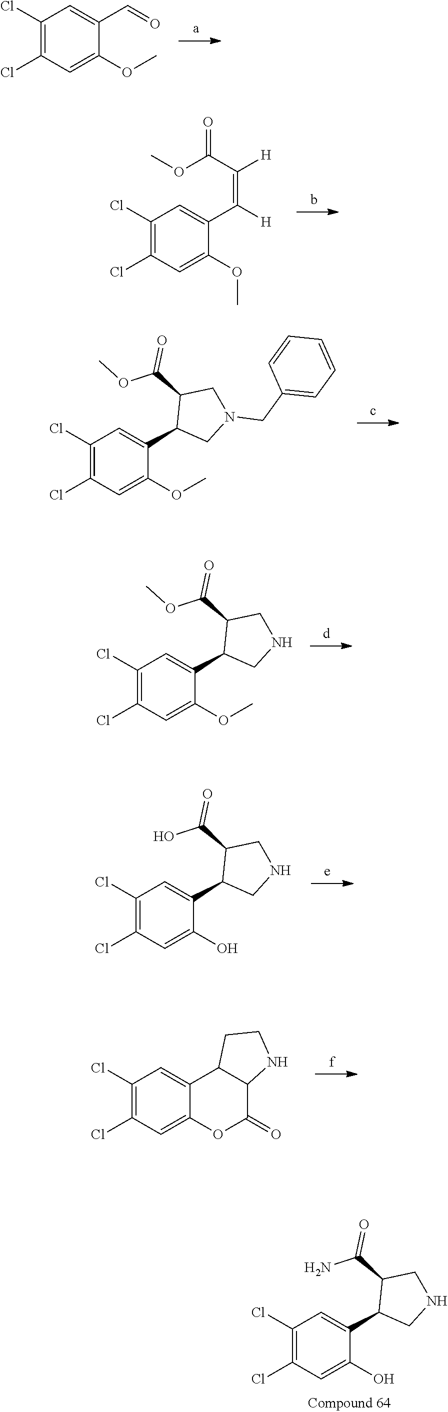

### Step a

To a stirred solution of methyl 2-[bis(2,2,2-trifluoroethoxy)phosphoryl]acetate (2.64 g, 8.29 mmol) in THE (25.0 mL) was added NaH (0.29 g, 7.32 mmol, 60% in mineral oil) at −78° C. under nitrogen atmosphere. The mixture was stirred for 0.5 h at −78° C. under nitrogen atmosphere. Then to the above mixture was added 4,5-dichloro-2-methoxybenzaldehyde (1.00 g, 4.88 mmol) at −78° C. under nitrogen atmosphere. The mixture was stirred for 1.5 h at −78° C. under nitrogen atmosphere. The reaction was quenched with water (50 mL) at room temperature. The resulting mixture was extracted with EA (3×50 mL). The combined organic layers were washed with brine (3×50 mL) and dried over anhydrous Na₂SO₄. After filtration, the filtrate was concentrated under reduced pressure. The residue was purified by silica gel column chromatography, eluted with PE/EA (5/1) to afford methyl (2Z)-3-(4,5-dichloro-2-methoxyphenyl)prop-2-enoate as a dark yellow solid (1.14 g, 90%): LCMS (ESI) calc'd for C₁₁H₁₀Cl₂O₃ [M+H]⁺: 261, 263 (3:2), found 261, 263 (3:2); ¹H NMR (400 MHz, CD₃OD) δ 7.69 (s, 1H), 7.17 (s, 1H), 7.05 (d, J=12.5 Hz, 1H), 6.05 (d, J=12.5 Hz, 1H), 3.86 (s, 3H), 3.70 (s, 3H).

### Step b

To a stirred solution of methyl (2Z)-3-(4,5-dichloro-2-methoxyphenyl)prop-2-enoate (1.14 g, 4.37 mol) in DCM (15 mL) were added benzyl(methoxymethyl)[(trimethylsilyl)methyl]amine (1.24 g, 5.24 mol) and TFA (0.10 g, 0.87 mmol) dropwise at room temperature under nitrogen atmosphere. The reaction solution was stirred for 16 h at room temperature. The reaction was diluted with water (30 mL) at room temperature. The resulting mixture was extracted with DCM (3×30 mL). The combined organic layers were washed with brine (2×20 mL) and dried over anhydrous Na₂SO₄. After filtration, the filtrate was concentrated under reduced pressure. The residue was purified by reverse phase chromatography, eluted with 50% ACN in water (plus 0.05% TFA) to afford methyl 1-benzyl-4-(4,5-dichloro-2-methoxyphenyl)pyrrolidine-3-carboxylate cis isomer as a colorless oil (0.74 g, 43%): LCMS (ESI) calc'd for C₂₀H₂₁Cl₂NO₃ [M+H]⁺: 394, 396 (3:2), found 394, 396 (3:2); ¹H NMR (400 MHz, CD₃OD) δ 7.66-7.59 (m, 2H), 7.59-7.47 (m, 3H), 7.29 (s, 1H), 7.23 (s, 1H), 4.61 (s, 2H), 4.13-4.01 (m, 1H), 3.93-3.85 (m, 6H), 3.85-3.72 (m, 4H), 3.72-3.67 (m, 1H).

### Step c

To a stirred solution of methyl 1-benzyl-4-(4,5-dichloro-2-methoxyphenyl)pyrrolidine-3-carboxylate cis isomer (0.50 g, 1.27 mol) in toluene (3 mL) were added 1-chloroethyl chloroformate (0.19 g, 1.52 mol) dropwise at room temperature under nitrogen atmosphere. The reaction was stirred for 16 h at 100° C. The reaction was diluted with water (30 mL) at room temperature. The resulting mixture was extracted with DCM (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with DCM/MeOH (10/1) to afford methyl 4-(4,5-dichloro-2-methoxyphenyl)pyrrolidine-3-carboxylate cis isomer as a dark yellow oil (0.30 g, 75%): LCMS (ESI) calc'd for C₁₃H₁₅Cl₂NO₃ [M+H]⁺: 304, 306 (3:2), found 304, 306 (3:2); ¹H NMR (400 MHz, CD₃OD) δ 7.25 (s, 1H), 7.22 (s, 1H), 4.09-3.98 (m, 1H), 3.92 (s, 3H), 3.73-3.62 (m, 3H), 3.62-3.52 (m, 2H), 3.37 (s, 3H).

### Step d

To a stirred solution of methyl 4-(4,5-dichloro-2-methoxyphenyl)pyrrolidine-3-carboxylate cis isomer (0.28 g, 0.93 mmol) in DCM (3 mL) were added BBr₃ (1.39 g, 5.56 mmol) at room temperature. The reaction was stirred at room temperature for 2 h. The reaction mixture was quenched with water (10 mL). The resulting solution was concentrated under reduced pressure. The residue was purified by reverse phase chromatography, eluted with 40% ACN in water (plus 0.05% TFA) to afford 4-(4,5-dichloro-2-hydroxyphenyl)pyrrolidine-3-carboxylic acid cis isomer as a dark yellow oil (0.26 g, crude): LCMS (ESI) calc'd for C₁₁H₁₁Cl₂NO₃ [M+H]⁺: 276, 278 (3:2), found 276, 278 (3:2);

### Step e

To a stirred mixture of 4-(4,5-dichloro-2-hydroxyphenyl)pyrrolidine-3-carboxylic acid cis isomer (0.20 g, 0.72 mmol) in DCM (1 mL) was added TFA (1 mL) at room temperature. The reaction was stirred for 2 h at 40° C. The reaction solution was concentrated under reduced pressure to afford 7,8-dichloro-2,3,3a,9b-tetrahydrochromeno[3,4-b]pyrrol-4(1H)-one as a dark yellow oil (0.20 g, crude), which was directly used in next step without further purification: LCMS (ESI) calc'd for C₁₁H₉Cl₂NO₂ [M+H]⁺: 258, 260 (3:2), found 258, 260 (3:2); ¹H NMR (400 MHz, DMSO-d₆) δ 7.29 (s, 1H), 6.85 (s, 1H), 4.03 (s, 2H), 3.77-3.70 (m, 1H), 3.25-3.11 (m, 2H), 2.60-2.51 (m, 1H).

### Step f

To a stirred solution of 7,8-dichloro-2,3,3a,9b-tetrahydrochromeno[3,4-b]pyrrol-4(1H)-one (0.20 g, 0.78 mmol) in THE (1 mL) was added NH₃.H₂O (1 mL, 30%) at room temperature. The reaction was stirred for 0.5 h at room temperature. The reaction mixture was concentrated under reduced pressure. The residue was purified by Prep-HPLC with the following conditions: Column X Bridge C₁₈ OBD Prep Column 100 Å, 10 μm, 19 mm×250 mm; Mobile Phase A: Water with 10 mmoL/L NH₄HCO₃, Mobile Phase B: ACN; Flow rate: 25 mL/min; Gradient: 20% B to 30% B in 6 min; Detector: UV: 254/210 nm; Retention Time: 4.72 min. The fractions containing the desired product were collected and concentrated under reduced pressure to afford to afford Compound 64 ((3R,4R)-rel-4-(4,5-dichloro-2-hydroxyphenyl)pyrrolidine-3-carboxamide (cis isomer)) as an off-white solid (35 mg, 16% overall two steps): LCMS (ESI) calc'd for C₁₁H₁₂Cl₂N₂O₂ [M+H]⁺: 275, 277 (3:2), found 275, 277 (3:2); ¹H NMR (300 MHz, CD₃OD) δ 7.15 (s, 1H), 6.86 (s, 1H), 3.82 (q, J=8.3 Hz, 1H), 3.52-3.36 (m, 3H), 3.29-3.22 (m, 2H).

### Example 47. Compound 66 (4,5-dichloro-2-(piperidin-4-yl)phenol)

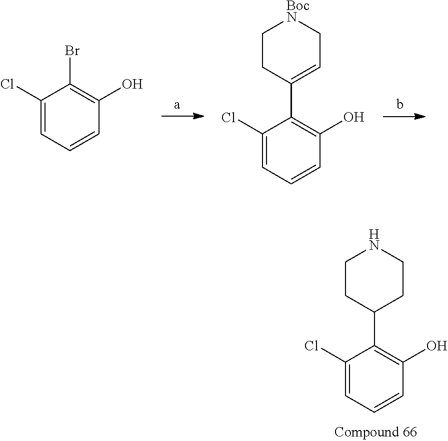 [SMILES: Oc1cccc(Cl)c1Br ; CC(C)(C)OC(=O)N1CC=C(c2c(O)cccc2Cl)CC1 ; Oc1cccc(Cl)c1C1CCNCC1]

### Step a

To a stirred solution of 2-bromo-3-chlorophenol (0.40 g, 1.93 mmol) and tert-butyl 4-(4,4,5,5-tetramethyl-1,3,2-dioxaborolan-2-yl)-1,2,3,6-tetrahydropyridine-1-carboxylate (0.72 g, 2.33 mmol) in 1,4-dioxane (4 mL) and H₂O(1 mL) were added Na₂CO₃ (0.62 g, 5.82 mmol) and Pd(dppf)C₁₂ (0.14 g, 0.20 mmol) at room temperature under nitrogen atmosphere. The resulting mixture was stirred for 2 h at 80° C. under nitrogen atmosphere. The mixture was allowed to cool down to room temperature. The reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (5/1) to afford tert-butyl 4-(2-chloro-6-hydroxyphenyl)-1,2,3,6-tetrahydropyridine-1-carboxylate as an off-white solid (0.40 g, 67%): LCMS (ESI) calc'd for C₁₆H₂₀ClNO₃ [M+H−15]⁺: 295, 297 (3:1), found 295, 297 (3:1); ¹H NMR (400 MHz, CD₃OD) δ 7.05 (t, J=8.0 Hz, 1H), 6.88 (d, J=8.0 Hz, 1H), 6.76 (d, J=8.2 Hz, 1H), 5.57 (s, 1H), 4.10-4.02 (m, 2H), 3.68-3.62 (m, 2H), 2.36-2.28 (m, 2H), 1.52 (s, 9H).

### Step b

To a stirred solution of tert-butyl 4-(2-chloro-6-hydroxyphenyl)-1,2,3,6-tetrahydropyridine-1-carboxylate (0.40 g, 1.29 mmol) and aq. HCl (0.4 mL, 6 N) in MeOH (4 mL) was added PtO₂ (50 mg, 0.22 mmol) and at room temperature. The reaction mixture was degassed with hydrogen and stirred at room temperature under hydrogen atmosphere (1.5 atm) for 2 h. The mixture was filtered and the filtrate was concentrated under reduced pressure. The residue was purified by Prep-HPLC with the following conditions: Column: XBridge C₁₈ OBD Prep Column, 100Å, 10 μm, 19 mm×250 mm; Mobile Phase A: Water (plus 0.05% TFA), Mobile Phase B: ACN; Flow rate: 25 mL/min; Gradient: 5% B to 35% B in 6 min; Detector: UV 254/220 nm; Retention time: 4.71 min; The fractions containing desired product were collected and concentrated under reduced pressure to afford Compound 66 (3-chloro-2-(piperidin-4-yl)phenol) as an off-white solid (28.9 mg, 7%): LCMS (ESI) calc'd for C₁₁H₁₄ClNO [M+H]⁺: 212, 214 (3:1), found 212, 214 (3:1); ¹H NMR (400 MHz, CD₃OD) δ 7.03 (t, J=8.0 Hz, 1H), 6.89 (d, J=8.0 Hz, 1H), 6.74 (d, J=8.1 Hz, 1H), 3.71-3.59 (m, 1H), 3.53-3.44 (m, 2H), 3.17-3.04 (m, 2H), 2.88-2.73 (m, 2H), 1.87-1.74 (m, 2H).

### Example 48. Compound 32 (2-[(2R,4S)-rel-4-(2,3-dichloro-6-hydroxyphenyl)piperidin-2-yl]-1-(morpholin-4-yl)ethan-1-one isomer 1) and Compound 67 (2-[(2R,4S)-rel-4-(2,3-dichloro-6-hydroxyphenyl)piperidin-2-yl]-1-(morpholin-4-yl)ethan-1-one isomer 2)

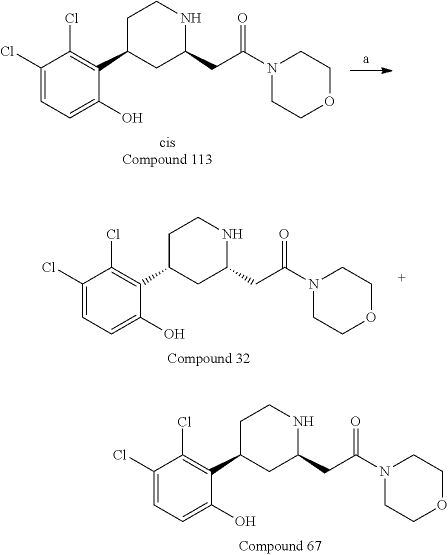 [SMILES: O=C(C[C@H]1C[C@@H](c2c(O)ccc(Cl)c2Cl)CCN1)N1CCOCC1 ; O=C(C[C@@H]1C[C@H](c2c(O)ccc(Cl)c2Cl)CCN1)N1CCOCC1 ; O=C(C[C@H]1C[C@@H](c2c(O)ccc(Cl)c2Cl)CCN1)N1CCOCC1]

### Step a

2-[4-(2,3-dichloro-6-hydroxyphenyl)piperidin-2-yl]-1-(morpholin-4-yl)ethan-1-one; (Example 76, Compound 113) (0.12 g, 0.24 mmol) was separated by Prep Chiral-HPLC with the following conditions: Column: Chiralpak ID-2, 2×25 cm, 5 μm; Mobile Phase A: Hex (plus 0.1% FA), Mobile Phase B: EtOH; Flow rate: 20 mL/min; Gradient: 20% B to 20% B in 30 min; Detector: UV: 220/254 nm; Retention time; RT₁: 7.86 min; RT₂: 19.10 min, Injection Volume: 0.7 mL; Number Of Runs: 5.

The faster-eluting enantiomer at 7.86 min was collected and concentrated under reduced pressure. The crude was purified by Prep-HPLC with following conditions: Column: Xselect CSH OBD Column 30×150 mm 5 m; Mobile Phase A: Water (plus 0.05% TFA), Mobile Phase B: ACN; Flow rate: 60 mL/min; Gradient: 10% B to 35% B in 7 min; Detector: UV: 254/220 nm; Retention time: 6.48 min. The fractions containing the desired product were collected and concentrated under reduced pressure to afford Compound 32 (2-[(2R,4S)-rel-4-(2,3-dichloro-6-hydroxyphenyl)piperidin-2-yl]-1-(morpholin-4-yl)ethan-1-one isomer 1) as an off-white solid (33.4 mg, 29%): LCMS (ESI) calc'd for C₁₇H₂₂Cl₂N₂O₃ [M+H]⁺: 373, 375 (3:2), found 373, 375 (3:2); ¹H NMR (400 MHz, CD₃OD) δ 7.26 (d, J=8.8 Hz, 1H), 6.76 (d, J=8.8 Hz, 1H), 3.92-3.71 (m, 1H), 3.71-3.58 (m, 7H), 3.58-3.48 (m, 3H), 3.27-3.13 (m, 1H), 3.02-2.86 (m, 1H), 2.83-2.60 (m, 3H), 1.87 (t, J=13.4 Hz, 2H).

The slower-eluting enantiomer at 19.10 min was collected and concentrated under reduced pressure. The crude was purified by Prep-HPLC with following conditions: Column: Xselect CSH OBD Column 30×150 mm 5 m; Mobile Phase A: Water (plus 0.05% TFA), Mobile Phase B: ACN; Flow rate: 60 mL/min; Gradient: 10% B to 35% B in 7 min; Detector: UV: 254/220 nm; Retention time: 6.48 min. The fractions containing the desired product were collected and concentrated under reduced pressure to afford Compound 67 (2-[(2R,4S)-rel-4-(2,3-dichloro-6-hydroxyphenyl)piperidin-2-yl]-1-(morpholin-4-yl)ethan-1-one isomer 2) as an off-white solid (20.9 mg, 18%): LCMS (ESI) calc'd for C₁₇H₂₂Cl₂N₂O₃ [M+H]⁺: 373, 375 (3:2), found 373, 375 (3:2); ¹H NMR (400 MHz, CD₃OD) δ 7.25 (d, J=8.8 Hz, 1H), 6.76 (d, J=8.8 Hz, 1H), 3.86-3.73 (m, 1H), 3.71-3.59 (m, 7H), 3.58-3.49 (m, 3H), 3.27-3.17 (m, 1H), 2.97-2.87 (m, 1H), 2.85-2.63 (m, 3H), 1.87 (t, J=13.8 Hz, 2H).

### Example 49. Compound 68 (3-chloro-4-fluoro-2-(piperidin-4-yl)phenol)

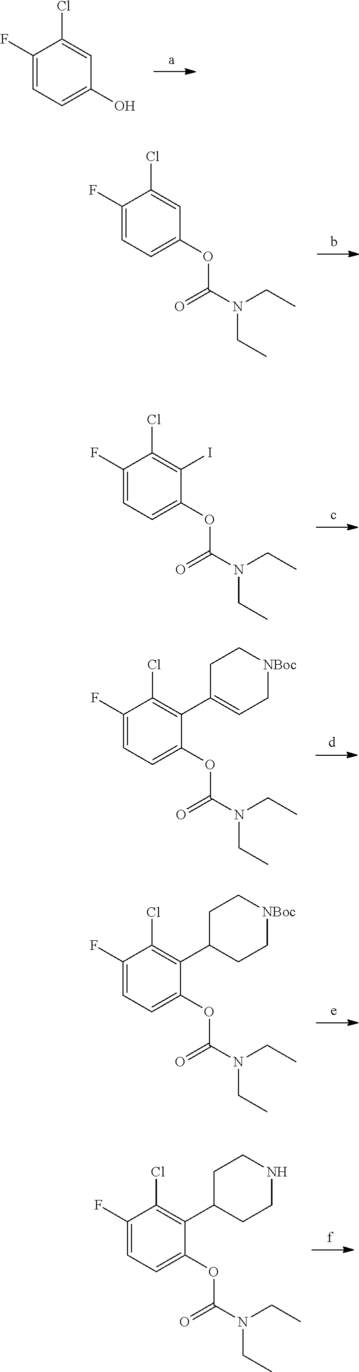

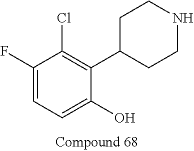 [SMILES: Oc1ccc(F)c(Cl)c1C1CCNCC1]

### Step a

A mixture of 3-chloro-4-fluorophenol (5.00 g, 34.12 mmol) and NaOH (3.40 g, 85.30 mmol) in THE (10 mL) was stirred for 10 min at room temperature. To the mixture was added N,N-diethylcarbamoyl chloride (6.90 g, 51.18 mmol) at room temperature. The reaction was stirred for 2 h at room temperature. The reaction mixture was diluted with water (80 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (8/1) to afford 3-chloro-4-fluorophenyl N,N-diethylcarbamate as a light yellow liquid (8.70 g, 83%): LCMS (ESI) calc'd for C₁₁H₁₃ClFNO₂ [M+H]⁺: 246, 248 (3:1), found 246, 248 (3:1); ¹H NMR (300 MHz, CDCl₃) δ 7.24 (dd, J=6.2, 2.8 Hz, 1H), 7.14 (t, J=8.7 Hz, 1H), 7.08-6.99 (m, 1H), 3.50-3.34 (m, 4H), 1.32-1.18 (m, 6H).

### Step b

To a stirred solution of 3-chloro-4-fluorophenyl N,N-diethylcarbamate (2.00 g, 8.14 mmol) in THE (5 mL) was added LDA (16 mL, 32.56 mmol, 2 M in THF) dropwise at −78° C. under nitrogen atmosphere. After stirred for 40 min, to the reaction was added a solution of 12 (2.50 g, 9.85 mmol) in THE (10 mL) dropwise at −78° C. Then the reaction was stirred at −78° C. for 0.5 h. The reaction mixture was quenched with saturated aq. NH₄Cl (30 mL) at −78° C. The resulting solution was extracted with EA (3×50 mL). The combined organic layers were washed with brine (2×30 mL) and dried over anhydrous Na₂SO₄. After filtration, the filtrate was concentrated under reduced pressure. The residue was purified by reverse phase chromatography, eluted with 70% ACN in water (plus 0.05% TFA) to afford 3-chloro-4-fluoro-2-iodophenyl N,N-diethylcarbamate as a light yellow solid (0.55 g, 14%): LCMS (ESI) calc'd for C₁₁H₁₂ClFINO₂ [M+H]⁺: 372, 374 (3:1), found 372, 374 (3:1); ¹H NMR (300 MHz, CDCl₃) δ 7.44 (dd, J=4.6, 2.7 Hz, 1H), 7.23 (dd, J=5.8, 2.8 Hz, 1H), 3.45-3.33 (m, 4H), 1.29-1.15 (m, 6H).

### Step c

A degassed mixture of 3-chloro-4-fluoro-2-iodophenyl N,N-diethylcarbamate (0.25 g, 0.66 mmol), tert-butyl 4-(4,4,5,5-tetramethyl-1,3,2-dioxaborolan-2-yl)-1,2,3,6-tetrahydropyridine-1-carboxylate (0.25 g, 0.80 mmol), Pd(dppf)Cl₂ (49 mg, 0.07 mmol) and Na₂CO₃ (0.21 g, 1.99 mmol) in 1,4-dioxane (3 mL) and water (0.75 mL) was stirred for 2.5 h at 80° C. under nitrogen atmosphere. The reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (3/1) to afford tert-butyl 4-[2-chloro-6-[(diethylcarbamoyl)oxy]-3-fluorophenyl]-1,2,3,6-tetrahydropyridine-1-carboxylate as a light yellow oil (0.20 g, 70%): LCMS (ESI) calc'd for C₂₁H₂₈ClFN₂O₄ [M+Na]⁺: 449, 451 (3:1), found 449, 451 (3:1); ¹H NMR (300 MHz, CDCl₃) δ 7.11 (dd, J=5.8, 2.9 Hz, 1H), 6.92 (dd, J=5.7, 2.9 Hz, 1H), 6.00-5.93 (m, 1H), 4.10-4.03 (m, 2H), 3.64-3.56 (m, 2H), 3.46-3.33 (m, 4H), 2.53-2.43 (m, 2H), 1.49 (s, 9H), 1.30-1.16 (m, 6H).

### Step d

A mixture of tert-butyl 4-[2-chloro-6-[(diethylcarbamoyl)oxy]-3-fluorophenyl]-1,2,3,6-tetrahydropyridine-1-carboxylate (0.20 g, 0.47 mmol) and PtO₂ (21 mg, 0.09 mmol) in MeOH (5 mL) was stirred for 3 h at 30° C. under hydrogen atmosphere (10 atm). The reaction mixture was filtered and the filtrate was concentrated under reduced pressure to afford tert-butyl 4-[2-chloro-6-[(diethylcarbamoyl)oxy]-3-fluorophenyl]piperidine-1-carboxylate as a light yellow oil (0.20 g, crude), which was directly used in next step without further purification: LCMS (ESI) calc'd for C₂₁H₃₀ClFN₂O₄ [M+Na]⁺: 451, 453 (3:1), found 451, 453 (3:1); ¹H NMR (300 MHz, CDCl₃) δ 7.08 (dd, J=6.0, 2.8 Hz, 1H), 6.87 (dd, J=5.5, 2.8 Hz, 1H), 4.25 (d, J=13.4 Hz, 2H), 3.47-3.34 (m, 4H), 3.08-2.95 (m, 1H), 2.88-2.72 (m, 2H), 1.86-1.74 (m, 2H), 1.68-1.51 (m, 2H), 1.48 (s, 9H), 1.29-1.17 (m, 6H).

### Step e

To a stirred solution of tert-butyl 4-[2-chloro-6-[(diethylcarbamoyl)oxy]-3-fluorophenyl]piperidine-1-carboxylate (0.20 g, 0.47 mmol) in DCM (4 mL) was added TFA (1 mL) at room temperature. The reaction was stirred at room temperature for 0.5 h. The resulting solution was concentrated under reduced pressure to afford 3-chloro-4-fluoro-2-(piperidin-4-yl)phenyl N,N-diethylcarbamate as a light yellow oil (0.20 g, crude), which was directly used in next step without further purification: LCMS (ESI) calc'd for C₁₆H₂₂C₁FN₂O₂ [M+H]⁺: 329, 331 (3:1), found 329, 331 (3:1).

### Step f

A mixture of 3-chloro-4-fluoro-2-(piperidin-4-yl)phenyl N,N-diethylcarbamate (0.20 g, 0.61 mmol) and NaOH (0.24 g, 6.08 mmol) in EtOH (4 mL) was stirred for 1.5 h at 80° C. under nitrogen atmosphere. The reaction mixture was concentrated under reduced pressure. The residue was purified by Prep-HPLC with the following conditions: Column: X Bridge C₁₈ OBD Prep Column 100 Å, 10 μm, 19 mm×250 mm; Mobile Phase A: Water with 10 mmol/L NH₄HCO₃ and 0.1% NH₃.H₂O, Mobile Phase B: ACN; Flow rate: 20 mL/min; Gradient: 28% B to 55% B in 6 min; Detector: UV 254/210 nm; Retention time: 4.42 min. The fractions containing the desired product were collected and concentrated under reduced pressure to afford Compound 68 (3-chloro-4-fluoro-2-(piperidin-4-yl)phenol) as an off-white solid (47 mg, 44% overall two steps): LCMS (ESI) calc'd for C₁₁H₁₃ClFNO [M+H]⁺: 230, 232 (3:1), found 230, 232 (3:1); ¹H NMR (400 MHz, CD₃OD) δ 6.71 (dd, J=5.9, 2.9 Hz, 1H), 6.64 (dd, J=5.4, 2.9 Hz, 1H), 3.30-3.23 (m, 2H), 3.08-2.98 (m, 1H), 2.93-2.83 (m, 2H), 1.91-1.83 (m, 2H), 1.82-1.68 (m, 2H).

### Example 50. Compound 69 (3-chloro-4-cyclopropyl-2-(piperidin-4-yl)phenol)

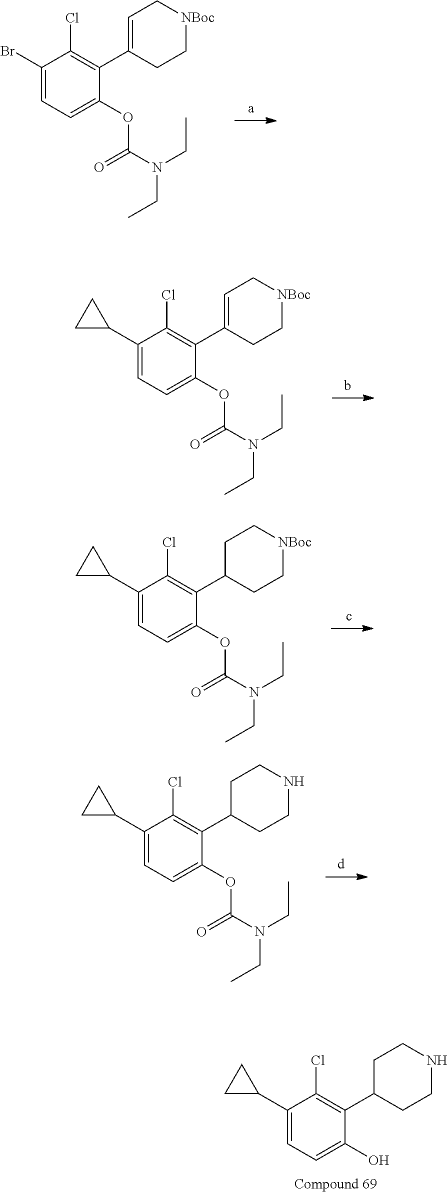

### Step a

A degassed mixture of tert-butyl 4-[3-bromo-2-chloro-6-[(diethylcarbamoyl)oxy]phenyl]-3,6-dihydro-2H-pyridine-1-carboxylate (Example 52, step c) (0.25 g, 0.51 mmoL), cyclopropylboronic acid (66 mg, 0.77 mmoL), Pd(dppf)C₁₂ (37 mg, 0.05 mmoL) and Na₂CO₃ (0.16 g, 1.53 mmoL) in 1,4-dioxane (3 mL) and water (0.75 mL) was stirred for 16 h at 80° C. under nitrogen atmosphere. After cooling to room temperature, the reaction mixture was diluted with water (20 mL). The resulting solution was extracted with EA (3×30 mL). Then the combined organic layers were washed with brine (2×20 mL), dried over anhydrous Na₂SO₄ and filtered. The filtrate was concentrated under reduced pressure. The residue was purified by Prep-TLC, eluted with PE/EA (5/1) to afford tert-butyl 4-[2-chloro-3-cyclopropyl-6-[(diethylcarbamoyl)oxy]phenyl]piperidine-1-carboxylate as a light yellow oil (0.17 g, 77%): LCMS (ESI) calc'd for C₂₄H₃₃C₁N₂O₄ [M−56+H]⁺: 393, 395 (3:1), found 393, 395 (3:1); ¹H NMR (300 MHz, CDCl₃) δ 7.01-6.85 (m, 1H), 5.59 (s, 2H), 4.09-3.94 (m, 2H), 3.81-3.75 (m, 1H), 3.52-3.46 (m, 1H), 3.38-3.30 (m, 4H), 2.43-2.37 (m, 1H), 2.31-2.25 (m, 1H), 2.21-2.09 (m, 1H), 1.49 (s, 9H), 1.22-1.13 (m, 6H), 1.03-0.94 (m, 2H), 0.71-0.63 (m, 2H).

### Step b

A degassed mixture of tert-butyl 4-[2-chloro-3-cyclopropyl-6-[(diethylcarbamoyl)oxy]phenyl]-1,2,3,6-tetrahydropyridine-1-carboxylate (0.12 g, 0.270 mmoL) and PtO₂ (18 mg, 0.080 mmoL) in MeOH (2 mL) was stirred for 16 h at room temperature under hydrogen atmosphere. The reaction mixture was filtered. The filtrate was concentrated under reduced pressure. The residue was purified by Prep-TLC, eluted with PE/EA (5/1) to afford tert-butyl 4-[2-chloro-3-cyclopropyl-6-[(diethylcarbamoyl)oxy]phenyl]piperidine-1-carboxylate as a light yellow oil (82 mg, 53%): LCMS (ESI) calc'd for C₂₄H₃₅C₁N₂O₄ [M−56+H]⁺: 395, 397 (3:1), found 395, 397 (3:1); ¹H NMR (300 MHz, CD₃OD) δ 6.96 (d, J=8.6 Hz, 1H), 6.86 (d, J=8.6 Hz, 1H), 4.22 (d, J=13.2 Hz, 2H), 3.55-3.45 (m, 2H), 3.41 (t, J=7.3 Hz, 2H), 2.83 (s, 2H), 2.15 (td, J=8.3, 4.1 Hz, 1H), 1.64 (d, J=13.3 Hz, 2H), 1.47 (s, 9H), 1.33-1.18 (m, 9H), 1.06-0.93 (m, 2H), 0.71-0.59 (m, 2H).

### Step c

To a stirred solution of tert-butyl 4-[2-chloro-3-cyclopropyl-6-[(diethylcarbamoyl)oxy]phenyl]piperidine-1-carboxylate (78 mg, 0.17 mmol) in DCM (1 mL) was added TFA (1 mL) at room temperature. The reaction was stirred for 0.5 h at room temperature. The resulting solution was concentrated under reduced pressure to afford 3-chloro-4-cyclopropyl-2-(piperidin-4-yl)phenyl N,N-diethylcarbamate as a light yellow oil, which was directly used in next step without further purification (0.11 g, crude): LCMS (ESI) calc'd for C₁₉H₂₇C₁N₂O₂ [M+H]⁺: 351, 353 (3:1), found 351, 353 (3:1).

### Step d

A solution of 3-chloro-4-cyclopropyl-2-(piperidin-4-yl)phenyl N,N-diethylcarbamate (0.11 g, 0.33 mmol) and NaOH (0.13 g, 3.28 mmol) in EtOH (4 mL) was stirred for 5.5 h at 80° C. under nitrogen atmosphere. After cooling to room temperature, the reaction mixture was concentrated under reduced pressure. The residue was purified by Prep-HPLC with the following conditions: Column: Sun Fire C₁₈ OBD Prep Column, 100 Å, 5 μm, 19 mm×250 mm; Mobile Phase A: Water (plus 0.05% TFA), Mobile Phase B: ACN; Flow rate: 20 mL/min; Gradient: 25% B to 55% B in 6 min; Detector: 210 nm; Retention time: 4.87 min, The fractions containing desired product were collected and concentrated under reduced pressure to afford Compound 69 (3-chloro-4-cyclopropyl-2-(piperidin-4-yl)phenol) as an off-white solid (17.8 mg, 14%): LCMS (ESI) calc'd for C₁₄H₁₈ClNO [M+H]⁺: 252, 254 (3:1), found 252, 254 (3:1); ¹H NMR (300 MHz, CD₃OD) δ 6.82 (d, J=8.5 Hz, 1H), 6.66 (d, J=8.4 Hz, 1H), 3.82-3.68 (m, 1H), 3.53-3.43 (m, 2H), 3.19-3.04 (m, 2H), 2.83 (qd, J=13.6, 4.1 Hz, 2H), 2.11-1.96 (m, 1H), 1.81 (d, J=14.1 Hz, 2H), 0.98-0.84 (m, 2H), 0.62-0.51 (m, 2H).

### Example 51. Compound 70 ((2R,4S)-rel-4-(2,3-dichloro-6-hydroxyphenyl)piperidine-2-carboxamide isomer 1) and Compound 73 ((2R,4S)-rel-4-(2,3-dichloro-6-hydroxyphenyl)piperidine-2-carboxamide isomer 2)

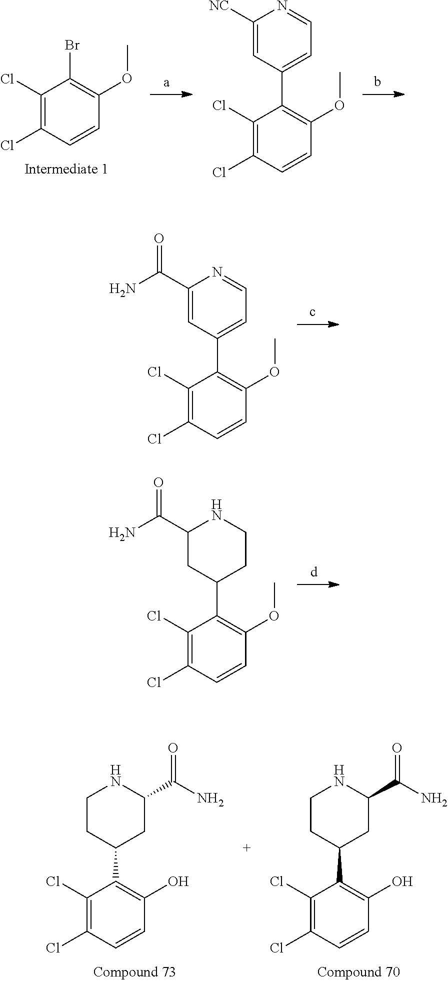

### Step a

To a solution of Intermediate 1 (1.00 g, 3.91 mmol) and 4-(4,4,5,5-tetramethyl-1,3,2-dioxaborolan-2-yl)pyridine-2-carbonitrile (1.00 g, 4.34 mmol) in 1,4-dioxane (20 mL) and H₂O (5 mL) were added Na₂CO₃ (1.24 g, 11.72 mmol) and Pd(dppf)Cl₂.CH₂Cl₂ (0.64 g, 0.78 mmol). After stirring for 3 h at 80° C. under a nitrogen atmosphere, the resulting mixture was concentrated under reduced pressure. The residue was purified by silica gel column chromatography, eluted with PE/EA (3/1) to afford 4-(2,3-dichloro-6-methoxyphenyl)pyridine-2-carbonitrile (0.80 g, 62%) as an off-white solid: LCMS (ESI) calc'd for C₁₃H₈Cl₂N₂O [M+H]⁺: 279, 281 (3:2), found 279, 281 (3:2); ¹H NMR (300 MHz, CDCl₃) δ 8.81 (dd, J=5.0, 0.8 Hz, 1H), 7.64 (dd, J=1.6, 0.8 Hz, 1H), 7.54 (d, J=9.0 Hz, 1H), 7.46 (dd, J=5.0, 1.6 Hz, 1H), 6.92 (d, J=9.0 Hz, 1H), 3.77 (s, 3H).

### Step b

To a stirred solution of 4-(2,3-dichloro-6-methoxyphenyl)pyridine-2-carbonitrile (0.80 g, 2.87 mmol) in THE were added NaOH (1.15 g, 28.66 mmol) and H₂O₂ (0.7 mL, 19.63 mmol, 30% in water) dropwise at 0° C. The resulting mixture was stirred for 2 h at room temperature. The reaction was quenched by the addition of saturated aq. Na₂SO₃ (20 mL) at 0° C. The resulting mixture was extracted with EA (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (2/1) to afford 4-(2,3-dichloro-6-methoxyphenyl)pyridine-2-carboxamide (0.70 g, 65%) as an off-white solid: LCMS (ESI) calc'd for C₁₃H₁₆Cl₂N₂O₂ [M+H]⁺: 297, 299 (3:2), found 297, 299 (3:2); ¹H NMR (400 MHz, CDCl₃) δ 8.67 (d, J=5.0 Hz, 1H), 8.16 (t, J=1.1 Hz, 1H), 7.97 (s, 1H), 7.51 (dd, J=9.0, 0.8 Hz, 1H), 7.40 (dd, J=5.0, 1.6 Hz, 1H), 6.89 (d, J=9.0 Hz, 1H), 5.69 (s, 1H), 3.74 (d, J=0.8 Hz, 3H).

### Step c

To a stirred solution of 4-(2,3-dichloro-6-methoxyphenyl)pyridine-2-carboxamide (0.68 g, 2.29 mmol) in MeOH (40 mL) and aq. HCl (6 N, 4 mL) was added PtO₂ (52 mg) at room temperature. The mixture was degassed with hydrogen three times and stirred at 30° C. under hydrogen atmosphere (50 atm) for 16 h. The reaction was filtered, and the filtrate was concentrated under reduced pressure to afford 4-(2,3-dichloro-6-methoxyphenyl)piperidine-2-carboxamide (0.68 g, crude) as an off-white solid: LCMS (ESI) calc'd for C₁₃H₁₆Cl₂N₂O₂ [M+H]⁺: 303, 305 (3:2), found 303, 305 (3:2).

### Step d

To a stirred solution of 4-(2,3-dichloro-6-methoxyphenyl)piperidine-2-carboxamide (50 mg, 0.16 mmol) in DCM (2 mL) was added BBr₃ (82 mg, 0.33 mmol) dropwise at room temperature. The resulting mixture was stirred for 1 h at room temperature. The reaction was quenched with water at 0° C. The resulting mixture was concentrated under reduced pressure. The crude product was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 28% B to 48% B in 6.5 min; Detector: 254/210 nm; Retention time: 5.70 min. The fractions containing the desired product were collected and concentrated under reduced pressure to afford 4-(2,3-dichloro-6-hydroxyphenyl)piperidine-2-carboxamide (60 mg, 48%) as an off-white solid.

4-(2,3-Dichloro-6-hydroxyphenyl)piperidine-2-carboxamide (60 mg, 0.208 mmol) was separated by Chiral Prep-HPLC with the following conditions: Column: Chiralpak ID-03, 2.0 cm I.D×25 cm L (5 m); Mobile Phase A: Hex (0.2% IPA), Mobile Phase B: IPA; Flow rate: 20 mL/min; Gradient: 10% B to 10% B in 20 min; Detector: 254/220 nm; Retention time: RT₁: 11.5 min; RT₂: 14.8 min.

The faster-eluting enantiomer Compound 70 ((2R,4S)-rel-4-(2,3-dichloro-6-hydroxyphenyl)piperidine-2-carboxamide isomer 1) was obtained at 11.5 min as a light yellow solid (17.9 mg, 30%): LCMS (ESI) calc'd for C₁₂H₁₄Cl₂N₂O₂ [M+H]⁺: 289, 291 (3:2), found 289, 291 (3:2); ¹H NMR (300 MHz, CD₃OD) δ 7.18 (d, J=8.7 Hz, 1H), 6.71 (d, J=8.8 Hz, 1H), 3.65-3.59 (m, 1H), 3.42-3.35 (m, 1H), 3.30-3.17 (m, 1H), 2.76 (td, J=12.6, 2.9 Hz, 1H), 2.58-2.37 (m, 2H), 1.84 (d, J=12.7 Hz, 1H), 1.53 (d, J=13.1 Hz, 1H).

The slower-eluting enantiomer Compound 73 ((2R,4S)-rel-4-(2,3-dichloro-6-hydroxyphenyl)piperidine-2-carboxamide isomer 2) was obtained at 14.8 min as a light yellow solid (17.2 mg, 29%): LCMS (ESI) calc'd for C₁₂H₁₄Cl₂N₂O₂ [M+H]⁺: 289, 291 (3:2), found 289, 291 (3:2); ¹H NMR (300 MHz, CD₃OD) δ 7.18 (d, J=8.7 Hz, 1H), 6.71 (d, J=8.8 Hz, 1H), 3.65-3.59 (m, 1H), 3.42-3.34 (m, 1H), 3.29-3.18 (m, 1H), 2.77 (td, J=12.7, 2.9 Hz, 1H), 2.58-2.37 (m, 2H), 1.84 (d, J=12.6 Hz, 1H), 1.53 (d, J=13.1 Hz, 1H).

### Example 52. Compound 71 (3-chloro-4-methyl-2-(piperidin-4-yl)phenol)

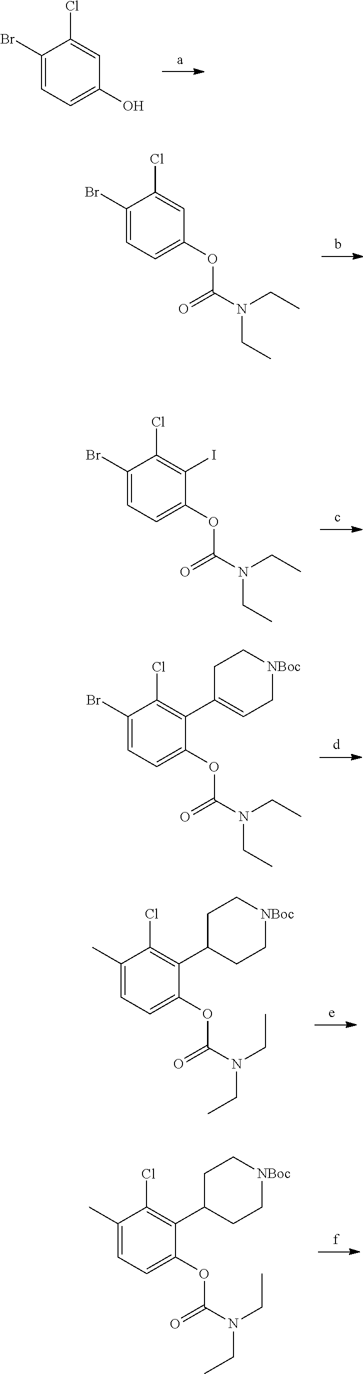

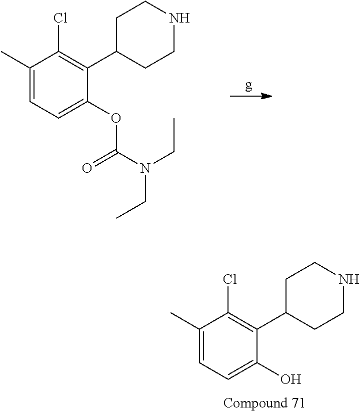 [SMILES: CCN(CC)C(=O)Oc1ccc(C)c(Cl)c1C1CCNCC1 ; Cc1ccc(O)c(C2CCNCC2)c1Cl]

### Step a

To a stirred mixture of 4-bromo-3-chlorophenol (19.47 g, 93.85 mmol) and N,N-diethylcarbamoyl chloride (25.5 g, 0.19 mmol) in THE (200 mL) was added NaOH (7.50 g, 0.19 mmol) in portions at room temperature under air atmosphere. The reaction mixture was stirred for 3 h at room temperature under nitrogen atmosphere. The resulting mixture was concentrated under reduced pressure. The residue was purified by silica gel column chromatography, eluted with PE/EA (5/1) to afford 4-bromo-3-chlorophenyl N,N-diethylcarbamate as a yellow oil (30.0 g, 94%): LCMS (ESI) calc'd for C₁₁H₁₃BrClNO₂ [M+H]⁺: 306, 308, 310 (2:3:1), found 306, 308, 310 (2:3:1); ¹H NMR (400 MHz, CD₃OD) δ 7.69 (dt, J=8.9, 2.0 Hz, 1H), 7.37 (d, J=2.6 Hz, 1H), 7.03 (dd, J=8.8, 2.8 Hz, 1H), 3.48 (q, J=7.2 Hz, 2H), 3.41 (q, J=7.3 Hz, 2H), 1.24 (dt, J=25.7, 7.1 Hz, 6H).

### Step b

To a stirred solution of DIPA (6.60 g, 65.24 mmol) in THF (100 mL) was added n-BuLi (26.1 mL, 65.24 mmol, 2.5 M in hexanes) at −78° C. under argon atmosphere. The resulting mixture was stirred for 30 min at −65° C. under argon atmosphere. To the above mixture was added 4-bromo-3-chlorophenyl N,N-diethylcarbamate (10.00 g, 32.62 mmol) in portions over 20 min at −78° C. The resulting mixture was stirred for additional 1 h at −78° C. To the above mixture was added a solution of 12 (9.93 g, 39.14 mmol) in THF (20 mL) dropwise over 20 min at −65° C. The resulting mixture was stirred for additional 30 min at −65° C. The reaction was quenched by the addition of water (300 mL) at room temperature. The resulting mixture was extracted with EA (2×300 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (7/1) to afford 4-bromo-3-chloro-2-iodophenyl N,N-diethylcarbamate as a yellow oil (4.00 g, 25%): LCMS (ESI) calc'd for C₁₁H₁₂BrClINO₂ [M+H]⁺: 432, 434, 436 (2:3:1), found 432, 434, 436 (2:3:1); ¹H NMR (300 MHz, CDCl₃) δ 7.62 (d, J=8.7 Hz, 1H), 6.98 (d, J=8.8 Hz, 1H), 3.53 (q, J=7.1 Hz, 2H), 3.40 (q, J=7.2 Hz, 2H), 1.27 (dt, J=27.9, 7.1 Hz, 6H).

### Step c

To a stirred solution of 4-bromo-3-chloro-2-iodophenyl N,N-diethylcarbamate (3.00 g, 6.937 mmol), Na₂CO₃ (2.21 g, 20.810 mmol), tert-butyl 4-(4,4,5,5-tetramethyl-1,3,2-dioxaborolan-2-yl)-3,6-dihydro-2H-pyridine-1-carboxylate (2.36 g, 7.630 mmol) and H₂O (7 mL) in 1,4-dioxane (30 mL) was added Pd(dppf)Cl₂.CH₂Cl₂(0.57 g, 0.694 mmol) in portions at room temperature under nitrogen atmosphere. The resulting mixture was degassed and stirred for 12 h at 80° C. under nitrogen atmosphere. The reaction was quenched with water (100 mL) at room temperature. The resulting mixture was extracted with EA (2×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (5/1) to afford tert-butyl 4-[3-bromo-2-chloro-6-[(diethylcarbamoyl)oxy]phenyl]-3,6-dihydro-2H-pyridine-1-carboxylate as a yellow solid (2.50 g, 66%): LCMS (ESI) calc'd for C₂₁H₂₈BrClN₂O₄ [M+H]⁺: 487, 489, 491 (2:3:1), found 487, 489, 491 (2:3:1); ¹H NMR (400 MHz, CDCl₃) δ 7.57 (d, J=8.7 Hz, 1H), 6.99 (d, J=8.7 Hz, 1H), 5.62 (s, 1H), 4.09 (s, 1H), 4.01 (d, J=15.8 Hz, 1H), 3.80 (s, 1H), 3.47 (s, 1H), 3.36 (d, J=7.3 Hz, 4H), 2.46-2.23 (m, 2H), 1.51 (s, 9H), 1.29-1.15 (m, 6H).

### Step d

To a stirred solution of tert-butyl 4-[3-bromo-2-chloro-6-[(diethylcarbamoyl)oxy]phenyl]-3,6-dihydro-2H-pyridine-1-carboxylate (0.30 g, 0.61 mmol), Pd(dppf)Cl₂ (45 mg, 0.06 mmol) and methylboronic acid (0.11 g, 1.85 mmol) in 1,4-dioxane was added Na₂CO₃ (0.20 g, 1.84 mmol) in portions at room temperature under nitrogen atmosphere. The resulting mixture was degassed with nitrogen three times and stirred for 2 h at 80° C. under nitrogen atmosphere. The reaction was quenched by the addition of water (4 mL) at room temperature. The residue was purified by Prep-HPLC with the following conditions: Column: XBridge C₁₈ OBD Prep Column 100 Å, 10 μm, 19 mm×250 mm; Mobile Phase A: water with 10 mmol/L NH₄HCO₃, Mobile Phase B: ACN; Flow rate: 25 mL/min; Gradient: 60% B to 89% B in 15 min; Detector: 254/210 nm; Retention time: 8 min. The fractions containing desired product were collected and concentrated under reduced pressure to afford tert-butyl 4-[2-chloro-6-[(diethylcarbamoyl)oxy]-3-methylphenyl]-1,2,3,6-tetrahydropyridine-1-carboxylate as a light yellow oil (0.11 g, 29%): LCMS (ESI) calc'd for C₂₂H₃₁C₁N₂O₄ [M+H]⁺: 423, 425 (3:1), found 423, 425 (3:1); ¹H NMR (300 MHz, CDCl₃) δ 7.17 (d, J=8.3 Hz, 1H), 6.98 (d, J=8.3 Hz, 1H), 5.60 (s, 1H), 4.07 (s, 1H), 3.96 (d, J=17.6 Hz, 1H), 3.79 (s, 1H), 3.48 (s, 1H), 3.41-3.32 (m, 4H), 2.38 (s, 3H), 2.27 (d, J=14.7 Hz, 2H), 1.51 (s, 9H), 1.31-1.13 (m, 6H).

### Step e

A degassed solution of tert-butyl 4-[2-chloro-6-[(diethylcarbamoyl)oxy]-3-methylphenyl]-1,2,3,6-tetrahydropyridine-1-carboxylate (0.16 g, 0.33 mmol) and PtO₂ (15 mg, 0.07 mmol) in MeOH (3 mL) was stirred for 2 h at room temperature under hydrogen atmosphere. The mixture was filtered, and then filter cake was washed with MeOH (2×10 mL). The filtrate was concentrated under reduced pressure to afford tert-butyl 4-[2-chloro-6-[(diethylcarbamoyl)oxy]-3-methylphenyl]piperidine-1-carboxylate as a light yellow solid (0.16 g, crude): LCMS (ESI) calc'd for C₂₂H₃₃C₁N₂O₄ [M−100+H]⁺: 325, 327 (3:1), found 325, 327 (3:1); ¹H NMR (300 MHz, CDCl₃) δ 7.09 (d, J=8.4 Hz, 1H), 6.85 (d, J=8.3 Hz, 1H), 4.24 (d, J=13.0 Hz, 2H), 3.52-3.33 (m, 4H), 2.83-2.73 (m, 2H), 2.36 (s, 3H), 2.04-1.98 (m, 1H), 1.71-1.60 (m, 2H), 1.48 (s, 9H), 1.32-1.20 (m, 8H).

### Step f

A solution of tert-butyl 4-[3-bromo-2-chloro-6-[(diethylcarbamoyl)oxy]phenyl]piperidine-1-carboxylate (0.16 g, 0.33 mmol) and TFA (3 mL) in DCM (3 mL) was stirred for 2 h at room temperature under nitrogen atmosphere. The resulting mixture was concentrated under reduced pressure to afford 3-chloro-4-methyl-2-(piperidin-4-yl)phenyl N,N-diethylcarbamate as a light yellow solid (0.16 g, crude): LCMS (ESI) calc'd for C₁₇H₂₅C₁N₂O₂ [M+H]⁺: 325, 327 (3:1), found 325, 327 (3:1).

### Step g

A solution of 3-chloro-4-methyl-2-(piperidin-4-yl)phenyl N,N-diethylcarbamate (0.15 g, 0.460 mmol) and NaOH (0.40 g, 10.00 mmol) in EtOH (8 mL) was stirred for 2 h at 80° C. under nitrogen atmosphere. The mixture was basified to pH 8 with aq. HCl (1 N). The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 10 mmol/L NH₄HCO₃, Mobile Phase B: ACN; Flow rate: 25 mL/min; Gradient: 10% B to 58% B in 6 min; Detector: 254/210 nm; Retention time: 4.22 min. The fractions containing desired product were collected and concentrated under reduced pressure to afford Compound 71 (3-chloro-4-methyl-2-(piperidin-4-yl)phenol) as an off-white solid (6.5 mg, 9%): LCMS (ESI) calc'd for C₁₂H₁₆ClNO [M+H]⁺: 226, 228 (3:1), found 226, 228 (3:1); ¹H NMR (400 MHz, CD₃OD) δ 7.00 (d, J=8.3 Hz, 1H), 6.66 (d, J=8.3 Hz, 1H), 3.72-3.65 (m, 1H), 3.48 (d, J=12.7 Hz, 2H), 3.11 (td, J=13.2, 3.2 Hz, 2H), 2.86-2.75 (m, 2H), 2.29 (s, 3H), 1.81 (d, J=14.2 Hz, 2H).

### Example 53. Compound 72 (4-(2,3-dichloro-6-hydroxyphenyl)-N-methylpiperidine-2-carboxamide)

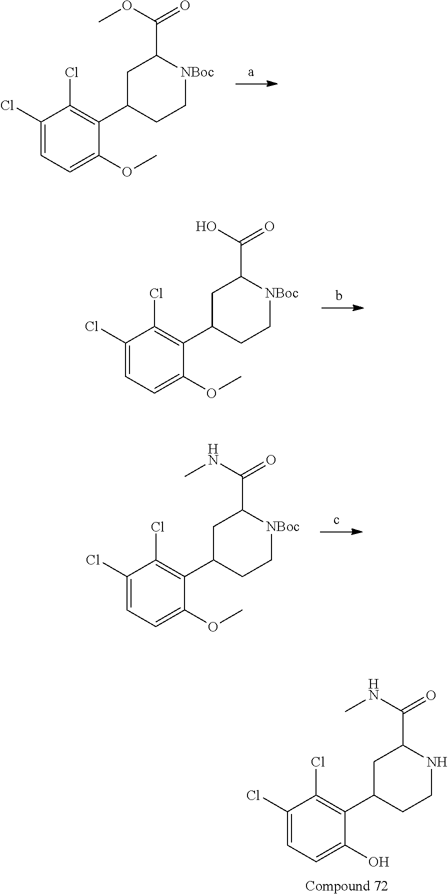 [SMILES: COC(=O)C1CC(c2c(OC)ccc(Cl)c2Cl)CCN1C(=O)OC(C)(C)C ; COc1ccc(Cl)c(Cl)c1C1CCN(C(=O)OC(C)(C)C)C(C(=O)O)C1 ; CNC(=O)C1CC(c2c(OC)ccc(Cl)c2Cl)CCN1C(=O)OC(C)(C)C ; CNC(=O)C1CC(c2c(O)ccc(Cl)c2Cl)CCN1]

### Step a

To a stirred solution of 1-tert-butyl 2-methyl 4-(2,3-dichloro-6-methoxyphenyl)piperidine-1,2-dicarboxylate (Example 61, step c) (1.00 g, 2.391 mmol) in MeOH (10 mL) was added NaOH (0.19 g, 4.781 mmol) at room temperature under air atmosphere. The resulting mixture was stirred for 1 h at room temperature under air atmosphere. The reaction mixture was acidified with citric acid to pH=4. Then the reaction mixture was extracted with EA (2×20 mL). The organic phase was combined, dried over anhydrous Na₂SO₄, filtered and concentrated under reduced pressure. 1-[(tert-butoxy)carbonyl]-4-(2,3-dichloro-6-methoxyphenyl)piperidine-2-carboxylic acid was afforded as a colorless oil without further purification (1.00 g, crude): LCMS (ESI) calc'd for C₁₈H₂₃Cl₂NO₅ [M+H]⁺: 404, 406 (3:2), found 404, 406 (3:2); ¹H NMR (400 MHz, CDCl₃) δ 7.36-7.30 (m, 1H), 6.76 (dd, J=9.0, 6.1 Hz, 1H), 4.17-4.07 (m, 1H), 3.84 (s, 3H), 3.68-3.46 (m, 1H), 3.22-3.03 (m, 1H), 2.72-2.53 (m, 1H), 2.46-2.25 (m, 1H), 2.25-2.12 (m, 1H), 2.02-1.92 (m, 1H), 1.70-1.56 (m, 1H), 1.52 (s, 9H).

### Step b

To a stirred solution of 1-[(tert-butoxy)carbonyl]-4-(2,3-dichloro-6-methoxyphenyl)piperidine-2-carboxylic acid (90 mg, 0.22 mmol) and EDC HCl (96 mg, 0.50 mmol) in DMF (2 mL) was added CH₃NH₂ (25 mg, 0.80 mmol) and Et₃N (76 mg, 0.75 mmol) in portions at room temperature under air atmosphere. The resulting mixture was stirred for 2.5 h at room temperature. The resulting mixture was diluted with water (20 mL) and extracted with EA (3×20 mL). The combined organic layers were washed with brine (3×10 mL), dried over anhydrous Na₂SO₄. After filtration, the filtrate was concentrated under reduced pressure. The residue was purified by Prep-HPLC with the following conditions: Column: XBridge C₁₈ OBD Prep Column, 100Å, 10 μm, 19 mm×250 mm; Mobile Phase A: Water with 10 mmol/L NH₄HCO₃, Mobile Phase B: ACN; Flow rate: 25 mL/min; Gradient: 30% B to 70% B in 6 min; Detector: 254/210 nm; Retention time: 5.83 min. The fractions containing the desired product were collected and concentrated under reduced pressure to afford tert-butyl 4-(2,3-dichloro-6-methoxyphenyl)-2-(methylcarbamoyl)piperidine-1-carboxylate as a light yellow oil (20 mg, 21%): LCMS (ESI) calc'd for C₁₉H₂₆Cl₂N₂O₄ [M+H]⁺: 417, 419 (3:2), found 417, 419 (3:2).

### Step c

To a stirred solution of tert-butyl 4-(2,3-dichloro-6-methoxyphenyl)-2-(methylcarbamoyl)piperidine-1-carboxylate (0.10 g, 0.24 mmol) in DCM (2 mL) was added BBr₃ (2.0 mL, 7.98 mmol) at room temperature. The resulting mixture was stirred for 2 h at room temperature under nitrogen atmosphere. The resulting mixture was quenched with water (2 mL) at room temperature and concentrated under reduced pressure. The residue was purified by Prep-HPLC with the following conditions: Column: XBridge C₁₈ OBD Prep Column, 100Å, 10 μm, 19 mm×250 mm; Mobile Phase A: Water with 10 mmol/L NH₄HCO₃, Mobile Phase B: ACN; Flow rate: 25 mL/min; Gradient: 30% B to 63% B in 6 min; Detector: 254/210 nm; Retention time: 4.98 min. The fractions containing the desired product were collected and concentrated under reduced pressure to afford Compound 72 (4-(2,3-dichloro-6-hydroxyphenyl)-N-methylpiperidine-2-carboxamide) as an off-white solid (8 mg, 11%): LCMS (ESI) calc'd for C₁₃H₁₆Cl₂N₂O₂ [M+H]⁺: 303, 305 (3:2), found 303, 305 (3:2); ¹H NMR (400 MHz, CD₃OD) δ 7.18 (d, J=8.7 Hz, 1H), 6.70 (dd, J=8.8, 5.5 Hz, 1H), 3.63-3.58 (m, 1H), 3.40-3.36 (m, 1H), 3.28-3.20 (m, 1H), 2.86-2.71 (m, 1H), 2.76 (s, 3H), 2.47 (q, J=12.3 Hz, 2H), 1.78 (d, J=12.7 Hz, 1H), 1.53 (d, J=13.3 Hz, 1H).

The compound in Table 1D below was prepared in an analogous fashion to that described for Compound 72, starting from 1-[(tert-butoxy)carbonyl]-4-(2,3-dichloro-6-methoxyphenyl)piperidine-2-carboxylic acid (Example 53, step a) and the corresponding commercially available amine.

TABLE 1D

| Compound | | | |
|---|---|---|---|
| Number | Structure | Chemical Name | MS: (M + H)⁺ & ¹H MNR |
| 75 | 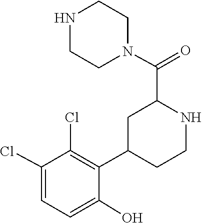 [SMILES: O=C(C1CC(c2c(O)ccc(Cl)c2Cl)CCN1)N1CCNCC1] | 3,4-dichloro-2-[2- (piperazine-1- carbonyl)piperidin- 4-yl]phenol | [M + H] 358, 360 (3:2); ¹H NMR (400 MHz, CD₃OD) δ 7.19 (dd, J = 8.8, 3.7 Hz, 1H), 6.71 (dd, J = 8.8, 2.2 Hz, 1H), 4.20 − 3.87 (m, 1H), 3.81 − 3.44 (m, 5H), 3.28 − 3.24 (m, 1H), 2.98 (d, J = 12.2 Hz, 1H), 2.89 − 2.71 (m, 5H), 2.54 − 2.37 (m, 1H), 1.79 − 1.66 (m, 1H), 1.60 − 1.56 (m, 1H). |

### Example 54. Compound 74 ((2R,4S)-rel-2-(aminomethyl)piperidin-4-yl]-3,4-dichlorophenol)

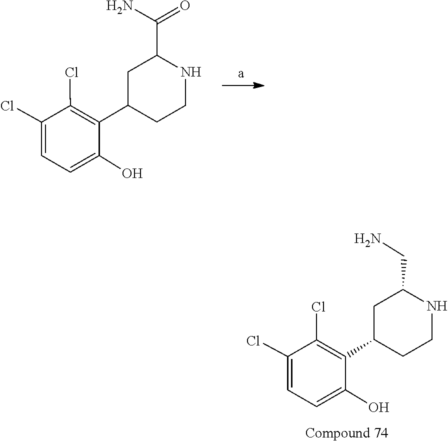 [SMILES: NC(=O)C1CC(c2c(O)ccc(Cl)c2Cl)CCN1 ; NC[C@H]1C[C@@H](c2c(O)ccc(Cl)c2Cl)CCN1]

### Step a

A stirred solution of 4-(2,3-dichloro-6-hydroxyphenyl)piperidine-2-carboxamide (40 mg, 0.14 mmol) in BH₃-THF (2 mL) was stirred for 2 h at 50° C. under nitrogen atmosphere. The mixture was allowed to cool down to room temperature. The reaction was quenched with water at room temperature. The resulting mixture was concentrated under reduced pressure. The residue was purified by Prep-HPLC with the following conditions: Column: XBridge C₁₈ OBD Prep Column, 100 Å, 10 μm, 19 mm×250 mm; Mobile Phase A: Water with 10 mmol/L NH₄HCO₃, Mobile Phase B: ACN; Flow rate: 25 mL/min; Gradient: 15 B to 55 B in 6 min; Detector: 254/210 nm; Retention time: 4.30 min. The fractions containing the desired product were collected and concentrated under reduced pressure to afford Compound 74 ((2R,4S)-rel-2-(aminomethyl)piperidin-4-yl]-3,4-dichlorophenol (cis isomer)) (14.8 mg, 39%) as an off-white solid. LCMS (ESI) calc'd for C₁₂H₁₆Cl₂N₂O [M+H]⁺: 275, 277 (3:2), found 275, 277 (3:2); ¹H NMR (300 MHz, CD₃OD) δ 7.19 (d, J=8.8 Hz, 1H), 6.72 (d, J=8.8 Hz, 1H), 3.66-3.60 (m, 1H), 3.33-3.21 (m, 1H), 2.96-2.68 (m, 4H), 2.59 (qd, J=12.8, 4.2 Hz, 1H), 2.32 (q, J=12.2 Hz, 1H), 1.67 (dd, J=22.4, 13.3 Hz, 2H).

### Example 55. Compound 77 ((2R,4S)-rel-4-(2,3-dichloro-6-hydroxyphenyl)-N,N-dimethylpiperidine-2-carboxamide) and Compound 82 ((2R,4R)-rel-4-(2,3-dichloro-6-hydroxyphenyl)-N,N-dimethylpiperidine-2-carboxamide)

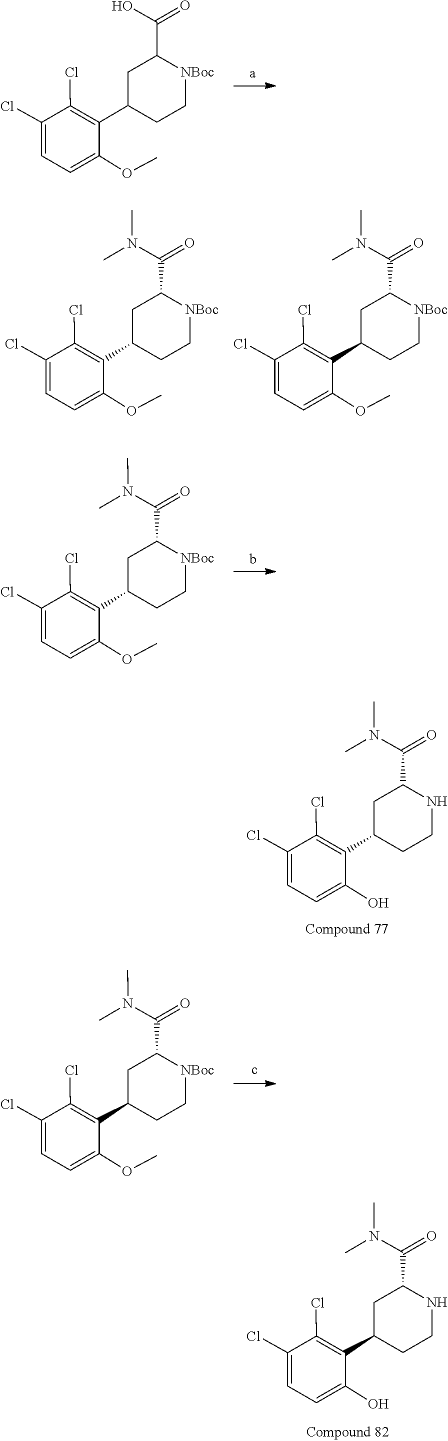

### Step a

To a stirred mixture of 1-[(tert-butoxy)carbonyl]-4-(2,3-dichloro-6-methoxyphenyl)piperidine-2-carboxylic acid (Example 53, step a) (0.28 g, 0.693 mmol) and EDCI (0.40 g, 2.08 mmol) in DMF (5 mL) was added Et₃N (0.21 g, 2.08 mmol) and dimethylamine (94 mg, 2.08 mmol) dropwise at room temperature. The resulting mixture was stirred for 2 h at room temperature. The reaction was quenched with water (20 mL). The resulting mixture was extracted with EA (3×20 mL). The combined organic layers was washed with brine (3×20 mL), dried over anhydrous Na₂SO₄. After filtration, the filtrate was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 70% B to 90% B in 6 min; Detector: 254/210 nm; Retention time: 4.87 min, 5.96 min. The fractions containing the desired product were collected at 4.87 min was concentrated under reduced pressure to afford tert-butyl 4-(2,3-dichloro-6-methoxyphenyl)-2-(dimethylcarbamoyl)piperidine-1-carboxylate cis isomer as an off-white solid (0.13 g, 44%): LCMS (ESI) calc'd for C₂₀H₂₈Cl₂N₂O₄ [M+H]⁺: 431, 433 (3:2), found 431, 433 (3:2); ¹H NMR (300 MHz, CDCl₃) δ 7.30 (d, J=13.2 Hz, 1H), 6.76 (d, J=8.9 Hz, 1H), 4.63-4.41 (m, 1H), 3.83 (s, 3H), 3.74-3.68 (m, 2H), 3.10 (s, 3H), 2.99 (s, 3H), 2.65-2.55 (m, 1H), 2.05-1.99 (m, 3H), 1.78-1.67 (m, 1H), 1.48 (d, J=2.8 Hz, 9H).

The fractions containing the desired product were collected at 5.96 min was concentrated under reduced pressure to afford tert-butyl 4-(2,3-dichloro-6-methoxyphenyl)-2-(dimethylcarbamoyl)piperidine-1-carboxylate trans isomer as an off-white solid (58 mg, 19%): LCMS (ESI) calc'd for C₂₀H₂₈Cl₂N₂O₄ [M+H]⁺: 431, 433 (3:2), found 431, 433 (3:2); ¹H NMR (300 MHz, CDCl₃) δ 7.18 (s, 1H), 6.92 (s, 1H), 4.09 (dd, J=27.7, 13.1 Hz, 1H), 3.81 (s, 3H), 3.68-3.32 (m, 2H), 3.04 (d, J=25.8 Hz, 6H), 2.24-1.95 (m, 2H), 1.95-1.70 (m, 2H), 1.62-1.42 (m, 10H).

### Step b

To a stirred solution of tert-butyl 4-(2,3-dichloro-6-methoxyphenyl)-2-(dimethylcarbamoyl)piperidine-1-carboxylate cis isomer (0.13 g, 0.30 mmol) in DCM (3 mL) was added BBr₃ (0.15 g, 0.60 mmol) dropwise at room temperature. The resulting mixture was stirred for 3 h at room temperature. The reaction was quenched with water at 0° C. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 20% B to 80% B in 6 min; Detector: 254/210 nm; Retention time: 5.07 min. The fractions containing the desired product were collected and concentrated under reduced pressure to afford Compound 77 ((2R,4S)-rel-4-(2,3-dichloro-6-hydroxyphenyl)-N,N-dimethylpiperidine-2-carboxamide (cis isomer)) as an off-white solid (48.2 mg, 48%): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.18 (d, J=8.8 Hz, 1H), 6.71 (d, J=8.8 Hz, 1H), 3.88 (dd, J=11.7, 2.8 Hz, 1H), 3.73-3.68 (m, 1H), 3.30-3.22 (m, 1H), 3.12 (s, 3H), 2.96 (s, 3H), 2.82 (td, J=13.0, 2.9 Hz, 1H), 2.50-2.36 (m, 2H), 1.73 (d, J=13.1 Hz, 1H), 1.56 (d, J=13.4 Hz, 1H).

### Step c

To a stirred solution of tert-butyl 4-(2,3-dichloro-6-methoxyphenyl)-2-(dimethylcarbamoyl)piperidine-1-carboxylate trans isomer (58 mg, 0.13 mmol) in DCM (2 mL) was added BBr₃ (67 mg, 0.27 mmol) dropwise at room temperature. The resulting mixture was stirred for 3 h at room temperature. The reaction was quenched with water at 0° C. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water (10 mmol/L NH₄HCO₃), Mobile Phase B: ACN; Flow rate: 25 mL/min; Gradient: 20% B to 80% B in 6 min; Detector: 254/210 nm; Retention time: 5.07 min. The fractions containing the desired product were collected and concentrated under reduced pressure to afford Compound 82 ((2R,4R)-rel-4-(2,3-dichloro-6-hydroxyphenyl)-N,N-dimethylpiperidine-2-carboxamide (trans isomer)) as an off-white solid (19.3 mg, 43%): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.17 (d, J=8.8 Hz, 1H), 6.70 (d, J=8.7 Hz, 1H), 4.18 (dd, J=6.2, 2.4 Hz, 1H), 3.91-3.80 (m, 1H), 3.53 (td, J=12.3, 3.5 Hz, 1H), 3.06 (s, 3H), 2.98 (s, 3H), 2.96-2.90 (m, 1H), 2.83-2.70 (m, 1H), 2.55 (qd, J=12.4, 4.6 Hz, 1H), 1.76 (d, J=13.4 Hz, 1H), 1.58 (d, J=12.6 Hz, 1H).

### Example 56. Compound 78 (2-chloro-4-hydroxy-3-(piperidin-4-yl)benzonitrile)

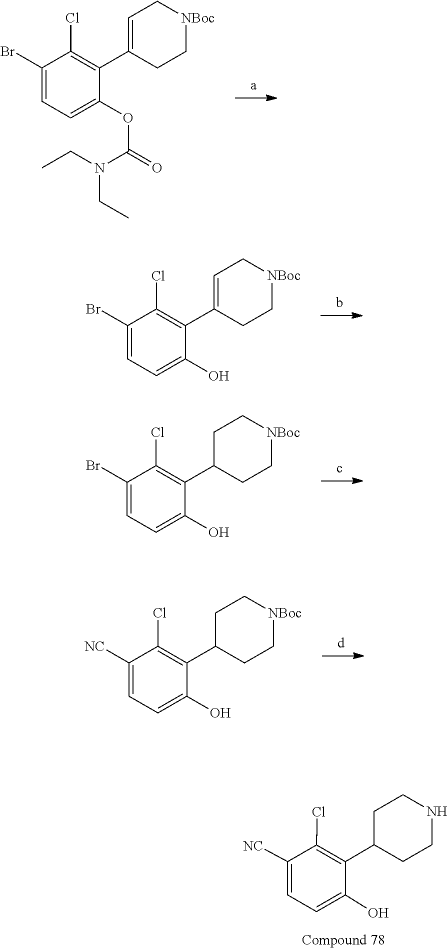 [SMILES: CCN(CC)C(=O)Oc1ccc(Br)c(Cl)c1C1=CCN(C(=O)OC(C)(C)C)CC1 ; CC(C)(C)OC(=O)N1CC=C(c2c(O)ccc(Br)c2Cl)CC1 ; CC(C)(C)OC(=O)N1CCC(c2c(O)ccc(Br)c2Cl)CC1 ; CC(C)(C)OC(=O)N1CCC(c2c(O)ccc(C#N)c2Cl)CC1 ; N#Cc1ccc(O)c(C2CCNCC2)c1Cl]

### Step a

To a solution of tert-butyl 4-[3-bromo-2-chloro-6-[(diethylcarbamoyl)oxy]phenyl]-1,2,3,6-tetrahydropyridine-1-carboxylate (0.30 g, 0.61 mmol) in EtOH (20 mL) was added NaOH (0.25 g, 6.15 mmol) at room temperature. The reaction was refluxed for 5 h and then concentrated under reduced pressure. The residue was dissolved in EA (20 mL). The resulting solution was washed with saturated aq. citric acid (10 mL) and brine (10 mL). The organic phase was then dried over anhydrous Na₂SO₄, filtered and concentrated to afford tert-butyl 4-(3-bromo-2-chloro-6-hydroxyphenyl)-1,2,3,6-tetrahydropyridine-1-carboxylate as light brown solid (0.20 g, 84%): LCMS (ESI) calc'd for C₁₆H₁₉BrClNO₃ [M−56+H]⁺: 332, 334 (2:3), found 332, 334 (2:3).

### Step b

To a solution of tert-butyl 4-(3-bromo-2-chloro-6-hydroxyphenyl)-1,2,3,6-tetrahydropyridine-1-carboxylate (50 mg, 0.13 mmol) in EtOH (5 mL) was added PtO₂ (10 mg, 0.04 mmol) under nitrogen atmosphere at room temperature. The suspension was degassed under reduced pressure and purged with H₂ for three times. The reaction mixture was stirred for 6 h at room temperature under H₂ (1.5 atm). The reaction mixture was filtered through Celite and washed with MeOH (2×3 mL). The filtrate was concentrated under reduced pressure. The residue was purified with Prep-HPLC with the following conditions: Column: XBridge C₁₈ OBD Prep Column, 19 mm×250 mm; Mobile Phase A: Water (plus 0.05% TFA), Mobile Phase B: ACN; Flow rate: 25 mL/min; Gradient: 80% B to 83% B in 6 min; Detector: UV 254/210 nm; retention time: 4.15 min. The fractions containing the desired product were combined and concentrated under reduced pressure to afford tert-butyl 4-(3-bromo-2-chloro-6-hydroxyphenyl)piperidine-1-carboxylate as an off-white solid (25 mg, 65%): LCMS (ESI) calc'd for C₁₆H₂₁BrClNO₃ [M+H]⁺: 390, 392 (2:3), found 390, 392 (2:3). ¹H NMR (400 MHz, CDCl₃) δ 11.33 (d, J=8.8 Hz, 1H), 11.26 (d, J=8.8 Hz, 1H), 10.64 (d, J=8.8 Hz, 1H), 10.58 (d, J=8.8 Hz, 1H), 9.30 (t, J=4.9 Hz, 0H), 8.13 (d, J=13.7 Hz, 1H), 7.66 (q, J=10.3, 8.2 Hz, 1H), 7.55-7.33 (m, 3H), 7.06 (t, J=12.8 Hz, 2H), 6.75 (qd, J=13.5, 4.2 Hz, 3H), 6.40 (qd, J=12.8, 4.4 Hz, 1H), 5.99 (d, J=6.1 Hz, 1H), 5.77 (d, J=14.2 Hz, 2H), 5.44 (d, J=4.9 Hz, 8H).

### Step c

A degassed solution of tert-butyl 4-(3-bromo-2-chloro-6-hydroxyphenyl)piperidine-1-carboxylate (50 mg, 0.13 mmol), Pd(PPh₃)₄ (59 mg, 0.05 mmol) and Zn(CN)₂ (7.5 mg, 0.06 mmol) in DMF (3 mL) was stirred at 90° C. for 4 h. After cooling to room temperature, the mixture was diluted with water (30 mL) and extracted with EA (3×20 mL). The combined organic layers were washed with brine (2×10 mL), dried over anhydrous Na₂SO₄. After filtration, the filtrate was concentrated under reduced pressure. The residue was purified by Prep-TLC, eluted with PE/EA (10/1) to afford tert-butyl 4-(2-chloro-3-cyano-6-hydroxyphenyl)piperidine-1-carboxylate as a yellow solid (20 mg, 46%): LCMS (ESI) calc'd for C₁₇H₂₁C₁N₂O₃ [M−H]⁺: 335, 357 (3:1), found 335, 357 (3:1).

### Step d

A solution of tert-butyl 4-(2-chloro-3-cyano-6-hydroxyphenyl)piperidine-1-carboxylate (50 mg, 0.045 mmol) in TFA(1 mL) and DCM (4 mL) was stirred at room temperature for 1 h. The resulting solution was concentrated under reduced pressure. The residue was purified by Prep-IPLC with the following conditions: Column: XBridge C₁₈ OBD Prep Column 100 Å, 10 μm, 19 mm×250 mm; Mobile Phase A: water with 10 mmol/L NH₄HCO₃, Mobile Phase B: ACN; Flow rate: 25 mL/min; Gradient: 15% B to 55% B in 6 min; Detector: 254/210 nm; Retention time: 4.30 min. The fractions containing desired product were collected and concentrated under reduced pressure to afford Compound 78 (2-chloro-4-hydroxy-3-(piperidin-4-yl)benzonitrile) as an off-white solid (3.4 mg, 24%): LCMS (ESI) calc'd for C₁₂H₁₃ClN₂O [M+H]⁺: 237, 239 (3:1), found 237, 239 (3:1); ¹H NMR (300 MHz, CD₃OD) δ 7.25 (d, J=8.7 Hz, 1H), 6.53 (d, J=8.7 Hz, 1H), 3.63-3.57 (m, 1H), 3.39 (d, J=12.8 Hz, 2H), 3.07-2.91 (m, 2H), 2.93-2.70 (m, 2H), 1.73-1.62 (m, 2H).

### Example 57. Compound 79 (N-[[4-(2,3-dichloro-6-hydroxyphenyl)piperidin-2-yl]methyl]acetamide)

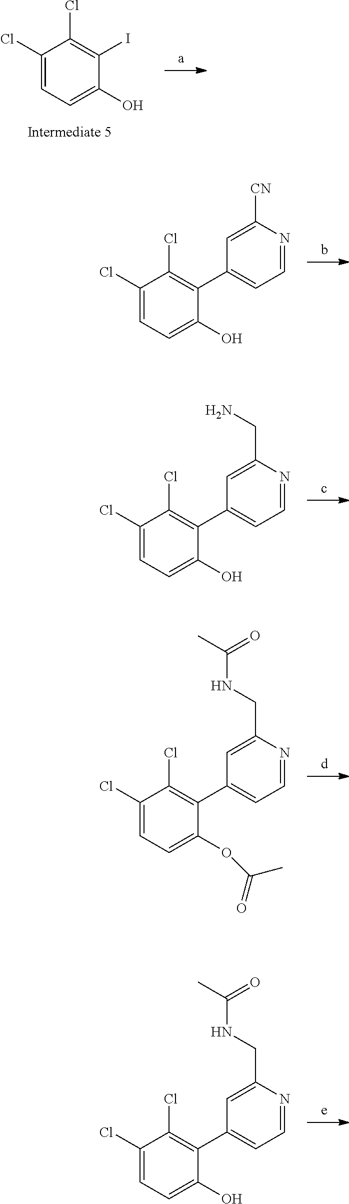 [SMILES: Oc1ccc(Cl)c(Cl)c1I ; N#Cc1cc(-c2c(O)ccc(Cl)c2Cl)ccn1 ; NCc1cc(-c2c(O)ccc(Cl)c2Cl)ccn1 ; CC(=O)NCc1cc(-c2c(OC(C)=O)ccc(Cl)c2Cl)ccn1 ; CC(=O)NCc1cc(-c2c(O)ccc(Cl)c2Cl)ccn1]

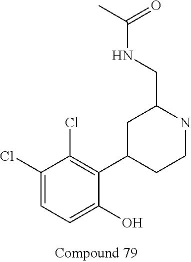 [SMILES: CC(=O)NCC1CC(c2c(O)ccc(Cl)c2Cl)CCN1]

Step a:

To a solution of Intermediate 5 (0.50 g, 1.73 mmol) and 4-(4,4,5,5-tetramethyl-1,3,2-dioxaborolan-2-yl)pyridine-2-carbonitrile (0.48 g, 2.08 mmol) in 1,4-dioxane and water were added Na₂CO₃ (0.55 g, 5.19 mmol) and Pd(dppf)Cl₂.CH₂C₁₂ (0.28 g, 0.35 mmol) at room temperature. After stirring for 3 h at 80° C. under a nitrogen atmosphere and cooling to room temperature, the resulting mixture was concentrated under reduced pressure. The residue was purified by silica gel column chromatography, eluted with PE/EA (1/1) to afford 4-(2,3-dichloro-6-hydroxyphenyl)pyridine-2-carbonitrile as an off-white solid (0.35 g, 61%): LCMS (ESI) calc'd for C₁₂H₆Cl₂N₂O [M+H]⁺: 265, 267 (3:2), found 265, 267 (3:2).

### Step b

To a stirred solution of 4-(2,3-dichloro-6-hydroxyphenyl)pyridine-2-carbonitrile (0.30 g, 1.13 mmol) in THE (3 mL) was added BH₃-Me₂S (0.8 mL, 8.36 mmol) at room temperature under air atmosphere. The reaction mixture was stirred for 12 h at 50° C. under nitrogen atmosphere. The mixture was allowed to cool down to room temperature. The reaction was quenched with water (10 mL) at room temperature. The resulting mixture was concentrated under reduced pressure. The residue was purified by Prep-TLC, eluted with PE/EA (1/2) to afford 2-[2-(aminomethyl)pyridin-4-yl]-3,4-dichlorophenol as a light yellow oil (0.28 g, 92%): LCMS (ESI) calc'd for C₁₂H₁₀Cl₂N₂O [M+H]⁺: 269, 271 (3:2), found 269, 271 (3:2); ¹H NMR (400 MHz, CD₃OD) δ 8.72 (dd, J=5.1, 0.9 Hz, 1H), 7.46-7.39 (m, 2H), 7.34 (dd, J=5.1, 1.6 Hz, 1H), 6.91 (d, J=8.9 Hz, 1H), 4.34 (s, 2H).

### Step c

To a stirred solution of 2-[2-(aminomethyl)pyridin-4-yl]-3,4-dichlorophenol (0.28 g, 1.04 mmol) and Ac₂O (0.11 g, 1.06 mmol) in DCM (3 mL) was added Et₃N (0.32 g, 3.18 mmol) in portions at room temperature under air atmosphere. The resulting mixture was stirred for 1.5 h at room temperature under nitrogen atmosphere. The resulting mixture was diluted with water (20 mL) and extracted with EA (3×20 mL). The combined organic layers were washed with brine (3×20 mL), dried over anhydrous Na₂SO₄. After filtration, the filtrate was concentrated under reduced pressure. The residue was purified by Prep-TLC, eluted with PE/EA (5/1) to afford 3,4-dichloro-2-[2-(acetamidomethyl)pyridin-4-yl]phenyl acetate as a light yellow oil (0.11 g, 30%): LCMS (ESI) calc'd for C₁₆H₁₄Cl₂N₂O₃ [M+H]⁺: 353, 355 (3:2), found 353, 355 (3:2).

### Step d

To a stirred solution of 3,4-dichloro-2-[2-(acetamidomethyl)pyridin-4-yl]phenyl acetate (20 mg, 0.06 mmol) and K₂CO₃ (40 mg, 0.29 mmol) in MeOH (1 mL) was added at room temperature under air atmosphere. The resulting mixture was stirred for overnight at room temperature under nitrogen atmosphere. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 25 mL/min; Gradient: 33% B to 50% B in 6 min; Detector: 254/210 nm; Retention time: 5 min. The fractions containing desired product was collected and concentrated under reduced pressure to afford N-[[4-(2,3-dichloro-6-hydroxyphenyl)pyridin-2-yl]methyl]acetamide as a light yellow solid (8 mg, 45%): LCMS (ESI) calc'd for C₁₄H₁₂Cl₂N₂O₂ [M+H]⁺: 311, 313 (3:2), found 311, 313 (3:2); ¹H NMR (400 MHz, CD₃OD) δ 8.56 (dd, J=5.1, 0.9 Hz, 1H), 7.40 (d, J=8.8 Hz, 1H), 7.31 (s, 1H), 7.25 (dd, J=5.1, 1.6 Hz, 1H), 6.89 (d, J=8.9 Hz, 1H), 4.56 (s, 2H), 2.05 (s, 3H).

### Step e

To a stirred solution of N-[[4-(2,3-dichloro-6-hydroxyphenyl)pyridin-2-yl]methyl]acetamide (40 mg, 0.129 mmol) and aq. HCl (5 N, 0.5 mL) in MeOH (5 mL) was added PtO₂ (40 mg, 0.178 mmol) in portions at room temperature under air atmosphere. The resulting mixture was stirred for 6 h at 30° C. under hydrogen atmosphere at 50 atm. The mixture was allowed to cool down to room temperature. After the filtration, the filter cake was washed with MeOH (3×10 mL). The filtrate was concentrated under reduced pressure. The residue was purified by Prep-HPLC with the following conditions Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 25 mL/min; Gradient: 30% B to 50% B in 6 min; Detector: 254 nm; Retention time: 4.38 min The fractions containing the desired product were collected and concentrated under reduced pressure to afford Compound 79 (N-[[4-(2,3-dichloro-6-hydroxyphenyl)piperidin-2-yl]methyl]acetamide) as an off-white solid (35 mg, 86%): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.17 (d, J=8.7 Hz, 1H), 6.70 (d, J=8.8 Hz, 1H), 3.55-3.51 (m, 1H), 3.37-3.33 (m, 1H), 3.22 (d, J=6.3 Hz, 2H), 3.18 (s, 1H), 2.84-2.71 (m, 1H), 2.59-2.44 (m, 1H), 2.23 (q, J=12.2 Hz, 1H), 1.97 (s, 3H), 1.61 (d, J=12.9 Hz, 1H), 1.54 (d, J=12.9 Hz, 1H).

### Example 58. Compound 80 ((2R,4S)-rel-3,4-dichloro-2-[2-(morpholine-4-carbonyl)piperidin-4-yl]phenol) and Compound 76 ((2R,4R)-rel-3,4-dichloro-2-[2-(morpholine-4-carbonyl)piperidin-4-yl]phenol)

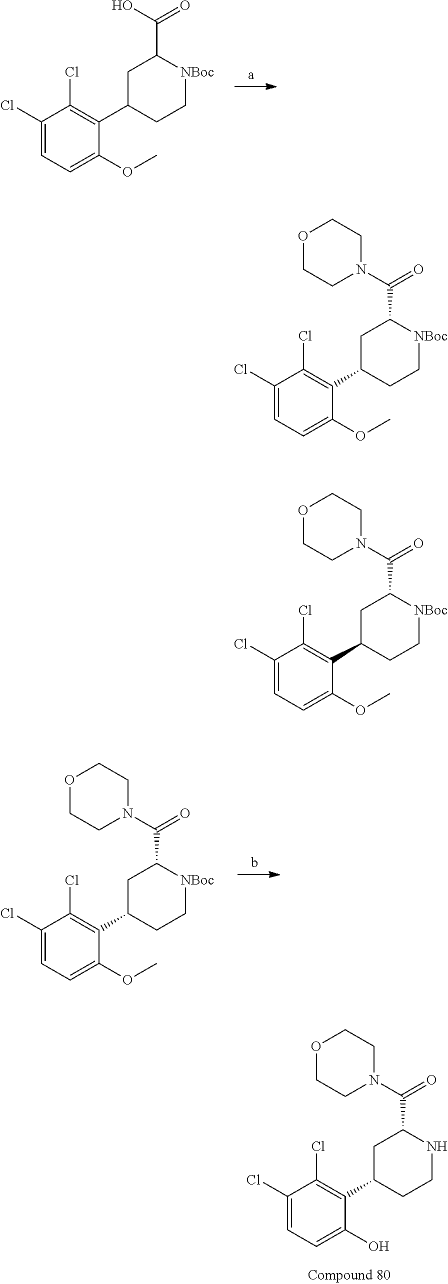 [SMILES: COc1ccc(Cl)c(Cl)c1C1CCN(C(=O)OC(C)(C)C)C(C(=O)O)C1 ; COc1ccc(Cl)c(Cl)c1[C@H]1CCN(C(=O)OC(C)(C)C)[C@@H](C(=O)N2CCOCC2)C1 ; COc1ccc(Cl)c(Cl)c1[C@@H]1CCN(C(=O)OC(C)(C)C)[C@@H](C(=O)N2CCOCC2)C1 ; COc1ccc(Cl)c(Cl)c1[C@H]1CCN(C(=O)OC(C)(C)C)[C@@H](C(=O)N2CCOCC2)C1 ; O=C([C@H]1C[C@@H](c2c(O)ccc(Cl)c2Cl)CCN1)N1CCOCC1]

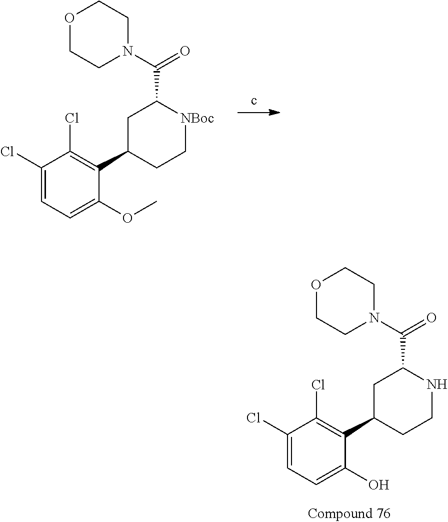 [SMILES: COc1ccc(Cl)c(Cl)c1[C@@H]1CCN(C(=O)OC(C)(C)C)[C@@H](C(=O)N2CCOCC2)C1 ; O=C([C@H]1C[C@H](c2c(O)ccc(Cl)c2Cl)CCN1)N1CCOCC1]

### Step a

To a stirred solution of 1-[(tert-butoxy)carbonyl]-4-(2,3-dichloro-6-methoxyphenyl)piperidine-2-carboxylic acid (Example 53, step a) (0.20 g, 0.496 mmol) and HATU (0.37 g, 0.99 mmol) in DMF (5 mL) were added morpholine (87 mg, 0.99 mmol) and Et₃N (0.15 g, 1.48 mmol) dropwise at room temperature under nitrogen atmosphere. The resulting mixture was stirred for 1 h at room temperature under nitrogen atmosphere. The resulting mixture was diluted with water (30 mL) and extracted with EA (3×30 mL). The combined organic layers was washed with brine (3×20 mL) and dried over anhydrous Na₂SO₄. After filtration, the filtrate was concentrated under reduced pressure. The residue was purified by Prep-HPLC with the following conditions: Column: XSelect CSH Prep C₁₈ OBD Column, 19×250 mm, 5 m; Mobile Phase A: Water (plus 0.05% TFA), Mobile Phase B: ACN; Flow rate: 25 mL/min; Gradient: 28% B to 30% B in 6 min; Detector: 210 nm; Retention time: RT₁: 4.53 min, RT₂: 5.50 min. The fractions containing the desired product were collected at 4.53 min was concentrated under reduced pressure to afford tert-butyl 4-(2,3-dichloro-6-methoxyphenyl)-2-(morpholine-4-carbonyl)piperidine-1-carboxylate cis isomer as a yellow oil (0.11 g, 47%): LCMS (ESI) calc'd for C₂₂H₃₀Cl₂N₂O₅ [M+H]⁺: 473, 475 (3:2), found 473, 475 (3:2).

The fractions containing the desired product were collected at 5.50 min was concentrated under reduced pressure to afford tert-butyl 4-(2,3-dichloro-6-methoxyphenyl)-2-(morpholine-4-carbonyl)piperidine-1-carboxylate trans isomer as a yellow oil (58 mg, 25%): LCMS (ESI) calc'd for C₂₂H₃₀Cl₂N₂O₅ [M+H]⁺: 473, 475 (3:2), found 473, 475 (3:2).

### Step b

To a stirred solution of tert-butyl 4-(2,3-dichloro-6-methoxyphenyl)-2-(morpholine-4-carbonyl)piperidine-1-carboxylate cis isomer (0.11 g, 0.233 mmol) in DCM (3 mL) was added BBr₃ (0.12 g, 0.47 mmol) dropwise at room temperature. The resulting mixture was stirred for 3 h at room temperature. The reaction was quenched with water at 0° C. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 20% B to 60% B in 8 min; Detector: 254/210 nm; Retention time: 6.25 min. The fractions containing the desired product were collected and concentrated under reduced pressure to afford Compound 80 ((2R,4S)-rel-3,4-dichloro-2-[2-(morpholine-4-carbonyl)piperidin-4-yl]phenol (cis isomer)) as an off-white solid (22 mg, 22%): LCMS (ESI) calc'd for C₁₆H₂₀Cl₂N₂O₃ [M+H]⁺: 359, 361 (3:2), found 359, 361 (3:2); ¹H NMR (400 MHz, CD₃OD) δ 7.19 (d, J=8.8 Hz, 1H), 6.71 (d, J=8.7 Hz, 1H), 3.90 (dd, J=11.8, 2.9 Hz, 1H), 3.69-3.64 (m, 7H), 3.59-3.54 (m, 2H), 3.29-3.24 (m, 1H), 2.89-2.77 (m, 1H), 2.55-2.41 (m, 2H), 1.70 (d, J=13.4 Hz, 1H), 1.56 (d, J=13.4 Hz, 1H).

### Step c

To a stirred solution of tert-butyl 4-(2,3-dichloro-6-methoxyphenyl)-2-(morpholine-4-carbonyl)piperidine-1-carboxylate trans isomer (58 mg, 0.122 mmol) in DCM (3 mL) was added BBr₃ (62 mg, 0.25 mmol) dropwise at room temperature. The resulting mixture was stirred for 3 h at room temperature. The reaction was quenched with water at 0° C. The resulting mixture was concentrated under reduced pressure. The residue was purified by Prep-HPLC with the following conditions: Column: XBridge C₁₈ OBD Prep Column 100 Å, 10 m, 19 mm×250 mm; Mobile Phase A: Water (plus 0.05% TFA), Mobile Phase B: ACN; Flow rate: 25 mL/min; Gradient: 10% B to 50% B in 8 min; Detector: 254/210 nm; Retention time: 5.80 min. The fractions containing the desired product were collected and concentrated under reduced pressure to afford Compound 76 ((2R,4R)-rel-3,4-dichloro-2-[2-(morpholine-4-carbonyl)piperidin-4-yl]phenol (trans isomer)) as an off-white solid (1.9 mg, 3%): LCMS (ESI) calc'd for C₁₆H₂₀Cl₂N₂O₃ [M+H]⁺: 359, 361 (3:2), found 359, 361 (3:2); ¹H NMR (400 MHz, CD₃OD) δ 7.27 (d, J=8.8 Hz, 1H), 6.77 (d, J=8.8 Hz, 1H), 3.89 (td, J=12.7, 5.1 Hz, 2H), 3.78 (t, J=5.4 Hz, 3H), 3.74-3.59 (m, 4H), 3.57-3.49 (m, 1H), 3.48-3.43 (m, 1H), 3.26-3.15 (m, 1H), 2.92-2.67 (m, 2H), 2.43 (d, J=14.3 Hz, 1H), 1.87 (d, J=14.3 Hz, 1H).

### Example 59. Compound 81 (4-bromo-3-chloro-2-(piperidin-4-yl)phenol)

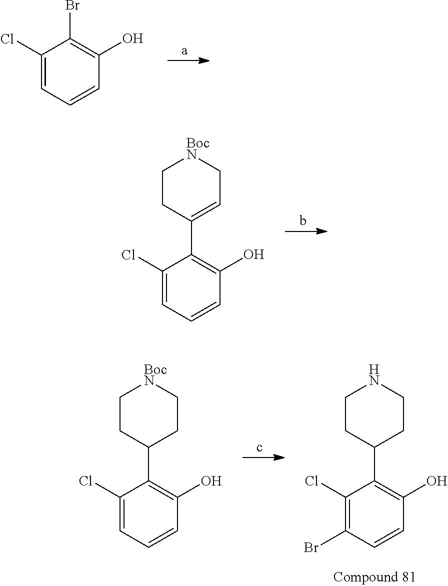 [SMILES: Oc1cccc(Cl)c1Br ; CC(C)(C)OC(=O)N1CC=C(c2c(O)cccc2Cl)CC1 ; CC(C)(C)OC(=O)N1CCC(c2c(O)cccc2Cl)CC1 ; Oc1ccc(Br)c(Cl)c1C1CCNCC1]

### Step a

To a stirred mixture of 2-bromo-3-chlorophenol (4.50 g, 21.69 mmol) and tert-butyl 4-(4,4,5,5-tetramethyl-1,3,2-dioxaborolan-2-yl)-1,2,3,6-tetrahydropyridine-1-carboxylate (7.50 g, 24.26 mmol) in 1,4-dioxane (80 mL) and H₂O (20 mL) were added Pd(dppf)Cl₂.CH₂C₂ (0.60 g, 0.73 mmol) and Na₂CO₃ (6.80 g, 64.16 mmol) at room temperature under argon atmosphere. The reaction was stirred at 80° C. for 3 h. After cooling to room temperature, the reaction was concentrated under reduced pressure. The residue was dissolved in EA (80 mL) and water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified with silica gel column chromatography, eluted with PE/EA (3/1) to afford tert-butyl 4-(2-chloro-6-hydroxyphenyl)-1,2,3,6-tetrahydropyridine-1-carboxylate as a light yellow solid (5.00 g, 74%): LCMS (ESI) calc'd for C₁₆H₂₀ClNO₃ [M+H−56]⁺: 254, 256 (3:1), found 254, 256 (3:1); ¹H NMR (400 MHz, CDCl₃) δ 7.12 (t, J=8.1 Hz, 1H), 6.97 (dd, J=8.0, 1.1 Hz, 1H), 6.87 (dd, J=8.2, 1.1 Hz, 1H), 5.87-5.77 (m, 1H), 5.64 (s, 1H), 4.36-3.98 (m, 2H), 3.94-3.37 (m, 2H), 2.56-2.25 (m, 2H), 1.53 (s, 9H).

### Step b

To a stirred solution of tert-butyl 4-(2-chloro-6-hydroxyphenyl)-1,2,3,6-tetrahydropyridine-1-carboxylate (4.00 g, 12.91 mmol) in EtOH (200 mL) and AcOH (20 mL) was added PtO₂ (0.30 g, 1.32 mmol). The reaction mixture was degassed with hydrogen three times and stirred under hydrogen atmosphere (1.5 atm) at room temperature for 5 h. The reaction mixture was filtered and the filtrate was concentrated under reduced pressure. The residue was purified by reverse phase chromatography, eluted with 70% ACN in water (plus 0.05% TFA) to afford tert-butyl 4-(2-chloro-6-hydroxyphenyl)piperidine-1-carboxylate as off-white solid (1.50 g, 37%): LCMS (ESI) calc'd for C₁₆H₂₂C₁NO₃ [M+H−15]⁺: 297, 299 (3:1), found 297, 299 (3:1); ¹H NMR (400 MHz, CD₃OD) δ 6.96 (t, J=8.1 Hz, 1H), 6.87-6.79 (m, 1H), 6.68 (d, J=7.7 Hz, 1H), 4.25-4.10 (m, 2H), 3.59-3.40 (m, 1H), 2.82 (s, 2H), 2.54-2.36 (m, 2H), 1.57-1.43 (m, 11H).

### Step c

To a stirred solution of tert-butyl 4-(2-chloro-6-hydroxyphenyl)piperidine-1-carboxylate (40 mg, 0.13 mmol) in DCM (2 mL) was added Br₂ (20 mg, 0.13 mmol) over 10 min at 0° C. under nitrogen atmosphere. The reaction was stirred at room temperature for 2 h. The reaction mixture was quenched with saturated aq. Na₂S203 (0.5 mL) and concentrated under reduced pressure. The residue was purified by Prep-HPLC with the following conditions: Column: XBridge C₁₈ OBD Prep Column 19 mm×250 mm, 10 μm; Mobile Phase A: water (plus 0.05% TFA), Mobile Phase B: ACN; Flow rate: 25 mL/min; Gradient: 20% B to 45% B in 6 min; Detector: UV 210/254 nm; Retention time: 5.16 min. The fractions containing the desired product were collected and concentrated under reduced pressure to afford Compound 81 (4-bromo-3-chloro-2-(piperidin-4-yl)phenol) as off-white solid (3.8 mg, 10%): LCMS (ESI) calc'd for C₁₁H₁₃BrClNO [M+H]⁺: 290, 292, 294 (2:3:1), found 290, 292, 294 (2:3:1); ¹H NMR (400 MHz, CD₃OD) δ 7.39 (d, J=8.8 Hz, 1H), 6.70 (d, J=8.7 Hz, 1H), 3.81-3.66 (m, 1H), 3.54-3.41 (m, 2H), 3.18-3.04 (m, 2H), 2.90-2.71 (m, 2H), 1.89-1.74 (m, 2H).

### Example 60. Compound 83 (4-(2,3-dichloro-6-hydroxyphenyl)-2-methylpiperidine-2-carboxamide)

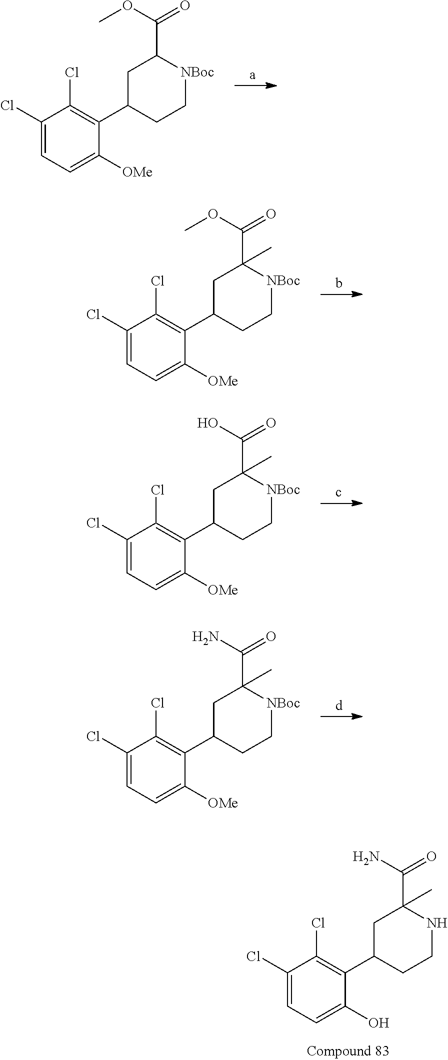

### Step a

To a stirred solution of diisopropylamine (97 mg, 0.96 mmol) in THE (2 mL) was added n-BuLi (0.38 mL, 0.96 mmol, 2.5 M in hexane) at −78° C. under argon atmosphere. The solution was stirred at −78° C. for 20 min. Then a solution of 1-tert-butyl 2-methyl 4-(2,3-dichloro-6-methoxyphenyl)piperidine-1,2-dicarboxylate (Example 61, step c) (0.20 g, 0.48 mmol) in THE (2 mL) was added into the above solution. The reaction was stirred at −78° C. to −65° C. for 40 min. A solution of CH₃I (0.14 g, 0.96 mmol) in THE (1 mL) was added into. The resulting solution was stirred at −65° C. for 2 h. The reaction was quenched with water (1 mL) at −65° C. and diluted with water (30 mL). The isolated aqueous layer was extracted with EA (3×30 mL). The combined organic layers were washed with brine (3×20 mL) and evaporated under reduced pressure. The residue was purified by reverse phase chromatography, eluted with 79% ACN in water (plus 0.1% TFA) to afford 1-tert-butyl 2-methyl 4-(2,3-dichloro-6-methoxyphenyl)-2-methylpiperidine-1,2-dicarboxylate as a light yellow oil (0.15 g, 72%): LCMS (ESI) calc'd for C₂₀H₂₇Cl₂NO₅ [M+H]⁺: 432, 434 (3:2), found 432, 434 (3:2); ¹H NMR (300 MHz, CDCl₃) δ 7.26 (d, J=13.9 Hz, 1H), 6.73 (d, J=8.8 Hz, 1H), 4.41 (s, 2H), 3.80 (d, J=9.9 Hz, 6H), 3.51 (s, 1H), 3.36 (s, 1H), 2.50 (d, J=15.2 Hz, 1H), 2.13 (s, 1H), 1.87 (s, 1H), 1.57 (s, 3H), 1.45 (s, 9H).

### Step b

To a stirred solution of 1-tert-butyl 2-methyl 4-(2,3-dichloro-6-methoxyphenyl)-2-methylpiperidine-1,2-dicarboxylate (0.14 g, 0.32 mmol) in 1,4-dioxane (3 mL) and water (0.5 mL) was added NaOH (0.13 g, 3.24 mmol) at room temperature. The reaction was stirred at 90° C. for 16 h. The reaction was acidified with citric acid to pH 4. The solution was diluted with EA (20 mL) and water (20 mL). The aqueous layer was extracted with EA (3×20 mL). The combined organic layers were washed with brine (3×20 mL) and evaporated under reduced pressure. The residue was purified by reverse phase chromatography, eluted with 67% ACN in water (plus 0.1% TFA) to afford 1-[(tert-butoxy)carbonyl]-4-(2,3-dichloro-6-methoxyphenyl)-2-methylpiperidine-2-carboxylic acid as an off-white solid (60 mg, 44%): LCMS (ESI) calc'd for C₁₉H₂₅Cl₂NO₅ [M+H]⁺: 418, 420 (3:2), found 418, 420 (3:2); ¹H NMR (300 MHz, CDCl₃) δ 7.26 (d, J=13.9 Hz, 1H), 6.73 (d, J=8.8 Hz, 1H), 4.19-3.99 (m, 2H), 3.83 (s, 3H), 3.51-3.45 (m, 1H), 2.63-2.45 (m, 1H), 2.25-2.15 (m, 1H), 1.95-1.75 (m, 2H), 1.61 (s, 3H), 1.48 (s, 9H).

### Step c

To a stirred solution of 1-[(tert-butoxy)carbonyl]-4-(2,3-dichloro-6-methoxyphenyl)-2-methylpiperidine-2-carboxylic acid (45 mg, 0.11 mmol) and HATU (61 mg, 0.16 mmol) in DMF (2 mL) was added Et₃N (22 mg, 0.22 mmol) and NH₄Cl (58 mg, 1.08 mmol) at room temperature. The reaction was stirred at room temperature for 1 h. The reaction was quenched with water (1 mL) and diluted with EA (30 mL) and water (30 mL). The partitioned aqueous solution was extracted with EA (3×30 mL). The combined organic layers were washed with brine (3×20 mL) and evaporated under reduced pressure. The residue was purified by reverse phase chromatography, eluted with 60% ACN in water with 5 mmol/L NH₄HCO₃ to afford tert-butyl 2-carbamoyl-4-(2,3-dichloro-6-methoxyphenyl)-2-methylpiperidine-1-carboxylate as a light yellow oil (30 mg, 67%): LCMS (ESI) calc'd for C₁₉H₂₆Cl₂N₂O₄ [M+H]⁺: 417, 419 (3:2), found 417, 419 (3:2).

### Step d

To a stirred solution of tert-butyl 2-carbamoyl-4-(2,3-dichloro-6-methoxyphenyl)-2-methylpiperidine-1-carboxylate (50 mg, 0.12 mmol) in DCM (2 mL) was added BBr₃ (0.18 g, 0.72 mmol) at room temperature. The reaction was stirred at room temperature for 1 h. The reaction was quenched with water (1 mL) and concentrated under reduced pressure. The residue was purified by Prep-HPLC with following conditions: Column: XBridge C₁₈ OBD Prep Column 100 Å, 10 μm, 19 mm×250 mm; Mobile Phase A: Water with 10 mmol/L NH₄HCO₃, Mobile Phase B: ACN; Flow rate: 25 mL/min; Gradient: 31% B to 49% B in 6 min; Detector: 210 nm; Retention time: 5.43 min. The fractions containing desired product were collected and concentrated under reduced pressure to afford Compound 83 (4-(2,3-dichloro-6-hydroxyphenyl)-2-methylpiperidine-2-carboxamide) as an off-white solid (7.8 mg, 27%): LCMS (ESI) calc'd for C₁₃H₁₆Cl₂N₂O₂ [M+H]⁺: 303, 305 (3:2), found 303, 305 (3:2); ¹H NMR (400 MHz, CD₃OD) δ 7.16 (d, J=8.8 Hz, 1H), 6.69 (d, J=8.8 Hz, 1H), 3.59-3.46 (m, 1H), 3.06-2.97 (m, 1H), 2.86 (td, J=12.8, 3.1 Hz, 1H), 2.52-2.33 (m, 2H), 2.25-2.16 (m, 1H), 1.46 (d, J=12.9 Hz, 1H), 1.31 (s, 3H).

### Example 61. Compound 84 (4-(2,3-dichloro-6-hydroxyphenyl)piperidine-2-carboxylic acid)

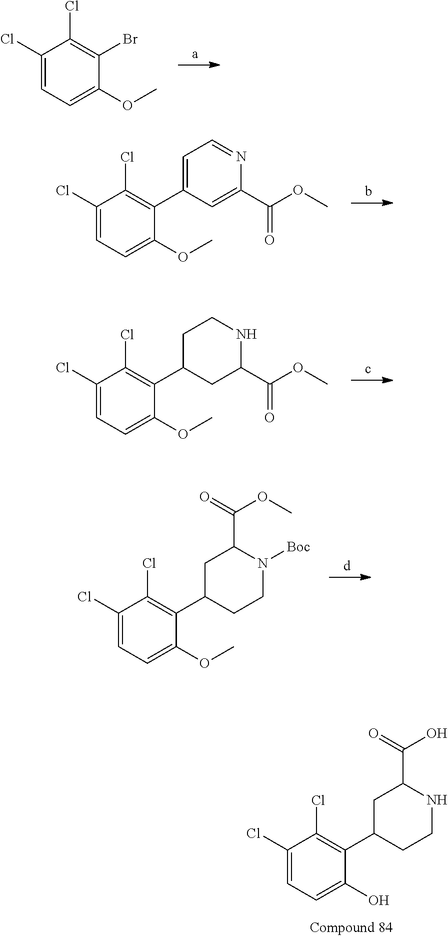 [SMILES: COc1ccc(Cl)c(Cl)c1Br ; COC(=O)c1cc(-c2c(OC)ccc(Cl)c2Cl)ccn1 ; COC(=O)C1CC(c2c(OC)ccc(Cl)c2Cl)CCN1 ; COC(=O)C1CC(c2c(OC)ccc(Cl)c2Cl)CCN1C(=O)OC(C)(C)C ; O=C(O)C1CC(c2c(O)ccc(Cl)c2Cl)CCN1]

### Step a

To a solution of 2-bromo-3,4-dichloro-1-methoxybenzene (5 g, 0.02 mmol, 1 equiv) and methyl 4-(4,4,5,5-tetramethyl-1,3,2-dioxaborolan-2-yl)pyridine-2-carboxylate (6.2 g, 0.02 mmol, 1.2 equiv) in dioxane and water were added Na₂CO₃ (6.2 g, 0.06 mmol, 3 equiv.) and Pd(dppf)Cl₂.CH₂Cl₂ (3.2 g, 0.2 equiv). After stirring for 3 h at 80° C. under a nitrogen atmosphere, the resulting mixture was concentrated under reduced pressure. The residue was purified by silica gel column chromatography, eluted with PE/EtOAc (3:1) to afford methyl 4-(2,3-dichloro-6-methoxyphenyl)pyridine-2-carboxylate (1 g, 16.4%) as a light-yellow solid. LCMS (ESI) calc'd for C₁₄H₁₁Cl₂NO₃ [M+H]⁺: 312, 314 (3:2), found 312, 314 (3:2). ¹H NMR (400 MHz, CD₃OD) δ 8.78 (d, J=5.0 Hz, 1H), 8.08 (s, 1H), 7.66-7.57 (m, 2H), 7.16 (d, J=9.0 Hz, 1H), 4.01 (s, 3H), 3.78 (s, 3H).

### Step b

To a solution of PtO₂ (65.5 mg, 0.29 mmol, 0.3 equiv) and methyl 4-(2,3-dichloro-6-methoxyphenyl)pyridine-2-carboxylate (300 mg, 0.96 mmol, 1 equiv) in MeOH was added HCl (6 M, 1 mL) in portions at room temperature. The resulting mixture was stirred for 4 days at 30° C. under hydrogen atmosphere. The solid was filtered out and washed with MeOH (3×10 mL). The filtrate was concentrated under reduced pressure to afford methyl 4-(2,3-dichloro-6-methoxyphenyl)piperidine-2-carboxylate (200 mg, 52.32%) as a yellow oil. LCMS (ESI) calc'd for C₁₄H₁₇Cl₂NO₃ [M+H]⁺: 318, 320 (3:2), found 318, 320 (3:2). ¹H NMR (400 MHz, CD₃OD) δ 7.38 (d, J=8.9 Hz, 1H), 6.96 (d, J=9.0 Hz, 1H), 3.86 (s, 3H), 3.74 (s, 3H), 3.67-3.58 (m, 1H), 3.47 (dd, J=11.9, 3.0 Hz, 1H), 3.26-3.16 (m, 1H), 2.76 (td, J=12.4, 2.9 Hz, 1H), 2.45-2.27 (m, 2H), 1.90 (d, J=12.7 Hz, 1H), 1.51 (d, J=13.1 Hz, 1H).

### Step c

To a stirred solution of methyl 4-(2,3-dichloro-6-methoxyphenyl)piperidine-2-carboxylate (100.00 mg, 0.314 mmol, 1.00 equiv) and Et₃N (95.41 mg, 0.943 mmol, 3.00 equiv) in DCM (1.00 mL) were added Boc₂O (102.89 mg, 0.471 mmol, 1.50 equiv) dropwise at room temperature under air atmosphere. The resulting solution was stirred for 1 h at room temperature under air atmosphere. The resulting mixture was concentrated under reduced pressure. The residue was purified by Prep-TLC (PE/EtOAc 5:1) to afford 1-tert-butyl 2-methyl 4-(2,3-dichloro-6-methoxyphenyl)piperidine-1,2-dicarboxylate (100 mg, 68.46%) as an off-white solid. LCMS (ESI) calc'd for C₁₉H₂₅Cl₂NO₅ [M+H]⁺: 418, 420 (3:2), found 418, 420 (3:2); ¹H NMR (400 MHz, CDCl₃) δ 7.31 (d, J=8.9 Hz, 1H), 6.76 (d, J=8.9 Hz, 1H), 4.23 (dd, J=12.0, 5.5 Hz, 1H), 3.83 (s, 3H), 3.76 (s, 3H), 3.70-3.59 (m, 2H), 2.64-2.47 (m, 1H), 2.11-2.00 (m, 1H), 1.98-1.86 (m, 2H), 1.52-1.46 (m, 10H).

### Step d

A mixture of 1-tert-butyl 2-methyl 4-(2,3-dichloro-6-methoxyphenyl)piperidine-1,2-dicarboxylate (38 mg, 0.09 mmol) and BBr₃ (0.16 g, 0.64 mmol) in DCM (3 mL) was stirred for 1 h at room temperature under nitrogen atmosphere. The reaction was quenched with MeOH at room temperature. The resulting mixture was concentrated under reduced pressure. The crude product was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 10% B to 50% B in 6 min; Detector: UV 254/210 nm; Retention time: 5.96 min. The fractions containing desired product were collected and concentrated under reduced pressure to afford Compound 84 (4-(2,3-dichloro-6-hydroxyphenyl)piperidine-2-carboxylic acid) as an off-white solid (10.6 mg, 40%): LCMS (ESI) calc'd for C₁₂H₁₃Cl₂NO₃ [M+H]⁺: 290, 292 (3:2), found 290, 292 (3:2). ¹H NMR (400 MHz, CD₃OD) δ 7.27 (d, J=8.7 Hz, 1H), 6.77 (d, J=8.8 Hz, 1H), 4.08 (dd, J=12.9, 3.4 Hz, 1H), 3.89-3.80 (m, 1H), 3.57-3.50 (m, 1H), 3.20 (dd, J=13.2, 3.2 Hz, 1H), 2.87-2.69 (m, 2H), 2.24 (d, J=13.9 Hz, 1H), 1.82 (d, J=14.2 Hz, 1H).

### Example 62. Compound 85 ((2R,4S)-rel-4-(4,5-dichloro-2-hydroxyphenyl)piperidine-2-carboxamide)

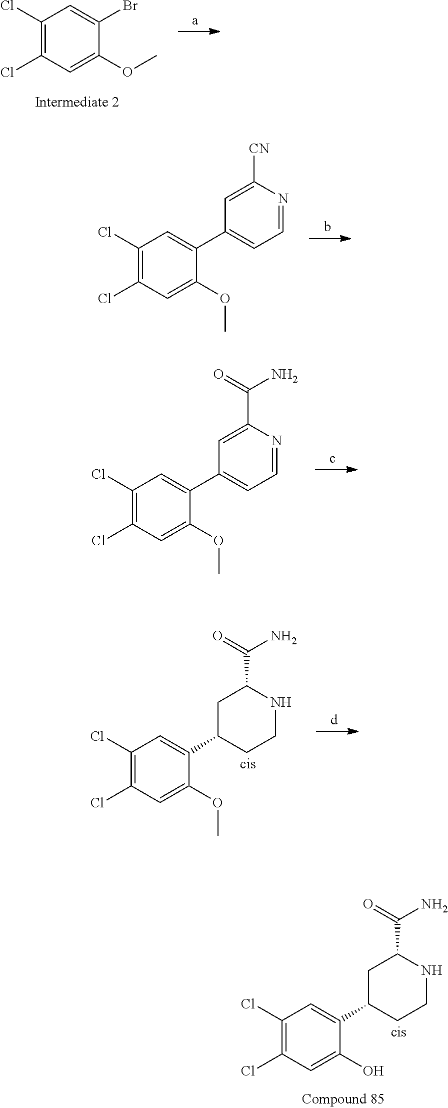

### Step a

To a stirred solution of Intermediate 2 (0.15 g, 0.59 mmol) in 1,4-dioxane (4 mL) and H₂O (1 mL) was added Pd(dppf)Cl₂.CH₂C₂ (96 mg, 0.12 mmol), 4-(4,4,5,5-tetramethyl-1,3,2-dioxaborolan-2-yl)pyridine-2-carbonitrile (0.16 g, 0.70 mmol) and Na₂CO₃ (0.19 g, 1.76 mmol) at room temperature. The resulting mixture was stirred for 2 h at 80° C. under nitrogen atmosphere. After cooling to room temperature, the reaction was quenched with water (30 mL) at room temperature. The resulting mixture was extracted with EA (3×30 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (5/1) to afford 4-(4,5-dichloro-2-methoxyphenyl)pyridine-2-carbonitrile as a yellow solid (0.16 g, 89%): LCMS (ESI) calc'd for C₁₃H₈Cl₂N₂O [M+H]⁺: 279, 281 (3:2), found 279, 281 (3:2); ¹H NMR (300 MHz, CDCl₃) δ 8.74 (d, J=5.1 Hz, 1H), 7.88 (s, 1H), 7.61 (dd, J=5.2, 1.8 Hz, 1H), 7.42 (s, 1H), 7.12 (s, 1H), 3.87 (s, 3H).

### Step b

To a stirred solution 4-(4,5-dichloro-2-methoxyphenyl)pyridine-2-carbonitrile (0.16 g, 0.58 mmol) in MeOH (2 mL) and THE (2 mL) was added H₂O₂ (0.5 mL, 30% in water) dropwise at room temperature under air atmosphere. The resulting mixture was stirred for 3 h at room temperature under air atmosphere. The reaction was quenched with saturated aq. Na₂SO₃ (30 mL) at room temperature. The aqueous layer was extracted with EA (3×20 mL). The resulting mixture was concentrated under reduced pressure to afford 4-(4,5-dichloro-2-methoxyphenyl)pyridine-2-carboxamide as a yellow solid (0.15 g, 69%): LCMS (ESI) calc'd for C₁₃H₁₀Cl₂N₂O₂ [M+H]⁺: 297, 299 (3:2), found 418, 420 (3:2).

### Step c

To a stirred mixture of 4-(4,5-dichloro-2-methoxyphenyl)pyridine-2-carboxamide (0.20 g, 0.67 mmol) in MeOH (13 mL) were added aq. HCl (6 N, 1.3 mL) and PtO₂ (20 mg, 0.09 mmol) in portions at room temperature. The resulting mixture was degassed with hydrogen three times and stirred for 6 h at 30° C. under hydrogen atmosphere (1.5 atm). After filtration, the filter cake was washed with EA (3×10 mL). The filtrate was concentrated under reduced pressure to afford 4-(4,5-dichloro-2-methoxyphenyl)piperidine-2-carboxamide (cis isomer) as a yellow solid (0.15 g, 59%): LCMS (ESI) calc'd for C₁₃H₁₆Cl₂N₂O₂ [M+H]⁺: 303, 305 (3:2), found 303, 305 (3:2); ¹H NMR (300 MHz, CD₃OD) δ 7.29 (s, 1H), 7.17 (s, 1H), 4.03-3.93 (m, 1H), 3.87 (s, 3H), 3.51 (d, J=12.8 Hz, 1H), 3.36-3.11 (m, 2H), 2.36 (d, J=13.8 Hz, 1H), 2.07-1.80 (m, 3H).

### Step d

A solution 4-(4,5-dichloro-2-methoxyphenyl)piperidine-2-carboxamide (0.15 g, 0.49 mmol) and BBr₃ (1.24 g, 4.95 mmol) in DCM (2 mL) was stirred for 1 h at room temperature under air atmosphere. The reaction was quenched by the addition of water (5 mL) at room temperature. The pH value of the reaction system was adjusted to 9 with saturated aq. NaHCO₃ at 0° C. The resulting mixture was concentrated under reduced pressure. The crude product was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 22% B to 27% B in 6 min; Detector: UV 254/210 nm; Retention time: 5.05 min. The fractions containing desired product was collected and concentrated under reduced pressure to afford Compound 85 ((2R,4S)-rel-4-(4,5-dichloro-2-hydroxyphenyl)piperidine-2-carboxamide (cis isomer)) as an off-white solid (47 mg, 22%): LCMS (ESI) calc'd for C₁₂H₁₄Cl₂N₂O₂ [M+H]⁺: 289, 291 (3:2), found 289, 291 (3:2); ¹H NMR (400 MHz, CD₃OD) δ 7.25 (s, 1H), 6.97 (s, 1H), 4.00 (dd, J=12.5, 3.1 Hz, 1H), 3.54 (d, J=12.6 Hz, 1H), 3.30 (d, J=12.3 Hz, 1H), 3.21 (td, J=12.8, 3.3 Hz, 1H), 2.41 (d, J=13.8 Hz, 1H), 2.14-1.90 (m, 3H).

### Example 63. Compound 86 (N-[[(2R,4S)-rel-4-(2,3-dichloro-6-hydroxyphenyl)piperidin-2-yl]methyl]acetamide isomer 1) and Compound 89 (N-[[(2R,4S)-rel-4-(2,3-dichloro-6-hydroxyphenyl)piperidin-2-yl]methyl]acetamide isomer 2)

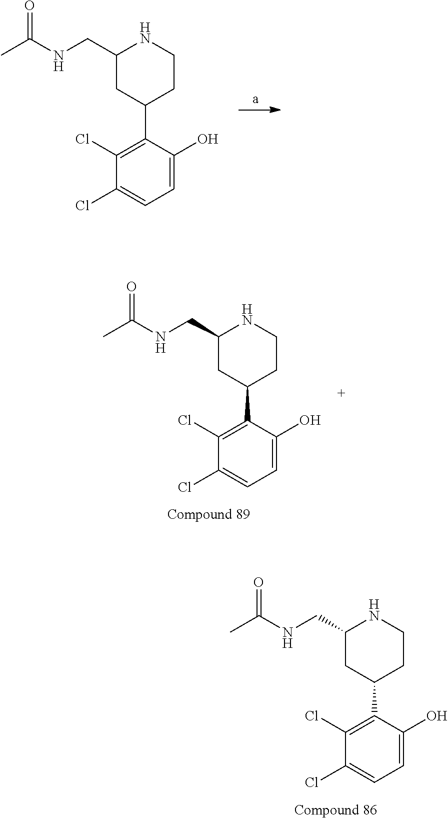 [SMILES: CC(=O)NCC1CC(c2c(O)ccc(Cl)c2Cl)CCN1 ; CC(=O)NC[C@@H]1C[C@H](c2c(O)ccc(Cl)c2Cl)CCN1 ; CC(=O)NC[C@H]1C[C@@H](c2c(O)ccc(Cl)c2Cl)CCN1]

### Step a

N-[[4-(2,3-dichloro-6-hydroxyphenyl)piperidin-2-yl]methyl]acetamide (Compound 79, Example 57) (28 mg, 0.088 mmol) was separated by Chiral Prep-HPLC with the following conditions: Column: Chiralpak IG, 20×250 mm, 5 m; Mobile Phase A: Hex (0.1% IPA), Mobile Phase B: EtOH; Flow rate: 20 mL/min; Gradient: 10 B to 10 B in 13 min; Detector: 254/220 nm; Retention time: RT₁: 7.478 min, RT₂: 10.103 min.

The faster-eluting enantiomer Compound 89 (N-[[(2R,4S)-rel-4-(2,3-dichloro-6-hydroxyphenyl)piperidin-2-yl]methyl]acetamide isomer 2) was obtained as an off-white solid (5.6 mg, 20%) at 7.478 min: LCMS (ESI) calc'd for C₁₄H₁₈Cl₂N₂O₂ [M+H]⁺: 317, 319 (3:2), found 317, 319 (3:2); ¹H NMR (300 MHz, CD₃OD) δ 7.15 (d, J=8.8 Hz, 1H), 6.68 (d, J=8.8 Hz, 1H), 3.21 (d, J=6.1 Hz, 3H), 2.77 (dd, J=12.1, 2.8 Hz, 3H), 2.60-2.42 (m, 1H), 2.22 (q, J=12.1 Hz, 1H), 1.95 (s, 3H), 1.65-1.47 (m, 2H).

The slower-eluting enantiomer Compound 86 (N-[[(2R,4S)-rel-4-(2,3-dichloro-6-hydroxyphenyl)piperidin-2-yl]methyl]acetamide isomer 1) was obtained as an off-white solid (7.2 mg, 26%) at 10.103 min: LCMS (ESI) calc'd for C₁₄H₁₈Cl₂N₂O₂ [M+H]⁺: 317, 319 (3:2), found 317, 319 (3:2); ¹H NMR (300 MHz, CD₃OD) δ 7.15 (d, J=8.8 Hz, 1H), 6.68 (d, J=8.8 Hz, 1H), 3.53 (s, 1H), 3.20 (d, J=6.1 Hz, 3H), 2.74 (dd, J=14.2, 11.5 Hz, 2H), 2.58-2.41 (m, 1H), 2.21 (q, J=12.1 Hz, 1H), 1.95 (s, 3H), 1.56 (dd, J=23.1, 13.0 Hz, 2H).

### Example 64. Compound 87 (9-(2,3-dichloro-6-hydroxyphenyl)-2,6-diazaspiro[4.5]decan-1-one)

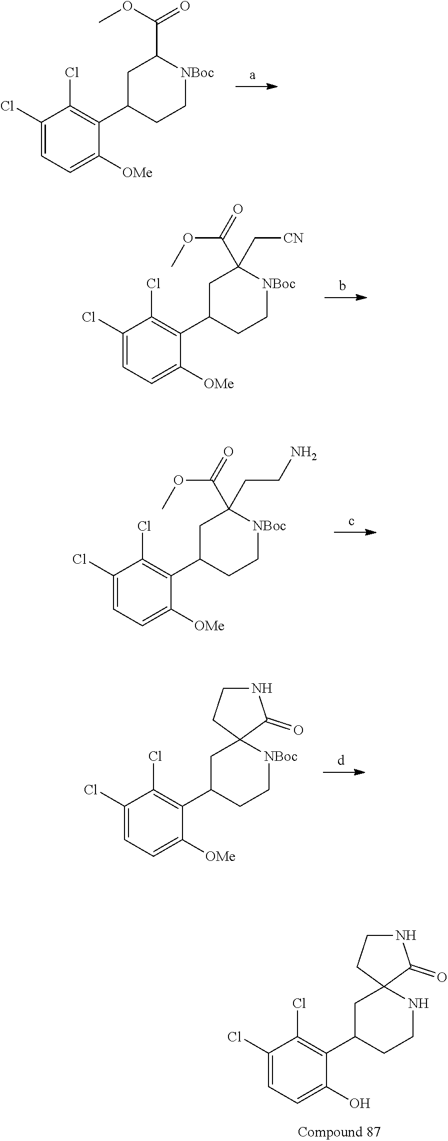 [SMILES: COC(=O)C1CC(c2c(OC)ccc(Cl)c2Cl)CCN1C(=O)OC(C)(C)C ; COC(=O)C1(CC#N)CC(c2c(OC)ccc(Cl)c2Cl)CCN1C(=O)OC(C)(C)C ; COC(=O)C1(CCN)CC(c2c(OC)ccc(Cl)c2Cl)CCN1C(=O)OC(C)(C)C ; COc1ccc(Cl)c(Cl)c1C1CCN(C(=O)OC(C)(C)C)C2(CCNC2=O)C1 ; O=C1NCCC12CC(c1c(O)ccc(Cl)c1Cl)CCN2]

### Step a

To a stirred solution of 1-tert-butyl 2-methyl 4-(2,3-dichloro-6-methoxyphenyl)piperidine-1,2-dicarboxylate (Example 61, step c) (0.28 g, 0.67 mmol) in THE (3 mL) was added LiHMDS (0.8 mL, 0.80 mmol, 1 M in THF) at −78° C. under argon atmosphere. The reaction was stirred at −78° C. for 0.5 h. Then a solution of 2-bromoacetonitrile (0.12 g, 1.00 mmol) in THE (2 mL) was added into. The reaction solution was stirred at −78° C. for 1 h. Then reaction was warmed to room temperature and stirred for 1 h. The reaction was quenched with water (20 mL) at room temperature and extracted with EA (3×20 mL). Then the combined organic layers were washed with brine (2×20 mL) and dried over anhydrous Na₂SO₄. After filtration, the filtrate was concentrated under reduced pressure. The residue was purified by Prep-HPLC with following conditions: Column: XBridge C₁₈ OBD Prep Column 100 Å, 10 m, 19 mm×250 mm; Mobile Phase A: water with 20 mmmoL/L NH₄HCO₃, Mobile Phase B: ACN; Flow rate: 25 mL/min; Gradient: 75% B to 95% B in 6 min; 210 nm; Retention time: 4.98 min to afford 1-tert-butyl 2-methyl 2-(cyanomethyl)-4-(2,3-dichloro-6-methoxyphenyl)piperidine-1,2-dicarboxylate as a light yellow oil (0.10 g, 33%): LCMS (ESI) calc'd for C₂₁H₂₆Cl₂N₂O₅ [M+Na]⁺ 479, 481 (3:2), found 479, 481 (3:2); ¹H NMR (400 MHz, CDCl₃) δ 7.33 (d, J=8.9 Hz, 1H), 6.79 (d, J=8.9 Hz, 1H), 4.01-3.95 (m, 1H), 3.90 (s, 3H), 3.85 (s, 3H), 3.72-3.60 (m, 3H), 3.01 (t, J=14.0 Hz, 1H), 2.93 (d, J=17.1 Hz, 1H), 2.26-2.14 (m, 1H), 2.01-1.88 (m, 1H), 1.74-1.67 (m, 1H), 1.50 (s, 9H).

### Step b

To a stirred solution of 1-tert-butyl 2-methyl 2-(cyanomethyl)-4-(2,3-dichloro-6-methoxyphenyl)piperidine-1,2-dicarboxylate (0.10 g, 0.22 mmol) in MeOH (2 mL) and HOAc (2 mL) was added PtO₂ (50 mg, 0.22 mmol) at room temperature. The reaction was degassed with hydrogen three times and stirred at room temperature for 3 h under hydrogen atmosphere (1.5 atm). The reaction was filtered and the filtrate was concentrated under reduced pressure to afford 1-tert-butyl 2-methyl 2-(2-aminoethyl)-4-(2,3-dichloro-6-methoxyphenyl)piperidine-1,2-dicarboxylate as an off-white semi-solid (0.10 g, 99%). LCMS (ESI) calc'd for C₂₁H₃₀Cl₂N₂O₅ [M+H]⁺ 461, 463 (3:2), found 461, 463 (3:2); ¹H NMR (400 MHz, CDCl₃) δ 7.39 (d, J=8.9 Hz, 1H), 6.74 (d, J=8.9 Hz, 1H), 3.80 (s, 3H), 3.88-3.85 (m, 2H), 3.76-3.3.70 (m, 5H), 3.15-3.10 (m, 3H), 2.0-1.95 (m, 2H), 1.55-1.49 (m, 2H), 1.50 (s, 9H).

### Step c

To a stirred solution of 1-tert-butyl 2-methyl 2-(2-aminoethyl)-4-(2,3-dichloro-6-methoxyphenyl)piperidine-1,2-dicarboxylate (80 mg, 0.17 mmol) in toluene (3 mL) was added TEA (0.18 g, 1.74 mmol) at room temperature. The reaction was stirred at 110° C. for 16 h. After cooling to room temperature, the reaction solution was concentrated under reduced pressure and the residue was purified by silica gel column chromatography, eluted with EA to afford tert-butyl 9-(2,3-dichloro-6-methoxyphenyl)-1-oxo-2,6-diazaspiro[4.5]decane-6-carboxylate as a light yellow oil (30 mg, 40%); LCMS (ESI) calc'd for C₂₀H₂₆Cl₂N₂O₄ [M+H]⁺ 429, 431 (3:2), found 429, 431 (3:2).

### Step d

To a stirred solution of tert-butyl 9-(2,3-dichloro-6-methoxyphenyl)-1-oxo-2,6-diazaspiro[4.5]decane-6-carboxylate (30 mg, 0.07 mmol) in DCM (1 mL) was added BBr₃ (0.11 g, 0.42 mmol) at room temperature. The reaction was stirred at room temperature for 1 h. The reaction was quenched with water (1 mL) and concentrated under reduced pressure. The residue was purified by Prep-HPLC with following conditions: Column: XBridge C₁₈ OBD Prep Column 100 Å, 10 m, 19 mm×250 mm; Mobile Phase A: Water (plus 0.05% TFA), Mobile Phase B: ACN; Flow rate: 25 mL/min; Gradient: 21% B to 30% B in 6 min; Detector: UV 254/220 nm; Retention time: 5.91 min. The fractions containing the desired product were collected and concentrated under reduced pressure to afford Compound 87 (9-(2,3-dichloro-6-hydroxyphenyl)-2,6-diazaspiro[4.5]decan-1-one) as an off-white solid (4 mg, 13%); LCMS (ESI) calc'd for C₁₄H₁₆Cl₂N₂O₂ [M+H]⁺ 315, 317 (3:2), found 315, 317 (3:2); ¹H NMR (400 MHz, CD₃OD) δ 7.27 (d, J=8.8 Hz, 1H), 6.77 (d, J=8.8 Hz, 1H), 3.98-3.85 (m, 1H), 3.58-3.41 (m, 3H), 3.30-3.21 (m, 1H), 3.01-2.81 (m, 2H), 2.79-2.70 (m, 1H), 2.45-2.31 (m, 1H), 1.91-1.79 (m, 2H).

### Example 65. Compound 88 (1-(2,3-dichloro-6-hydroxyphenyl)piperidine-3-carboxamide)

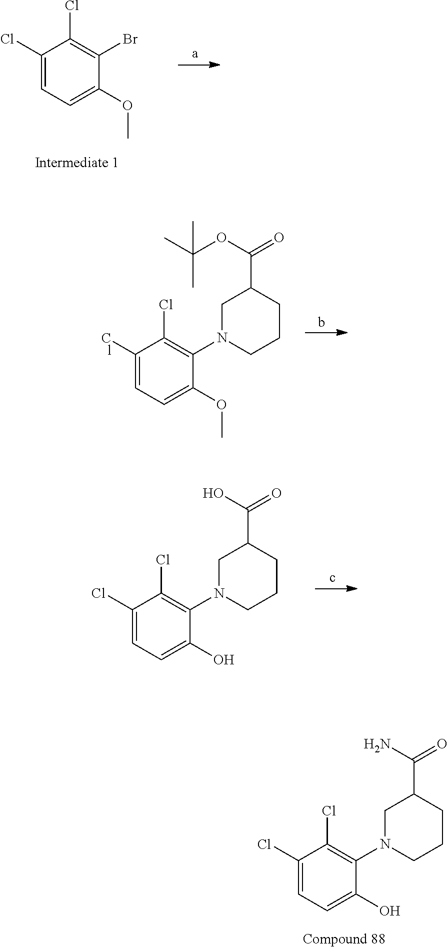 [SMILES: COc1ccc(Cl)c(Cl)c1Br ; COc1ccc(Cl)c(Cl)c1N1CCCC(C(=O)OC(C)(C)C)C1 ; O=C(O)C1CCCN(c2c(O)ccc(Cl)c2Cl)C1 ; NC(=O)C1CCCN(c2c(O)ccc(Cl)c2Cl)C1]

### Step a

To a stirred solution of Intermediate 1 (0.30 g, 1.17 mmol) and tert-butyl piperidine-3-carboxylate (0.26 g, 1.41 mmol) in 1, 4-dioxane (4 mL) was added Pd₂(dba)₃.CHCl₃ (0.11 g, 0.12 mmol) and XantPhos (0.14 g, 0.23 mmol) and Cs₂CO₃ (1.15 g, 3.52 mmol) in portions at room temperature. The resulting mixture was stirred for 12 h at 90° C. under argon atmosphere. After cooling to room temperature, the reaction was diluted with water (30 mL) and extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (10/1) to afford tert-butyl 1-(2,3-dichloro-6-methoxyphenyl)piperidine-3-carboxylate as a light yellow oil (0.20 g, 47%): LCMS (ESI) calc'd for C₁₇H₂₃Cl₂NO₃ [M+H−56]⁺: 304, 306 (3:2), found 304, 306 (3:2); ¹H NMR (400 MHz, CDCl₃) δ 7.18 (dd, J=9.3, 3.2 Hz, 1H), 6.73 (dd, J=9.0, 3.1 Hz, 1H), 3.83 (s, 3H), 3.31-3.06 (m, 3H), 3.03-2.89 (m, 1H), 2.73-2.57 (m, 1H), 2.16-2.02 (m, 1H), 1.93-1.67 (m, 3H), 1.46 (s, 9H).

### Step b

To a stirred solution of tert-butyl 1-(2,3-dichloro-6-methoxyphenyl)piperidine-3-carboxylate (0.20 g, 0.56 mmol) in DCM (1 mL) was added BBr₃ (1.33 g, 5.31 mmol) at room temperature. The resulting mixture was stirred for 2 h at 40° C. The reaction was quenched with water (1 mL) at 0° C. The resulting mixture was concentrated under reduced pressure. The residue was purified by reverse phase chromatography, eluted with 20% ACN in water (plus 0.05% TFA) to afford 1-(2,3-dichloro-6-hydroxyphenyl)piperidine-3-carboxylic acid as a light yellow oil (80 mg, 50%); LCMS (ESI) calc'd for C₁₂H₁₃Cl₂NO₃ [M+H]⁺: 290, 292 (3:2), found 290, 292 (3:2);

### Step c

To a stirred solution of 1-(2,3-dichloro-6-hydroxyphenyl)piperidine-3-carboxylic acid (80 mg, 0.28 mmol) and CDI (47 mg, 0.29 mmol) in DMF (2 mL) was added NH₄Cl (30 mg, 0.56 mmol) at room temperature. The resulting mixture was stirred for 5 h at room temperature. The reaction solution was purified by Prep-HPLC with the following conditions: Column: XBridge C₁₈ OBD Prep Column, 100 Å, 10 μm, 19 mm×250 mm; Mobile Phase A: Water (plus 0.05% TFA), Mobile Phase B: ACN; Flow rate: 25 mL/min; Gradient: 55% B to 56% B in 6 min; Detector: UV 254/220 nm; Retention time: 5.30 min. The fractions containing the desired product were collected and concentrated under reduced pressure to afford Compound 88 (1-(2,3-dichloro-6-hydroxyphenyl)piperidine-3-carboxamide) as a light yellow solid (32 mg, 29%): LCMS (ESI) calc'd for C₁₂H₁₄Cl₂N₂O₂ [M+H]⁺: 289, 291 (3:2), found 289, 291 (3:2); 1H NMR (400 MHz, DMSO-d₆+D₂O) δ 7.21 (d, J=8.8 Hz, 1H), 6.80 (d, J=8.8 Hz, 1H), 3.26-2.98 (m, 3H), 2.98-2.84 (m, 1H), 2.51-2.42 (m, 1H), 1.83-1.48 (m, 4H).

### Example 66. Compound 90 ((2R)-4-(2,3-dichloro-6-hydroxyphenyl)piperazine-2-carboxamide)

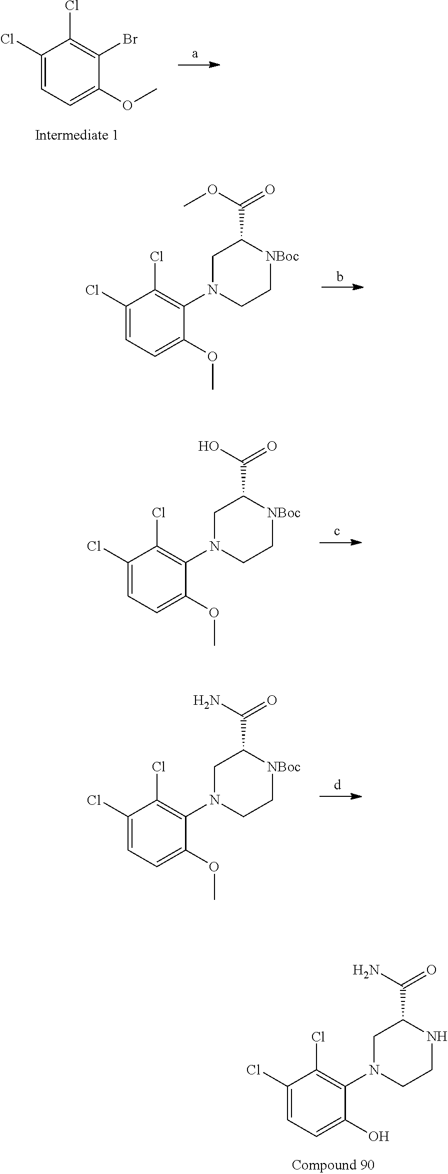 [SMILES: COc1ccc(Cl)c(Cl)c1Br ; COC(=O)[C@H]1CN(c2c(OC)ccc(Cl)c2Cl)CCN1C(=O)OC(C)(C)C ; COc1ccc(Cl)c(Cl)c1N1CCN(C(=O)OC(C)(C)C)[C@@H](C(=O)O)C1 ; COc1ccc(Cl)c(Cl)c1N1CCN(C(=O)OC(C)(C)C)[C@@H](C(N)=O)C1 ; NC(=O)[C@H]1CN(c2c(O)ccc(Cl)c2Cl)CCN1]

### Step a

To a stirred solution of Intermediate 1 (0.40 g, 1.56 mmol) and 1-tert-butyl 2-methyl (2R)-piperazine-1,2-dicarboxylate (0.46 g, 1.88 mmol) in 1,4-dioxane (5 mL) were added Pd₂(dba)₃.CHCl3 (0.14 g, 0.16 mmol), XantPhos (0.18 g, 0.31 mmol) and Cs₂CO₃ (1.53 g, 4.69 mmol) at room temperature. The resulting mixture was degassed with argon three times and stirred for 16 h at 90° C. under argon atmosphere. The reaction was diluted with water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (5/1) to afford 1-tert-butyl 2-methyl (2R)-4-(2,3-dichloro-6-methoxyphenyl)piperazine-1,2-dicarboxylate as a light yellow oil (0.15 g, 18%): LCMS (ESI) calc'd for C₁₈H₂₄Cl₂N₂O₅ [M+H]⁺: 419, 421 (3:2), found 419, 421 (3:2); ¹H NMR (300 MHz, CDCl₃) δ 7.21 (d, J=9.0 Hz, 1H), 6.71 (d, J=9.0 Hz, 1H), 4.70 (d, J=51.1 Hz, 1H), 3.99-3.84 (m, 1H), 3.80 (s, 3H), 3.75 (d, J=7.8 Hz, 3H), 3.64-3.53 (m, 1H), 3.52-3.16 (m, 3H), 2.93-2.73 (m, 1H), 1.49 (d, J=12.8 Hz, 9H).

### Step b

To a stirred solution of 1-tert-butyl 2-methyl (2R)-4-(2,3-dichloro-6-methoxyphenyl)piperazine-1,2-dicarboxylate (0.15 g, 0.36 mmol) in MeOH (3 mL) and H₂O (0.5 mL) was added NaOH (0.14 g, 3.58 mmol) at room temperature. The resulting mixture was stirred for 3 h at room temperature. The mixture was acidified to pH 4 with saturated aq. citric acid. The resulting mixture was extracted with EA (3×20 mL). The combined organic layers were washed with brine (3×20 mL) and dried over anhydrous Na₂SO₄. After filtration, the filtrate was concentrated under reduced pressure to afford (2R)-1-[(tert-butoxy)carbonyl]-4-(2,3-dichloro-6-methoxyphenyl)piperazine-2-carboxylic acid as a yellow oil (0.15 g, crude), which was used in the next step directly without further purification: LCMS (ESI) calc'd for C₁₇H₂₂Cl₂N₂O₅ [M+H]⁺: 405, 407 (3:2), found 405, 407 (3:2);

### Step c

To a stirred solution of (2R)-1-[(tert-butoxy)carbonyl]-4-(2,3-dichloro-6-methoxyphenyl)piperazine-2-carboxylic acid (0.15 g, 0.37 mmol) and HATU (0.28 g, 0.74 mmol) in DMF (2 mL) was added NH₄Cl (40 mg, 0.74 mmol) at room temperature. The reaction was stirred for 2 h at room temperature. The resulting mixture was diluted with water (30 mL). The mixture was extracted with EA (3×20 mL). The combined organic layers were washed with brine (3×20 mL) and dried over anhydrous Na₂SO₄. After filtration, the filtrate was concentrated under reduced pressure. The residue was purified by Prep-TLC, eluted with PE/EA (5/1) to afford tert-butyl (2R)-2-carbamoyl-4-(2,3-dichloro-6-methoxyphenyl)piperazine-1-carboxylate as a yellow solid (80 mg, 53% overall two steps): LCMS (ESI) calc'd for C₁₇H₂₃Cl₂N₃O₄ [M+H]⁺: 404, 406 (3:2), found 404, 406 (3:2); ¹H NMR (300 MHz, CDCl₃) δ 7.20 (d, J=8.7 Hz, 1H), 6.70 (d, J=9.0 Hz, 1H), 4.75 (d, J=53.4 Hz, 1H), 3.87-3.72 (m, 4H), 3.69-3.57 (m, 1H), 3.52-3.37 (m, 2H), 3.34-3.17 (m, 1H), 3.00-2.75 (m, 1H), 1.48 (d, J=5.1 Hz, 9H).

### Step d

To a stirred solution of tert-butyl (2R)-2-carbamoyl-4-(2,3-dichloro-6-methoxyphenyl)piperazine-1-carboxylate (80 mg, 0.20 mmol) in DCM (1 mL) was added BBr₃ (0.50 g, 2.00 mmol at room temperature. The resulting mixture was stirred for 8 h at 40° C. The reaction was quenched with water (1 mL) at 0° C. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 25% B to 30% B in 6 min; Detector: UV 254/220 nm; Retention time: 5.12 min. The fractions containing the desired product were collected and concentrated under reduced pressure to Compound 90 ((2R)-4-(2,3-dichloro-6-hydroxyphenyl)piperazine-2-carboxamide) as an off-white solid (15 mg, 18%); LCMS (ESI) calc'd for C₁₁H₁₃Cl₂N₃O₂ [M+H]⁺: 290, 292 (3:2), found 290, 292 (3:2); ¹H NMR (400 MHz, CD₃OD) δ 7.35-7.14 (m, 1H), 6.91-6.66 (m, 1H), 4.30-4.00 (m, 1H), 3.76 (t, J=12.2 Hz, 1H), 3.64 (t, J=13.2 Hz, 1H), 3.57-3.35 (m, 3H), 3.18-3.07 (m, 1H).

The compound in Table 1E below was prepared in an analogous fashion to that described for Compound 90, starting from Intermediate 1 and the commercially available 1-tert-butyl 2-methyl (2S)-piperazine-1,2-dicarboxylate.

TABLE IE

| Compound | | | |
|---|---|---|---|
| Number | Structure | Chemical Name | MS: (M + H)⁺ & ¹H MNR |
| 92 | 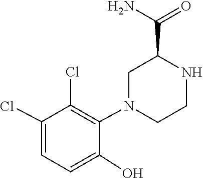 [SMILES: NC(=O)[C@@H]1CN(c2c(O)ccc(Cl)c2Cl)CCN1] | (2S)-4-(2,3- dichloro-6- hydroxyphenyl)pip- erazine-2- carboxamide | [M + H]⁺: 290, 292 (3:2); ¹H NMR (400 MHz, CD₃OD) δ 7.32-7.09 (m, 1H), 6.90 − 6.72 (m, 1H), 4.30 − 4.03 (m, 1H), 3.76 (t, J = 12.1 Hz, 1H), 3.63 (t, J = 13.2 Hz, 1H), 3.57 − 3.35 (m, 3H), 3.18 − 3.04 (m, 1H). |

### Example 67. Compound 91 (3,4,5-trichloro-2-[1-[(3S)-pyrrolidine-3-carbonyl]piperidin-4-yl]phenol)

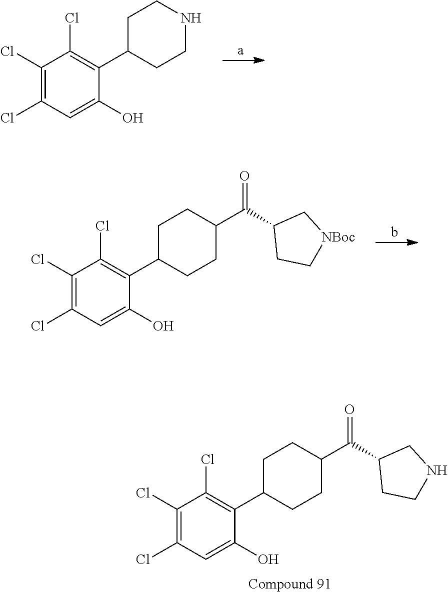 [SMILES: Oc1cc(Cl)c(Cl)c(Cl)c1C1CCNCC1 ; CC(C)(C)OC(=O)N1CC[C@H](C(=O)C2CCC(c3c(O)cc(Cl)c(Cl)c3Cl)CC2)C1 ; O=C(C1CCC(c2c(O)cc(Cl)c(Cl)c2Cl)CC1)[C@H]1CCNC1]

### Step a

To a stirred solution of (S)-1-[(tert-butoxy)carbonyl]pyrrolidine-3-carboxylic acid (46 mg, 0.21 mmol) and EDCI (55 mg, 0.29 mmol) in DMF (1 mL) were added 3,4,5-trichloro-2-(piperidin-4-yl)phenol (Example 31, Compound 44's free base) (40 mg, 0.14 mmol) and Et₃N (29 mg, 0.29 mmol) at room temperature. The resulting mixture was stirred for 2 h at room temperature. The reaction was diluted with EA (20 mL) and water (20 mL). The aqueous solution was extracted with EA (3×30 mL). The combined organic layers were washed with brine (2×30 mL) and dried over anhydrous Na₂SO₄. After filtration, the filtrate was concentrated under reduced pressure to afford tert-butyl (3S)-3-(4-(2,3,4-trichloro-6-hydroxyphenyl)piperidine-1-carbonyl)pyrrolidine-1-carboxylate as a yellow oil (40 mg, crude), which was used to next step directly without further purification: LCMS (ESI) calc'd for C₂₁H₂₇Cl₃N₂O₄ [M+H]⁺: 477, 479, 481 (3:3:1), found 477, 479, 481 (3:3:1); ¹H NMR (300 MHz, CDCl₃) δ 7.09 (d, J=11.8 Hz, 1H), 4.78-4.62 (m, 1H), 4.05 (d, J=13.5 Hz, 1H), 3.71-3.47 (m, 4H), 3.47-3.28 (m, 2H), 3.28-3.08 (m, 1H), 2.67 (t, J=12.7 Hz, 1H), 2.51-2.32 (m, 2H), 2.22-2.05 (m, 2H), 1.75-1.53 (m, 2H), 1.49 (s, 9H);

### Step b

To a stirred solution of tert-butyl (3S)-3-[4-(2,3,4-trichloro-6-hydroxyphenyl)piperidine-1-carbonyl]pyrrolidine-1-carboxylate (40 mg, 0.08 mmol) in DCM (1 mL) was added TFA (1 mL) at room temperature. The reaction was stirred at room temperature for 1 h. The reaction solution was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 40% B to 90% B in 5.5 min; Detector: UV 254/210 nm; Retention time: 3.92 min. The fractions containing the desired product were collected and concentrated under reduced pressure to afford Compound 91 (3,4,5-trichloro-2-[1-[(3S)-pyrrolidine-3-carbonyl]piperidin-4-yl]phenol) as an off-white solid (18.7 mg, 35% overall two steps); LCMS (ESI) calc'd for C₁₆H₁₉C₁₃N₂O₂ [M+H]⁺: 377, 379, 381 (3:3:1), found 377, 379, 381 (3:3:1); ¹H NMR (300 MHz, CD₃OD) δ 6.89 (s, 1H), 4.67 (d, J=13.0 Hz, 1H), 4.20 (d, J=13.2 Hz, 1H), 3.86-3.51 (m, 1H), 3.48-3.34 (m, 1H), 3.27-2.91 (m, 5H), 2.80-2.60 (m, 1H), 2.59-2.32 (m, 2H), 2.23-1.89 (m, 2H), 1.73-1.53 (m, 2H).

The compounds in Table 1F below were prepared in an analogous fashion to that described for Compound 91, starting from Compound 44's free base (Example 31) and the corresponding carboxylic acids, which were available from commercial sources.

TABLE 1F

| Compound | | | MS: (M + H)⁺ & ¹H |
|---|---|---|---|
| Number | Structure | Chemical Name | MNR |
| 93 | 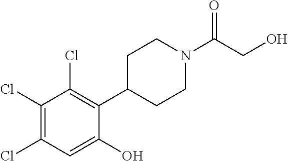 [SMILES: O=C(CO)N1CCC(c2c(O)cc(Cl)c(Cl)c2Cl)CC1] | 2-hydroxy-1-[4- (2,3,4-trichloro-6- hydroxyphenyl)piperidin- 1-yl]ethan-1-one | [M + H]⁺ 338, 340, 342 (3:3:1); ¹H NMR (300 MHz, CD₃OD) δ 6.92 (s, 1H), 4.65 (d, J = 12.9 Hz, 1H), 4.34-4.15 (m, 2H), 3.85 (d, J = 13.9 Hz, 1H), 3.69-3.49 (m, 1H), 3.14 (t, J = 13.1 Hz, 1H), 2.77 (t, J = 13.2 Hz, 1H), 2.60-2.33 (m, 2H), 1.69- |
| | | | 1.49 (m, 2H). |
| 98 | 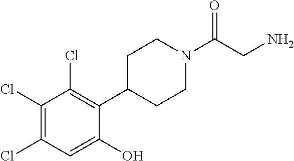 [SMILES: NCC(=O)N1CCC(c2c(O)cc(Cl)c(Cl)c2Cl)CC1] | 2-amino-1-[4- (2,3,4-trichloro-6- hydroxyphenyl) piperidin-1-yl]ethan-1- one | [M + H]⁺: 337, 339, 341 (3:3:1); ¹H NMR (400 MHz, CD₃OD) δ 6.94 (s, 1H), 4.71-4.59 (m, 1H), 4.06-3.90 (m, 2H), 3.88- 3.81 (m, 1H), 3.72-3.60 (m, 1H), 3.27-3.18 (m, 1H), 2.86-2.76 (m, 1H), 2.57-2.37 (m, 2H), 1.66 (t, J = 13.2 Hz, 2H). |

### Example 68. Compound 95 (3,4,5-trichloro-2-[1-(pyrrolidine-3-sulfonyl)piperidin-4-yl]phenol

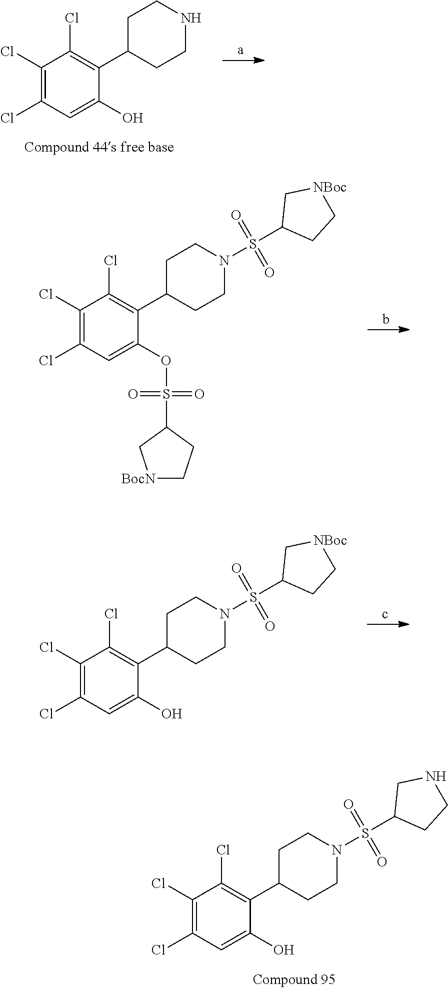 [SMILES: Oc1cc(Cl)c(Cl)c(Cl)c1C1CCNCC1 ; CC(C)(C)OC(=O)N1CCC(S(=O)(=O)Oc2cc(Cl)c(Cl)c(Cl)c2C2CCN(S(=O)(=O)C3CCN(C(=O)OC(C)(C)C)C3)CC2)C1 ; CC(C)(C)OC(=O)N1CCC(S(=O)(=O)N2CCC(c3c(O)cc(Cl)c(Cl)c3Cl)CC2)C1 ; O=S(=O)(C1CCNC1)N1CCC(c2c(O)cc(Cl)c(Cl)c2Cl)CC1]

### Step a

To a stirred solution of 3,4,5-trichloro-2-(piperidin-4-yl)phenol (Example 31, Compound 44's free base) (0.11 g, 0.39 mmol) and tert-butyl 3-(chlorosulfonyl)pyrrolidine-1-carboxylate (0.13 g, 0.47 mmol) in DCM (2 mL) was added Et₃N (79 mg, 0.78 mmol) at room temperature. The resulting mixture was stirred for 3 h at room temperature under nitrogen atmosphere. The reaction was diluted with water (20 mL). The resulting mixture was extracted with EA (3×20 mL). The combined organic layers were washed with brine (3×20 mL) and dried over anhydrous Na₂SO₄. After filtration, the filtrate was concentrated under reduced pressure. The residue was purified by Prep-TLC, eluted with PE/EA (2/1) to afford tert-butyl 3-([2-[1-([1-[(tert-butoxy)carbonyl]pyrrolidin-3-yl]sulfonyl)piperidin-4-yl]-3,4,5-trichlorophenoxy]sulfonyl)pyrrolidine-1-carboxylate as an off-white solid (0.10 g, 34%): LCMS (ESI) calc'd for C₂₉H₄₂Cl₃N₃O₉S₂ [M+H]⁺: 746, 748, 750 (3:3:1), found 746, 748, 750 (3:3:1);

### Step b

To a stirred solution of tert-butyl 3-([2-[1-([1-[(tert-butoxy)carbonyl]pyrrolidin-3-yl]sulfonyl)piperidin-4-yl]-3,4,5-trichlorophenoxy]sulfonyl)pyrrolidine-1-carboxylate (0.10 g, 0.13 mmol) in MeOH (2 mL) was added K₂CO₃ (56 mg, 0.40 mmol) at room temperature. The resulting mixture was stirred for overnight at room temperature. The reaction was diluted with water (20 mL). The resulting mixture was extracted with EA (3×20 mL). The combined organic layers were washed with brine (3×20 mL) and dried over anhydrous Na₂SO₄. After filtration, the filtrate was concentrated under reduced pressure to afford tert-butyl 3-[[4-(2,3,4-trichloro-6-hydroxyphenyl)piperidin-1-yl]sulfonyl]pyrrolidine-1-carboxylate as a light yellow oil (90 mg, crude), which was used to next step directly without further purification: LCMS (ESI) calc'd for C₂₀H₂₇C₁₃N₂O₅S [M+H−56]⁺: 457, 459, 461 (3:3:1), found 457, 459, 461 (3:3:1); ¹H NMR (300 MHz, CDCl₃) δ 6.92 (s, 1H), 4.06-.80 (m, 2H), 3.71-.56 (m, 4H), 3.54-3.32 (m, 2H), 3.07-.83 (m, 2H), 2.65-.14 (m, 4H), 1.70-.54 (m, 2H), 1.47 (d, J=2.9 Hz, 9H).

### Step c

To a stirred solution of tert-butyl 3-[[4-(2,3,4-trichloro-6-hydroxyphenyl)piperidin-1-yl]sulfonyl]pyrrolidine-1-carboxylate (90 mg, 0.18 mmol) in DCM (1 mL) was added TFA (1 mL) at room temperature. The reaction was stirred at room temperature for 1 h. The reaction solution was concentrated under reduced pressure. The residue was purified by Prep-HPLC with the following conditions: Column: XBridge C₁₈ OBD Prep Column 100 Å, 10 m, 19 mm×250 mm; Mobile Phase A: Water with 10 mmol/L NH₄HCO₃, Mobile Phase B: ACN; Flow rate: 25 mL/min; Gradient: 25% B to 70% B in 5.5 min; Detector: UV 254/210 nm; Retention time: 5.30 min. The fractions containing the desired product were collected and concentrated under reduced pressure to afford Compound 95 (3,4,5-trichloro-2-[1-(pyrrolidine-3-sulfonyl)piperidin-4-yl]phenol) as an off-white solid (30 mg, 55% overall two steps): LCMS (ESI) calc'd for C₁₅H₁₉C₁₃N₂O₃S [M+H]⁺: 413, 415, 417 (3:3:1), found 413, 415, 417 (3:3:1); ¹H NMR (400 MHz, CD₃OD) δ 6.92 (s, 1H), 4.00-.86 (m, 2H), 3.86-.74 (m, 1H), 3.58-.44 (m, 1H), 3.26-.19 (m, 2H), 3.11-.86 (m, 4H), 2.65-.51 (m, 2H), 2.27-.10 (m, 2H), 1.62 (d, J=13.4 Hz, 2H).

### Example 69. Compound 96 ((2R)-4-(2,3,4-trichloro-6-hydroxyphenyl)piperazine-2-carboxamide)

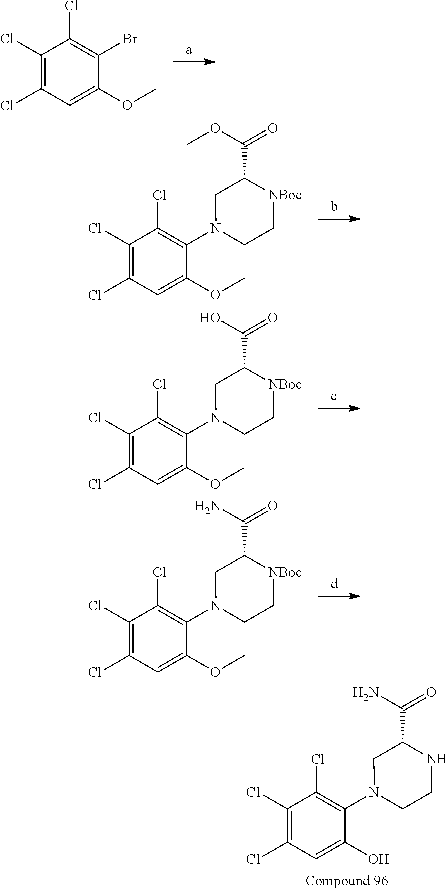

### Step a

To a stirred solution of 2-bromo-3,4,5-trichloro-1-methoxybenzene (Example 31, step c) (0.55 g, 1.89 mmol) and 1-tert-butyl 2-methyl (2R)-piperazine-1,2-dicarboxylate (0.56 g, 2.27 mmol) in 1,4-dioxane (6 mL) were added Pd₂(dba)₃.CHCl3 (0.20 g, 0.19 mmol), XantPhos (0.22 g, 0.38 mmol) and Cs₂CO₃ (1.85 g, 5.68 mmol) at room temperature. The resulting mixture was stirred for 3 h at 90° C. under argon atmosphere. After cooling to room temperature, the reaction was diluted with EA (30 mL) and water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (3/1) to afford 1-tert-butyl 2-methyl (2R)-4-(2,3,4-trichloro-6-methoxyphenyl)piperazine-1,2-dicarboxylate as a yellow oil (0.30 g, 31%): LCMS (ESI) calc'd for C₁₈H₂₃Cl₃N₂O₅ [M+H]⁺: 453, 455, 457 (3:3:1), found 453, 455, 457 (3:3:1); ¹H NMR (300 MHz, CDCl₃) δ 6.89 (s, 1H), 4.70 (d, J=51.4 Hz, 1H), 3.96-3.84 (m, 1H), 3.81 (s, 3H), 3.75 (d, J=7.8 Hz, 3H), 3.63-3.51 (m, 1H), 3.51-3.32 (m, 2H), 3.32-3.15 (m, 1H), 2.91-2.68 (m, 1H), 1.48 (d, J=12.5 Hz, 9H).

### Step b

To a stirred solution of 1-tert-butyl 2-methyl (2R)-4-(2,3,4-trichloro-6-methoxyphenyl)piperazine-1,2-dicarboxylate (0.30 g, 0.66 mmol) in MeOH (3 mL) and H₂O (0.5 mL) was added NaOH (0.26 g, 6.61 mmol) at room temperature. The resulting mixture was stirred for 2 h at room temperature. The mixture was acidified to pH=3 with saturated aq. citric acid. The resulting mixture was extracted with EA (3×20 mL). The combined organic layers were washed with brine (3×20 mL) and dried over anhydrous Na₂SO₄. After filtration, the filtrate was concentrated under reduced pressure to afford (2R)-1-[(tert-butoxy)carbonyl]-4-(2,3,4-trichloro-6-methoxyphenyl)piperazine-2-carboxylic acid as a yellow oil (0.15 g, crude), which was used in the next step directly without further purification: LCMS (ESI) calc'd for C₁₇H₂₁C₁₃N₂O₅ [M+H]⁺: 439, 441, 443 (3:3:1), found 439, 441, 443 (3:3:1);

### Step c

To a stirred solution of (2R)-1-[(tert-butoxy)carbonyl]-4-(2,3,4-trichloro-6-methoxyphenyl)piperazine-2-carboxylic acid (0.15 g, 0.34 mmol) and HATU (0.26 g, 0.68 mmol) in DMF (2 mL) was added NH₄Cl (37 mg, 0.68 mmol) at room temperature. The reaction was stirred for 3 h at room temperature. The resulting mixture was diluted with water (30 mL). The mixture was extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluted with 50% ACN in water (plus 0.05% TFA) to afford tert-butyl (2R)-2-carbamoyl-4-(2,3,4-trichloro-6-methoxyphenyl)piperazine-1-carboxylate as an off-white solid (0.10 g, 34% overall two steps): LCMS (ESI) calc'd for C₁₇H₂₂C₁₃N₃O₄ [M+H]⁺: 438, 440, 442 (3:3:1), found 438, 440, 442 (3:3:1);

### Step d

To a stirred solution of tert-butyl (2R)-2-carbamoyl-4-(2,3,4-trichloro-6-methoxyphenyl)piperazine-1-carboxylate (0.10 g, 0.23 mmol) in DCM (1 mL) was added BBr₃ (0.57 g, 2.28 mmol at room temperature. The resulting mixture was stirred for 3 h at room temperature. The reaction was quenched with water (1 mL) at 0° C. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: Water (plus 0.05% TFA), Mobile Phase B: ACN; Flow rate: 25 mL/min; Gradient: 25% B to 28% B in 10 min; Detector: UV 254/210 nm; Retention time: 8.21 min. The fractions containing the desired product were collected and concentrated under reduced pressure to afford Compound 96 ((2R)-4-(2,3,4-trichloro-6-hydroxyphenyl)piperazine-2-carboxamide) as an off-white solid (7.2 mg, 7%); LCMS (ESI) calc'd for C₁₁H₁₂Cl₃N₃O₂ [M+H]⁺: 324, 326, 328 (3:3:1), found 324, 326, 328 (3:3:1); ¹H NMR (400 MHz, CD₃OD) δ 7.00 (s, 1H), 4.31-4.08 (m, 1H), 3.78-3.68 (m, 1H), 3.68-3.56 (m, 1H), 3.53-3.39 (m, 3H), 3.18-3.07 (m, 1H).

### Example 70. Compound 97 (N-(2R,4S)-rel-[[4-(2,3-dichloro-6-hydroxyphenyl)piperidin-2-yl]methyl]cyclopropanesulfonamide)

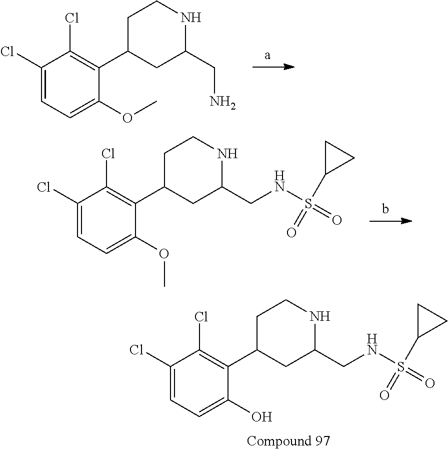

### Step a

To a stirred solution of 1-[4-(2,3-dichloro-6-methoxyphenyl)piperidin-2-yl]methanamine cis isomer (Example 72, step b, free base compound) (0.20 g, 0.69 mmol) and Et₃N (0.56 g, 3.46 mmol) in DCM (1 mL) was added a solution of cyclopropanesulfonyl chloride (49 mg, 0.35 mmol) in DCM (1 mL) dropwise at 0° C. The solution was stirred at 0° C. for 3 h. The reaction solution was concentrated under reduced pressure. The residue was purified by reverse phase chromatography, eluted with 50% ACN in water (plus 0.05% TFA) to afford N-[[4-(2,3-dichloro-6-methoxyphenyl)piperidin-2-yl]methyl]cyclopropanesulfonamide cis isomer as an off-white solid (0.12 g, 44%): LCMS (ESI) calc'd for C₁₆H₂₂Cl₂N₂O₃S [M+H]⁺: 393, 395 (3:2), found 393, 395 (3:2).

### Step b

To a stirred solution of N-[[4-(2,3-dichloro-6-methoxyphenyl)piperidin-2-yl]methyl]cyclopropanesulfonamide cis isomer (0.10 g, 0.25 mmol) in DCM (3 mL) was added BBr₃ (0.32 g, 1.27 mmol) at room temperature. The reaction was stirred at room temperature for 3 h. The reaction was quenched with MeOH (3 mL). The mixture was concentrated under reduced pressure. The residue was purified by Prep-HPLC with the following conditions: Column: Xselect CSH OBD Column 30×150 mm 5 m; Mobile Phase A: Water (plus 0.1% FA), Mobile Phase B: ACN; Flow rate: 60 mL/min; Gradient: 5% B to 28% B in 7 min; Detector: UV 254/220 nm; Retention time: 6.90 min. The fractions containing the desired product were collected and concentrated under reduced pressure to afford Compound 97 (N-(2R,4S)-rel-[[4-(2,3-dichloro-6-hydroxyphenyl)piperidin-2-yl]methyl]cyclopropanesulfonamide (cis isomer)) as an off-white solid (16 mg, 15%): LCMS (ESI) calc'd for C₁₅H₂₀Cl₂N₂O₃S [M+H]⁺: 379, 381 (3:2), found 379, 381 (3:2); ¹H NMR (400 MHz, CD₃OD) δ 8.54 (s, 1H), 7.25 (d, J=8.8 Hz, 1H), 6.76 (d, J=8.8 Hz, 1H), 3.83-3.69 (m, 1H), 3.54-3.39 (m, 2H), 3.31-3.24 (m, 1H), 3.19-3.06 (m, 1H), 2.86-2.71 (m, 1H), 2.68-2.53 (m, 2H), 1.91-1.74 (m, 2H), 1.16-0.98 (m, 5H).

The compounds in Table 1G below were prepared in an analogous fashion to that described for Compound 97, starting from 1-[4-(2,3-dichloro-6-methoxyphenyl)piperidin-2-yl]methanamine cis isomer (Example 72, step b, free base compound) and the corresponding sulfonyl chlorides, which were available from commercial sources.

TABLE 1G

| Compound | | | |
|---|---|---|---|
| Number | Structure | Chemical Name | MS: (M + H)⁺ & ¹H MNR |
| 6 | 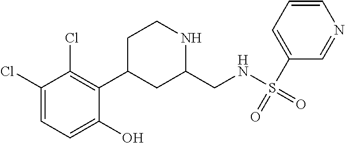 [SMILES: O=S(=O)(NCC1CC(c2c(O)ccc(Cl)c2Cl)CCN1)c1cccnc1] | N-(2R,4S)-rel-[[4- (2,3-dichloro-6- hydroxyphenyl) piperidin-2- yl]methyl]pyridine- 3-sulfonamide | [M + H]⁺ 416, 418 (3:2); ¹H NMR (400 MHz, CD₃OD) δ 9.05 (d, J =2.3 Hz, 1H), 8.83 (dd, J = 4.9, 1.5 Hz, 1H), 8.31 (d, J = 8.2 Hz, 1H), 7.68 (dd, J = 8.1, 4.9 Hz, 1H), 7.25 (d, J = 8.8 Hz, 1H), 6.76 (d, J = 8.8 Hz, 1H), 3.86-3.65 (m, 1H), 3.62-3.48 (m, 1H), 3.43-3.34 |
| | | | (m, 1H), 3.23-3.06 (m, 3H), |
| | | | 2.89-2.74 (m, 1H), 2.69-2.52 |
| | | | (m, 1H), 1.91-1.73 (m, 2H). |
| 94 | 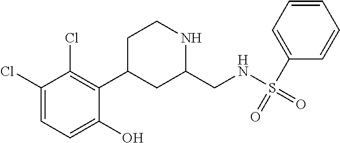 [SMILES: O=S(=O)(NCC1CC(c2c(O)ccc(Cl)c2Cl)CCN1)c1ccccc1] | N-(2R,4S)-rel-[[4- (2,3-dichloro-6- hydroxyphenyl) piperidin-2- yl]methyl] benzenesulfonamide | [M + H]⁺: 415, 417 (3:2); ¹H NMR (400 MHz, CD₃OD) δ 7.95-7.89 (m, 2H), 7.72-7.65 (m, 1H), 7.65-7.57 (m, 2H), 7.25 (d, J = 8.8 Hz, 1H), 6.75 (d, J = 8.8 Hz, 1H), 3.80-3.67 (m, 1H), 3.57-3.48 (m, 1H), 3.38-3.34 (m, 1H), 3.22-3.00 (m, 3H), 2.89-2.74 (m, 1H), |
| | | | 2.59 (q, J= 12.6 Hz, 1H), |
| | | | 1.80 (t, J = 14.6 Hz, 2H). |

### Example 71. Compound 99 ((2-(2-(5-amino-1,3,4-oxadiazol-2-yl)piperidin-4-yl)-3,4-dichlorophenol), Compound 103 (2-((2R,4S)-rel-2-(5-amino-1,3,4-oxadiazol-2-yl)piperidin-4-yl)-3,4-dichlorophenol isomer 1), and Compound 105 (2-((2R,4S)-rel-2-(5-amino-1,3,4-oxadiazol-2-yl)piperidin-4-yl)-3,4-dichlorophenol isomer 2)

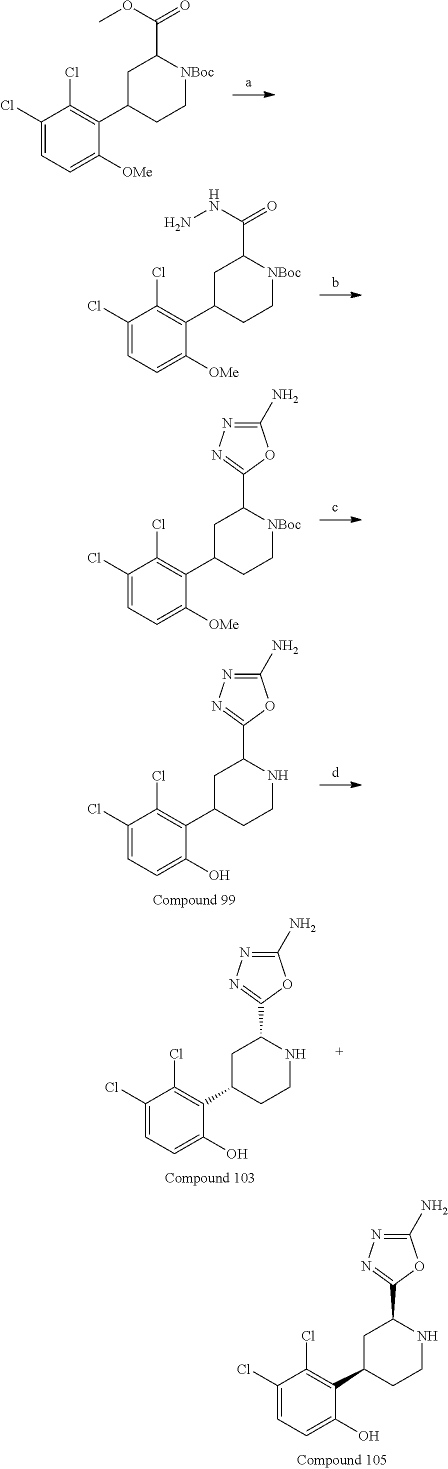 [SMILES: COC(=O)C1CC(c2c(OC)ccc(Cl)c2Cl)CCN1C(=O)OC(C)(C)C ; COc1ccc(Cl)c(Cl)c1C1CCN(C(=O)OC(C)(C)C)C(C(=O)NN)C1 ; COc1ccc(Cl)c(Cl)c1C1CCN(C(=O)OC(C)(C)C)C(c2nnc(N)o2)C1 ; Nc1nnc(C2CC(c3c(O)ccc(Cl)c3Cl)CCN2)o1 ; Nc1nnc([C@H]2C[C@@H](c3c(O)ccc(Cl)c3Cl)CCN2)o1 ; Nc1nnc([C@@H]2C[C@H](c3c(O)ccc(Cl)c3Cl)CCN2)o1]

### Step a

A mixture of 1-tert-butyl 2-methyl 4-(2,3-dichloro-6-methoxyphenyl)piperidine-1,2-dicarboxylate (Example 61, step c) (0.15 g, 0.36 mmol) and NH₂NH₂.H₂O (0.36 g, 7.19 mmol) in MeOH (4 mL) was stirred for 4 h at 75° C. under nitrogen atmosphere. After cooling to room temperature, the reaction was diluted with EA (30 mL) and water (30 mL). The aqueous solution was extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluted with 70% ACN in water (plus 0.05% TFA) to afford tert-butyl 4-(2,3-dichloro-6-methoxyphenyl)-2-(hydrazinecarbonyl)piperidine-1-carboxylate as a yellow oil (0.11 g, 73%): LCMS (ESI) calc'd for C₁₈H₂₅Cl₂N₃O₄ [M+H]⁺: 418, 420 (3:2), found 418, 420 (3:2); ¹H NMR (400 MHz, CD₃OD) δ 7.39 (d, J=8.9 Hz, 1H), 6.98 (d, J=9.0 Hz, 1H), 4.16 (dd, J=12.5, 5.5 Hz, 1H), 3.93-3.84 (m, 4H), 3.76-3.55 (m, 2H), 2.70-2.54 (m, 1H), 2.03-1.93 (m, 2H), 1.80-1.71 (m, 1H), 1.49 (s, 9H).

### Step b

A solution of tert-butyl 4-(2,3-dichloro-6-methoxyphenyl)-2-(hydrazinecarbonyl)piperidine-1-carboxylate (0.11 g, 0.26 mmol) and BrCN (56 mg, 0.53 mmol) in MeOH (3 mL) was stirred for 4 h at room temperature under nitrogen atmosphere. The reaction was quenched with saturated aq. Na₂CO₃ (3 mL) at room temperature. The precipitated solids were filtered and washed with MeOH (3×8 mL) and dried under vacuum to afford tert-butyl 2-(5-amino-1,3,4-oxadiazol-2-yl)-4-(2,3-dichloro-6-methoxyphenyl)piperidine-1-carboxylate as a yellow solid (90 mg, 77%): LCMS (ESI) calc'd for C₁₉H₂₄Cl₂N₄O₄ [M+H]⁺: 443, 445 (3:2), found 443, 445 (3:2); ¹H NMR (400 MHz, CDCl₃) δ 7.32 (d, J=9.2 Hz, 1H), 6.76 (d, J=8.9 Hz, 1H), 5.09-4.95 (m, 2H), 4.95-4.86 (m, 1H), 3.84 (s, 3H), 3.78-3.64 (m, 4H), 2.90-2.74 (m, 1H), 2.16-1.87 (m, 2H), 1.46 (s, 9H).

### Step c

To a stirred mixture of tert-butyl 2-(5-amino-1,3,4-oxadiazol-2-yl)-4-(2,3-dichloro-6-methoxyphenyl)piperidine-1-carboxylate (90 mg, 0.20 mmol) in DCM (3 mL) was added BBr₃ (0.25 g, 1.00 mmol) at room temperature. The resulting mixture was stirred for 1 h at room temperature. The reaction was quenched with water (1 mL) at room temperature. The resulting mixture was concentrated under reduced pressure. The residue was purified by Prep-HPLC with the following conditions: Column: Xselect CSH OBD Column 30×150 mm 5 m; Mobile Phase A: water (plus 0.05% TFA), Mobile Phase B: ACN; Flow rate: 60 mL/min; Gradient: 7% B to 30% B in 7 min; Detector: UV 254/210 nm; Retention time: 6.82 min. The fractions containing the desired product were collected and concentrated under reduced pressure to afford Compound 99 ((2R,4S)-rel-2-(2-(5-amino-1,3,4-oxadiazol-2-yl)piperidin-4-yl)-3,4-dichlorophenol) as an off-white solid (56.9 mg, 50%): LCMS (ESI) calc'd for C₁₃H₁₄Cl₂N₄O₂ [M+H]⁺: 329, 331 (3:2), found 329, 331 (3:2); ¹H NMR (400 MHz, CD₃OD) δ 7.28 (d, J=8.8 Hz, 1H), 6.78 (d, J=8.8 Hz, 1H), 4.74 (dd, J=12.6, 3.2 Hz, 1H), 3.97-3.85 (m, 1H), 3.65-3.57 (m, 1H), 3.40-3.36 (m, 1H), 3.11-2.99 (m, 1H), 2.91-2.78 (m, 1H), 2.29-2.21 (m, 1H), 1.90 (d, J=14.4 Hz, 1H).

### Step d

2-(2-(5-Amino-1,3,4-oxadiazol-2-yl)piperidin-4-yl)-3,4-dichlorophenol (56.9 mg, 0.17 mmol) was separated by Prep Chiral HPLC with following conditions: Column: CHIRALPAK IE, 2×25 cm, 5 μm; Mobile Phase A: Hex (plus 0.2% IPA), Mobile Phase B: EtOH; Flow rate: 20 mL/min; Gradient: 10% B to 10% B in 23 min; Detector: UV 210/254 nm; Retention Time: RT₁: 15.49 min; RT₂: 18.69 min; Injection Volume: 0.5 mL; Number Of Runs: 8.

The faster-eluting enantiomer at 15.49 min was collected and concentrated under reduced pressure. The crude was purified by Prep-HPLC with following conditions: Column: XBridge Prep C₁₈ OBD Column, 19×150 mm 5 m; Mobile Phase A: Water (plus 0.05% TFA), Mobile Phase B: ACN; Flow rate: 20 mL/min; Gradient: 13% B to 40% B in 7 min; Detector: UV 220/254 nm; Retention Time: 5.78 min. The fractions containing the desired product were collected and concentrated under reduced pressure to afford Compound 103 (2-((2R,4S)-rel-2-(5-amino-1,3,4-oxadiazol-2-yl)piperidin-4-yl)-3,4-dichlorophenol isomer 1) as an off-white solid (14.4 mg, 25%): LCMS (ESI) calc'd for C₁₃H₁₄Cl₂N₄O₂ [M+H]⁺: 329, 331 (3:2), found 329, 331 (3:2); ¹H NMR (400 MHz, CD₃OD) δ 7.28 (d, J=8.8 Hz, 1H), 6.78 (d, J=8.8 Hz, 1H), 4.74 (dd, J=12.6, 3.3 Hz, 1H), 3.96-3.86 (m, 1H), 3.66-3.57 (m, 1H), 3.40-3.34 (m, 1H), 3.12-2.99 (m, 1H), 2.92-2.77 (m, 1H), 2.31-2.20 (m, 1H), 1.89 (d, J=14.2 Hz, 1H).

The slower-eluting enantiomer at 18.69 min was collected and concentrated under reduced pressure. The crude was purified by Prep-HPLC with following conditions: Column: XBridge Prep C₁₈ OBD Column, 19×150 mm 5 m; Mobile Phase A: Water (plus 0.05% TFA), Mobile Phase B: ACN; Flow rate: 20 mL/min; Gradient: 13% B to 40% B in 7 min; Detector: UV 220/254 nm; Retention Time: 5.78 min. The fractions containing the desired product were collected and concentrated under reduced pressure to afford Compound 105 (2-((2R,4S)-rel-2-(5-amino-1,3,4-oxadiazol-2-yl)piperidin-4-yl)-3,4-dichlorophenol isomer 2) as an off-white solid (13.4 mg, 24%): LCMS (ESI) calc'd for C₁₃H₁₄Cl₂N₄O₂ [M+H]⁺: 329, 331 (3:2), found 329, 331 (3:2); ¹H NMR (400 MHz, CD₃OD) δ 7.28 (d, J=8.8 Hz, 1H), 6.78 (d, J=8.8 Hz, 1H), 4.74 (dd, J=12.6, 3.3 Hz, 1H), 3.97-3.85 (m, 1H), 3.66-3.55 (m, 1H), 3.41-3.34 (m, 1H), 3.11-2.97 (m, 1H), 2.92-2.76 (m, 1H), 2.32-2.18 (m, 1H), 1.89 (d, J=14.3 Hz, 1H).

### Example 72. Compound 100 ((2R,4S)-rel-N-[[4-(2,3-dichloro-6-hydroxyphenyl)piperidin-2-yl]methyl]methanesulfonamide), Compound 110 ((2R,4S)-rel-N-[[4-(2,3-dichloro-6-hydroxyphenyl)piperidin-2-yl]methyl]methanesulfonamide isomer 1), and Compound 107 ((2R,4S)-rel-N-[[4-(2,3-dichloro-6-hydroxyphenyl)piperidin-2-yl]methyl]methanesulfonamide isomer 2)

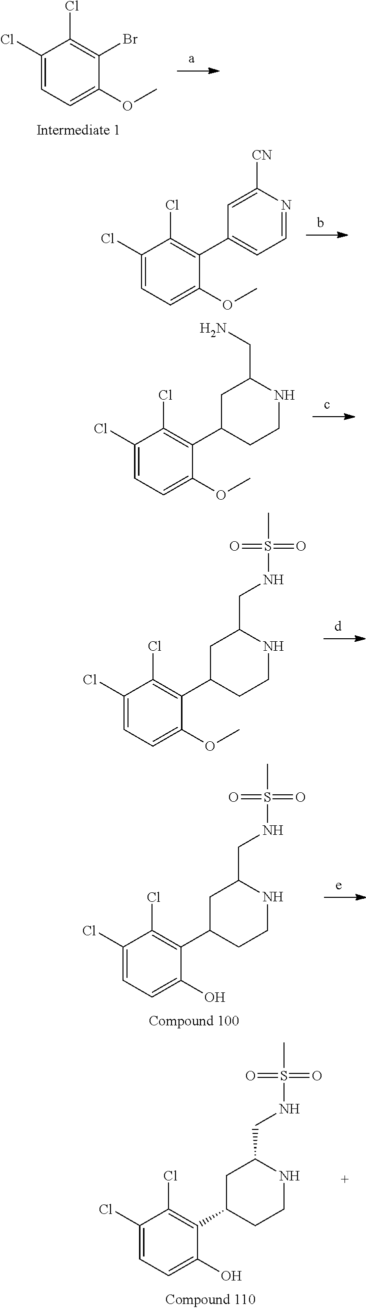

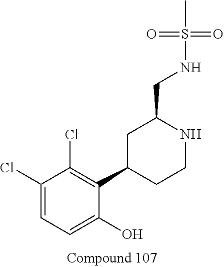 [SMILES: CS(=O)(=O)NC[C@@H]1C[C@H](c2c(O)ccc(Cl)c2Cl)CCN1]

### Step a

To a solution of Intermediate 1 (5.00 g, 16.51 mmol) and 4-(4,4,5,5-tetramethyl-1,3,2-dioxaborolan-2-yl)pyridine-2-carbonitrile (3.80 g, 16.51 mmol) in 1,4-dioxane (80 mL) and H₂O (20 mL) were added Na₂CO₃ (5.25 g, 49.53 mmol) and Pd(dppf)Cl₂.CH₂Cl₂ (0.67 g, 0.83 mmol) under nitrogen atmosphere. The reaction mixture was stirred 80° C. for 3 h under nitrogen atmosphere. The reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (3/1) to afford 4-(2,3-dichloro-6-methoxyphenyl)pyridine-2-carbonitrile as an off-white solid (3.00 g, 65%): LCMS (ESI) calc'd for C₁₃H₈Cl₂N₂O [M+H]⁺: 279, 281 (3:2), found 279, 281 (3:2); H NMR (400 MHz, CDCl₃) δ 8.80 (dd, J=5.0, 0.9 Hz, 1H), 7.64 (s, 1H), 7.54 (d, J=8.9 Hz, 1H), 7.46 (dd, J=5.0, 1.7 Hz, 1H), 6.92 (d, J=9.0 Hz, 1H), 3.77 (s, 3H).

### Step b

To a stirred mixture of 4-(2,3-dichloro-6-methoxyphenyl)pyridine-2-carbonitrile (3.00 g, 10.75 mmol) in MeOH (400 mL) and conc. HCl (12 M, 40.00 mL) was added PtO₂ (0.50 g, 2.16 mmol) in portions at room temperature. The reaction mixture was degassed and stirred at 30° C. under hydrogen atmosphere (50 atm) for 48 h. The mixture was filtered and the filtrate was concentrated under reduced pressure. The residue was purified by reverse phase chromatography, eluted with 40% ACN in water (plus 0.05% TFA) to afford 1-[4-(2,3-dichloro-6-methoxyphenyl)piperidin-2-yl]methanamine as an off-white solid (2.8 g, 50%): LCMS (ESI) calc'd for C₁₃H₁₈Cl₂N₂O [M+H]⁺: 289, 291 (3:2), found 289, 291 (3:2); ¹H NMR (400 MHz, CD₃OD) δ 7.36 (d, J=9.0 Hz, 1H), 6.95 (d, J=9.0 Hz, 1H), 3.85 (s, 3H), 3.66-3.52 (m, 1H), 3.25-3.16 (m, 1H), 2.83-2.73 (m, 1H), 2.73-2.62 (m, 3H), 2.48-2.33 (m, 1H), 2.16-1.98 (m, 1H), 1.58 (dd, J=31.4, 12.8 Hz, 2H).

### Step c

To a stirred mixture of 1-[4-(2,3-dichloro-6-methoxyphenyl)piperidin-2-yl]methanamine (0.20 g, 0.39 mmol) in DCM (2 mL) were added MsCl (44 mg, 0.39 mmol) and Et₃N (59 g, 0.58 mmol) at −40° C. The resulting mixture was stirred for 2 h at −40° C. The reaction was quenched with water (20 mL) at 0° C. The resulting mixture was extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluted with 50% ACN in water (plus 0.05% TFA) afford N-[[4-(2,3-dichloro-6-methoxyphenyl)piperidin-2-yl]methyl]methanesulfonamide as a light yellow oil (0.12 g, 85%): LCMS (ESI) calc'd for C₁₄H₂₀Cl₂N₂O₃S [M+H]⁺: 367, 369 (3:2), found 367, 369 (3:2); ¹H NMR (400 MHz, DMSO-d₆) δ 8.60 (br, 2H), 7.50 (d, J=9.0 Hz, 1H), 7.40 (s, 1H) 7.08-7.02 (m, 1H), 3.81 (s, 3H), 3.59-3.53 (m, 1H), 3.36 (d, J=12.5 Hz, 1H), 3.31-3.20 (m, 1H), 3.21-3.12 (m, 2H), 3.09-2.99 (m, 1H), 2.96 (s, 3H), 2.49-2.42 (m, 1H), 2.32-2.18 (m, 1H), 1.81-1.60 (m, 2H).

### Step d

To a stirred solution of 4-(2,3-dichloro-6-methoxyphenyl)-2-(methanesulfonylmethyl)piperidine (0.12 g, 0.34 mmol) in DCM (2 mL) was added BBr₃ (0.51 g, 2.04 mmol) at room temperature. The reaction was stirred at room temperature for 10 h. The reaction was quenched with MeOH (1 mL). The mixture was concentrated under reduced pressure. The residue was purified by Prep-HPLC with the following conditions: Column: Xselect CSH OBD Column 30×150 mm 5 m; Mobile Phase A: Water (plus 0.05% TFA), Mobile Phase B: MeOH; Flow rate: 60 mL/min; Gradient: 10% B to 50% B in 7 min; Detector: UV 254/220 nm; Retention time: 5.57 min. The fractions containing the desired product were collected and concentrated under reduced pressure to afford Compound 100 ((2R,4S)-rel-N-[[4-(2,3-dichloro-6-hydroxyphenyl)piperidin-2-yl]methyl]methanesulfonamide (cis isomer)) as an off-white solid (67.8 mg, 42%): LCMS (ESI) calc'd for C₁₃H₁₈Cl₂N₂O₃S [M+H]⁺: 353, 355 (3:2), found 353, 355 (3:2); ¹H NMR (400 MHz, DMSO-d₆) δ 10.34 (s, 1H), 8.75 (s, 1H), 8.46 (s, 1H), 7.43-7.36 (m, 1H), 7.34 (d, J=8.8 Hz, 1H), 6.84 (d, J=8.7 Hz, 1H), 3.71-3.47 (m, 1H), 3.44-3.21 (m, 2H), 3.21-3.00 (m, 2H), 2.97 (s, 3H), 2.69-2.53 (m, 1H), 2.41-2.28 (m, 1H), 1.76 (d, J=13.7 Hz, 1H), 1.66 (d, J=13.8 Hz, 1H).

### Step e

N-[[4-(2,3-dichloro-6-hydroxyphenyl)piperidin-2-yl]methyl]methanesulfonamide (60 mg, 0.13 mmol) was separated by Prep Chiral HPLC with following conditions: Column: CHIRALPAK IE, 2×25 cm, 5 μm; Mobile Phase A: Hex (plus 0.2% DEA), Mobile Phase B: EtOH; Flow rate: 18 mL/min; Gradient: 20% B to 20% B in 10 min; Detector: UV 220/254 nm; Retention Time: RT₁: 6.22 min; RT₂: 7.86 min.

The faster-eluting enantiomer at 6.22 min was collected and concentrated under reduced pressure. The crude was purified by Prep-HPLC with following conditions: Column: XBridge Prep C₁₈ OBD Column, 19×150 mm 5 m; Mobile Phase A: Water with 10 mmol/L NH₄HCO₃, Mobile Phase B: ACN; Flow rate: 20 mL/min; Gradient: 15% B to 52% B in 7 min; Detector: UV 254/220 nm; Retention Time: 6.58 min. The fractions containing the desired product were collected and concentrated under reduced pressure to afford Compound 110 ((2R,4S)-rel-N-[[4-(2,3-dichloro-6-hydroxyphenyl)piperidin-2-yl]methyl]methanesulfonamide isomer 1) as an off-white solid (11.2 mg, 25%): LCMS (ESI) calc'd for C₁₃H₁₈Cl₂N₂O₃S [M+H]⁺: 353, 355 (3:2), found 353, 355 (3:2); ¹H NMR (400 MHz, DMSO-d₆) δ 7.27 (d, J=8.8 Hz, 1H), 6.79 (d, J=8.8 Hz, 1H), 3.36-3.21 (m, 1H), 3.04 (d, J=11.8 Hz, 1H), 2.91-2.83 (m, 5H), 2.62-2.54 (m, 2H), 2.35-2.20 (m, 1H), 2.02-1.82 (m, 1H), 1.46 (d, J=12.2 Hz, 1H), 1.36 (d, J=12.4 Hz, 1H);

The slower-eluting enantiomer at 7.86 min was collected and concentrated under reduced pressure. The crude was purified by Prep-HPLC with following conditions: Column: XBridge Prep C₁₈ OBD Column, 19×150 mm 5 m; Mobile Phase A: Water with 10 mmol/L NH₄HCO₃, Mobile Phase B: ACN; Flow rate: 20 mL/min; Gradient: 15% B to 55% B in 7 min; Detector: UV 220/254 nm; Retention Time: 6.42 min. The fractions containing the desired product were collected and concentrated under reduced pressure to afford Compound 107 ((2R,4S)-rel-N-[[4-(2,3-dichloro-6-hydroxyphenyl)piperidin-2-yl]methyl]methanesulfonamide isomer 2) as an off-white solid (11.0 mg, 24%): LCMS (ESI) calc'd for C₁₃H₁₈Cl₂N₂O₃S [M+H]⁺: 353, 355 (3:2), found 353, 355 (3:2); ¹H NMR (400 MHz, DMSO-d₆) δ 7.26 (d, J=8.8 Hz, 1H), 6.79 (d, J=8.8 Hz, 1H), 3.37-3.21 (m, 1H), 3.07-2.98 (m, 1H), 2.91-2.84 (m, 5H), 2.61-2.53 (m, 2H), 2.36-2.19 (m, 1H), 1.99-1.87 (m, 1H), 1.40 (dd, J=40.4, 12.3 Hz, 2H).

### Example 73. Compound 101 (2-[(2R,4S)-rel-4-(2,3-dichloro-6-hydroxyphenyl)piperidin-2-yl]acetamide), Compound 108 (2-[(2R,4S)-rel-4-(2,3-dichloro-6-hydroxyphenyl)piperidin-2-yl]acetamide isomer 1) and Compound 109 (2-[(2R,4S)-rel-4-(2,3-dichloro-6-hydroxyphenyl)piperidin-2-yl]acetamide isomer 2)

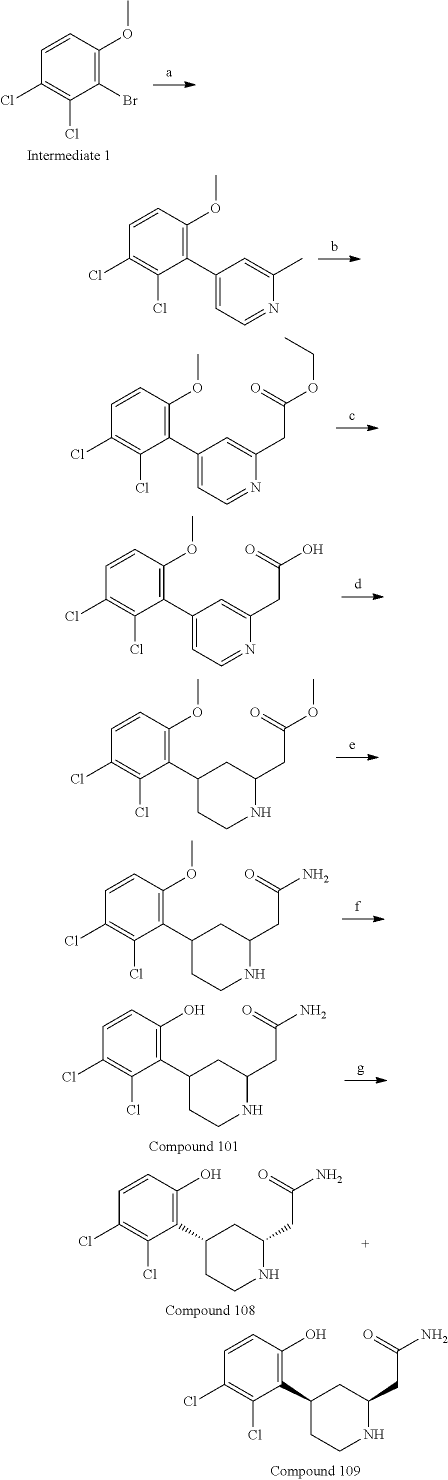

### Step a

To a stirred solution of Intermediate 1 (4.00 g, 15.63 mmol) and 2-methyl-4-(4,4,5,5-tetramethyl-1,3,2-dioxaborolan-2-yl)pyridine (4.10 g, 18.76 mmol) in dioxane (20 mL) were added Pd(dppf)C₁₂ (2.29 g, 3.13 mmol) and Na₂CO₃ (4.97 g, 46.89 mmol) at room temperature under argon atmosphere. The resulting mixture was stirred at 80° C. for 16 h. After cooling to room temperature, the reaction was diluted with water (50 mL). The aqueous layer was extracted with EA (3×50 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (6/1) to afford 4-(2,3-dichloro-6-methoxyphenyl)-2-methylpyridine as a light yellow oil (3.50 g, 84%): LCMS (ESI) calc'd for C₁₃H₁₁Cl₂NO [M+H]⁺: 268, 270 (3:2), found 268, 270 (3:2); ¹H NMR (400 MHz, CDCl₃) δ 8.61 (s, 1H), 7.48 (dd, J=8.9, 2.6 Hz, 1H), 7.15-6.99 (m, 2H), 6.88 (dd, J=8.9, 2.5 Hz, 1H), 3.75 (s, 3H), 2.65 (s, 3H).

### Step b

To a stirred solution of DIPA (0.94 g, 9.32 mmol) in THE (10 mL) was added n-BuLi (3.7 mL, 9.32 mmol, 2.5 M in hexanes) dropwise at −78° C. under argon atmosphere. The reaction was stirred at −78° C. for 15 min. To the above solution was added a solution of 4-(2,3-dichloro-6-methoxyphenyl)-2-methylpyridine (1.00 g, 3.73 mmol) in THE (10 mL) dropwise at −78° C. for 10 min under argon atmosphere. The reaction was stirred at −78° C. for 1 h. Then diethyl carbonate (0.66 g, 5.59 mmol) was added. The reaction was stirred at −78° C. to −65° C. for 1 h. The reaction was quenched with water (3 mL) at −65° C. and diluted with water (50 mL). The aqueous solution was extracted with EA (3×50 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (3/2) to afford ethyl 4-(2,3-dichloro-6-methoxyphenyl)pyridine-2-carboxylate as a light yellow oil (0.87 g, 72%): LCMS (ESI) calc'd for C₁₆H₁₅Cl₂NO₃ [M+H]⁺ 340, 342 (3:2), found 340, 342 (3:2); ¹H NMR (400 MHz, CDCl₃) δ 8.65 (d, J=5.1 Hz, 1H), 7.48 (d, J=9.0 Hz, 1H), 7.24 (s, 1H), 7.13 (dd, J=5.1, 1.6 Hz, 1H), 6.88 (d, J=9.0 Hz, 1H), 4.22 (q, J=7.2 Hz, 2H), 3.93 (s, 2H), 3.74 (s, 3H), 1.27 (t, 3H).

### Step c

To a stirred solution of ethyl 2-[4-(2,3-dichloro-6-methoxyphenyl)pyridin-2-yl]acetate (0.87 g, 2.56 mmol) in MeOH (10 mL) was added NaOH (0.51 g, 12.79 mmol) in water (1 mL) at room temperature. The reaction was stirred at 30° C. for 1 h. The reaction was adjusted to pH 3 with aq. HCl (2 M). Then the solution was concentrated under reduced pressure. The residue was purified by reverse phase chromatography, eluted with 25% ACN in water (plus 0.05% TFA) to afford 2-[4-(2,3-dichloro-6-methoxyphenyl)pyridin-2-yl]acetic acid as a light yellow semi-solid (0.90 g, 83%): LCMS (ESI) calc'd for C₁₄H₁₁Cl₂NO₃ [M+H]⁺: 312, 313 (3:2), found 312, 313 (3:2); ¹H NMR (400 MHz, DMSO-d₆) δ 8.70 (d, J=5.3 Hz, 1H), 7.74 (d, J=9.1 Hz, 1H), 7.46 (s, 1H), 7.41 (dd, J=5.3, 1.7 Hz, 1H), 7.23 (d, J=9.2 Hz, 1H), 3.91 (s, 2H), 3.74 (s, 3H).

### Step d

To a solution of 2-[4-(2,3-dichloro-6-methoxyphenyl)pyridin-2-yl]acetic acid (0.50 g, 1.17 mmol) in MeOH (10 mL) and aq. HCl (6 N, 1 mL) was added PtO₂ (0.18 g, 0.80 mmol) at room temperature. The reaction was degassed with hydrogen three times and stirred at 30° C. for 48 h under hydrogen atmosphere (50 atm). The reaction mixture was filtered. The filtrate was concentrated under reduced pressure. The residue purified by reverse phase chromatography, eluted with 37% ACN in (plus 0.05% TFA) to afford methyl 2-[4-(2,3-dichloro-6-methoxyphenyl)piperidin-2-yl]acetate as a light yellow oil (0.30 g, 57%): LCMS (ESI) calc'd for C₁₅H₁₉Cl₂NO₃ [M+H]⁺ 332, 334 (3:2), found 332, 334 (3:2); ¹H NMR (400 MHz, CD₃OD) δ 7.43 (dd, J=9.1, 2.0 Hz, 1H), 7.01 (d, J=9.0 Hz, 1H), 3.89 (s, 3H), 3.77 (s, 3H), 3.74-3.65 (m, 1H), 3.59-3.50 (m, 1H), 3.40-3.34 (m, 1H), 3.29-3.15 (m, 1H), 2.87-2.71 (m, 2H), 2.71-2.43 (m, 2H), 1.89-1.83 (m, 2H).

### Step e

A solution of methyl 2-[4-(2,3-dichloro-6-methoxyphenyl)piperidin-2-yl]acetate (0.30 g, 0.67 mmol) in NH₃ (g) in MeOH (5 mL) was added stirred at 70° C. for 16 h in sealed tube. After cooling to room temperature, the reaction solution was concentrated under reduced pressure to afford 2-[4-(2,3-dichloro-6-methoxyphenyl)piperidin-2-yl]acetamide as an off-white solid (0.26 g, 87%): LCMS (ESI) calc'd for C₁₄H₁₈Cl₂N₂O₂ [M+H]⁺ 317, 319 (3:2), found 317, 319 (3:2); ¹H NMR (400 MHz, CD₃OD) δ 7.43 (d, J=9.0 Hz, 1H), 7.01 (d, J=9.0 Hz, 1H), 3.89 (s, 3H), 3.87-3.76 (m, 2H), 3.71-3.59 (m, 1H), 3.59-3.48 (m, 1H), 3.28-3.11 (m, 1H), 2.73-2.43 (m, 3H), 1.92-1.73 (m, 2H).

### Step f

To a stirred solution of 2-[4-(2,3-dichloro-6-methoxyphenyl)piperidin-2-yl]acetamide (0.26 g, 0.82 mmol) in DCM (5 mL) was added BBr₃ (1.23 g, 4.92 mmol) at room temperature. The reaction was stirred at room temperature for 1 h. The reaction was quenched with water (1 ml) and neutralized with saturated aq. NaHCO₃ to pH 7-8. The resulting mixture was concentrated under reduced pressure. The residue was purified by Prep-HPLC with the following conditions: Column: Xselect CSH OBD Column 30×150 mm 5 m; Mobile Phase A: water with 10 mmoL/L NH₄HCO₃, Mobile Phase B: ACN; Flow rate: 60 mL/min; Gradient: 18% B to 28% B in 10 min; Detector: UV 254/220 nm; Retention time: 9.27 min. The fractions containing the desired product were collected and concentrated under reduced pressure to afford Compound 101 (2-[(2R,4S)-rel-4-(2,3-dichloro-6-hydroxyphenyl)piperidin-2-yl]acetamide (cis isomer)) as an off-white solid (84.7 mg, 34%): LCMS (ESI) calc'd for C₁₃H₁₆Cl₂N₂O₂ [M+H]⁺: 303, 305 (3:2), found 303, 305 (3:2); ¹H NMR (400 MHz, CD₃OD) δ 7.18 (d, J=8.8 Hz, 1H), 6.71 (d, J=8.8 Hz, 1H), 3.69-3.50 (m, 1H), 3.24 (d, J=12.3 Hz, 1H), 3.20-3.05 (m, 1H), 2.86 (t, J=12.3 Hz, 1H), 2.65-2.44 (m, 1H), 2.44-2.29 (m, 3H), 1.68-1.53 (m, 2H).

### Step g

2-[4-(2,3-Dichloro-6-hydroxyphenyl)piperidin-2-yl]acetamide (80 mg, 0.26 mmol) was separated by Prep Chiral HPLC with following conditions: Column: CHIRALPAK IG UL001, 20×250 mm, 5 m; Mobile Phase A: Hex-HPLC, Mobile Phase B: EtOH-HPLC; Flow rate: 20 mL/min; Gradient: 30% B to 30% B in 9 min; Detector: UV 210/254 nm; Retention Time: RT₁: 4.60 min; RT₂: 7.07 min; Injection Volume: 0.7 mL; Number Of Runs: 19.

The faster-eluting enantiomer at 4.60 min was collected and concentrated under reduced pressure. The crude was purified by Prep-HPLC with following conditions: Column: XBridge Prep C₁₈ OBD Column, 19×150 mm 5 m; Mobile Phase A: Water with 10 mmol/L NH₄HCO₃, Mobile Phase B: ACN; Flow rate: 20 mL/min; Gradient: 5% B to 45% B in 7 min; Detector: UV 220/254 nm; Retention Time: 6.40 min. The fractions containing the desired product were collected and concentrated under reduced pressure to afford Compound 108 (2-[(2R,4S)-rel-4-(2,3-dichloro-6-hydroxyphenyl)piperidin-2-yl]acetamide isomer 1) as an off-white solid (22 mg, 26%): LCMS (ESI) calc'd for C₁₃H₁₆Cl₂N₂O₂ [M+H]⁺ 303, 305 (3:2), found 303, 305 (3:2); ¹H NMR (400 MHz, CD₃OD) δ 7.17 (d, J=8.6, 1H), 6.70 (d, J=8.8 Hz, 1H), 3.68-3.50 (m, 1H), 3.25-3.13 (m, 1H), 3.13-3.01 (m, 1H), 2.89-2.73 (m, 1H), 2.64-2.47 (m, 1H), 2.42-2.18 (m, 3H), 1.69-1.42 (m, 2H).

The slower-eluting enantiomer at 7.07 min was collected and concentrated under reduced pressure. The crude was purified by Prep-HPLC with following conditions: Column: XBridge Prep C₁₈ OBD Column, 19×150 mm 5 m; Mobile Phase A: Water with 10 mmol/L NH₄HCO₃, Mobile Phase B: ACN; Flow rate: 20 mL/min; Gradient: 5% B to 50% B in 7 min; Detector: UV 254/220 nm; Retention Time: 6.55 min. The fractions containing the desired product were collected and concentrated under reduced pressure to afford Compound 109 (2-[(2R,4S)-rel-4-(2,3-dichloro-6-hydroxyphenyl)piperidin-2-yl]acetamide isomer 2) as an off-white solid (27.5 mg, 33%): LCMS (ESI) calc'd for C₁₃H₁₆Cl₂N₂O₂ [M+H]⁺ 303, 305 (3:2), found 303, 305 (3:2); ¹H NMR (400 MHz, CD₃OD) δ 7.21-7.13 (m, 1H), 6.70 (d, J=8.7 Hz, 1H), 3.66-3.51 (m, 1H), 3.24-3.15 (m, 1H), 3.12-3.01 (m, 1H), 2.89-2.71 (m, 1H), 2.60-2.43 (m, 1H), 2.41-2.25 (m, 3H), 1.66-1.51 (m).

### Example 74. Compound 102 ((2R,4S)-rel-[4-(2,3-dichloro-6-hydroxyphenyl)piperidin-2-yl]methylurea)

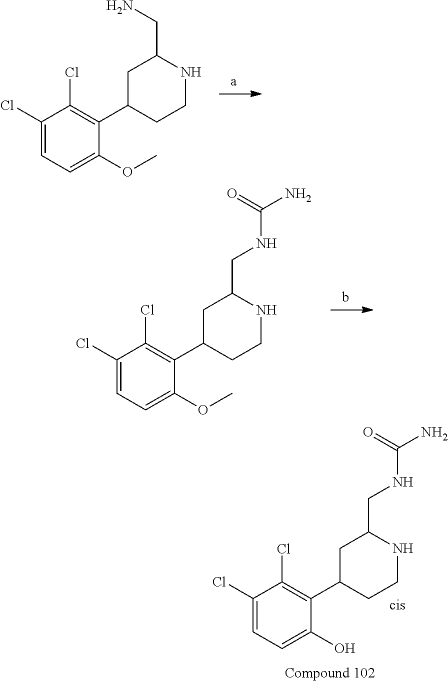 [SMILES: COc1ccc(Cl)c(Cl)c1C1CCNC(CN)C1 ; COc1ccc(Cl)c(Cl)c1C1CCNC(CNC(N)=O)C1 ; NC(=O)NCC1CC(c2c(O)ccc(Cl)c2Cl)CCN1]

### Step a

To a stirred solution of 1-[4-(2,3-dichloro-6-methoxyphenyl)piperidin-2-yl]methanamine cis isomer (Example 72, step b, free base compound) (0.15 g, 0.50 mmol) and Et₃N (0.15 g, 1.50 mmol) in DCM (2 mL) was added isocyanatotrimethylsilane (58 mg, 0.50 mmol) in portions at −40° C. under nitrogen atmosphere. The resulting mixture was stirred for 1 h at −40° C. under nitrogen atmosphere. The reaction was quenched with water (20 mL) at room temperature. The resulting mixture was extracted with EA (2×30 mL). The combined organic layers were washed with brine (2×20 mL) and dried over anhydrous Na₂SO₄. After filtration, the filtrate was concentrated under reduced pressure. The residue was purified by Prep-TLC, eluted with PE/EA (1/1) to afford (2R,4S)-rel-[4-(2,3-dichloro-6-methoxyphenyl)piperidin-2-yl]methyl]urea (cis isomer) as a yellow oil (60 mg, 32%): LCMS (ESI) calc'd for C₁₄H₁₉Cl₂N₃O₂ [M+H]⁺: 332, 334 (3:2), found 332, 334 (3:2); ¹H NMR (400 MHz, CD₃OD) δ 7.45 (d, J=9.0 Hz, 1H), 7.02 (d, J=9.0 Hz, 1H), 3.92 (s, 3H), 3.65-3.57 (m, 2H), 3.40-3.34 (m, 2H), 3.29-3.18 (m, 2H), 2.80-2.57 (m, 2H), 1.99 (d, J=13.8 Hz, 1H), 1.84 (d, J=14.6 Hz, 1H).

### Step b

To a stirred solution of [[4-(2,3-dichloro-6-methoxyphenyl)piperidin-2-yl]methyl]urea cis isomer (50 mg, 0.15 mmol) in DCM (2 mL) was added BBr₃ (0.30 g, 1.20 mmol) at room temperature. The reaction was stirred at room temperature for 10 h. The reaction was quenched with water (1 mL). The mixture was neutralized to pH 9 with saturated aq. NaHCO₃. The mixture was concentrated under reduced pressure. The residue was purified by Prep-HPLC with the following conditions: Column: Xselect CSH OBD Column 30×150 mm 5 m; Mobile Phase A: Water (plus 0.05% TFA), Mobile Phase B: ACN; Flow rate: 60 mL/min; Gradient: 5% B to 33% B in 7 min; Detector: UV 254/220 nm; Retention time: 6.40 min. The fractions containing the desired product were collected and concentrated under reduced pressure to afford Compound 102 ((2R,4S)-rel-[4-(2,3-dichloro-6-hydroxyphenyl)piperidin-2-yl]methylurea (cis isomer)) as an off-white solid (19.1 mg, 28%): LCMS (ESI) calc'd for C₁₃H₁₇Cl₂N₃O₂ [M+H]⁺: 318, 320 (3:2), found 318, 320 (3:2); ¹H NMR (400 MHz, CD₃OD) δ 7.25 (d, J=8.8 Hz, 1H), 6.76 (d, J=8.8 Hz, 1H), 4.04-3.80 (m, 1H), 3.80-3.66 (m, 1H), 3.54-3.44 (m, 1H), 3.40-3.34 (m, 2H), 3.17-3.07 (m, 1H), 2.95-2.70 (m, 1H), 2.66-2.53 (m, 1H), 1.89-1.75 (m, 2H).

### Example 75. Compound 104 ((2R,4S)-rel-N-[[4-(2,3-dichloro-6-hydroxyphenyl)piperidin-2-yl]methyl]oxetane-3-carboxamide)

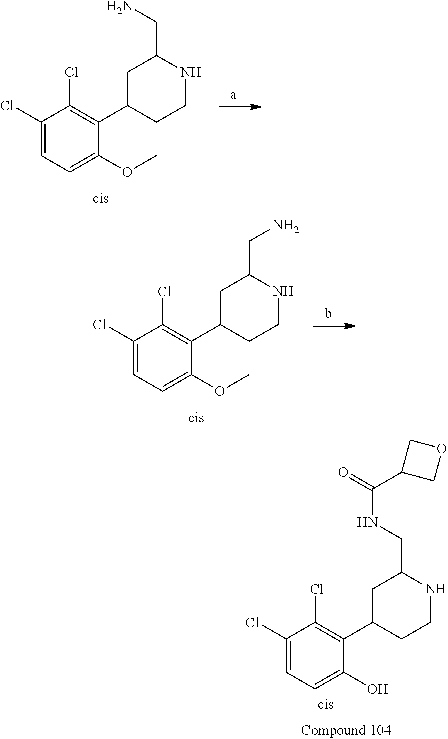 [SMILES: COc1ccc(Cl)c(Cl)c1C1CCNC(CN)C1 ; COc1ccc(Cl)c(Cl)c1C1CCNC(CN)C1 ; O=C(NCC1CC(c2c(O)ccc(Cl)c2Cl)CCN1)C1COC1]

### Step a

To a mixture of 1-[4-(2,3-dichloro-6-methoxyphenyl)piperidin-2-yl]methanamine cis isomer (Example 72, step b, free base compound) (0.20 g, 0.69 mmol) in DCM (5 mL) was added BBr₃ (1.04 g, 4.15 mmol) at 0° C. The reaction mixture was stirred at room temperature for 1 h. The reaction mixture was quenched with MeOH (2 mL) at 0° C. Then the mixture was concentrated under reduced pressure. The residue was purified with reverse phase chromatography, eluted with 20% ACN in water (plus 0.05% TFA) to afford 2-[2-(aminomethyl)piperidin-4-yl]-3,4-dichlorophenol cis isomer as a colorless oil (0.20 g, 57%): LCMS (ESI) calc'd for C₁₂H₁₆Cl₂N₂O [M+H]⁺: 275, 277 (3:2), found 275, 277 (3:2);

### Step b

To a stirred mixture of oxetane-3-carboxylic acid (41 mg, 0.40 mmol) and HATU (0.23 g, 0.61 mmol) in DMF (1 mL) were added 2-[2-(aminomethyl)piperidin-4-yl]-3,4-dichlorophenol cis isomer (0.20 g, 0.40 mmol) and Et₃N (81 mg, 0.80 mmol) at −30° C. The reaction mixture was allowed to warm to 0° C. and stirred for 2 h. The reaction solution was filtered and the filtrate was purified with Prep-HPLC with the following conditions: Column: Xselect CSH OBD Column 30×150 mm 5 m; Mobile Phase A: Water (plus 0.05% TFA), Mobile Phase B: ACN; Flow rate: 60 mL/min; Gradient: 5% B to 35% B in 7 min; Detector: UV 254/220 nm; Retention time: 6.42 min. The fractions containing the desired product were collected and concentrated under reduced pressure to afford Compound 104 ((2R,4S)-rel-N-[[4-(2,3-dichloro-6-hydroxyphenyl)piperidin-2-yl]methyl]oxetane-3-carboxamide (cis isomer)) as an off-white solid (5 mg, 3%): LCMS (ESI) calc'd for C₁₆H₂₀Cl₂N₂O₃ [M+H]⁺: 359, 361 (3:2), found 359, 361 (3:2); ¹H NMR (400 MHz, DMSO-d₆) δ 10.33 (s, 1H), 8.77-8.60 (m, 1H), 8.53-8.34 (m, 1H), 8.20-8.10 (m, 1H), 7.33 (d, J=8.8 Hz, 1H), 6.84 (d, J=8.8 Hz, 1H), 4.71-4.59 (m, 4H), 3.83-3.69 (m, 1H), 3.64-3.46 (m, 2H), 3.31-3.17 (m, 2H), 3.17-2.97 (m, 1H), 2.63-2.51 (m, 1H), 2.41-2.27 (m, 1H), 1.68 (dd, J=28.1, 13.6 Hz, 2H).

The compound in Table 1H below was prepared in an analogous fashion to that described for Compound 104, utilizing the corresponding acid, which was available from commercial sources.

TABLE 1H

| Compound | | | |
|---|---|---|---|
| Number | Structure | Chemical Name | MS: (M + H)⁺ & ¹H MNR |
| 106 | 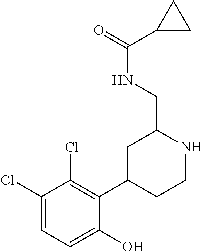 [SMILES: O=C(NCC1CC(c2c(O)ccc(Cl)c2Cl)CCN1)C1CC1] | (2R,4S)-rel-N-[[4-(2,3- dichloro-6- hydroxyphenyl) piperidin-2- yl]methyl]cyclopropane carboxamide | [M + H]⁺: 343, 345 (3:2); ¹H NMR (400 MHz, DMSO-d₆) δ 10.36 (s, 1H), 8.75 (d, J = 11.0 Hz, 1H), 8.50-8.36 (m, 1H), 8.36- 8.20 (m, 1H), 7.34 (d, J = 8.8 Hz, 1H), 6.84 (d, J = 8.8 Hz, 1H), 3.43-3.31 (m, 2H), 3.31-3.15 (m, 2H), 3.12- 2.97 (m, 1H), 2.64-2.53 (m, 1H), 2.40-2.26 (m, 1H), 1.68 (dd, J = 30.4, 13.6 Hz, 2H), 1.61-1.50 (m, 1H), 0.77- 0.62 (m, 4H). |

### Example 76. Compound 111 (N-cyclobutyl-(2R,4S)-rel-2-[4-(2,3-dichloro-6-hydroxyphenyl)piperidin-2-yl]acetamide)

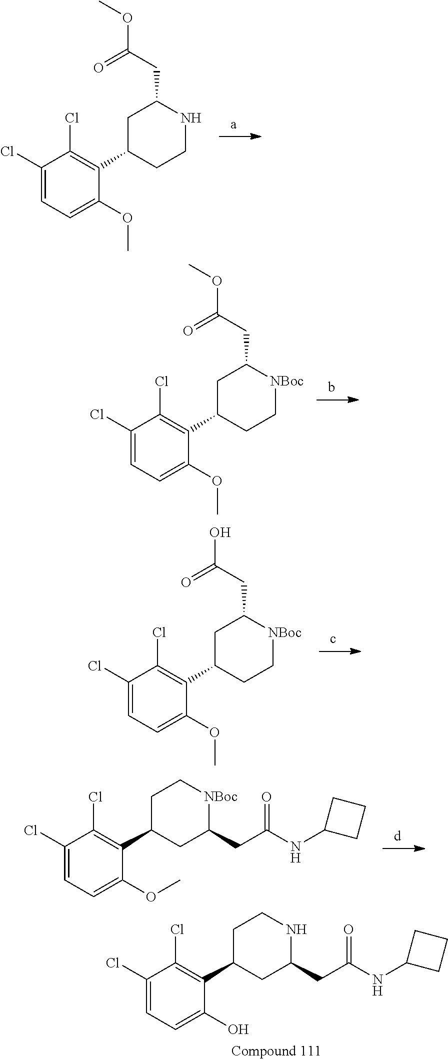

### Step a

To a stirred solution of methyl 2-[4-(2,3-dichloro-6-methoxyphenyl)piperidin-2-yl]acetate (Example 73, step d) (2.00 g, 6.02 mmol) and Et₃N (1.22 g, 12.08 mmol) in DCM (15 mL) was added Boc₂O (1.97 g, 9.04 mmol) at room temperature. The reaction was stirred for 16 h at room temperature. The reaction mixture was diluted with water (30 mL). The resulting mixture was extracted with DCM (3×30 mL). The combined organic layer was washed with brine (2×20 mL) and dried over anhydrous Na₂SO₄. After filtration, the filtrate was concentrated under reduced pressure. The residue was purified by silica gel chromatography, eluted with PE/EA 2/1 to afford tert-butyl 4-(2,3-dichloro-6-methoxyphenyl)-2-(2-methoxy-2-oxoethyl)piperidine-1-carboxylate as a light yellow oil (1.65 g, 61%): LCMS (ESI) calc'd for C₂₀H₂₇Cl₂NO₅ [M+H]⁺: 432, 434 (3:2), found 432, 434 (3:2).

### Step b

A solution of tert-butyl 4-(2,3-dichloro-6-methoxyphenyl)-2-(2-methoxy-2-oxoethyl)piperidine-1-carboxylate (1.65 g, 3.83 mmol) and NaOH (0.38 g, 9.50 mmol) in water (3 mL) and MeOH (10 mL) was stirred for 3 h at room temperature. The reaction solution was adjusted pH to 4 with saturate aq. citric acid. Then the resulting mixture was extracted with EA (3×30 mL). The combined organic layer was washed with brine (2×20 mL) and dried over anhydrous Na₂SO₄. After filtration, the filtrate was concentrated under reduced pressure to afford 2-[1-[(tert-butoxy)carbonyl]-4-(2,3-dichloro-6-methoxyphenyl)piperidin-2-yl]acetic acid as a light yellow solid (1.30 g, 82%): LCMS (ESI) calc'd for C₁₉H₂₅Cl₂NO₅ [M+H]⁺: 418, 420 (3:2), found 418, 420 (3:2); ¹H NMR (400 MHz, DMSO-d₆) δ 12.16 (s, 1H), 7.48 (d, J=8.9 Hz, 1H), 7.05 (d, J=9.0 Hz, 1H), 3.99-3.88 (m, 1H), 3.82 (s, 3H), 3.81-3.71 (m, 1H), 3.65-3.54 (m, 1H), 3.53-3.43 (m, 1H), 3.41-3.33 (m, 1H), 2.64 (dd, J=15.2, 4.8 Hz, 1H), 2.39-2.19 (m, 1H), 1.93-1.79 (m, 1H), 1.79-1.69 (m, 1H), 1.64-1.53 (m, 1H), 1.43 (s, 9H).

### Step c

To a solution of 2-[1-[(tert-butoxy)carbonyl]-4-(2,3-dichloro-6-methoxyphenyl)piperidin-2-yl]acetic acid (0.35 g, 0.84 mmol) and HATU (0.48 g, 1.26 mmol) were added cyclobutanamine (71 mg, 1.00 mmol) and Et₃N (0.17 g, 1.67 mmol) at room temperature. The reaction was stirred at room temperature for 2 h. The reaction was quenched with water (20 mL) at room temperature. The resulting mixture was extracted with EA (2×30 mL). The combined organic layers were washed with brine (2×20 mL) and dried over anhydrous Na₂SO₄. After filtration, the filtrate was concentrated under reduced pressure. The residue was purified by reverse phase chromatography, eluted with 45% ACN in water (plus 0.05% TFA) to afford tert-butyl 2-[(cyclobutylcarbamoyl)methyl]-4-(2,3-dichloro-6-methoxyphenyl)piperidine-1-carboxylate cis isomer as a light yellow solid (0.36 g, 72%): LCMS (ESI) calc'd for C₂₃H₃₂Cl₂N₂O₄ [M+H]⁺: 471, 473 (3:2), found 471, 473 (3:2); ¹H NMR (400 MHz, CDCl₃) δ 7.31-7.29 (m, 1H), 6.74 (d, J=8.9 Hz, 1H), 4.45-4.32 (m, 1H), 4.11-4.00 (m, 1H), 3.83 (s, 3H), 3.65-3.53 (m, 1H), 3.46-3.36 (m, 1H), 2.82 (dd, J=14.6, 7.7 Hz, 1H), 2.44-2.26 (m, 4H), 1.961.80 (m, 5H), 1.771.63 (m, 3H), 1.54 (s, 9H).

### Step d

To a stirred solution of tert-butyl 2-[(cyclobutylcarbamoyl)methyl]-4-(2,3-dichloro-6-methoxyphenyl)piperidine-1-carboxylate cis isomer (0.36 g, 0.75 mmol) in DCM (5 mL) was added BBr₃ (1.13 g, 4.52 mmol) at room temperature. The reaction was stirred at room temperature for 1 h. The reaction was quenched with water (1 mL). The mixture was neutralized to pH 9 with saturated aq. NaHCO₃. The mixture was concentrated under reduced pressure. The residue was purified by Prep-HPLC with the following conditions: SunFire Prep C₁₈ OBD Column 19×150 mm 5 m 10 nm; Mobile Phase A: Water (plus 0.05% TFA), Mobile Phase B: ACN; Flow rate: 25 mL/min; Gradient: 15% B to 30% B in 20 min; Detector: UV 254/220 nm; Retention time: 18.33 min. The fractions containing the desired product were collected and concentrated under reduced pressure to afford Compound 111 (N-cyclobutyl-(2R,4S)-rel-2-[4-(2,3-dichloro-6-hydroxyphenyl)piperidin-2-yl]acetamide (cis isomer)) as an off-white solid (82 mg, 23%): LCMS (ESI) calc'd for C₁₇H₂₂Cl₂N₂O₂ [M+H]⁺: 357, 359 (3:2), found 357, 359 (3:2); ¹H NMR (400 MHz, CD₃OD) δ 7.25 (d, J=8.8 Hz, 1H), 6.76 (d, J=8.8 Hz, 1H), 4.38-4.27 (m, 1H), 3.84-3.69 (m, 1H), 3.65-3.56 (m, 1H), 3.56-3.48 (m, 1H), 3.24-3.12 (m, 1H), 2.81-2.68 (1H), 2.68-2.58 (m, 1H), 2.58-2.48 (m, 2H), 2.34-2.23 (m, 2H), 2.03-1.89 (m 2H), 1.87-1.69 (m, 4H).

The Examples in Table 11 below were prepared in an analogous fashion to that described for Compound 111, starting from 2-[1-[(tert-butoxy)carbonyl]-4-(2,3-dichloro-6-methoxyphenyl)piperidin-2-yl]acetic acid (Example 76, step b) and the corresponding amines, which were available from commercial sources.

TABLE 1I

| Compound | | | |
|---|---|---|---|
| Number | Structure | Chemical Name | MS: (M + H)⁺ & ¹H MNR |
| 113 | 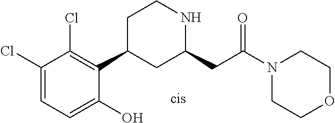 [SMILES: O=C(C[C@H]1C[C@@H](c2c(O)ccc(Cl)c2Cl)CCN1)N1CCOCC1] | (2R,4S)-rel-2-[4- (2,3-dichloro-6- hydroxyphenyl) piperidin-2-yl]-1- (morpholin-4- yl)ethan-1-one | [M + H]⁺: 373, 375 (3:2); 1H NMR (400 MHz, CD3OD) δ 7.26 (d, J = 8.8 Hz, 1H), 6.76 (d, J = 8.8 Hz, 1H), 3.92-3.71 (m, 1H), 3.71-3.58 (m, 7H), 3.58-3.48 (m, 3H), 3.27- 3.13 (m, 1H), 3.02-2.86 (m, |
| | | | 1H), 2.83-2.60 (m, 3H), |
| | | | 1.87 (t, J = 13.4 Hz, 2H). |
| 115 | 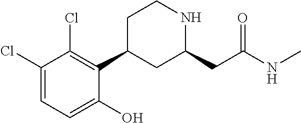 [SMILES: CNC(=O)C[C@H]1C[C@@H](c2c(O)ccc(Cl)c2Cl)CCN1] | (2R,4S)-rel-2-[4- (2,3-dichloro-6- hydroxyphenyl) piperidin-2-yl]-N- methylacetamide | [M + H]⁺: 317, 319 (3:2); ¹H NMR (400 MHz, CD₃OD) δ 7.25 (d, J = 8.8 Hz, 1H), 6.76 (d, J = 8.8 Hz, 1H), 3.83-3.70 (m, 1H), 3.67-3.57 (m, 1H), 3.57-3.49 (m, 1H), 3.25- 3.12 (m, 1H), 2.83-2.66 (m, |
| | | | 4H), 2.66-2.50 (m, 3H), |
| | | | 1.88-1.79 (m, 2H). |
| 114 | 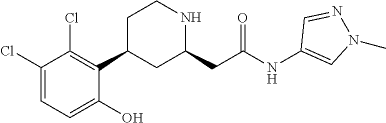 [SMILES: Cn1cc(NC(=O)C[C@H]2C[C@@H](c3c(O)ccc(Cl)c3Cl)CCN2)cn1] | (2R,4S)-rel-2-[4- (2,3-dichloro-6- hydroxyphenyl) piperidin-2-yl]-N-(1- methyl-1H- pyrazol-4- yl)acetamide | [M + H]⁺: 383, 385 (3:2); ¹H NMR (400 MHz, CD₃OD) δ 8.53 (s, 1H), 7.91 (s, 1H), 7.52 (s, 1H), 7.25 (d, J = 8.8 Hz, 1H), 6.75 (d, J = 8.8 Hz, 1H), 3.87 (s, 3H), 3.84-3.74 (m, 1H), 3.74-3.63 (m, 1H), |
| | | | 3.58-3.47 (m, 1H), 3.26- |
| | | | 3.14 (m, 1H), 2.84-2.62 (m, |
| | | | 4H), 1.93-1.76 (m, 2H). |

### Example 77. Compound 112 (2-[[4-(2,3-dichloro-6-hydroxyphenyl)piperidin-2-yl]methyl]-1,2-thiazolidine-1,1-dione cis isomer)

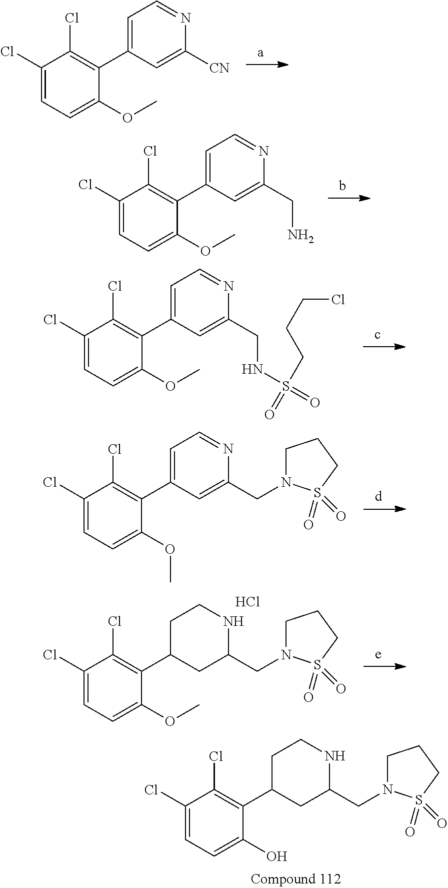

### Step a

To a stirred mixture of 4-(2,3-dichloro-6-methoxyphenyl)pyridine-2-carbonitrile (Example 51, step a) (2.20 g, 7.91 mmol) in MeOH (20 mL) and aq. HCl (12 M, 1 mL) was added PtO₂ (0.50 g, 2.16 mmol) in portions at room temperature. The reaction mixture was stirred at 30° C. under hydrogen atmosphere (50 atm) for 24 h. The mixture was filtered and the filtrate was neutralized with saturated aq. NaHCO₃ to pH 7. The mixture was diluted with EA (50 mL) and water (50 mL). The aqueous solution was extracted with EA (3×30 mL). The combined organic layers were washed with brine (3×30 mL) and dried over anhydrous Na₂SO₄. After filtration, the filtrate was concentrated under reduced pressure to afford 1-[4-(2,3-dichloro-6-methoxyphenyl)pyridin-2-yl]methanamine as a yellow oil (1.8 g, 81%): LCMS (ESI) calc'd for C₁₃H₁₂Cl₂N₂O [M+H]⁺: 283, 285 (3:2), found 283, 285 (3:2);

### Step b

To a stirred solution of 1-[4-(2,3-dichloro-6-methoxyphenyl)pyridin-2-yl]methanamine (0.40 g, 1.41 mmol) and 3-chloropropane-1-sulfonyl chloride (0.30 g, 1.70 mmol) in DCM (4 mL) was added Et₃N (0.29 g, 2.83 mmol) at room temperature. The reaction solution was stirred at room temperature for 1 h. The reaction solution was concentrated under reduced pressure. The residue was purified by silica gel column chromatography, eluted with PE/EA (1/1) to afford 3-chloro-N-[[4-(2,3-dichloro-6-methoxyphenyl)pyridin-2-yl]methyl]propane-1-sulfonamide as a light yellow solid (0.20 g, 33%): LCMS (ESI) calc'd for C₁₆H₁₇C₁₃N₂O₃S [M+H]⁺: 423, 425, 427 (3:3:1), found 423, 425, 427 (3:3:1); ¹H NMR (400 MHz, CDCl₃) δ 8.66 (d, J=5.1 Hz, 1H), 7.51 (d, J=9.0 Hz, 1H), 7.22 (s, 1H), 7.20 (dd, J=5.1, 1.6 Hz, 1H), 6.90 (d, J=9.0 Hz, 1H), 5.77 (t, J=5.4 Hz, 1H), 4.53 (d, J=5.2 Hz, 2H), 3.75 (s, 3H), 3.64 (t, J=6.2 Hz, 2H), 3.19 (dd, J=8.6, 6.4 Hz, 2H), 2.37-2.26 (m, 2H).

### Step c

A solution of 3-chloro-N-[[4-(2,3-dichloro-6-methoxyphenyl)pyridin-2-yl]methyl]propane-1-sulfonamide (0.18 g, 0.43 mmol) and NaOMe (69 mg, 1.27 mmol, 30% in MeOH) in EtOH (10 mL) was stirred 80° C. for 3 h. After cooling to room temperature, the reaction mixture was concentrated under reduced pressure. The residue was purified by silica gel column chromatography, eluted with PE/EA (3/7) to afford 2-[[4-(2,3-dichloro-6-methoxyphenyl)pyridin-2-yl]methyl]-1,2-thiazolidine-1,1-dione as a light yellow solid (0.13 g, 79%): LCMS (ESI) calc'd for C₁₆H₁₆Cl₂N₂O₃S [M+H]⁺: 387, 389 (3:2), found 387, 389 (3:2); ¹H NMR (400 MHz, CDCl₃) δ 8.66 (d, J=5.1 Hz, 1H), 7.50 (d, J=9.0 Hz, 1H), 7.46-7.40 (m, 1H), 7.21 (d, J=4.9 Hz, 1H), 6.89 (d, J=9.0 Hz, 1H), 4.48 (s, 2H), 3.76 (s, 3H), 3.37 (t, J=6.7 Hz, 2H), 3.29-3.18 (m, 2H), 2.46-2.35 (m, 2H).

### Step d

A degassed mixture of 2-[[4-(2,3-dichloro-6-methoxyphenyl)pyridin-2-yl]methyl]-1,2-thiazolidine-1,1-dione (0.10 g, 0.26 mmol) and PtO₂ (59 mg, 0.26 mmol) in MeOH (10 mL) and aq. HCl (6 N, 0.5 mL) was stirred at 30° C. under hydrogen atmosphere (50 atm) for 15 h. The reaction mixture was filtrated and the filtrate was concentered under reduced pressure to afford 2-[[4-(2,3-dichloro-6-methoxyphenyl)piperidin-2-yl]methyl]-12-thiazolidine-1,1-dione cis isomer hydrochloride as an white solid (80 mg, 72%): LCMS (ESI) calc'd for C₁₆H₂₂Cl₂N₂O₃S [M+H]⁺: 393, 395 (3:2), found 393, 395 (3:2); ¹H NMR (400 MHz, CDCl₃) δ 7.34 (d, J=8.9 Hz, 1H), 6.77 (d, J=8.9 Hz, 1H), 3.95 (s, 3H), 3.81-3.64 (m, 2H), 3.64-3.46 (m, 1H), 3.42-3.23 (m, 2H), 3.12-2.96 (m, 1H), 2.96-2.78 (m, 1H), 2.53 (s, 2H), 2.13-1.99 (m, 1H), 1.89-1.55 (m, 6H).

### Step e

To a stirred solution of 2-[[4-(2,3-dichloro-6-methoxyphenyl)piperidin-2-yl]methyl]-1,2-thiazolidine-1,1-dione cis isomer (80 mg, 0.20 mmol) in DCM (5 mL) was added BBr₃ (0.25 g, 1.02 mmol) at room temperature. The reaction was stirred at room temperature for 1 h. The reaction was quenched with water (1 mL). The mixture was neutralized to pH 9 with saturated aq. NaHCO₃. The mixture was concentrated under reduced pressure. The residue was purified by Prep-HPLC with the following conditions: Column: Sunfire Prep C₁₈ OBD Column, 10 m, 19×250 mm; Mobile Phase A: Water (plus 0.1% TFA), Mobile Phase B: ACN; Flow rate: 25 mL/min; Gradient: 50% B to 85% B in 7 min; Detector: UV 254/220 nm; Retention time: 6.52 min. The fractions containing the desired product were collected and concentrated under reduced pressure to afford Compound 112 (2-[[4-(2,3-dichloro-6-hydroxyphenyl)piperidin-2-yl]methyl]-1,2-thiazolidine-1,1-dione cis isomer) as an off-white solid (28 mg, 27%): LCMS (ESI) calc'd for C₁₅H₂₀Cl₂N₂O₃S [M+H]⁺: 379, 381 (3:2), found 379, 381 (3:2); ¹H NMR (400 MHz, CD₃OD) δ 7.26 (d, J=8.8 Hz, 1H), 6.76 (d, J=8.8 Hz, 1H), 3.84-3.71 (m, 1H), 3.58-3.48 (m, 3H), 3.30-3.23 (m, 3H), 3.23-3.13 (m, 3H), 2.87-2.71 (m, 1H), 2.65-2.51 (m, 1H), 2.46-2.37 (m, 2H), 1.97-1.79 (m, 2H).

### Example 78. Compound 116 (3,4-dichloro-2-[2-(1H-pyrazol-4-yl)piperidin-4-yl]phenol)

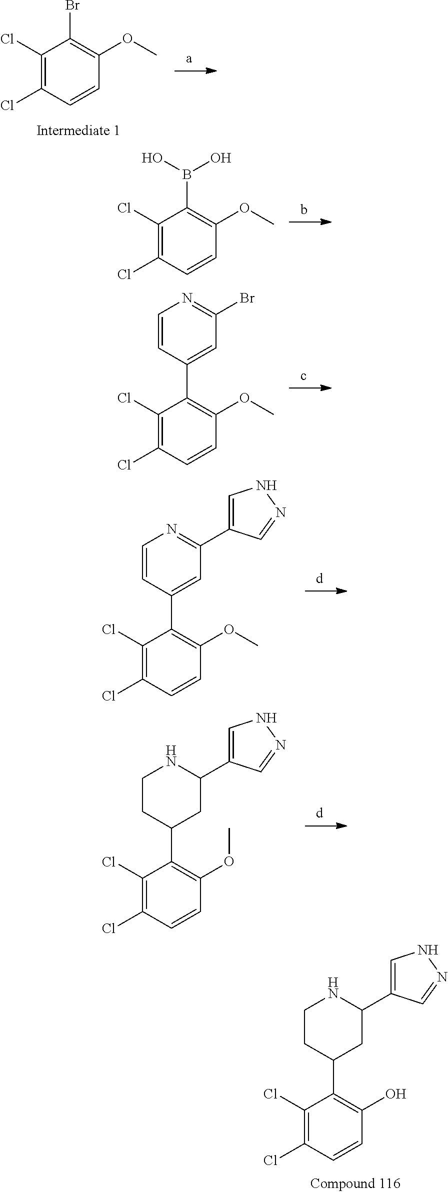

### Step a

To a stirred solution of Intermediate 1 (200 mg, 0.78 mmol, 1 equiv) in THE (3 mL) was added n-BuLi (0.09 mL, 1.379 mmol, 1.2 equiv) at −78° C. under nitrogen atmosphere. The resulting mixture was stirred for 30 min at −78° C. under nitrogen atmosphere. To the above mixture was added triethyl borate (136.9 mg, 0.94 mmol, 1.20 equiv) over 10 min at −78° C. The resulting mixture was stirred for an additional 2 h at room temperature. The reaction was quenched with water at room temperature. The resulting mixture was extracted with EtOAc (3×50 mL). The combined organic layers were washed with brine (2×10 mL) and dried over anhydrous Na₂SO₄. After filtration, the filtrate was concentrated under reduced pressure. The residue was purified by reverse flash chromatography with the following conditions: column, C₁₈ silica gel; mobile phase, CAN in water, 35% to 60% gradient in 15 min; detector, UV 254 nm to afford 2,3-dichloro-6-methoxyphenylboronic acid (70 mg, 40.56%) as an off-white solid. ¹H NMR (400 MHz, CD₃OD) δ 7.46 (d, J=8.8 Hz, 1H), 6.93 (d, J=8.8 Hz, 1H), 3.82 (s, 3H).

### Step b

To a stirred mixture of 2,3-dichloro-6-methoxyphenylboronic acid (0.60 g, 2.72 mmol), 2-bromo-4-iodopyridine (0.93 g, 3.26 mmol) and K₂CO₃ (1.13 g, 8.15 mmol) in toluene (6 mL), EtOH (3 mL) and H₂O (3 mL) was added Pd(dppf)Cl₂ (0.20 g, 0.27 mmol) at room temperature under nitrogen atmosphere. The resulting mixture was stirred for 12 h at 80° C. under nitrogen atmosphere. The mixture was allowed to cool to room temperature. The reaction was diluted with water at room temperature. The resulting mixture was extracted with EA (3×25 mL). The combined organic layers were washed with brine (3×20 mL) and dried over anhydrous Na₂SO₄. After filtration, the filtrate was concentrated under reduced pressure. The residue was purified by Prep-TLC, eluted with PE/EA (1/1) to afford 2-bromo-4-(2,3-dichloro-6-methoxyphenyl)pyridine as a yellow oil (0.47 g, 47%): LCMS (ESI) calc'd for C₁₂H₈BrCl₂NO [M+H]⁺: 332, 334, 336 (3:3:2), found 332, 334, 336 (3:3:2);

### Step c

To a stirred mixture of 2-bromo-4-(2,3-dichloro-6-methoxyphenyl)pyridine (0.58 g, 1.74 mmol), 4-(4,4,5,5-tetramethyl-1,3,2-dioxaborolan-2-yl)-1H-pyrazole (0.41 g, 2.09 mmol) and Na₂CO₃ (0.55 g, 5.23 mmol) in 1,4-dioxane (8 mL) and H₂O (2 mL) was added Pd(PPh₃)₄ (0.20 g, 0.17 mmol) at room temperature under nitrogen atmosphere. The resulting mixture was stirred for 12 h at 80° C. under nitrogen atmosphere. The mixture was allowed to cool down to room temperature. The reaction was diluted with water at room temperature. The resulting mixture was extracted with EA (3×25 mL). The combined organic layers were washed with brine (3×20 mL) and dried over anhydrous Na₂SO₄. After filtration, the filtrate was concentrated under reduced pressure. The residue was purified by Prep-TLC, eluted with PE/EA (1/1) to afford 4-(2,3-dichloro-6-methoxyphenyl)-2-(1H-pyrazol-4-yl)pyridine as a light yellow solid (0.30 g, 48%): LCMS (ESI) calc'd for C₁₅H₁₁Cl₂N₃O [M+H]⁺: 320, 322 (3:2), found 320, 322 (3:2); ¹H NMR (400 MHz, CDCl₃) δ 8.72-8.60 (m, 1H), 8.23 (s, 2H), 7.56-7.40 (m, 2H), 7.15-7.04 (m, 1H), 6.91 (d, J=8.9 Hz, 1H), 3.77 (s, 3H).

### Step d

To a stirred solution of 4-(2,3-dichloro-6-methoxyphenyl)-2-(1H-pyrazol-4-yl)pyridine (0.11 g, 0.34 mmol) in MeOH (5 mL) and aq. HCl (6 N, 0.5 mL) was added PtO₂ (78 mg, 0.34 mmol) at room temperature. The mixture was stirred at room temperature for 48 h under hydrogen atmosphere (1.5 atm). The reaction mixture was filtered through a Celite and the filtrate was concentrated under reduced pressure. The residue was purified by Prep-HPLC with the following conditions: Column: Xselect CSH OBD Column 30×150 mm 5 m; Mobile Phase A: Water (plus 0.05% TFA), Mobile Phase B: ACN; Flow rate: 60 mL/min; Gradient: 10% B to 33% B in 9 min; Detector: UV 254/220 nm; Retention time: 8.73 min. The fractions containing the desired product were collected and concentrated under reduced pressure to afford 4-(2,3-dichloro-6-methoxyphenyl)-2-(1H-pyrazol-4-yl)piperidine as an off-white solid (25 mg, 17%): LCMS (ESI) calc'd for C₁₅H₁₇Cl₂N₃O [M+H]⁺: 326, 328 (3:2), found 326, 328 (3:2); ¹H 1H NMR (400 MHz, CD₃OD) δ 7.80 (s, 2H), 7.45 (d, J=8.9 Hz, 1H), 7.04 (d, J=9.0 Hz, 1H), 4.56-4.46 (m, 1H), 3.99-3.90 (m, 4H), 3.59-3.46 (m, 1H), 3.46-3.35 (m, 1H), 3.01-2.82 (m, 1H), 2.79-2.60 (m, 1H), 2.11 (d, J=14.1 Hz, 1H), 1.89 (d, J=14.3 Hz, 1H).

### Step e

To a stirred solution of 4-(2,3-dichloro-6-methoxyphenyl)-2-(1H-pyrazol-4-yl)piperidine (25 mg, 0.06 mmol) in DCM (1 mL) was added BBr₃ (0.14 g, 0.57 mmol) at 0° C. The resulting solution was stirred for 1 h at room temperature. The reaction was quenched with MeOH (1 mL). The mixture was concentrated under reduced pressure. The residue was purified by Prep-HPLC with the following conditions: Column: Xselect CSH OBD Column 30×150 mm 5 m; Mobile Phase A: Water (plus 0.05% TFA), Mobile Phase B: ACN; Flow rate: 60 mL/min; Gradient: 8% B to 34% B in 7 min; Detector: UV 254/220 nm; Retention time: 6.77 min. The fractions containing the desired product were collected and concentrated under reduced pressure to afford Compound 116 (3,4-dichloro-2-[2-(1H-pyrazol-4-yl)piperidin-4-yl]phenol) as an off-white solid (13.5 mg, 53%): LCMS (ESI) calc'd for C₁₄H₁₅Cl₂N₃O [M+H]⁺: 312, 314 (3:2), found 312, 314 (3:2); ¹H NMR (400 MHz, CD₃OD) δ 7.79 (s, 2H), 7.27 (d, J=8.7 Hz, 1H), 6.79 (d, J=8.8 Hz, 1H), 4.58-4.45 (m, 1H), 3.96-3.77 (m, 1H), 3.59-3.47 (m, 1H), 3.31-3.27 (m, 1H), 3.17-2.99 (m, 1H), 2.95-2.78 (m, 1H), 2.10 (d, J=14.2 Hz, 1H), 1.89 (d, J=14.2 Hz, 1H).

### Example 79. Compound 117 ((2R)—N-(azetidin-3-yl)-5-(2,3-dichloro-6-hydroxyphenyl)pyrrolidine-2-carboxamide isomer 1) and Compound 118 ((2R)—N-(azetidin-3-yl)-5-(2,3-dichloro-6-hydroxyphenyl)pyrrolidine-2-carboxamide isomer 2)

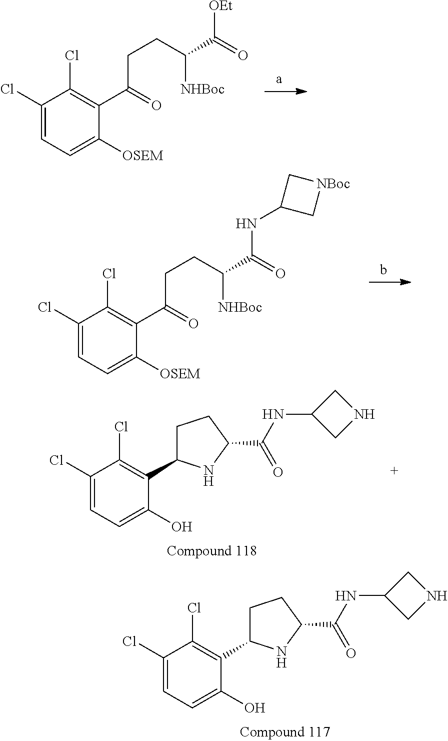

### Step a

To a stirred mixture of ethyl (2R)-2-[(tert-butoxycarbonyl)amino]-5-(2,3-dichloro-6-[[2-(trimethylsilyl)ethoxy]methoxy]phenyl)-5-oxopentanoate (Intermediate 7, Example 6) (0.220 g, 0.40 mmol) in MeOH (3 mL) and H₂O (0.50 mL) was added LiOH.H₂O (50.0 mg, 1.20 mmol) at room temperature. The reaction mixture was stirred for 1 h and concentrated under reduced pressure. To the resulting crude in DMF (3.00 mL) were added HATU (0.230 g, 0.60 mmol), tert-butyl 3-aminoazetidine-1-carboxylate (0.100 g, 0.60 mmol), and TEA (0.120 g, 1.20 mmol). The reaction mixture was stirred for 1 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 65% ACN in water (plus 0.05% TFA) to afford tert-butyl 3-[(2R)-2-[(tert-butoxycarbonyl)amino]-5-(2,3-dichloro-6-[[2-(trimethylsilyl)ethoxy]methoxy]phenyl)-5-oxopentanamido]azetidine-1-carboxylate as light-yellow oil (0.190 g, 70%): LCMS (ESI) calc'd for C₃₀H₄₇Cl₂N₃O₈Si [M+H]⁺: 676, 678 (3:2) found 676, 678 (3:2); ¹H NMR (400 MHz, CDCl₃) δ 7.41 (d, J=8.9 Hz, 1H), 7.10 (d, J=9.0 Hz, 1H), 6.90 (s, 1H), 5.21 (s, 2H), 4.68-4.57 (m, 1H), 4.27 (t, J=8.5 Hz, 2H), 4.20-4.11 (m, 1H), 3.81-3.69 (m, 4H), 3.05 (dt, J=19.0, 6.9 Hz, 1H), 2.86 (dt, J=19.1, 6.4 Hz, 1H), 2.33-2.21 (m, 1H), 2.12-2.00 (m, 1H), 1.47 (d, J=2.2 Hz, 18H), 0.97-0.89 (m, 2H), 0.02 (d, J=1.3 Hz, 9H).

### Step b

To a stirred solution of tert-butyl 3-[2-[(tert-butoxycarbonyl)amino]-5-(2,3-dichloro-6-[[2-(trimethylsilyl)ethoxy]methoxy]phenyl)-5-oxopentanamido]azetidine-1-carboxylate (0.190 g, 0.28 mmol) in DCM (2 mL) was added TFA (2 mL) at room temperature. The reaction mixture was stirred for 1 h and concentrated under reduced pressure. Then, to the resulting crude in EA (3 mL) was added PtO₂ (64.0 mg, 0.28 mmol). The reaction mixture was stirred for 1 h under hydrogen atmosphere (1.5 atm), filtered, and the filtrate concentrated under reduced pressure. The residue was purified by Prep-HPLC with the following conditions: Column: Sun Fire Prep C₁₈ OBD Column, 19×150 mm 5 μm 10 nm; Mobile Phase A: Water (plus 0.05% TFA), Mobile Phase B: ACN; Flow rate: 20 mL/min; Gradient: 25% B to 35% B in 4.3 min; Detector: UV 254/210 nm; Retention Time: 4.23 min. The fractions containing the desired product were collected and concentrated under reduced pressure to afford the desired product as TFA salt (100 mg). The product was separated by Prep Chiral HPLC with the following conditions: Column: CHIRALPAK IG, 3×25 cm, 5 μm; Mobile Phase A: MTBE (plus 0.2% IPA)-HPLC, Mobile Phase B: EtOH-HPLC; Flow rate: 40 mL/min; Gradient: 30% B to 30% B in 22 min; Detector: UV 220/254 nm; Retention Time 1:10.10 min; Retention Time 2: 20.70 min. The faster-eluting isomer at 10.10 min was obtained as Compound 117 ((2R)—N-(azetidin-3-yl)-5-(2,3-dichloro-6-hydroxyphenyl)pyrrolidine-2-carboxamide isomer 1) as a brown solid (2.80 mg, 2.24%): LCMS (ESI) calc'd C₁₄H₁₇Cl₂N₃O₂ for [M+H]⁺: 330, 332 (3:2) found 330, 332 (3:2); ¹H NMR (400 MHz, CD₃OD) δ 7.35 (d, J=8.8 Hz, 1H), 6.82 (d, J=8.9 Hz, 1H), 5.36-5.31 (m, 1H), 4.72-4.62 (m, 1H), 4.55 (dd, J=10.1, 7.1 Hz, 1H), 4.29-4.18 (m, 2H), 4.18-4.08 (m, 2H), 2.65-2.54 (m, 1H), 2.40-2.25 (m, 2H), 2.08 (dt, J=12.0, 9.1 Hz, 1H). The slower-eluting isomer at 20.70 min was obtained as Compound 118 ((2R)—N-(azetidin-3-yl)-5-(2,3-dichloro-6-hydroxyphenyl)pyrrolidine-2-carboxamide isomer 2) as an off-white solid (22.7 mg, 18.2%): LCMS (ESI) calc'd C₁₄H₁₇Cl₂N₃O₂ for [M+H]⁺: 330, 332 (3:2) found 330, 332 (3:2); ¹H NMR (400 MHz, CD₃OD) δ 7.36 (d, J=8.9 Hz, 1H), 6.81 (d, J=8.9 Hz, 1H), 5.28-5.16 (m, 1H), 4.71-4.64 (m, 1H), 4.49-4.31 (m, 1H), 4.31-4.06 (m, 4H), 2.69-2.47 (m, 1H), 2.40-2.28 (m, 1H), 2.28-2.13 (m, 2H).

The Examples in Table 1J below were prepared in an analogous fashion to that described for Compound 117, starting from substituted ethyl (2S)-2-[(tert-butoxycarbonyl)amino]-5-(2,3-dichloro-6-[[2-(trimethylsilyl)ethoxy]methoxy]phenyl)-5-oxopentanoate and tert-butyl 3-aminoazetidine-1-carboxylate, which was available from commercial sources.

TABLE 1J

| Compound | | | |
|---|---|---|---|
| Number | Structure | Chemical Name | MS: (M + H)⁺ & ¹H MNR |
| 119 | 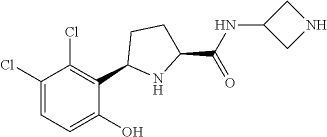 [SMILES: O=C(NC1CNC1)[C@@H]1CC[C@H](c2c(O)ccc(Cl)c2Cl)N1] | (2S)-N-(azetidin-3-yl)-5- (2,3-dichloro-6- hydroxyphenyl)pyrrolidine- 2-carboxamide isomer 1 | [M + H]⁺: 330, 332 (3:2); ¹H NMR (400 MHz, CD₃OD) δ 7.34 (d, J = 8.9 Hz, 1H), 6.80 (d, J = 8.9 Hz, 1H), 5.36-5.28 (m, 1H), 4.71-4.60 (m, 1H), 4.49 (dd, J = 10.0, 7.3 Hz, 1H), 4.27- 4.19 (m, 2H), 4.18-4.09 (m, 2H), 2.63-2.53 (m, 1H), |
| | | | 2.39-2.25 (m, 2H), 2.13-1.99 |
| | | | (m, 1H). |
| 120 | 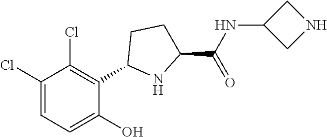 [SMILES: O=C(NC1CNC1)[C@@H]1CC[C@@H](c2c(O)ccc(Cl)c2Cl)N1] | (2S)-N-(azetidin-3-yl)-5- (2,3-dichloro-6- hydroxyphenyl)pyrrolidine- 2-carboxamide isomer 2 | [M + H]⁺: 330, 332 (3:2); ¹H NMR (400 MHz, CD₃OD) δ 7.36 (d, J = 8.9 Hz, 1H), 6.80 (d, J = 8.9 Hz, 1H), 5.22 (dd, J = 10.3, 7.1 Hz, 1H), 4.73-4.64 (m, 1H), 4.38 (dd, J = 10.4, 5.4 Hz, 1H), 4.27-4.19 (m, 2H), 4.18-4.09 (m, 2H), 2.62-2.49 |
| | | | (m, 1H), 2.38-2.27 (m, 1H), |
| | | | 2.25-2.11 (m, 2H). |

### Example 80. Compound 121 ((5R)—N-(azetidin-3-yl)-6-(2,3-dichloro-6-hydroxyphenyl)piperidine-3-carboxamide isomer 1)

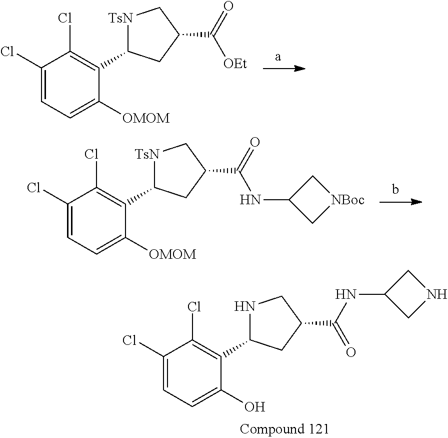

### Step a

To a stirred mixture of ethyl (5R)-5-[2,3-dichloro-6-(methoxymethoxy)phenyl]-1-(4-methylbenzenesulfonyl)pyrrolidine-3-carboxylate isomer 1 (Intermediate 10, Example 8) (0.150 g, 0.30 mmol) in MeOH (1 mL) and H₂O (0.5 mL) was added LiOH.H₂O (25.0 mg, 0.60 mmol) at room temperature. The reaction mixture was stirred for 1 h and concentrated under reduced pressure. Then, to the crude in DMF (2 mL) were added tert-butyl 3-aminoazetidine-1-carboxylate (78.0 mg, 0.45 mmol), HATU (0.170 g, 0.45 mmol), and TEA (61.0 mg, 0.60 mmol). The reaction mixture was stirred for 2 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 60% ACN in water (plus 0.05% TFA) to afford tert-butyl 3-[(5R)-5-[2,3-dichloro-6-(methoxymethoxy)phenyl]-1-(4-methylbenzenesulfonyl)pyrrolidine-3-amido]azetidine-1-carboxylate isomer 1 as a light-yellow oil (0.190 g, 89%): LCMS (ESI) calc'd for C₂₈H₃₅Cl₂N₃O₇S [M+Na]⁺: 650, 652 (3:2) found 650, 652 (3:2); ¹H NMR (400 MHz, CDCl₃) δ 7.57 (d, J=7.9 Hz, 2H), 7.30 (d, J=8.6 Hz, 1H), 7.23 (d, J=7.9 Hz, 2H), 6.91 (d, J=9.0 Hz, 1H), 6.24 (d, J=7.4 Hz, 1H), 5.51 (d, J=9.1 Hz, 1H), 5.13-4.97 (m, 2H), 4.65-4.58 (m, 1H), 4.24 (q, J=8.3 Hz, 2H), 4.10-3.98 (m, 1H), 3.82-3.67 (m, 4H), 3.48 (s, 3H), 2.69-2.49 (m, 1H), 2.42 (s, 3H), 2.40-2.28 (m, 1H), 1.45 (s, 9H).

### Step b

A solution of tert-butyl 3-[(5R)-5-[2,3-dichloro-6-(methoxymethoxy)phenyl]-1-(4-methylbenzenesulfonyl)pyrrolidine-3-amido]azetidine-1-carboxylate isomer 1 (0.190 g, 0.30 mmol) in HBr (2.00 mL, 33% in AcOH) was stirred at room temperature for 2 h. The resulting mixture was concentrated under reduced pressure. The residue was purified by Prep-HPLC with the following conditions: Column: Sun Fire Prep C₁₈ OBD Column, 19×150 mm, 5 m, 10 nm; Mobile Phase A: water (plus 0.05% TFA), Mobile Phase B: ACN; Flow rate: 20 mL/min; Gradient: 5% B to 30% B in 4.30 min. Detector: UV 254/210 nm; Retention time: 4.20 min. The fractions containing the desired product were collected and concentrated under reduced pressure to afford the desired product. The product (40.0 mg) was purified by Prep Chiral HPLC with the following conditions: Column: CHIRALPAK IG, 2×25 cm, 5 μm; Mobile Phase A: Hex (plus 0.3% IPA)-HPLC, Mobile Phase B: EtOH-HPLC; Flow rate: 20 mL/min; Gradient: 40% B to 40% B in 27 min; Detector: UV 220/254 nm; Retention time: 9.24 min. The fractions containing the desired product were collected and concentrated under reduced pressure to afford the desired product. Then, the product (15 mg) was purified by Prep-HPLC with the following conditions: Column: Sun Fire Prep C₁₈ OBD Column, 19×150 mm, 5 m, 10 nm; Mobile Phase A: water (plus 0.05% TFA), Mobile Phase B: ACN; Flow rate: 20 mL/min; Gradient: 25% B to 50% B in 4.30 min; Detector: UV 254/210 nm; Retention time: 4.20 min. The fractions containing the desired product were collected and concentrated under reduced pressure to afford Compound 121 ((5R)—N-(azetidin-3-yl)-5-(2,3-dichloro-6-hydroxyphenyl)pyrrolidine-3-carboxamide isomer 1) as a purple solid (8.20 mg, 8%): LCMS (ESI) calc'd for C₁₄H₁₇Cl₂N₃O₂ [M+H]⁺: 330, 332 (3:2) found 330, 332 (3:2); ¹H NMR (300 MHz, CD₃OD) δ 7.47 (d, J=8.9 Hz, 1H), 6.92 (d, J=8.9 Hz, 1H), 5.33 (dd, J=11.4, 7.3 Hz, 1H), 4.75-4.61 (m, 1H), 4.31 (dd, J=11.2, 8.4 Hz, 2H), 4.20 (dd, J=11.3, 7.4 Hz, 2H), 3.90 (dd, J=11.5, 8.4 Hz, 1H), 3.62 (dd, J=11.5, 8.2 Hz, 1H), 3.51-3.37 (m, 1H), 2.73-2.46 (m, 2H).

The Examples in Table 1K below were prepared in an analogous fashion to that described for Compound 121, starting from the corresponding ethyl 5-(2,3-dichloro-6-(methoxymethoxy)phenyl)-1-tosylpyrrolidine-3-carboxylate and tert-butyl 3-aminoazetidine-1-carboxylate, which was available from commercial sources.

TABLE 1K

| Compound | | | |
|---|---|---|---|
| Number | Structure | Chemical Name | MS: (M + H)⁺ & ¹H MNR |
| 122 | 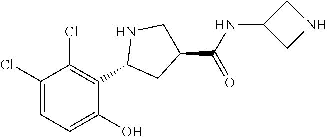 [SMILES: O=C(NC1CNC1)[C@@H]1CN[C@@H](c2c(O)ccc(Cl)c2Cl)C1] | (5R)-N-(azetidin-3-yl)-5- (2,3-dichloro-6- hydroxyphenyl)pyrrolidine- 3-carboxamide isomer 2 | [M + H]⁺: 330, 332 (3:2); ¹H NMR (300 MHz, CD₃OD) δ 7.48 (d, J = 8.9 Hz, 1H), 6.94 (d, J = 8.9 Hz, 1H), 5.49 (dd, J = 10.8, 7.8 Hz, 1H), 4.79-4.66 (m, 1H), 4.34 (dd, J = 11.1, 8.2 Hz, 2H), 4.28-4.16 (m, 2H), 3.81 (dd, J = 11.7, 7.1 Hz, 1H), |
| | | | 3.68 (dd, J = 11.6, 3.7 Hz, |
| | | | 1H), 3.53-3.43 (m, 1H), |
| | | | 2.78-2.61 (m, 1H), 2.52-2.39 |
| | | | (m, 1H). |
| 123 | 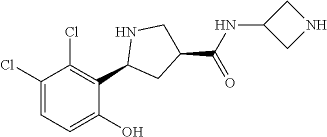 [SMILES: O=C(NC1CNC1)[C@@H]1CN[C@H](c2c(O)ccc(Cl)c2Cl)C1] | (5S)-N-(azetidin-3-yl)-5- (2,3-dichloro-6- hydroxyphenyl)pyrrolidine- 3-carboxamide isomer 1 | [M + H]⁺: 330, 332 (3:2); ¹H NMR (400 MHz, CD₃OD) δ 7.47 (d, J = 8.9 Hz, 1H), 6.93 (d, J = 8.9 Hz, 1H), 5.34 (dd, J = 11.5, 7.2 Hz, 1H), 4.76-4.64 (m, 1H), 4.36-4.27 (m, 2H), 4.24-4.16 (m, 2H), 3.90 (dd, J = 11.5, 8.5 Hz, 1H), 3.63 (dd, J = |
| | | | 11.5, 8.3 Hz, 1H), 3.47-3.39 |
| | | | (m, 1H), 2.68-2.50 (m, 2H). |
| 124 | 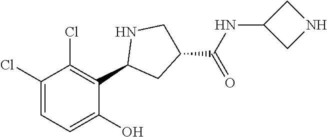 [SMILES: O=C(NC1CNC1)[C@H]1CN[C@H](c2c(O)ccc(Cl)c2Cl)C1] | (5S)-N-(azetidin-3-yl)-5- (2,3-dichloro-6- hydroxyphenyl)pyrrolidine- 3-carboxamide isomer 2 | [M + H]⁺: 330, 332 (3:1); ¹H NMR (400 MHz, CD₃OD) δ 7.47 (d, J = 8.9 Hz, 1H), 6.95 (d, J = 8.9 Hz, 1H), 5.50 (dd, J = 10.7, 7.8 Hz, 1H), 4.79-4.66 (m, 1H), 4.39-4.29 (m, 2H), 4.27-4.18 (m, 2H), 3.82 (dd, J = 11.7, 7.1 Hz, 1H), 3.68 (dd, J = |
| | | | 11.7, 3.9 Hz, 1H), 3.55-3.45 |
| | | | (m, 1H), 2.75-2.64 (m, 1H), |
| | | | 2.53-2.43 (m, 1H). |

### Example 81. Compound 125 (N-[2-amino-2-(5-chloro-2-hydroxy-4-methylphenyl)ethyl]azetidine-3-carboxamide)

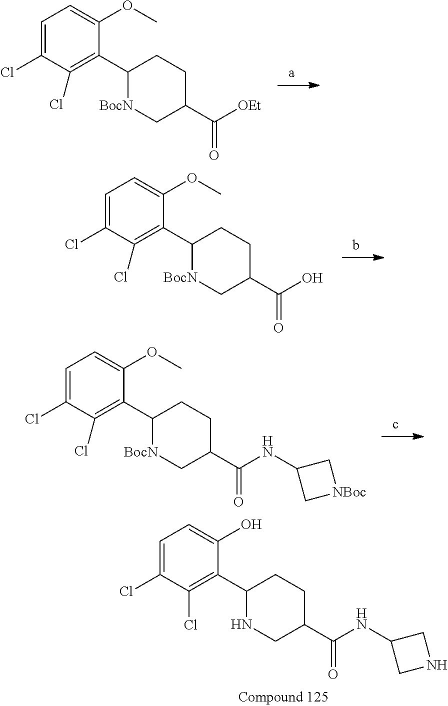

### Step a

To a stirred solution of 1-tert-butyl 3-ethyl 6-(2,3-dichloro-6-methoxyphenyl)piperidine-1,3-dicarboxylate (Intermediate 12, Example 9) (0.260 g, 0.60 mmol) in MeOH (2 mL) was added LiOH.H₂O (51.0 mg, 1.20 mmol) at room temperature. The reaction mixture was stirred for 1 h and concentrated under reduced pressure. The residue was purified by reverse phase chromatography, eluting with 48% ACN in water (plus 0.05% TFA) to afford 1-(tert-butoxycarbonyl)-6-(2,3-dichloro-6-methoxyphenyl)piperidine-3-carboxylic acid as a yellow oil (0.120 g, 49%): LCMS (ESI) calc'd for C₁₈H₂₃Cl₂NO₅ [M+H]⁺: 404, 406 (3:2) found 404, 406 (3:2): ¹H NMR (400 MHz, CDCl₃) δ 7.33 (d, J=8.9 Hz, 1H), 6.79 (d, J=8.9 Hz, 1H), 5.27 (dd, J=11.9, 5.2 Hz, 1H), 4.36 (dd, J=13.7, 6.7 Hz, 1H), 3.86-3.83 (m, 1H), 3.61-3.55 (m, 1H), 3.56-3.51 (m, 2H), 3.10-2.99 (m, 1H), 2.23-2.08 (m, 1H), 2.08-1.98 (m, 1H), 1.98-1.85 (m, 1H), 1.85-1.72 (m, 1H), 1.21 (s, 9H).

### Step b

To a stirred solution of 1-(tert-butoxycarbonyl)-6-(2,3-dichloro-6-methoxyphenyl)piperidine-3-carboxylic acid (0.120 g, 0.28 mmol) and HATU (0.170 g, 0.45 mmol) in DMF (1.50 mL) were added TEA (90.0 mg, 0.89 mmol) and tert-butyl 3-aminoazetidine-1-carboxylate (77.0 mg, 0.45 mmol) at room temperature. The reaction solution was stirred for 1 h, diluted with water (30 mL), and extracted with EA (3×30 mL). The combined organic layers were washed with brine (3×5 mL) and dried over anhydrous Na₂SO₄. After filtration, the filtrate was concentrated under reduced pressure. The residue was purified by reverse phase chromatography, eluting with 60% ACN in water (plus 0.05% TFA) to afford tert-butyl 5-[[1-(tert-butoxycarbonyl)azetidin-3-yl]carbamoyl]-2-(2,3-dichloro-6-methoxyphenyl)piperidine-1-carboxylate) as a yellow oil (0.120 g, 72%): LCMS (ESI) calc'd for C₂₆H₃₇Cl₂N₃O₆ [M+H]⁺: 558, 560 (3:2) found 558, 560 (3:2); ¹H NMR (300 MHz, CDCl₃) δ 7.35 (d, J=8.9 Hz, 1H), 6.80 (d, J=9.0 Hz, 1H), 5.23 (dd, J=12.1, 4.9 Hz, 1H), 4.72-4.55 (m, 1H), 4.33-4.16 (m, 3H), 3.89-3.71 (m, 5H), 3.61-3.48 (m, 1H), 2.86-2.74 (m, 1H), 2.31-2.05 (m, 2H), 1.92-1.68 (m, 2H), 1.47 (s, 9H), 1.19 (s, 9H).

### Step c

To a stirred solution of tert-butyl 5-[[1-(tert-butoxycarbonyl)azetidin-3-yl]carbamoyl]-2-(2,3-dichloro-6-methoxyphenyl)piperidine-1-carboxylate (0.100 g, 0.03 mmol) in DCM (2 mL) was added BBr₃ (90.0 mg, 0.36 mmol) at room temperature. The reaction mixture was stirred for 16 h, quenched with MeOH (2 mL), and concentrated under reduced pressure. The residue was purified by Prep-HPLC with the following conditions: Column: Atlantis Prep T3 OBD Column, 19×250 mm 10 μm; Mobile Phase A: Water (plus 0.05% TFA), Mobile Phase B: ACN; Flow rate: 20 mL/min; Gradient: 20% to 30% in 6.5 min; Detector: UV 254/220 nm; Retention time: 6.20 min. The fractions containing the desired product were collected and concentrated under reduced pressure to afford Compound 125 (N-(azetidin-3-yl)-6-(2,3-dichloro-6-hydroxyphenyl)piperidine-3-carboxamide) as a purple solid (16.0 mg, 19%): LCMS (ESI) calc'd for C₁₅H₁₉Cl₂N₃O₂ [M+H]⁺: 344, 346 (3:2) found 344, 346 (3:2); ¹H NMR (300 MHz, CD₃OD) δ 7.44 (d, J=8.9 Hz, 1H), 6.90 (d, J=8.9 Hz, 1H), 4.85-4.68 (m, 2H), 4.40-4.27 (m, 2H), 4.26-4.16 (m, 2H), 3.75 (d, J=12.8 Hz, 1H), 3.38 (d, J=3.4 Hz, 1H), 3.01-2.95 (m, 1H), 2.44-2.17 (m, 3H), 1.97-1.82 (m, 1H).

The Examples in Table 1L below were prepared in an analogous fashion to that described for Compound 125, starting from the corresponding N-boc-ethyl-substituted phenyl-piperidine carboxylate, prepared in an analogous fashion to that described for Intermediate 12 (Example 9) and tert-butyl 3-aminoazetidine-1-carboxylate, which was available from commercial sources.

TABLE 1L

| Compound | | | |
|---|---|---|---|
| Number | Structure | Chemical Name | MS: (M + H)⁺ & ¹H MNR |
| 126 | 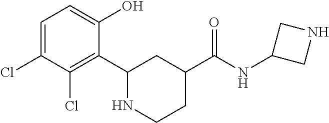 [SMILES: O=C(NC1CNC1)C1CCNC(c2c(O)ccc(Cl)c2Cl)C1] | N-(azetidin-3-yl)-2-(2,3- dichloro-6- hydroxyphenyl)piperidine- 4-carboxamide | [M + H]⁺: 344, 346 (3:2); ¹H NMR (400 MHz, CD₃OD) δ 7.47 (dd, J = 8.9, 5.1 Hz, 1H), 6.94 (dd, J = 8.9, 2.9 Hz, 1H), 4.96-4.90 (m, 1H), 4.75-4.63 (m, 1H), 4.38-4.23 (m, 2H), 4.23-4.14 (m, 2H), 3.63-3.52 (m, 1H), |
| | | | 3.27 (dd, J = 12.9, 3.2 Hz, |
| | | | 1H), 2.89-2.79 (m, 1H), |
| | | | 2.55-2.41 (m, 1H), 2.22-1.94 |
| | | | (m, 3H). |
| 127 | 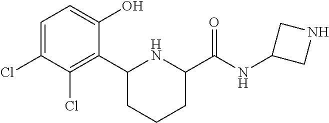 [SMILES: O=C(NC1CNC1)C1CCCC(c2c(O)ccc(Cl)c2Cl)N1] | N-(azetidin-3-yl)-6-(2,3- dichloro-6- hydroxyphenyl)piperidine- 2-carboxamide | [M + H]⁺: 344, 346 (3:2); ¹H NMR (300 MHz, CD₃OD) δ 7.48 (d, J = 8.9 Hz, 1H), 6.95 (d, J = 8.9 Hz, 1H), 5.00 (dd, J = 12.0, 3.5 Hz, 1H), 4.80-4.69 (m, 1H), 4.42-4.12 (m, 5H), 2.37-2.25 (m, 1H), 2.23-1.98 (m, 3H), |
| | | | 1.97-1.77 (m, 2H). |

### Example 82. Evaluation of Kv1.3 potassium channel blocker activities

This assay is used to evaluate the disclosed compounds' activities as Kv1.3 potassium channel blockers.

### Cell Culture

CHO-K₁ cells stably expressing Kv1.3 were grown in DMEM containing 10% heat-inactivated FBS, 1 mM sodium pyruvate, 2 mM L-glutamine, and G418 (500 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10-second breaks were used between pulse trains (see Table A below).

### Patch Clamp Recordings and Compound Application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound/control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 83. Evaluation of hERG Activities

This assay is used to evaluate the disclosed compounds's inhibition activities against the hERG channel.

hERG Electrophysiology

This assay is used to evaluate the disclosed compounds' inhibition activities against the hERG channel.

### Cell Culture

CHO-K₁ cells stably expressing hERG were grown in Ham's F-12 Medium with glutamine containing 10% heat-inactivated FBS, 1% penicillin/streptomycin, hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table B) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound/control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 1 provides a summary of the inhibition activities of certain selected compounds of the instant invention against Kv1.3 potassium channel and hERG channel.

TABLE 1
IC₅₀ (μM) values of certain exemplified compounds of the instant invention against
Kv1.3 potassium channel and hERG channel

| Compound | | | hERG |
|---|---|---|---|
| Number | Structure | Kv1.3 IC₅₀ | IC₅₀ |
| 1 | 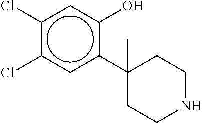 | <10 | * |
| 2 | 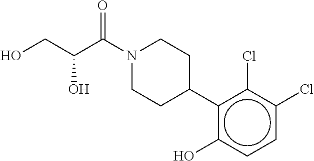 | <1 | >30 |
| 3 | 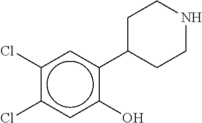 | <1 | <30 |
| 4 | 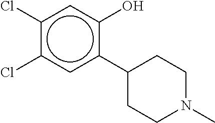 | <1 | <30 |
| 5 | 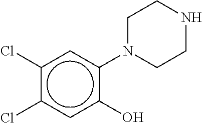 | <10 | <30 |
| 6 | 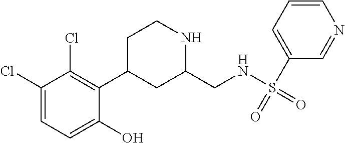 [SMILES: O=S(=O)(NCC1CC(c2c(O)ccc(Cl)c2Cl)CCN1)c1cccnc1] | <1 | >30 |
| 7 | 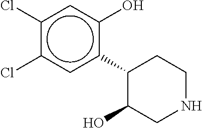 | <10 | * |
| 8 | 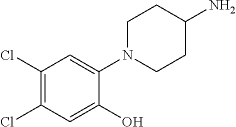 [SMILES: NC1CCN(c2cc(Cl)c(Cl)cc2O)CC1] | <1 | <30 |
| 9 | 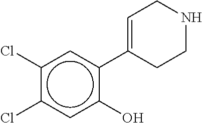 | <1 | <30 |
| 10 | 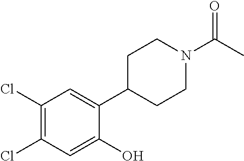 [SMILES: CC(=O)N1CCC(c2cc(Cl)c(Cl)cc2O)CC1] | <10 | * |
| 11 | 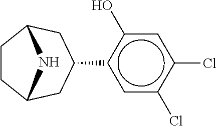 | <10 | * |
| 12 | 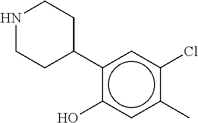 | <10 | <30 |
| 13 | 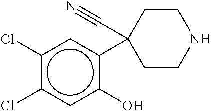 | <10 | * |
| 14 | 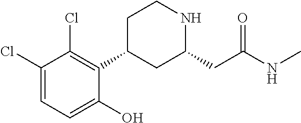 [SMILES: CNC(=O)C[C@@H]1C[C@H](c2c(O)ccc(Cl)c2Cl)CCN1] | <1 | >30 |
| 15 | 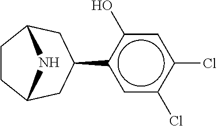 | <10 | * |
| 16 | 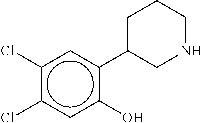 | <1 | <30 |
| 17 | 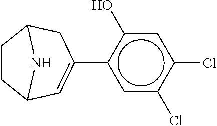 | <10 | * |
| 18 | 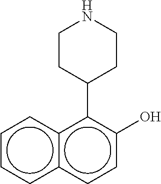 | <1 | <30 |
| 19 | 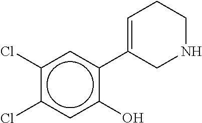 | <10 | * |
| 20 | 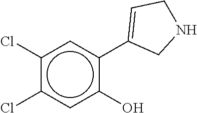 | <10 | * |
| 21 | 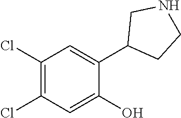 [SMILES: Oc1cc(Cl)c(Cl)cc1C1CCNC1] | <1 | <30 |
| 22 | 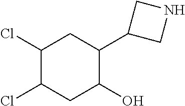 [SMILES: OC1CC(Cl)C(Cl)CC1C1CNC1] | <10 | * |
| 23 | 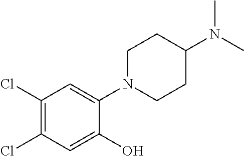 [SMILES: CN(C)C1CCN(c2cc(Cl)c(Cl)cc2O)CC1] | <1 | <30 |
| 24 | 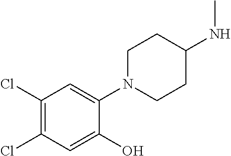 [SMILES: CNC1CCN(c2cc(Cl)c(Cl)cc2O)CC1] | <1 | <30 |
| 25 | 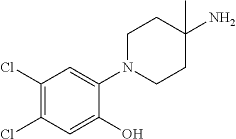 [SMILES: CC1(N)CCN(c2cc(Cl)c(Cl)cc2O)CC1] | <1 | <30 |
| 26 | 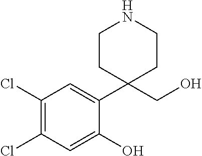 [SMILES: OCC1(c2cc(Cl)c(Cl)cc2O)CCNCC1] | <10 | * |
| 27 | 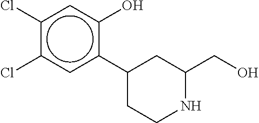 | <10 | >30 |
| 28 | 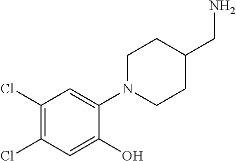 [SMILES: NCC1CCN(c2cc(Cl)c(Cl)cc2O)CC1] | <1 | <30 |
| 29 | 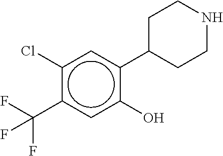 | <10 | * |
| 30 | 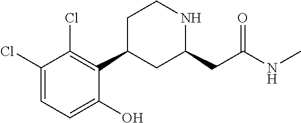 [SMILES: CNC(=O)C[C@H]1C[C@@H](c2c(O)ccc(Cl)c2Cl)CCN1] | <1 | >30 |
| 31 | 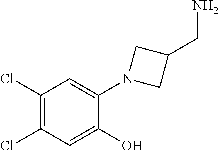 [SMILES: NCC1CN(c2cc(Cl)c(Cl)cc2O)C1] | <1 | <30 |
| 32 | 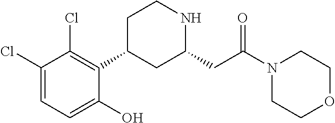 [SMILES: O=C(C[C@@H]1C[C@H](c2c(O)ccc(Cl)c2Cl)CCN1)N1CCOCC1] | <1 | >30 |
| 33 | 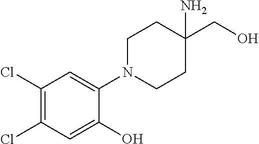 [SMILES: NC1(CO)CCN(c2cc(Cl)c(Cl)cc2O)CC1] | <10 | * |
| 34 | 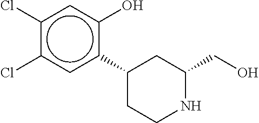 | <10 | * |
| 35 | 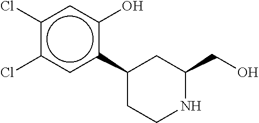 | <10 | * |
| 36 | 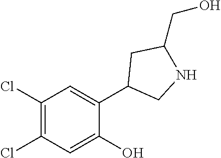 [SMILES: OCC1CC(c2cc(Cl)c(Cl)cc2O)CN1] | <10 | <30 |
| 37 | 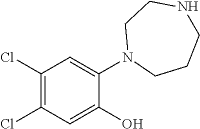 [SMILES: Oc1cc(Cl)c(Cl)cc1N1CCCNCC1] | <10 | * |
| 38 | 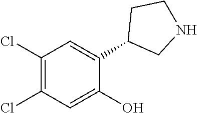 [SMILES: Oc1cc(Cl)c(Cl)cc1[C@@H]1CCNC1] | <1 | <30 |
| 39 | 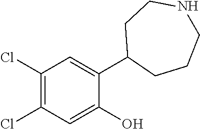 [SMILES: Oc1cc(Cl)c(Cl)cc1C1CCCNCC1] | <1 | <30 |
| 40 | 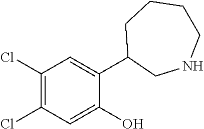 [SMILES: Oc1cc(Cl)c(Cl)cc1C1CCCCNC1] | <10 | <30 |
| 41 | 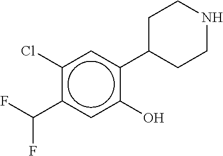 | <10 | * |
| 42 | 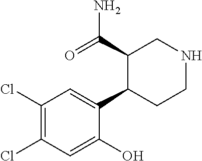 [SMILES: NC(=O)[C@H]1CNCC[C@H]1c1cc(Cl)c(Cl)cc1O] | <1 | >30 |
| 43 | 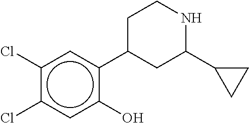 | <1 | <30 |
| 44 | 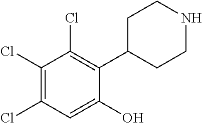 [SMILES: Oc1cc(Cl)c(Cl)c(Cl)c1C1CCNCC1] | <1 | <30 |
| 45 | 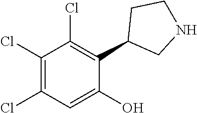 [SMILES: Oc1cc(Cl)c(Cl)c(Cl)c1[C@H]1CCNC1] | <10 | * |
| 46 | 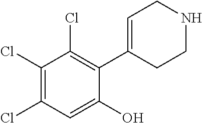 [SMILES: Oc1cc(Cl)c(Cl)c(Cl)c1C1=CCNCC1] | <10 | * |
| 47 | 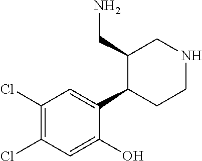 [SMILES: NC[C@H]1CNCC[C@H]1c1cc(Cl)c(Cl)cc1O] | <30 | * |
| 48 | 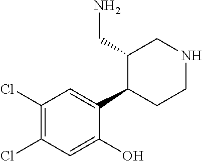 [SMILES: NC[C@@H]1CNCC[C@H]1c1cc(Cl)c(Cl)cc1O] | <10 | * |
| 49 | 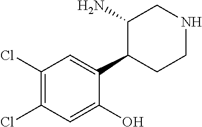 [SMILES: N[C@@H]1CNCC[C@H]1c1cc(Cl)c(Cl)cc1O] | <10 | * |
| 50 | 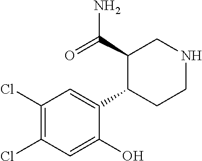 [SMILES: NC(=O)[C@H]1CNCC[C@@H]1c1cc(Cl)c(Cl)cc1O] | <10 | * |
| 51 | 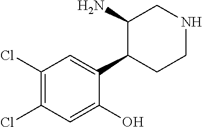 [SMILES: N[C@H]1CNCC[C@H]1c1cc(Cl)c(Cl)cc1O] | <10 | * |
| 52 | 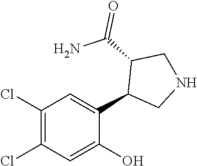 [SMILES: NC(=O)[C@@H]1CNC[C@H]1c1cc(Cl)c(Cl)cc1O] | <10 | * |
| 53 | 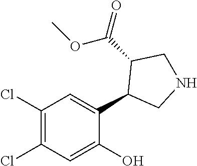 [SMILES: COC(=O)[C@@H]1CNC[C@H]1c1cc(Cl)c(Cl)cc1O] | <10 | * |
| 54 | 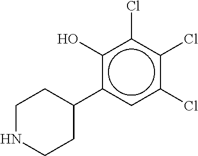 | <10 | <30 |
| 55 | 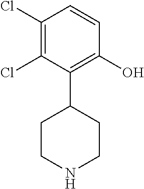 [SMILES: Oc1ccc(Cl)c(Cl)c1C1CCNCC1] | <1 | <30 |
| 56 | 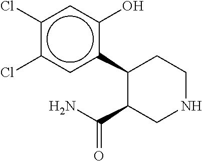 | <10 | * |
| 57 | 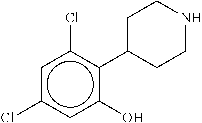 | <1 | <30 |
| 58 | 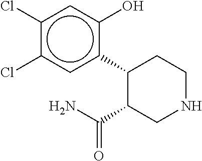 | <1 | * |
| 59 | 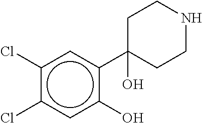 | <10 | * |
| 60 | 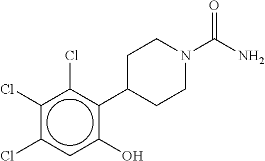 | <1 | <30 |
| 61 | 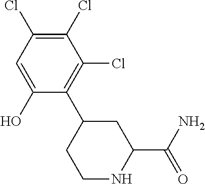 [SMILES: NC(=O)C1CC(c2c(O)cc(Cl)c(Cl)c2Cl)CCN1] | <1 | <30 |
| 62 | 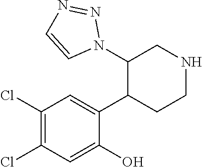 [SMILES: Oc1cc(Cl)c(Cl)cc1C1CCNCC1n1ccnn1] | <10 | * |
| 63 | 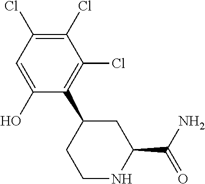 [SMILES: NC(=O)[C@@H]1C[C@H](c2c(O)cc(Cl)c(Cl)c2Cl)CCN1] | <1 | <30 |
| 64 | 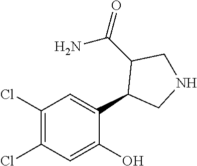 [SMILES: NC(=O)C1CNC[C@H]1c1cc(Cl)c(Cl)cc1O] | <10 | * |
| 65 | 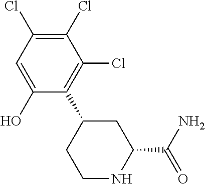 [SMILES: NC(=O)[C@H]1C[C@@H](c2c(O)cc(Cl)c(Cl)c2Cl)CCN1] | <1 | <30 |
| 66 | 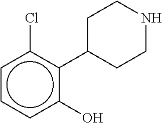 | <10 | * |
| 67 | 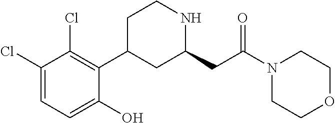 [SMILES: O=C(C[C@H]1CC(c2c(O)ccc(Cl)c2Cl)CCN1)N1CCOCC1] | <1 | >30 |
| 68 | 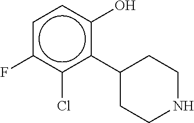 | <10 | * |
| 69 | 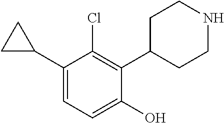 [SMILES: Oc1ccc(C2CC2)c(Cl)c1C1CCNCC1] | <30 | * |
| 70 | 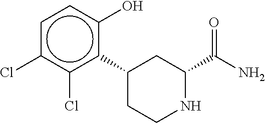 | <1 | >30 |
| 71 | 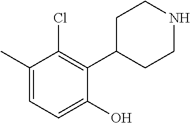 | <30 | * |
| 72 | 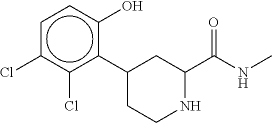 | <1 | >30 |
| 73 | 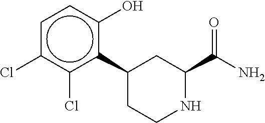 | <1 | >30 |
| 74 | 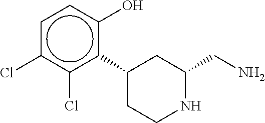 | <1 | >30 |
| 75 | 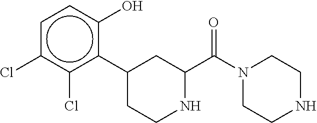 | <10 | * |
| 76 | 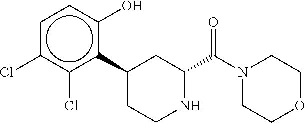 | <10 | * |
| 77 | 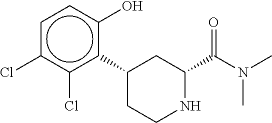 | <10 | * |
| 78 | 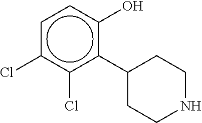 | <10 | * |
| 79 | 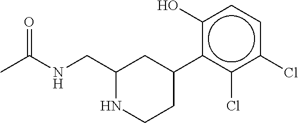 | <1 | >30 |
| 80 | 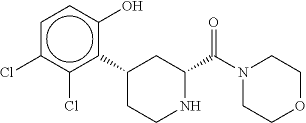 | <10 | * |
| 81 | 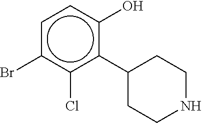 | <1 | * |
| 82 | 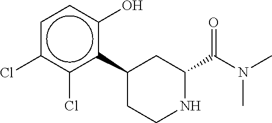 | <10 | * |
| 83 | 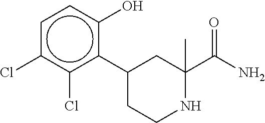 | <10 | * |
| 84 | 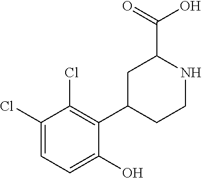 [SMILES: O=C(O)C1CC(c2c(O)ccc(Cl)c2Cl)CCN1] | <10 | * |
| 85 | 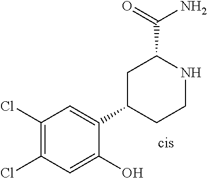 [SMILES: NC(=O)[C@H]1C[C@@H](c2cc(Cl)c(Cl)cc2O)CCN1] | <1 | >30 |
| 86 | 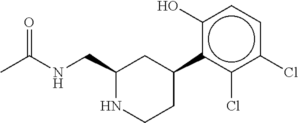 | <1 | >30 |
| 87 | 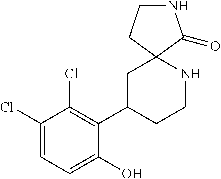 [SMILES: O=C1NCCC12CC(c1c(O)ccc(Cl)c1Cl)CCN2] | <10 | * |
| 88 | 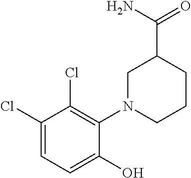 [SMILES: NC(=O)C1CCCN(c2c(O)ccc(Cl)c2Cl)C1] | <10 | * |
| 89 | 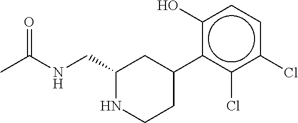 | <1 | >30 |
| 90 | 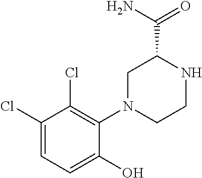 [SMILES: NC(=O)[C@H]1CN(c2c(O)ccc(Cl)c2Cl)CCN1] | <10 | * |
| 91 | 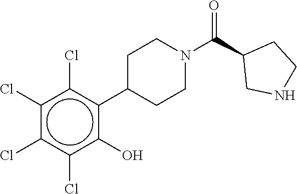 | <1 | <30 |
| 92 | 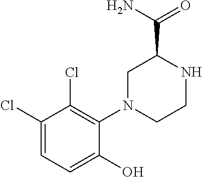 [SMILES: NC(=O)[C@@H]1CN(c2c(O)ccc(Cl)c2Cl)CCN1] | <10 | * |
| 93 | 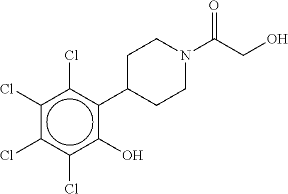 | <1 | <30 |
| 94 | 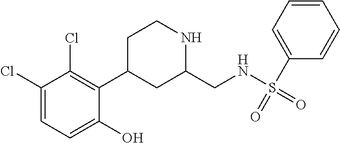 [SMILES: O=S(=O)(NCC1CC(c2c(O)ccc(Cl)c2Cl)CCN1)c1ccccc1] | <1 | <30 |
| 95 | 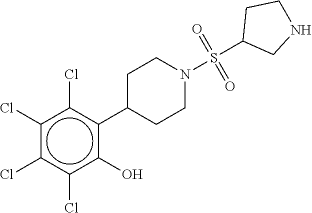 | <10 | * |
| 96 | 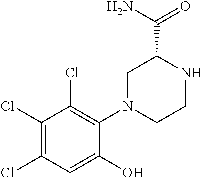 [SMILES: NC(=O)[C@H]1CN(c2c(O)cc(Cl)c(Cl)c2Cl)CCN1] | <1 | * |
| 97 | 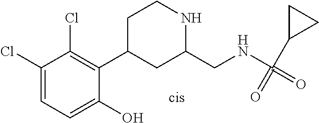 | <1 | >30 |
| 98 | 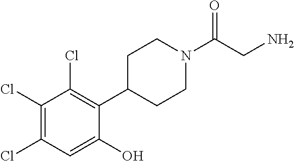 [SMILES: NCC(=O)N1CCC(c2c(O)cc(Cl)c(Cl)c2Cl)CC1] | <1 | <30 |
| 99 | 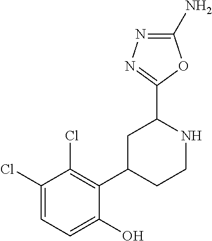 [SMILES: Nc1nnc(C2CC(c3c(O)ccc(Cl)c3Cl)CCN2)o1] | <1 | >30 |
| 100 | 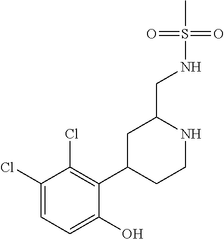 [SMILES: CS(=O)(=O)NCC1CC(c2c(O)ccc(Cl)c2Cl)CCN1] | <1 | >30 |
| 101 | 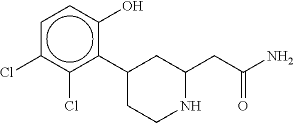 | <1 | >30 |
| 102 | 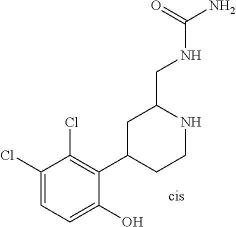 [SMILES: NC(=O)NCC1CC(c2c(O)ccc(Cl)c2Cl)CCN1] | <1 | >30 |
| 103 | 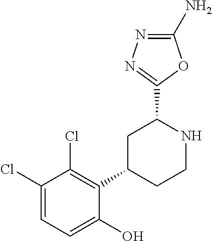 [SMILES: Nc1nnc([C@H]2C[C@@H](c3c(O)ccc(Cl)c3Cl)CCN2)o1] | <10 | * |
| 104 | 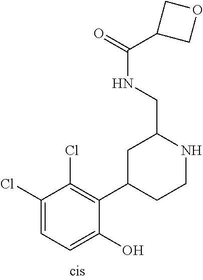 [SMILES: O=C(NCC1CC(c2c(O)ccc(Cl)c2Cl)CCN1)C1COC1] | <1 | >30 |
| 105 | 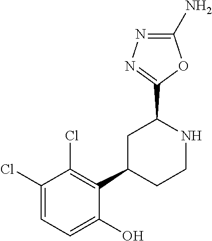 [SMILES: Nc1nnc([C@@H]2C[C@H](c3c(O)ccc(Cl)c3Cl)CCN2)o1] | <1 | >30 |
| 106 | 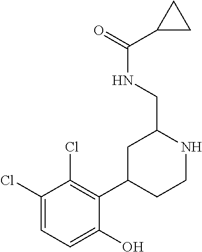 [SMILES: O=C(NCC1CC(c2c(O)ccc(Cl)c2Cl)CCN1)C1CC1] | <1 | >30 |
| 107 | 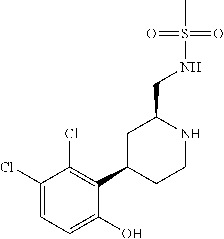 [SMILES: CS(=O)(=O)NC[C@@H]1C[C@H](c2c(O)ccc(Cl)c2Cl)CCN1] | <1 | >30 |
| 108 | 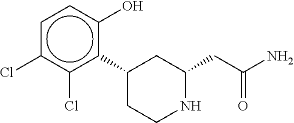 | <1 | >30 |
| 109 | 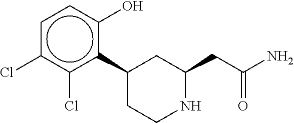 | <1 | >30 |
| 110 | 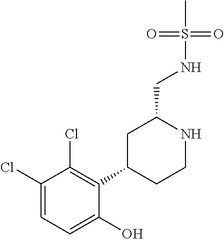 [SMILES: CS(=O)(=O)NC[C@H]1C[C@@H](c2c(O)ccc(Cl)c2Cl)CCN1] | <1 | >30 |
| 111 | 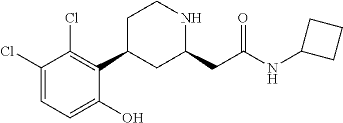 [SMILES: O=C(C[C@H]1C[C@@H](c2c(O)ccc(Cl)c2Cl)CCN1)NC1CCC1] | <1 | <30 |
| 112 | 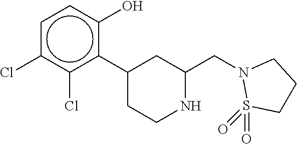 | <1 | <30 |
| 113 | 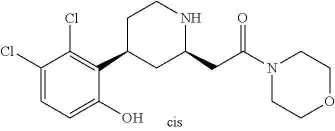 [SMILES: O=C(C[C@H]1C[C@@H](c2c(O)ccc(Cl)c2Cl)CCN1)N1CCOCC1] | <1 | >30 |
| 114 | 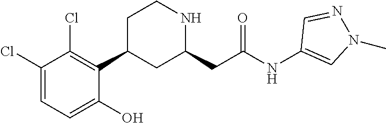 [SMILES: Cn1cc(NC(=O)C[C@H]2C[C@@H](c3c(O)ccc(Cl)c3Cl)CCN2)cn1] | <1 | >30 |
| 115 | 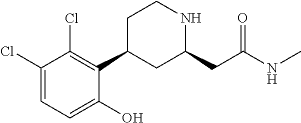 [SMILES: CNC(=O)C[C@H]1C[C@@H](c2c(O)ccc(Cl)c2Cl)CCN1] | <1 | >30 |
| 116 | 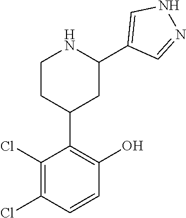 [SMILES: Oc1ccc(Cl)c(Cl)c1C1CCNC(c2cn[nH]c2)C1] | <1 | >30 |
| 117 | 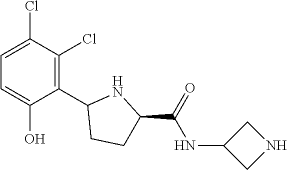 [SMILES: O=C(NC1CNC1)[C@H]1CCC(c2c(O)ccc(Cl)c2Cl)N1] | <30 | * |
| 118 | 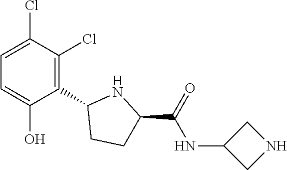 [SMILES: O=C(NC1CNC1)[C@H]1CC[C@H](c2c(O)ccc(Cl)c2Cl)N1] | <1 | >100 |
| 119 | 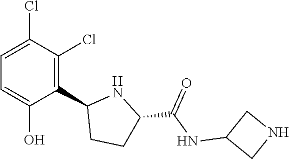 [SMILES: O=C(NC1CNC1)[C@@H]1CC[C@@H](c2c(O)ccc(Cl)c2Cl)N1] | <10 | * |
| 120 | 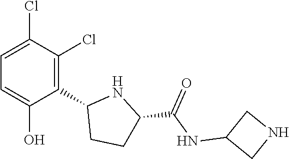 [SMILES: O=C(NC1CNC1)[C@@H]1CC[C@H](c2c(O)ccc(Cl)c2Cl)N1] | <10 | * |
| 121 | 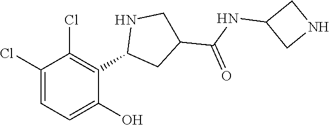 [SMILES: O=C(NC1CNC1)C1CN[C@@H](c2c(O)ccc(Cl)c2Cl)C1] | <1 | * |
| 122 | 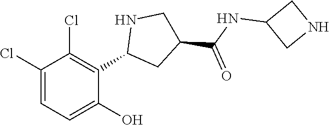 [SMILES: O=C(NC1CNC1)[C@@H]1CN[C@@H](c2c(O)ccc(Cl)c2Cl)C1] | <1 | >100 |
| 123 | 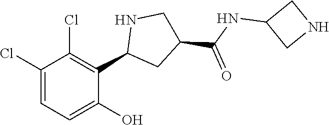 [SMILES: O=C(NC1CNC1)[C@@H]1CN[C@H](c2c(O)ccc(Cl)c2Cl)C1] | <10 | * |
| 124 | 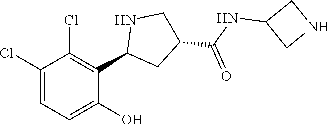 [SMILES: O=C(NC1CNC1)[C@H]1CN[C@H](c2c(O)ccc(Cl)c2Cl)C1] | <1 | * |
| 125 | 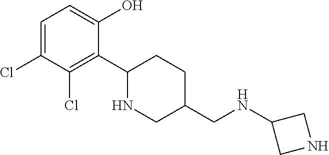 [SMILES: Oc1ccc(Cl)c(Cl)c1C1CCC(CNC2CNC2)CN1] | <10 | >100 |
| 126 | 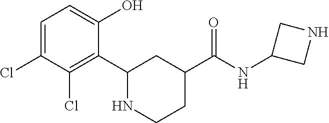 [SMILES: O=C(NC1CNC1)C1CCNC(c2c(O)ccc(Cl)c2Cl)C1] | <10 | >30 |
| 127 | 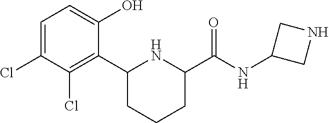 [SMILES: O=C(NC1CNC1)C1CCCC(c2c(O)ccc(Cl)c2Cl)N1] | <10 | >30 |

*Not Tested.

1. A compound of Formula I or a pharmaceutically-acceptable salt thereof, 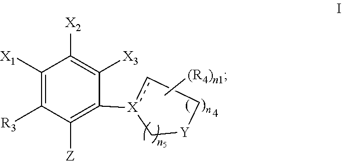
wherein [symbol]
refers to a single or double bond;
X is C, N, or CR₄ wherein valence permits;
Y is C(R₄)₂, NR₅, or O; wherein at least one of X and Y is N optionally substituted by R₅ where valence permits; wherein Y and either of its adjacent ring atoms are not linked together to form a fused ring system;
Z is ORₐ;
X₁ is H, halogen, CN, alkyl, cycloalkyl, halogenated cycloalkyl, or halogenated alkyl;
X₂ is H, halogen, CN, alkyl, cycloalkyl, halogenated cycloalkyl, or halogenated alkyl;
X₃ is H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₃ is independently H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, optionally substituted saturated heterocycle, optionally substituted aryl, optionally substituted heteroaryl, CN, CF₃, ORₐ, (CR₆R₇)ₙ₃ORₐ, oxo, (C═O)R_{b}, (C═O)OR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, (C═O)NRₐ(CR₆R₇)ₙ₃OR_{b}, (CR₆R₇)ₙ₃NRₓR_{b}, or (CR₆R₇)ₙ₃(C═O)NRₓR_{b}; wherein Rₓ is Rₐ, (C═O)Rₐ, (C═O)NRₐR_{b}, or SO₂Rₐ;
or two R₄ groups taken together with the carbon atom(s) that they are connected to form a 3-7 membered optionally substituted carbocycle or heterocycle;
each occurrence of R₅ is independently H, alkyl, cycloalkyl, optionally substituted saturated heterocycle, optionally substituted aryl, optionally substituted heteroaryl, Rₐ, NRₐR_{b}, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ;
each occurrence of R₆ and R₇ are independently H, alkyl, cycloalkyl, optionally substituted aryl, or optionally substituted heteroaryl;
each occurrence of Rₐ and R_{b} are independently H, alkyl, alkenyl, cycloalkyl, optionally substituted saturated heterocycle comprising 1-3 heteroatoms each selected from the group consisting of N, O, and S, optionally substituted aryl, or optionally substituted heteroaryl; or alternatively Rₐ and R_{b} together with the nitrogen atom that they are connected to form an optionally substituted heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, R₃, R₄, R₅, R₆, R₇, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, NR₈(C═O)R₈, and oxo where valence permits;
each occurrence of R₈ is independently H, alkyl, or optionally substituted heterocycle; or alternatively the two R₈ groups together with the nitrogen atom that they are connected to form an optionally substituted heterocycle comprising the nitrogen atom and 0-3 additional heteroatoms each selected from the group consisting of N, O, and S;
each occurrence of n₁ is independently an integer from 0-3 wherein valence permits;
each occurrence of n₃ is independently an integer from 0-3; and
each occurrence of n₄ and n₅ is independently 0, 1 or 2.

2. The compound of claim 1, wherein [symbol] is a single bond.

3. The compound of claim 1, wherein [symbol] is a double bond.

4. The compound of claim 1, wherein the structural moiety 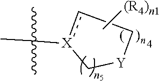
has the structure of

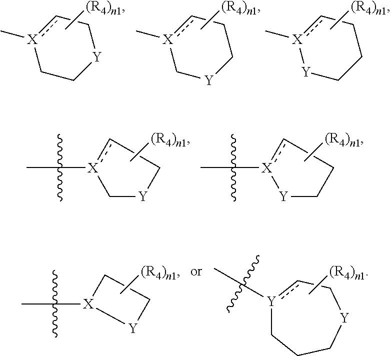

5. The compound of claim 1, wherein X is N and Y is C(R₄)₂.

6. The compound of claim 1, wherein X is CR₄ and Y is NR₅.

7. The compound of claim 1, wherein X is CR₄ and Y is O.

8. The compound of claim 1, wherein X is N and Y is NR₅.

9. The compound of claim 1, wherein the structural moiety 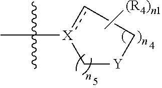
has the structure of

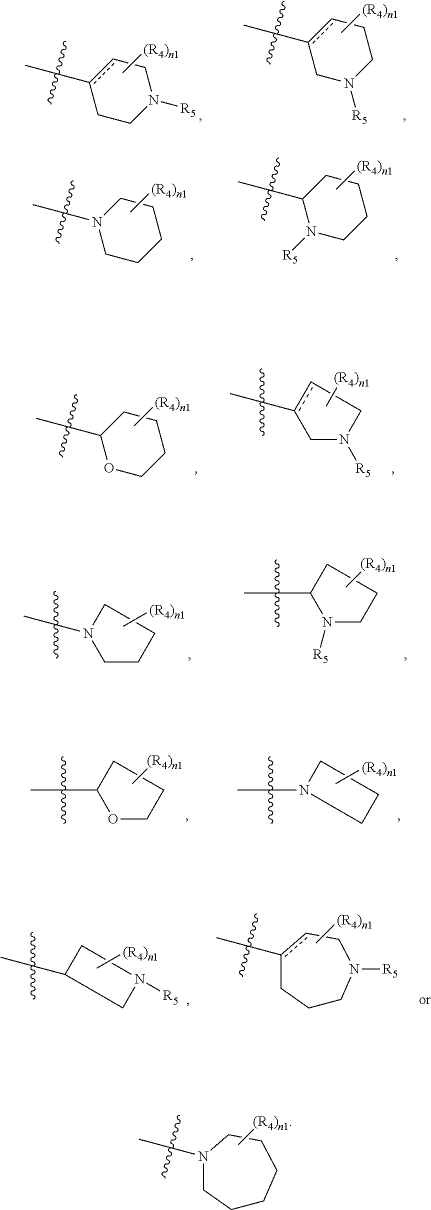

10. The compound of claim 1, wherein the structural moiety 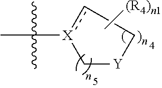
has the structure of

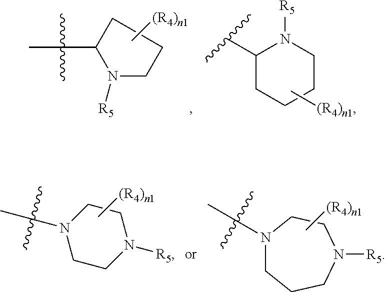

11. The compound of claim 9, wherein n₁ is 0 and R₅ is H or alkyl.

12. The compound of claim 9, wherein n₁ is 1 and R₅ is H or alkyl.

13. The compound of claim 11, wherein R₅ is H.

14. The compound of claim 1, wherein at least one occurrence of R₄ is H, CN, alkyl, cycloalkyl, aryl, heteroaryl, C₃, or ORₐ.

15. The compound of claim 1, wherein at least one occurrence of R₄ is (CR₆R₇)ₙ₃ORₐ, oxo, (C═O)R_{b}, (C═O)OR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, (CR₆R₇)ₙ₃(C═O)NRₐR_{b}, or a N-containing heterocycle.

16. The compound of claim 1, wherein one or more occurrences of R₄ are H or alkyl.

17. The compound of claim 1, wherein one or more occurrences of R₄ are (CR₆R₇)ₙ₃ORₐ or (CR₆R₇)ₙ₃NRₐR_{b}.

18. The compound of claim 1, wherein one or more occurrences of R₄ are ORₐ, NRₐR_{b}, —CH₂ORₐ, —CH₂NRₐR_{b}, —CH₂CH₂ORₐ, or —CH₂CH₂NRₐR_{b}.

19. The compound of claim 1, wherein at least one occurrence of R₄ is (CR₆R₇)ₙ₃(C═O)NRₐR_{b} or (C═O)NRₐ(CR₆R₇)ₙ₃OR_{b}.

20. The compound of claim 19, wherein at least one or more occurrences of R₄ is (C═O)NRₐR_{b} or —CH₂(C═O)NRₐR_{b}.

21. The compound of claim 1, wherein R₄ is H, Me, Et, Pr, Bu, or a saturated heterocycle or heteroaryl selected from the group consisting of 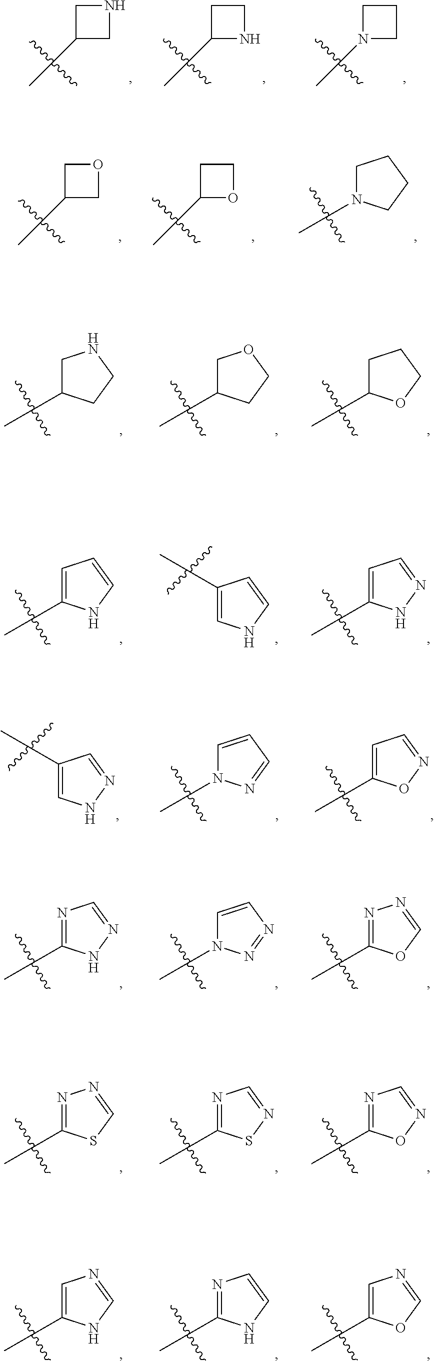 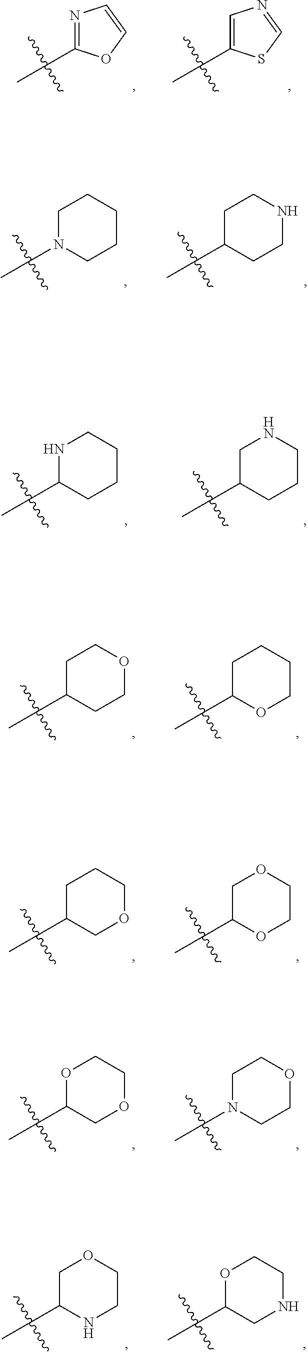 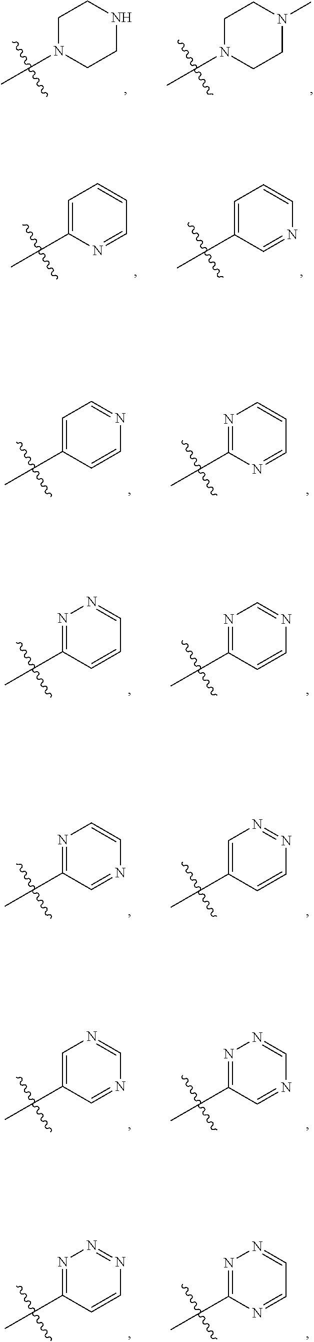 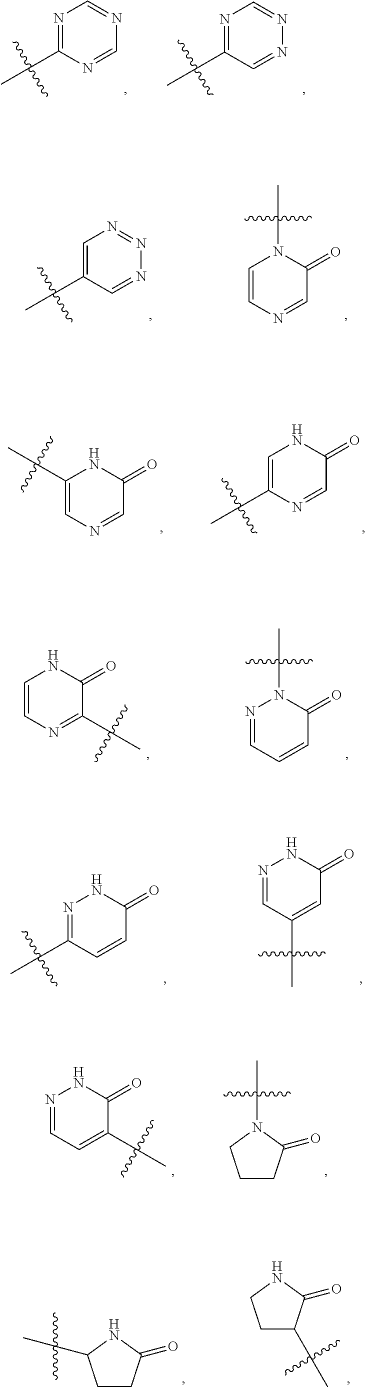 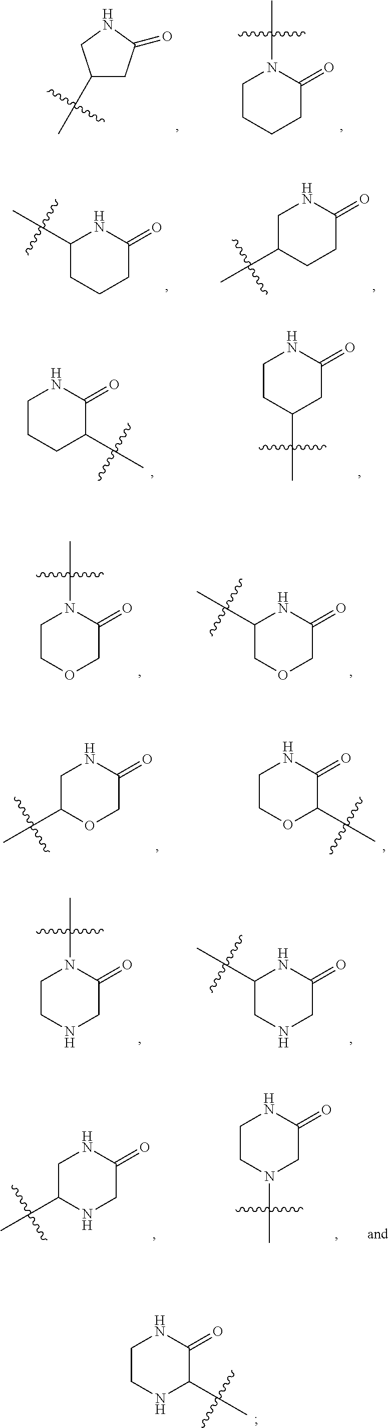
wherein the saturated heterocycle or heteroaryl is optionally substituted by cyano, cycloalkyl, fluorinated alkyl, fluorinated cycloalkyl, halogen, OH, NH₂, oxo, or (C═O)C₁₋₄alkyl where valence permits.

22. The compound of claim 19, wherein R₄ is

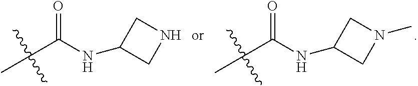

23. The compound of claim 19, wherein R₄ is

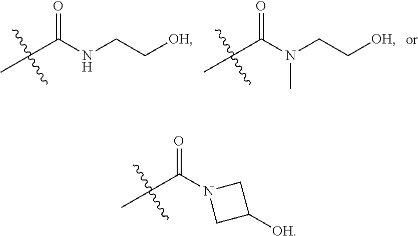

24. The compound of claim 1, wherein each occurrence of R₆ and R₇ are independently H or alkyl.

25. The compound of claim 1, wherein at least one occurrence of R₅ is H, alkyl, cycloalkyl, aryl, heteroaryl, (C═O)Rₐ, (C═O)(CR₆R₇)ₙ₃ORₐ, (C═O)(CR₆R₇)ₙ₃NRₐR_{b}, (C═O)NRₐR_{b}, or SO₂Rₐ.

26. The compound of claim 1, wherein at least one occurrence of R₅ is H, alkyl, or cycloalkyl.

27. The compound of claim 1, wherein at least one occurrence of R₅ is (C═O)Rₐ, (C═O)-alkyl-ORₐ, (C═O)-alkyl-NRₐR_{b}, (C═O)NRₐR_{b}, or SO₂Rₐ.

28. The compound of claim 27, wherein at least one occurrence of R₅ is (C═O)NRₐR_{b}, (C═O)CH₂NRₐR_{b}, or (C═O)CH₂CH₂NRₐR_{b}.

29. The compound of claim 1, wherein the compound has a structure of Formula Ia: 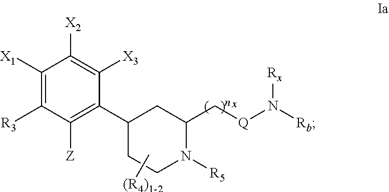
wherein
nₓ is 0, 1, or 2;
Q is CR₆R₇ or C═O; and
Rₓ is Rₐ, (C═O)Rₐ, (C═O)NRₐR_{b}, or SO₂Rₐ.

30. The compound of claim 29, wherein nₓ is 0 or 1.

31. The compound of claim 29, wherein R₅ is H or Me.

32. The compound of claim 29, wherein Q is C═O and NRₓR_{b} is NH₂, NHMe, NMe₂, NH(C═O)NH₂, NMe(C═O)NH₂, NH(C═O)NHMe, NMe(C═O)NMe, NH(C═O)NMe₂, NMe(C═O)NMe₂, or SO₂Me.

33. The compound of claim 1, wherein [symbol]
refers to a single bond;
X is CR₄;
Y is O or NR₅;
R₃ is H, alkyl, cycloalkyl, optionally substituted saturated heterocycle, optionally substituted aryl, optionally substituted heteroaryl, CN, CF₃, OCF₃, ORₐ, SRₐ, halogen, NRₐR_{b}, or NR_{b}(C═O)Rₐ;
R₄ is H, alkyl, or (C═O)NRₐR_{b};
R₅ is H or alkyl;
n₁ is 1, 2, or 3;
n₄ is 0, 1 or 2; and
n₅ is 0 or 1.

34. The compound of claim 33, wherein R₄ is (C═O)NRₐR_{b}.

35. The compound of claim 1, wherein the compound has the structure of Formula 1b:

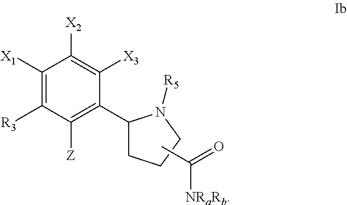

36. The compound of claim 35, wherein the compound has the structure of

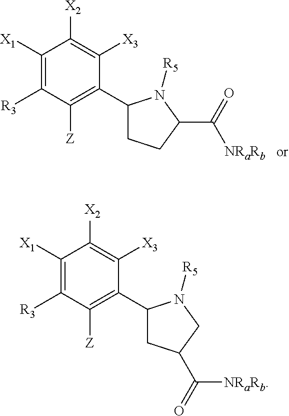

37. The compound of claim 1, wherein the compound has the structure of Formula 1c:

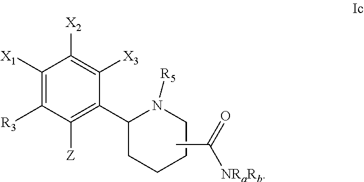

38. The compound of claim 37, wherein the compound has the structure of

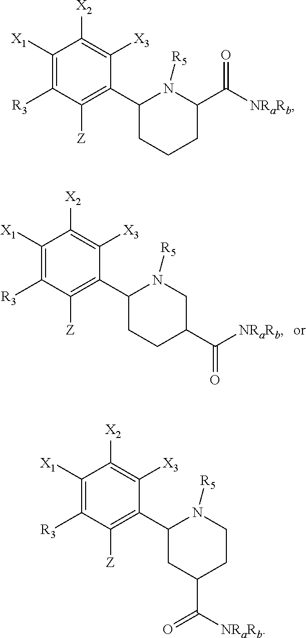

39. The compound of claim 1, wherein Z is OH or O(C₁-C₄ alkyl).

40. The compound of claim 39, wherein Z is OMe, OEt, or OH.

41. The compound of claim 39, wherein Z is OH.

42. The compound of claim 1, wherein X₁ is H, halogen, fluorinated alkyl, or alkyl.

43. The compound of claim 42, wherein X₁ is H, F, Cl, Br, Me, CF₂H, CF₂Cl, or CF₃.

44. The compound of claim 42, wherein X₁ is H or Cl.

45. The compound of claim 1, wherein X₂ is H, halogen, fluorinated alkyl, or alkyl.

46. The compound of claim 45, wherein X₂ is H, F, Cl, Br, Me, CF₂H, CF₂Cl, or CF₃.

47. The compound of claim 45, wherein X₂ is H or Cl.

48. The compound of claim 1, wherein X₃ is H, F, Cl, Br, Me, CF₂H, CF₂Cl, or CF₃.

49. The compound of claim 48, wherein X₃ is H or Cl.

50. The compound of claim 1, wherein the structural moiety 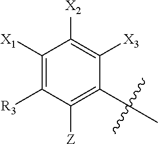
has the structure of

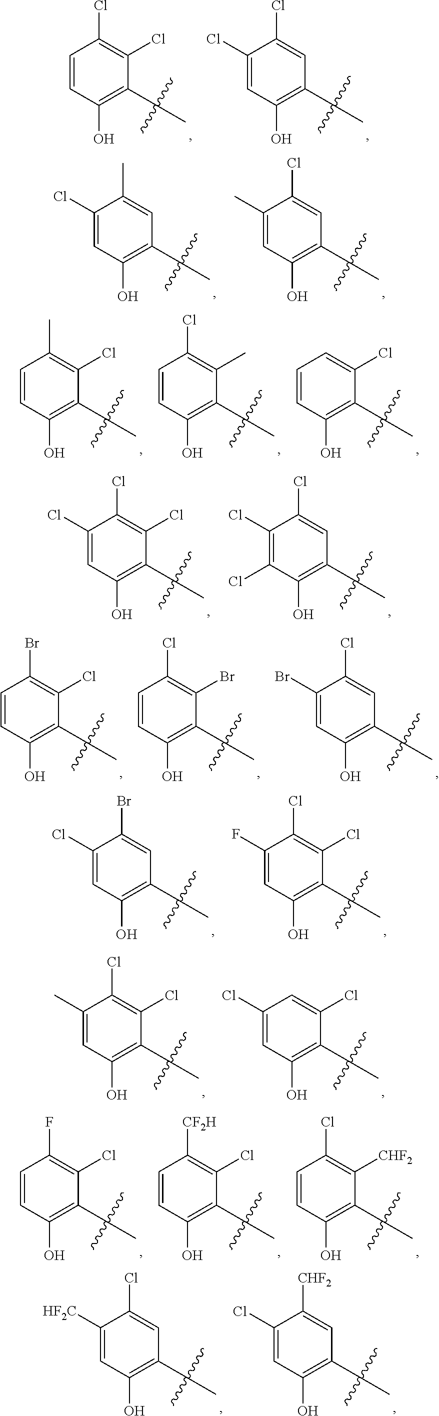

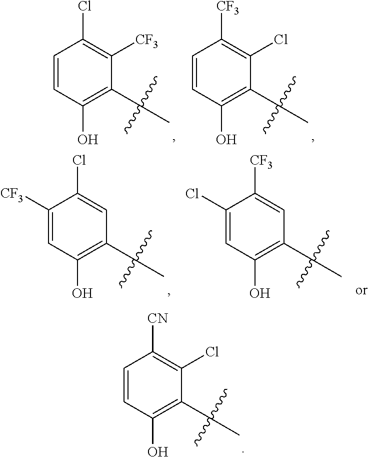

51. The compound of claim 1, wherein the compound has a structure of Formula II′ or II: 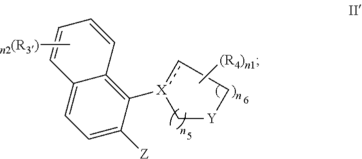 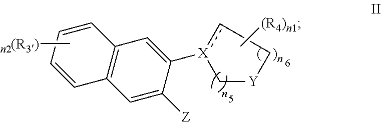
wherein R_{3′} is independently H, halogen, or alkyl; and
n₂ is an integer from 0-3.

52. The compound of claim 51, wherein n₂ is 0, 1, 2, or 3.

53. The compound of claim 51, wherein R_{3′} is H or alkyl.

54. The compound of claim 51, wherein R_{3′} is halogen.

55. The compound of claim 51, wherein Z is ORₐ.

56. The compound of claim 51, wherein Z is OH, OMe, or OEt.

57. The compound of claim 51, wherein Z is OH.

58. The compound of claim 1, wherein R₃ is H, alkyl, cycloalkyl, aryl, heteroaryl, CN, CF₃, ORₐ, SRₐ, halogen, NRₐR_{b}, or NR_{b}(C═O)Rₐ.

59. The compound of claim 1, wherein R₃ is H, alkyl, CF₃, ORₐ, SRₐ, halogen, NRₐR_{b}, or NR_{b}(C═O)Rₐ.

60. The compound of claim 1, wherein R₃ is H, halogen, fluorinated alkyl, or alkyl.

61. The compound of claim 1, wherein n₁ is 0, 1, or 2.

62. The compound of claim 1, wherein each occurrence of n₃ is independently 0, 1, or 2.

63. The compound of claim 1, wherein each occurrence of n₄ and n₅ are independently 0 or 1.

64. The compound of claim 1, wherein at least one occurrence of Rₐ or R_{b} is independently H, alkyl, cycloalkyl, saturated heterocycle, aryl, or heteroaryl.

65. The compound of claim 64, wherein at least one occurrence of Rₐ or R_{b} is independently H, Me, Et, Pr, or a heterocycle selected from the group consisting of 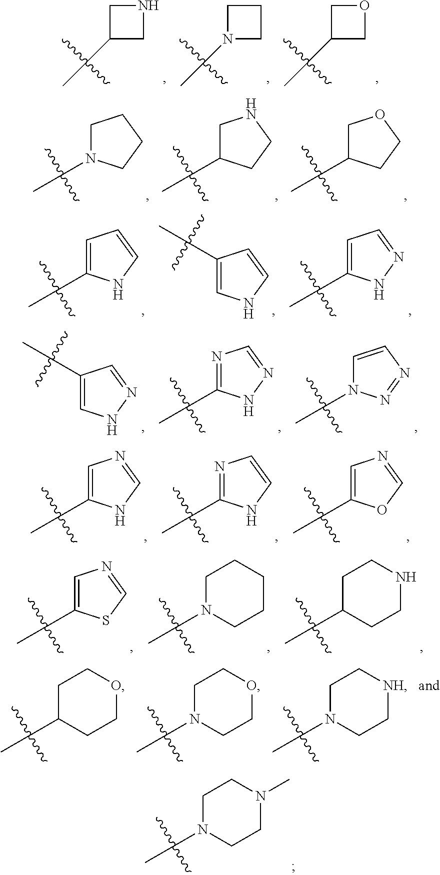
wherein the heterocycle is optionally substituted by alkyl, OH, oxo, or (C═O)C₁₋₄alkyl where valence permits.

66. The compound of claim 64, wherein at least one occurrence of Rₐ or R_{b} is H or

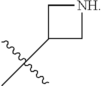 [SMILES: *C1CNC1]

67. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

68. The compound of claim 1, wherein the compound is selected from the group consisting of compounds 1-127 as shown in Table 1.

69. A pharmaceutical composition comprising at least one compound according to claim 1 or a pharmaceutically-acceptable salt thereof and a pharmaceutically-acceptable carrier or diluent.

70. A method of treating a condition in a mammalian species in need thereof, comprising administering to the mammalian species a therapeutically effective amount of at least one compound according to claim 1 or a pharmaceutically-acceptable salt thereof, wherein the condition is selected from the group consisting of cancer, an immunological disorder, a central nervous system disorder, an inflammatory disorder, a gastroenterological disorder, a metabolic disorder, a cardiovascular disorder, and a kidney disease.

71. The method of claim 70, wherein the immunological disorder is transplant rejection or an autoimmune disease.

72. The method of claim 70, wherein the autoimmune disease is rheumatoid arthritis, multiple sclerosis, systemic lupus erythematosus, or type I diabetes mellitus.

73. The method of claim 70, wherein the central nervous system disorder is Alzheimer's disease.

74. The method of claim 70, wherein the inflammatory disorder is an inflammatory skin condition, arthritis, psoriasis, spondylitis, parodontitits, or an inflammatory neuropathy.

75. The method of claim 70, wherein the gastroenterological disorder is an inflammatory bowel disease.

76. The method of claim 70, wherein the metabolic disorder is obesity or type II diabetes mellitus.

77. The method of claim 70, wherein the cardiovascular disorder is an ischemic stroke.

78. The method of claim 70, wherein the kidney disease is chronic kidney disease, nephritis, or chronic renal failure.

79. The method of claim 70, wherein the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

80. The method of claim 70, wherein the mammalian species is human.

81. A method of blocking Kv1.3 potassium channel in a mammalian species in need thereof, comprising administering to the mammalian species a therapeutically effective amount of at least one compound according to claim 1 or a pharmaceutically-acceptable salt thereof.

82. The method of claim 81, wherein the mammalian species is human.
